(19) United States
(12) Patent Application Publication
(10) Pub. No.: US 2025/0346871 A1
(43) Pub. Date: Nov. 13, 2025

(54) PRENYLTRANSFERASES AND METHODS OF MAKING AND USE THEREOF

(71) Applicant: Cellibre, Inc., San Diego, CA (US)

(72) Inventors: Spiros Kambourakis, San Diego, CA (US); Russell Scott Komor, San Diego, CA (US); Nicholas Donald Keul, La Jolla, CA (US)

(21) Appl. No.: 17/909,733

(22) PCT Filed: Mar. 8, 2021
(86) PCT No.: PCT/US2021/021413

§ 371 Date: Sep. 6, 2022

Related U.S. Application Data
(60) Provisional application No. 62/986,567, filed on Mar. 6, 2020.

(51) Int. Cl. C12N 9/10; C12N 15/52; C12P 7/22; C12P 7/42; C12P 17/06

(52) U.S. Cl. CPC C12N 9/1085; C12N 9/1029; C12N 15/52; C12P 7/22; C12P 7/42; C12P 17/06; C12Y 205/0101

(57) ABSTRACT

Disclosed herein are novel prenyltransferases for the production of cannabinoids, as well methods of making and using such prenyltransferases.

### RELATED APPLICATIONS

This application is a national stage filing under 35 U.S.C. 371 of International Application No. PCT/US2021/021413, filed Mar. 8, 2021, which claims the benefit of U.S. Provisional Application No. 62/986,567, filed Mar. 6, 2020, the entire teachings of which are incorporated herein by reference. International Application No. PCT/US2021/021413 was published under PCT Article 21 (2) in English.

### SEQUENCE LISTING

The instant application contains a Sequence Listing which has been submitted via Patent Center and is hereby incorporated by reference in its entirety. Said.txt copy, created on Dec. 8, 2023, is named CELB-001-301_ST25, and is 325,640 bytes in size.

### BACKGROUND OF THE INVENTION

Synthesis of the common cannabinoids tetrahydrocannabinolic acid (THCA), cannabidiolic acid (CBDA) and cannabichromenic acid (CBCA) in the cannabis plant is accomplished by the action of two major biosynthetic pathways 1) the mevalonic acid pathway that converts acetyl-CoA to geranyl pyrophosphate (GPP) and 2) the hexanoic acid/olivetolic acid pathway that also converts hexanoyl-CoA to olivetolic acid (OA) through iterative reactions with malonyl-CoA. Condensation of GPP with OA to form cannabigerolic acid (CBGA) is a key reaction that, in the plant, is catalyzed by an integral membrane prenyltransferase. The rate and efficiency (flux) of the intracellular formation of CBGA also defines the final titers of the most common cannabinoids (THCA, CBDA, and CBCA) because they are all synthesized from the precursor CBGA by the action of three different synthases (THCAS, CBDAS and CBCAS, respectively). As a result, achieving high titers in the biosynthesis of THC (A), CBD (A) and CBC (A) either in the plant or in a recombinant host organism requires 1) increasing the flux and availability of GPP and OA 2) increasing the activity of a CBGA synthase and 3) increasing the activity and selectivity of THCA/CBDA/CBCA synthases.

Recently, a number of academic labs and companies have explored the use of microorganisms (S. cerevisiae, E. coli, and various algae) as heterologous hosts for producing cannabinoids, mainly CBD (A) and THC (A). As described above, the first requirement in the biosynthesis of cannabinoids is increased flux to GPP and OA, which can mainly be addressed by strain and pathway engineering. However, the second step, their condensation to CBGA, is a major bottleneck because all the prenyltransferases that have been identified and characterized from the plant C. sativa (PT1 and PT4) suffer from low activity towards CBGA formation (turn-over number) and poor expression in recombinant microbial hosts. This is partly due to the fact that the native prenyltransferases from C. sativa are integral membrane proteins, rendering their heterologous expression and characterization difficult. Both limitations can potentially be circumvented if a soluble prenyltransferase with high activity and selectivity towards the formation of CBGA from OA and GPP can be identified.

Soluble aromatic prenyltransferases are ubiquitous in nature and are present in a variety of bacteria and fungi. One such enzyme is NphB, an aromatic prenyltransferase from Streptomyces sp. (strain CL190) (Uniprot ID: Q4R2T2), that can transfer GPP to a variety of aromatic compounds, including OA. Although in principal NphB can serve as an alternative to the Cannabis prenyltransferases for producing CBGA, it also suffers from low activity and selectivity. Specifically, it forms two products from the condensation of GPP and OA: CBGA and O-CBGA (prenylation at the adjacent hydroxyl) in a 1 to 2 ratio with O-CBGA being the major product (Zirpel, B et al J Biotechnol. 2017, 259, 2014). In order to address both the low activity and selectivity problems of NphB, there has been significant protein engineering efforts undertaken by both academic (Meaghan A. V, et al Nature 2019, 10, 565) and industrial groups (WO 2019173770A1 and WO 2019183152A1). However, there still remains a need for soluble prenyltransferases with high selectivity and activity for CBGA.

### SUMMARY OF THE INVENTION

By using machine learning/artificial intelligence algorithms and sequence homology analysis, numerous enzymes, the majority of which had unknown function or activity, were identified as possible soluble aromatic prenyltransferases (APT). These enzymes were cloned, expressed, purified, and characterized for activity in E. coli. Surprisingly, three of these identified enzymes shared significant sequence similarities that are not shared with previously identified soluble prenyltransferases, including NphB. Thus, disclosed herein are new soluble prenyltransferases as well as mutants thereof for use in microbial and plant expression systems to produce cannabinoids, their acids, and analogs thereof.

Cannabinoids are products that are produced from reacting olivetolic acid and its analogs (e.g., divarinic acid-DVA) with GPP or FPP. Cannabinoids further include the cyclization products of the previous CBGA analogs to produce CBDA, THCA and CBCA analogs in addition to producing other novel cyclization products. Some examples of these analogs are shown in FIG. 6.

Some aspects of the present disclosure are directed to a recombinant polypeptide comprising an amino acid sequence with at least 70% identity to SEQ ID NO: 1, 2, 3, or 4, wherein the amino acid sequence comprises at least one amino acid modification as compared to SEQ ID NO: 1, 3, or 4. Some aspects of the present disclosure are directed to a recombinant polypeptide comprising an amino acid sequence with at least 90% identity to SEQ ID NO: 5, wherein the amino acid sequence comprises at least one amino acid modification as compared to SEQ ID NO: 1, 2, 3, or 4. In some embodiments, the recombinant polypeptide further comprises one or more of a histidine tag sequence, TEV cleavage sequence, an addition of a glycine at the C-termini, and a deletion of 8 to 16 (e.g., 10-16) amino acids from the C-terminus. In some embodiments, the recombinant polypeptide comprises a histidine tag sequence, a TEV cleavage sequence, and a deletion of 8 to 16 (e.g., 10-16) amino acids from the C-terminus of the SEQ ID NO: 1, 2, 3, or 4. In some embodiments, the recombinant polypeptide comprises a deletion of 8 to 16 (e.g., 10-16) amino acids from the C-terminus of the SEQ ID NO: 1, 2, 3, or 4.

In some embodiments, the recombinant polypeptide comprises an amino acid sequence identical to SEQ ID NO: 1 with one to twenty amino acid substitutions and, optionally, one to twenty amino acids deleted from the C-terminus, wherein the amino acid substitutions are located in SEQ ID NO: 1 at positions selected from 49, 51, 52, 53, 65, 67, 68, 69, 111, 113, 122, 124, 125, 126, 164, 165, 166, 167, 170, 214, 215, 217, 219, 229, 231, 233, 235, 268, 269, 270, 282, 283, 284, 285, 286, 292, 293, 294, 295, 296, 297, and 298. In some embodiments, the recombinant polypeptide comprises an amino acid sequence identical to SEQ ID NO: 2 with one to twenty amino acid substitutions and, optionally, one to twenty amino acids deleted from the C-terminus, wherein the amino acid substitutions are located in SEQ ID NO: 2 at positions selected from 38, 39, 40, 41, 42, 43, 44, 55, 57, 58, 59, 101, 103, 111, 112, 114, 115, 116, 117, 155, 156, 157, 160, 167, 169, 204, 205, 207, 209, 219, 221, 223, 224, 225, 258, 259, 260, 272, 273, 274, 275, 276, 282, 283, 284, 285, 286, 287, and 288. In some embodiments, the recombinant polypeptide comprises an amino acid sequence identical to SEQ ID NO: 3 with one to twenty amino acid substitutions and, optionally, one to twenty amino acids deleted from the C-terminus, wherein the amino acid substitutions are located in SEQ ID NO: 3 at positions selected from 108, 109, 110, 111, 112, 113, 114, 125, 127, 128, 129, 171, 173, 181, 182, 184, 185, 186, 187, 225, 226, 227, 230, 237, 239, 274, 275, 277, 279, 289, 291, 293, 294, 295, 328, 329, 330, 342, 343, 344, 345, 346, 352, 353, 354, 355, 356, 357, and 358. In some embodiments, the recombinant polypeptide comprises an amino acid sequence identical to SEQ ID NO: 4 with one to twenty amino acid substitutions and, optionally, one to twenty amino acids deleted from the C-terminus, wherein the amino acid substitutions are located in SEQ ID NO: 4 at positions selected from 38, 39, 40, 41, 42, 43, 44, 55, 57, 58, 59, 101, 103, 111, 112, 114, 115, 116, 117, 155, 156, 157, 160, 167, 169, 204, 205, 207, 209, 219, 221, 223, 224, 225, 258, 259, 260, 272, 273, 274, 275, 276, 282, 283, 284, 285, 286, 287, and 288.

In some embodiments, the recombinant polypeptide comprises an amino acid sequence identical to SEQ ID NO: 1, 2, 3, or 4 with one to twenty amino acid substitutions, and ten to sixteen amino acids deleted from the C-terminus. In some embodiments, the recombinant polypeptide comprises an amino acid sequence 90% identical to a sequence selected from SEQ ID NOs: 23-79 and 82-88. In some embodiments, the recombinant polypeptide comprises an amino acid sequence identical to a sequence selected from SEQ ID NOs: 23-79 and 82-88. In some embodiments, the sequence is selected from SEQ ID NOs: 29-36, 43, 56, 67, 69, 70, and 74.

In some embodiments, the recombinant polypeptide converts olivetolic acid (OA) and geranyl diphosphate (GPP) to one or more products comprising cannabigerolic acid (CBGA). In some embodiments, at least about 50% of the one or more products is CBGA.

In some embodiments, the recombinant polypeptide has a rate of formation of cannabigerolic acid (CBGA) from olivetolic acid (OA) and geranyl diphosphate (GPP) that is greater than the rate of formation of CBGA produced by NphB under the same conditions. In some embodiments, the recombinant polypeptide converts olivetolic acid (OA) and farnesyl pyrophosphate (FPP) to one or more cannabinoids, cannabinoid derivatives, or cannabinoid analogues. In some embodiments, the recombinant polypeptide converts divarinic acid (DVA) and geranyl diphosphate (GPP) to CBGVA or one or more cannabinoids, cannabinoid derivatives, or cannabinoid analogues (e.g., O-CBGVA, F-CBGVA).

In some embodiments, the recombinant polypeptide has a rate of formation of CBGA from olivetolic acid (OA) and geranyl diphosphate (GPP) that is greater than the rate of formation of CBGA from OA and GPP by a polypeptide consisting of SEQ ID NO: 4 under the same conditions. In some embodiments, the recombinant polypeptide has a rate of formation of O-CBGA from OA and GPP that is greater than the rate of formation of O-CBGA from OA and GPP by a polypeptide consisting of SEQ ID NO: 4 under the same conditions. In some embodiments, recombinant polypeptide has a rate of formation of F-CBGA from OA and GPP that is greater than the rate of formation of F-CBGA from OA and GPP by a polypeptide consisting of SEQ ID NO: 4 under the same conditions.

In some embodiments, the recombinant polypeptide has a rate of formation of cannabigerovarinic acid (CBGVA) from divarinic acid (DVA) and geranyl diphosphate (GPP) that is greater than the rate of formation of CBGVA from DVA and GPP by a polypeptide consisting of SEQ ID NO: 4 under the same conditions. In some embodiments, the recombinant polypeptide has a rate of formation of O-CBGVA from DVA and GPP that is greater than the rate of formation of O-CBGVA from DVA and GPP by a polypeptide consisting of SEQ ID NO: 4 under the same conditions. In some embodiments, the recombinant polypeptide has a rate of formation of F-CBGVA from DVA and GPP that is greater than the rate of formation of F-CBGVA from DVA and GPP by a polypeptide consisting of SEQ ID NO: 4 under the same conditions.

In some embodiments, the recombinant polypeptide has a rate of formation of CBGA from OA and GPP that is at least 1.5-fold greater than the rate of formation of CBGA from OA and GPP by a polypeptide consisting of SEQ ID NO: 4 under the same conditions.

Some aspects of the present disclosure are directed to a cell comprising an exogenous nucleotide sequence coding for the recombinant polypeptide described herein. In some embodiments, the cell is a bacteria, an algae, a yeast, or a plant cell. In some embodiments, the yeast is an oleaginous yeast (e.g., a Yarrowia lipolytica strain). In some embodiments, the bacteria is Escherichia coli.

Some aspects of the present disclosure are directed to a cell comprising an exogenous nucleotide sequence coding for a recombinant polypeptide comprising an amino acid sequence having at least 70% identity to SEQ ID NO: 1, 2, 3, or 4. Some aspects of the present disclosure are directed to a cell comprising an exogenous nucleotide sequence coding for a recombinant polypeptide comprising an amino acid sequence having at least 90% identity to SEQ ID NO: 5. In some embodiments, the amino acid sequence comprises at least one amino acid modification as compared to SEQ ID NO: 1, 3, or 4. In some embodiments, the recombinant polypeptide comprises a histidine tag sequence.

In some embodiments, the cell comprises an exogenous nucleotide sequence coding for a recombinant polypeptide comprising an amino acid sequence identical to SEQ ID NO: 1 with one to twenty amino acid substitutions and, optionally, one to twenty amino acids deleted from the C-terminus, wherein the amino acid substitutions are located in SEQ ID NO: 1 at positions selected from 49, 51, 52, 53, 65, 67, 68, 69, 111, 113, 122, 124, 125, 126, 164, 165, 166, 167, 170, 214, 215, 217, 219, 229, 231, 233, 235, 268, 269, 270, 282, 283, 284, 285, 286, 292, 293, 294, 295, 296, 297, and 298

In some embodiments, the cell comprises an exogenous nucleotide sequence coding for a recombinant polypeptide comprising an amino acid sequence identical to SEQ ID NO: 2 with one to twenty amino acid substitutions and, optionally, one to twenty amino acids deleted from the C-terminus, wherein the amino acid substitutions are located in SEQ ID NO: 2 at positions selected from 38, 39, 40, 41, 42, 43, 44, 55, 57, 58, 59, 101, 103, 111, 112, 114, 115, 116, 117, 155, 156, 157, 160, 167, 169, 204, 205, 207, 209, 219, 221, 223, 224, 225, 258, 259, 260, 272, 273, 274, 275, 276, 282, 283, 284, 285, 286, 287, and 288.

In some embodiments, the cell comprises an exogenous nucleotide sequence coding for a recombinant polypeptide comprising an amino acid sequence identical to SEQ ID NO: 3 with one to twenty amino acid substitutions and, optionally, one to twenty amino acids deleted from the C-terminus, wherein the amino acid substitutions are located in SEQ ID NO: 3 at positions selected from 108, 109, 110, 111, 112, 113, 114, 125, 127, 128, 129, 171, 173, 181, 182, 184, 185, 186, 187, 225, 226, 227, 230, 237, 239, 274, 275, 277, 279, 289, 291, 293, 294, 295, 328, 329, 330, 342, 343, 344, 345, 346, 352, 353, 354, 355, 356, 357, and 358.

In some embodiments, the cell comprises an exogenous nucleotide sequence coding for a recombinant polypeptide comprising an amino acid sequence identical to SEQ ID NO: 4 with one to twenty amino acid substitutions and, optionally, one to twenty amino acids deleted from the C-terminus, wherein the amino acid substitutions are located in SEQ ID NO: 4 at positions selected from 38, 39, 40, 41, 42, 43, 44, 55, 57, 58, 59, 101, 103, 111, 112, 114, 115, 116, 117, 155, 156, 157, 160, 167, 169, 204, 205, 207, 209, 219, 221, 223, 224, 225, 258, 259, 260, 272, 273, 274, 275, 276, 282, 283, 284, 285, 286, 287 and 288.

In some embodiments, the recombinant polypeptide comprises an amino acid sequence identical to SEQ ID NO: 1, 2, 3, or 4 with one to twenty amino acid substitutions, and ten to sixteen amino acids deleted from the C-terminus. In some embodiments, the recombinant polypeptide comprises an amino acid sequence 90% identical to a sequence selected from SEQ ID NOs: 23-79 and 82-88. In some embodiments, the recombinant polypeptide comprises an amino acid sequence identical to a sequence selected from SEQ ID NOs: 23-79 and 82-88. In some embodiments, the sequence is selected from SEQ ID NOs: 29-36, 43, 56, 67, 69, 70, and 74.

In some embodiments, the recombinant polypeptide is capable of converting olivetolic acid (OA) and geranyl diphosphate (GPP) to one or more products comprising cannabigerolic acid (CBGA). In some embodiments, at least about 50% of the one or more products is CBGA. In some embodiments, more than about 90% of the one or more products is CBGA. In some embodiments, the recombinant polypeptide has a rate of formation of cannabigerolic acid (CBGA) from olivetolic acid (OA) and geranyl diphosphate (GPP) that is greater than the rate of formation of CBGA from OA and GPP by NphB under the same conditions.

In some embodiments, the recombinant polypeptide is capable of converting olivetolic acid (OA) and farnesyl pyrophosphate (FPP) to one or more cannabinoid, cannabinoid derivatives, or cannabinoid analogues. In some embodiments, the recombinant polypeptide is capable of converting divarinic acid (DVA) and geranyl diphosphate (GPP) to CBGVA or one or more cannabinoid, cannabinoid derivatives, or cannabinoid analogues.

In some embodiments, the recombinant polypeptide has a rate of formation of CBGA from olivetolic acid (OA) and geranyl diphosphate (GPP) that is greater than the rate of formation of CBGA from OA and GPP by a polypeptide consisting of SEQ ID NO: 4 under the same conditions. In some embodiments, the recombinant polypeptide has a rate of formation of O-CBGA from OA and GPP that is greater than the rate of formation of O-CBGA from OA and GPP by a polypeptide consisting of SEQ ID NO: 4 under the same conditions. In some embodiments, recombinant polypeptide has a rate of formation of F-CBGA from OA and GPP that is greater than the rate of formation of F-CBGA from OA and GPP by a polypeptide consisting of SEQ ID NO: 4 under the same conditions.

In some embodiments, the recombinant polypeptide has a rate of formation of cannabigerovarinic acid (CBGVA) from divarinic acid (DVA) and geranyl diphosphate (GPP) that is greater than the rate of formation of CBGVA from DVA and GPP by a polypeptide consisting of SEQ ID NO: 4 under the same conditions. In some embodiments, the recombinant polypeptide has a rate of formation of O-CBGVA from DVA and GPP that is greater than the rate of formation of O-CBGVA from DVA and GPP by a polypeptide consisting of SEQ ID NO: 4 under the same conditions. In some embodiments, the recombinant polypeptide has a rate of formation of F-CBGVA from DVA and GPP that is greater than the rate of formation of F-CBGVA from DVA and GPP by a polypeptide consisting of SEQ ID NO: 4 under the same conditions.

In some embodiments, the recombinant polypeptide has a rate of formation of CBGA from OA and GPP that is at least 1.5-fold greater than the rate of formation of CBGA from OA and GPP by a polypeptide consisting of SEQ ID NO: 4 under the same conditions.

In some embodiments, the cell comprises an olivetolic acid pathway. In some embodiments, the olivetolic acid pathway comprises a polyketide cyclase. In some embodiments, the olivetolic acid pathway comprises a polyketide synthase. In some embodiments, an exogenous nucleotide codes for the polyketide cyclase. In some embodiments, the cell comprises a geranyl pyrophosphate (GPP) pathway (e.g., comprising a non-native or mutant component). In some embodiments, the cell comprises an upregulated geranyl pyrophosphate (GPP) pathway. In some embodiments, the GPP pathway comprises geranyl pyrophosphate synthase. In some embodiments, an exogenous nucleotide codes for the geranyl pyrophosphate synthase. In some embodiments, the cell comprises a farnesyl pyrophosphate (FPP) pathway (e.g., comprising a non-native or mutant component). In some embodiments, the FPP pathway comprises a farnesyl pyrophosphate synthase. In some embodiments, an exogenous nucleotide codes for the farnesyl pyrophosphate synthase. In some embodiments, the cell comprises a divarinic acid (DVA) pathway. In some embodiments, the DVA pathway comprises divarinic acid synthase. In some embodiments, an exogenous nucleotide codes for the divarinic acid synthase.

In some embodiments, the cell is capable of producing a cannabinoid selected from tetrahydrocannabinol, cannabidiol, cannabigerol, or an acid, derivative, or analogue thereof. In some embodiments, production of the cannabinoid is under control of an inducible promoter. In some embodiments, the cell is a bacteria, an algae, or a yeast. In some embodiments, the yeast is an oleaginous yeast (e.g., a Yarrowia lipolytica strain). In some embodiments, the bacteria is Escherichia coli.

Some aspects of the present disclosure are related to a composition comprising cannabigerolic acid, tetrahydrocannabinol, cannabidiol, cannabigerol, or an acid, derivative, or an analogue thereof produced by a cell described herein.

Some aspects of the present disclosure are related to a method of producing a cannabinoid or an acid, derivative, or analogue thereof, the method comprising providing a cell comprising an exogenous nucleotide sequence coding for a recombinant polypeptide comprising an amino acid sequence having at least 70% identity to SEQ ID NO: 1, 2, 3, or 4 and culturing the cell to produce the cannabinoid or acid, derivative, or analogue thereof. Some aspects of the present disclosure are related to a method of producing a cannabinoid or an acid, derivative, or analogue thereof, the method comprising providing a cell comprising an exogenous nucleotide sequence coding for a recombinant polypeptide comprising an amino acid sequence having at least 90% identity to SEQ ID NO: 5 and culturing the cell to produce the cannabinoid or an acid, derivative, or analogue thereof. In some embodiments, the amino acid sequence comprises at least one amino acid modification as compared to SEQ ID NO: 1, 3, or 4. In some embodiments, the recombinant polypeptide further comprises a histidine tag sequence.

In some embodiments, the amino acid sequence is identical to SEQ ID NO: 1 with one to twenty amino acid substitutions and, optionally, one to twenty amino acids deleted from the C-terminus, wherein the amino acid substitutions are located in SEQ ID NO: 1 at positions selected from 49, 51, 52, 53, 65, 67, 68, 69, 111, 113, 122, 124, 125, 126, 164, 165, 166, 167, 170, 214, 215, 217, 219, 229, 231, 233, 235, 268, 269, 270, 282, 283, 284, 285, 286, 292, 293, 294, 295, 296, 297, and 298. In some embodiments, the amino acid sequence is identical to SEQ ID NO: 2 with one to twenty amino acid substitutions and, optionally, one to twenty amino acids deleted from the C-terminus, wherein the amino acid substitutions are located in SEQ ID NO: 2 at positions selected from 38, 39, 40, 41, 42, 43, 44, 55, 57, 58, 59, 101, 103, 111, 112, 114, 115, 116, 117, 155, 156, 157, 160, 167, 169, 204, 205, 207, 209, 219, 221, 223, 224, 225, 258, 259, 260, 272, 273, 274, 275, 276, 282, 283, 284, 285, 286, 287, and 288. In some embodiments, the amino acid sequence is identical to SEQ ID NO: 3 with one to twenty amino acid substitutions and, optionally, one to twenty amino acids deleted from the C-terminus, wherein the amino acid substitutions are located in SEQ ID NO: 3 at positions selected from 108, 109, 110, 111, 112, 113, 114, 125, 127, 128, 129, 171, 173, 181, 182, 184, 185, 186, 187, 225, 226, 227, 230, 237, 239, 274, 275, 277, 279, 289, 291, 293, 294, 295, 328, 329, 330, 342, 343, 344, 345, 346, 352, 353, 354, 355, 356, 357 and 358. In some embodiments, the amino acid sequence is identical to SEQ ID NO: 4 with one to twenty amino acid substitutions and, optionally, one to twenty amino acids deleted from the C-terminus, wherein the amino acid substitutions are located in SEQ ID NO: 4 at positions selected from 38, 39, 40, 41, 42, 43, 44, 55, 57, 58, 59, 101, 103, 111, 112, 114, 115, 116, 117, 155, 156, 157, 160, 167, 169, 204, 205, 207, 209, 219, 221, 223, 224, 225, 258, 259, 260, 272, 273, 274, 275, 276, 282, 283, 284, 285, 286, 287, and 288.

In some embodiments, the recombinant polypeptide comprises an amino acid sequence identical to SEQ ID NO: 1, 2, 3, or 4 with one to twenty amino acid substitutions, and ten to sixteen amino acids deleted from the C-terminus. In some embodiments, the recombinant polypeptide comprises an amino acid sequence 90% identical to a sequence selected from SEQ ID NOs: 23-79 and 82-88. In some embodiments, the recombinant polypeptide comprises an amino acid sequence identical to a sequence selected from SEQ ID NOs: 23-79 and 82-88. In some embodiments, the sequence is selected from SEQ ID NOs: 29-36, 43, 56, 67, 69, 70, and 74.

In some embodiments, the recombinant polypeptide converts olivetolic acid (OA) and geranyl diphosphate (GPP) to one or more products comprising cannabigerolic acid (CBGA). In some embodiments, at least about 50% of the one or more products is CBGA. In some embodiments, more than about 90% of the one or more products is CBGA. In some embodiments, the recombinant polypeptide has a rate of formation of cannabigerolic acid (CBGA) from olivetolic acid (OA) and geranyl diphosphate (GPP) that is greater than the rate of formation of CBGA from OA and GPP by NphB under the same conditions. In some embodiments, the recombinant polypeptide converts olivetolic acid (OA) and farnesyl pyrophosphate (FPP) to one or more cannabinoid, cannabinoid derivatives, or cannabinoid analogues.

In some embodiments, the recombinant polypeptide has a rate of formation of CBGA from olivetolic acid (OA) and geranyl diphosphate (GPP) that is greater than the rate of formation of CBGA from OA and GPP by a polypeptide consisting of SEQ ID NO: 4 under the same conditions. In some embodiments, the recombinant polypeptide has a rate of formation of O-CBGA from OA and GPP that is greater than the rate of formation of O-CBGA from OA and GPP by a polypeptide consisting of SEQ ID NO: 4 under the same conditions. In some embodiments, recombinant polypeptide has a rate of formation of F-CBGA from OA and GPP that is greater than the rate of formation of F-CBGA from OA and GPP by a polypeptide consisting of SEQ ID NO: 4 under the same conditions.

In some embodiments, the recombinant polypeptide has a rate of formation of cannabigerovarinic acid (CBGVA) from divarinic acid (DVA) and geranyl diphosphate (GPP) that is greater than the rate of formation of CBGVA from DVA and GPP by a polypeptide consisting of SEQ ID NO: 4 under the same conditions. In some embodiments, the recombinant polypeptide has a rate of formation of O-CBGVA from DVA and GPP that is greater than the rate of formation of O-CBGVA from DVA and GPP by a polypeptide consisting of SEQ ID NO: 4 under the same conditions. In some embodiments, the recombinant polypeptide has a rate of formation of F-CBGVA from DVA and GPP that is greater than the rate of formation of F-CBGVA from DVA and GPP by a polypeptide consisting of SEQ ID NO: 4 under the same conditions.

In some embodiments, the recombinant polypeptide has a rate of formation of CBGA from OA and GPP that is at least 1.5-fold greater than the rate of formation of CBGA from OA and GPP by a polypeptide consisting of SEQ ID NO: 4 under the same conditions.

In some embodiments, the cell comprises an olivetolic acid pathway. In some embodiments, the olivetolic acid pathway comprises a polyketide cyclase. In some embodiments, the olivetolic acid pathway comprises a polyketide synthase. In some embodiments, an exogenous nucleotide codes for the polyketide cyclase. In some embodiments, the cell comprises a geranyl pyrophosphate (GPP) pathway or an upregulated geranyl pyrophosphate (GPP) pathway. In some embodiments, the GPP pathway comprises geranyl pyrophosphate synthase. In some embodiments, an exogenous nucleotide codes for the geranyl pyrophosphate synthase. In some embodiments, the cell comprises a farnesyl pyrophosphate (FPP) pathway. In some embodiments, the FPP pathway comprises a farnesyl pyrophosphate synthase. In some embodiments, an exogenous nucleotide codes for the farnesyl pyrophosphate synthase. In some embodiments, the cell comprises a divarinic acid (DVA) pathway. In some embodiments, the DVA pathway comprises divarinic acid synthase. In some embodiments, an exogenous nucleotide codes for the divarinic acid synthase.

In some embodiments, the produced cannabinoid or analogue thereof is selected from cannabigerolic acid, tetrahydrocannabinol, cannabidiol, cannabigerol, or an acid, derivative or analogue thereof. In some embodiments, production of the cannabinoid or acid, derivative or analogue thereof is under control of an inducible promoter. In some embodiments, the cell is a bacteria, an algae, or a yeast. In some embodiments, the bacteria, algae, or yeast has been genetically modified to express an enzyme for a pathway described herein having one or more improved activities as compared to a wild type enzyme from the bacteria, algae, or yeast. In some embodiments, the bacteria, algae, or yeast has been genetically modified to express a genetically engineered enzyme for a pathway described herein having one or more improved activities as compared to a wild type enzyme. In some embodiments, the yeast is an oleaginous yeast (e.g., a Yarrowia lipolytica strain). In some embodiments, the bacteria is E. Coli.

In some embodiments, the method of production further comprises a step of purifying or isolating the cannabinoid or derivative or analogue thereof from the culture.

All patents, patent applications, and other publications (e.g., scientific articles, books, websites, and databases) mentioned herein are incorporated by reference in their entirety. In case of a conflict between the specification and any of the incorporated references, the specification (including any amendments thereof, which may be based on an incorporated reference), shall control. Standard art-accepted meanings of terms are used herein unless indicated otherwise. Standard abbreviations for various terms are used herein.

The above discussed, and many other features and attendant advantages of the present inventions will become better understood by reference to the following detailed description of the invention.

### BRIEF DESCRIPTION OF THE DRAWINGS

The patent or application file contains at least one drawing executed in color. Copies of this patent or patent application publication with color drawings will be provided by the Office upon request and payment of the necessary fee.

FIG. 1 shows the structures of cannabigerolic acid (CBGA) and related compounds.

FIG. 2 shows the activities with OA and GPP of APT29, APT73, APT89, and improved NphB variant Q161H relative to wild type NphB. Y-axis shows the relative activity of each enzyme compared to the wild type NphB that is set to 1.

FIG. 3 shows product distribution of APT29, APT73, and APT89 compared to wild type NphB and one of its improved mutants (NphB Q161H). Y-axis shows % total product for CGBA, O-CBGA, and an unknown product for each APT.

FIG. 4 shows product distribution of APT29, APT73, and APT89 using OA and FPP. Y-axis shows % total product for CBFA, O-CBFA, and an unknown product for each APT. The chemical structures for CBFA and O-CBFA are also shown.

FIG. 5 shows activities of APT29, APT73, APT89, and improved NphB variant Q161H with OA/GPP, OA/FPP, and DVA/GPP relative to wild type NphB with OA/GPP. Y-axis shows the relative activity of each enzyme compared to the wild type NphB that is set to 1.

FIG. 6 shows the structures of some cannabinoids that can be synthesized using CBGA synthases described herein and in combination with a CBDA, CBCA, THCA, or other synthase.

FIGS. 7A-7C show superimposed crystal structure models for APT29 and APT73 with olivetolic acid docked in the active site-highlighted in yellow (FIG. 7A), GPP-highlighted in yellow (FIG. 7B), and amino acids that are 5 Angstrom from any of the OA/GPP substrates-highlighted in yellow (FIG. 7C). Green balls shown in FIGS. 7A-7C are modeled Mg atoms that are required for enzyme activity.

FIG. 8 shows homology alignment (SEQ ID NO:158) for the amino acid sequences for APT29, APT89, APT88, and APT73 (SEQ ID NOS: 1-4, respectively).

FIG. 9 shows the activities and product distribution of purified APT29, APT73, APT89, and NphB, when reacting with OA or Div and GPP or FPP. Y-axis shows the relative activity of each enzyme compared to the wild type NphB with OA that is set to 1.

FIG. 10 shows the relative activity of selected APT73 mutants as calculated by product formation in in vivo assays. The activities shown are all relative to APT73 activity set to 1.

FIG. 11 shows overall activity of mutants per position around the active site after saturation mutagenesis of each position and screening.

FIG. 12 shows CBGA production of C-terminal truncations in APT73.1 (data from Table 5). All enzymes produced CBGA as the major product (>99%).

FIG. 13 shows the product ratio (CBGA/FCBGA) when purified APT73 and APT89 mutants react with OA and varying GPP and FPP substrate ratios.

### DETAILED DESCRIPTION OF THE INVENTION

### Recombinant Polypeptides

Some aspects of the present invention are related to aromatic prenyltransferases (APT) having at least one amino acid amino acid modification as compared to SEQ ID NO: 1, 3, or 4 for the production of cannabinoids, cannabinoid derivatives, and cannabinoid analogues. The disclosure further contemplates polypeptides having combinations of the various features described herein.

Amino acid modifications may be amino acid substitutions, amino acid deletions and/or amino acid insertions. Amino acid substitutions may be conservative amino acid substitutions or non-conservative amino acid substitutions. A conservative replacement (also called a conservative mutation, a conservative substitution or a conservative variation) is an amino acid replacement in a protein that changes a given amino acid to a different amino acid with similar biochemical properties (e.g. charge, hydrophobicity and size). As used herein, “conservative variations” refer to the replacement of an amino acid residue by another, biologically similar residue. Examples of conservative variations include the substitution of one hydrophobic residue such as isoleucine, valine, leucine or methionine for another; or the substitution of one polar residue for another, such as the substitution of arginine for lysine, glutamic for aspartic acids, or glutamine for asparagine, and the like. Other illustrative examples of conservative substitutions include the changes of: alanine to serine; arginine to lysine; asparagine to glutamine or histidine; aspartate to glutamate; cysteine to serine; glutamine to asparagine; glutamate to aspartate; glycine to proline; histidine to asparagine or glutamine; isoleucine to leucine or valine; leucine to valine or isoleucine; lysine to arginine, glutamine, or glutamate; methionine to leucine or isoleucine; phenylalanine to tyrosine, leucine or methionine; serine to threonine; threonine to serine; tryptophan to tyrosine; tyrosine to tryptophan or phenylalanine; valine to isoleucine or leucine, and the like.

In some embodiments, the recombinant polypeptide comprises an amino acid sequence with at least 70% identity to SEQ ID NO: 1, 2, 3, or 4, wherein the amino acid sequence comprises at least one amino acid modification as compared to SEQ ID NO: 1, 3, or 4. Some aspects of the present disclosure are related to a recombinant polypeptide comprising an amino acid sequence with at least 90% identity to SEQ ID NO: 5, wherein the amino acid sequence comprises at least one amino acid modification as compared to SEQ ID NO: 1, 2, 3, or 4. In some embodiments, the recombinant polypeptide comprises one or more of a histidine tag sequence, TEV cleavage sequence, an addition of a glycine at the C-termini, or a deletion of 10 to 16 amino acids from the C-terminus. In some embodiments, the recombinant polypeptide comprises a histidine tag sequence, a TEV cleavage sequence, and a deletion of 8 to 16 (e.g., 10-16) amino acids from the C-terminus of the SEQ ID NO: 1, 2, 3, or 4. In some embodiments, the recombinant polypeptide comprises a deletion of 8 to 16 (e.g., 10-16) amino acids from the C-terminus of the SEQ ID NO: 1, 2, 3, or 4.

“Identity” refers to the extent to which the sequence of two or more nucleic acids or polypeptides is the same. In some embodiments, percent identity between a sequence of interest and a second sequence over a window of evaluation, e.g., over the length of the sequence of interest, may be computed by aligning the sequences, determining the number of residues (nucleotides or amino acids) within the window of evaluation that are opposite an identical residue allowing the introduction of gaps to maximize identity, dividing by the total number of residues of the sequence of interest or the second sequence (whichever is greater) that fall within the window, and multiplying by 100. When computing the number of identical residues needed to achieve a particular percent identity, fractions are to be rounded to the nearest whole number. Percent identity can be calculated with the use of a variety of computer programs known in the art. For example, computer programs such as BLAST2, BLASTN, BLASTP, Gapped BLAST, etc., generate alignments and provide percent identity between sequences of interest. The algorithm of Karlin and Altschul (Karlin and Altschul, Proc. Natl. Acad. Sci. USA 87:22264-2268, 1990) modified as in Karlin and Altschul, Proc. Natl. Acad. Sci. USA 90:5873-5877, 1993 is incorporated into the NBLAST and XBLAST programs of Altschul et al. (Altschul, et al., J. Mol. Biol. 215:403-410, 1990). To obtain gapped alignments for comparison purposes, Gapped BLAST is utilized as described in Altschul et al. (Altschul, et al. Nucleic Acids Res. 25:3389-3402, 1997). When utilizing BLAST and Gapped BLAST programs, the default parameters of the respective programs may be used. A PAM250 or BLOSUM62 matrix may be used. Software for performing BLAST analyses is publicly available through the National Center for Biotechnology Information (NCBI). See the Web site having URL ncbi.nlm.nih.gov for these programs. In a specific embodiment, percent identity is calculated using BLAST2 with default parameters as provided by the NCBI.

In some embodiments, the amino acid sequence has at least 75% identity to SEQ ID NO: 1, 2, 3, or 4. In some embodiments, the amino acid sequence has at least 80% identity to SEQ ID NO: 1, 2, 3, or 4. In some embodiments, the amino acid sequence has at least 85% identity to SEQ ID NO: 1, 2, 3, or 4. In some embodiments, the amino acid sequence has at least 90% identity to SEQ ID NO: 1, 2, 3, or 4. In some embodiments, the amino acid sequence has at least 95% identity to SEQ ID NO: 1, 2, 3, or 4. In some embodiments, the amino acid sequence has at least 96% identity to SEQ ID NO: 1, 2, 3, or 4. In some embodiments, the amino acid sequence has at least 97% identity to SEQ ID NO: 1, 2, 3, or 4. In some embodiments, the amino acid sequence has at least 98% identity to SEQ ID NO: 1, 2, 3, or 4. In some embodiments, the amino acid sequence has at least 99% identity to SEQ ID NO: 1, 2, 3, or 4. In some embodiments, the amino acid sequence has at least 99.5% identity to SEQ ID NO: 1, 2, 3, or 4. In some embodiments, the amino acid sequence has at least 99.9% identity to SEQ ID NO: 1, 2, 3, or 4. In some embodiments, the amino acid sequence has at least 100% identity to SEQ ID NO: 1, 2, 3, or 4.

In some embodiments, the amino acid sequence has at least 90% identity to SEQ ID NO: 1. In some embodiments, the amino acid sequence has at least 90% identity to SEQ ID NO: 2. In some embodiments, the amino acid sequence has at least 90% identity to SEQ ID NO: 3. In some embodiments, the amino acid sequence has at least 90% identity to SEQ ID NO: 4. In some embodiments, the amino acid sequence has at least 95% identity to SEQ ID NO: 1. In some embodiments, the amino acid sequence has at least 95% identity to SEQ ID NO: 2. In some embodiments, the amino acid sequence has at least 95% identity to SEQ ID NO: 3. In some embodiments, the amino acid sequence has at least 95% identity to SEQ ID NO: 4.

In some embodiments, the amino acid sequence has at least 91% identity to SEQ ID NO: 5. In some embodiments, the amino acid sequence has at least 92% identity to SEQ ID NO: 5. In some embodiments, the amino acid sequence has at least 93% identity to SEQ ID NO: 5. In some embodiments, the amino acid sequence has at least 94% identity to SEQ ID NO: 5. In some embodiments, the amino acid sequence has at least 95% identity to SEQ ID NO: 5. In some embodiments, the amino acid sequence has at least 96% identity to SEQ ID NO: 5. In some embodiments, the amino acid sequence has at least 97% identity to SEQ ID NO: 5. In some embodiments, the amino acid sequence has at least 98% identity to SEQ ID NO: 5. In some embodiments, the amino acid sequence has at least 99% identity to SEQ ID NO: 5. In some embodiments, the amino acid sequence has at least 99.5% identity to SEQ ID NO: 5. In some embodiments, the amino acid sequence has at least 99.9% identity to SEQ ID NO: 5. In some embodiments, the amino acid sequence has 100% identity to SEQ ID NO: 5.

In some embodiments, the amino acid sequence has at least 91% identity to SEQ ID NO: 6. In some embodiments, the amino acid sequence has at least 92% identity to SEQ ID NO: 6. In some embodiments, the amino acid sequence has at least 93% identity to SEQ ID NO: 6. In some embodiments, the amino acid sequence has at least 94% identity to SEQ ID NO: 6. In some embodiments, the amino acid sequence has at least 95% identity to SEQ ID NO: 6. In some embodiments, the amino acid sequence has at least 96% identity to SEQ ID NO: 6. In some embodiments, the amino acid sequence has at least 97% identity to SEQ ID NO: 6. In some embodiments, the amino acid sequence has at least 98% identity to SEQ ID NO: 6. In some embodiments, the amino acid sequence has at least 99% identity to SEQ ID NO: 6. In some embodiments, the amino acid sequence has at least 99.5% identity to SEQ ID NO: 6. In some embodiments, the amino acid sequence has at least 99.9% identity to SEQ ID NO: 6. In some embodiments, the amino acid sequence has 100% identity to SEQ ID NO: 6.

In some embodiments, the amino acid sequence has 1-20 amino acid modification as compared to SEQ ID NO: 1. In some embodiments, the amino acid sequence has 1 amino acid modification as compared to SEQ ID NO: 1. In some embodiments, the amino acid sequence has 2 amino acid modifications as compared to SEQ ID NO: 1. In some embodiments, the amino acid sequence has 3 amino acid modifications as compared to SEQ ID NO: 1. In some embodiments, the amino acid sequence has 4 amino acid modifications as compared to SEQ ID NO: 1. In some embodiments, the amino acid sequence has 5 amino acid modifications as compared to SEQ ID NO: 1. In some embodiments, the amino acid sequence has 6 amino acid modifications as compared to SEQ ID NO: 1. In some embodiments, the amino acid sequence has 7 amino acid modifications as compared to SEQ ID NO: 1. In some embodiments, the amino acid sequence has 8 amino acid modifications as compared to SEQ ID NO: 1. In some embodiments, the amino acid sequence has 9 amino acid modifications as compared to SEQ ID NO: 1. In some embodiments, the amino acid sequence has 10 amino acid modifications as compared to SEQ ID NO: 1. In some embodiments, the amino acid sequence has 11 amino acid modifications as compared to SEQ ID NO: 1. In some embodiments, the amino acid sequence has 12 amino acid modifications as compared to SEQ ID NO: 1. In some embodiments, the amino acid sequence has 13 amino acid modifications as compared to SEQ ID NO: 1. In some embodiments, the amino acid sequence has 14 amino acid modifications as compared to SEQ ID NO: 1. In some embodiments, the amino acid sequence has 15 amino acid modifications as compared to SEQ ID NO: 1. In some embodiments, the amino acid sequence has 16 amino acid modifications as compared to SEQ ID NO: 1. In some embodiments, the amino acid sequence has 17 amino acid modifications as compared to SEQ ID NO: 1. In some embodiments, the amino acid sequence has 18 amino acid modifications as compared to SEQ ID NO: 1. In some embodiments, the amino acid sequence has 19 amino acid modifications as compared to SEQ ID NO: 1. In some embodiments, the amino acid sequence has 20 amino acid modifications as compared to SEQ ID NO: 1. In some embodiments, the amino acid sequence has between one to twenty amino acids deleted at the C-terminus as compared to SEQ ID NO: 1. In some embodiments, the amino acid sequence has between ten and sixteen amino acids deleted at the C-terminus as compared to SEQ ID NO: 1. In some embodiments, the amino acid sequence with one to twenty amino acids (e.g., 10-16 amino acids) deleted at the C-terminus as compared to SEQ ID NO: 1 further comprises one to twenty amino acid modifications as described herein.

In some embodiments, the amino acid sequence has 1-20 amino acid modification as compared to SEQ ID NO: 2. In some embodiments, the amino acid sequence has 1 amino acid modification as compared to SEQ ID NO: 2. In some embodiments, the amino acid sequence has 2 amino acid modifications as compared to SEQ ID NO: 2. In some embodiments, the amino acid sequence has 3 amino acid modifications as compared to SEQ ID NO: 2. In some embodiments, the amino acid sequence has 4 amino acid modifications as compared to SEQ ID NO: 2. In some embodiments, the amino acid sequence has 5 amino acid modifications as compared to SEQ ID NO: 2. In some embodiments, the amino acid sequence has 6 amino acid modifications as compared to SEQ ID NO: 2. In some embodiments, the amino acid sequence has 7 amino acid modifications as compared to SEQ ID NO: 2. In some embodiments, the amino acid sequence has 8 amino acid modifications as compared to SEQ ID NO: 2. In some embodiments, the amino acid sequence has 9 amino acid modifications as compared to SEQ ID NO: 2. In some embodiments, the amino acid sequence has 10 amino acid modifications as compared to SEQ ID NO: 2. In some embodiments, the amino acid sequence has 11 amino acid modifications as compared to SEQ ID NO: 2. In some embodiments, the amino acid sequence has 12 amino acid modifications as compared to SEQ ID NO: 2. In some embodiments, the amino acid sequence has 13 amino acid modifications as compared to SEQ ID NO: 2. In some embodiments, the amino acid sequence has 14 amino acid modifications as compared to SEQ ID NO: 2. In some embodiments, the amino acid sequence has 15 amino acid modifications as compared to SEQ ID NO: 2. In some embodiments, the amino acid sequence has 16 amino acid modifications as compared to SEQ ID NO: 2. In some embodiments, the amino acid sequence has 17 amino acid modifications as compared to SEQ ID NO: 2. In some embodiments, the amino acid sequence has 18 amino acid modifications as compared to SEQ ID NO: 2. In some embodiments, the amino acid sequence has 19 amino acid modifications as compared to SEQ ID NO: 2. In some embodiments, the amino acid sequence has 20 amino acid modifications as compared to SEQ ID NO: 2. In some embodiments, the amino acid sequence has between one to twenty amino acids deleted at the C-terminus as compared to SEQ ID NO: 2. In some embodiments, the amino acid sequence has between ten and sixteen amino acids deleted at the C-terminus as compared to SEQ ID NO: 2. In some embodiments, the amino acid sequence with one to twenty amino acids (e.g., 10-16 amino acids) deleted at the C-terminus as compared to SEQ ID NO: 2 further comprises one to twenty amino acid modifications as described herein.

In some embodiments, the amino acid sequence has 1-20 amino acid modification as compared to SEQ ID NO: 3. In some embodiments, the amino acid sequence has 1 amino acid modification as compared to SEQ ID NO: 3. In some embodiments, the amino acid sequence has 2 amino acid modifications as compared to SEQ ID NO: 3. In some embodiments, the amino acid sequence has 3 amino acid modifications as compared to SEQ ID NO: 3. In some embodiments, the amino acid sequence has 4 amino acid modifications as compared to SEQ ID NO: 3. In some embodiments, the amino acid sequence has 5 amino acid modifications as compared to SEQ ID NO: 3. In some embodiments, the amino acid sequence has 6 amino acid modifications as compared to SEQ ID NO: 3. In some embodiments, the amino acid sequence has 7 amino acid modifications as compared to SEQ ID NO: 3. In some embodiments, the amino acid sequence has 8 amino acid modifications as compared to SEQ ID NO: 3. In some embodiments, the amino acid sequence has 9 amino acid modifications as compared to SEQ ID NO: 3. In some embodiments, the amino acid sequence has 10 amino acid modifications as compared to SEQ ID NO: 3. In some embodiments, the amino acid sequence has 11 amino acid modifications as compared to SEQ ID NO: 3. In some embodiments, the amino acid sequence has 12 amino acid modifications as compared to SEQ ID NO: 3. In some embodiments, the amino acid sequence has 13 amino acid modifications as compared to SEQ ID NO: 3. In some embodiments, the amino acid sequence has 14 amino acid modifications as compared to SEQ ID NO: 3. In some embodiments, the amino acid sequence has 15 amino acid modifications as compared to SEQ ID NO: 3. In some embodiments, the amino acid sequence has 16 amino acid modifications as compared to SEQ ID NO: 3. In some embodiments, the amino acid sequence has 17 amino acid modifications as compared to SEQ ID NO: 3. In some embodiments, the amino acid sequence has 18 amino acid modifications as compared to SEQ ID NO: 3. In some embodiments, the amino acid sequence has 19 amino acid modifications as compared to SEQ ID NO: 3. In some embodiments, the amino acid sequence has 20 amino acid modifications as compared to SEQ ID NO: 3. In some embodiments, the amino acid sequence has between one to twenty amino acids deleted at the C-terminus as compared to SEQ ID NO: 3. In some embodiments, the amino acid sequence has between ten and sixteen amino acids deleted at the C-terminus as compared to SEQ ID NO: 3. In some embodiments, the amino acid sequence with one to twenty amino acids (e.g., 10-16 amino acids) deleted at the C-terminus as compared to SEQ ID NO: 3 further comprises one to twenty amino acid modifications as described herein.

In some embodiments, the amino acid sequence has 1-20 amino acid modification as compared to SEQ ID NO: 4. In some embodiments, the amino acid sequence has 1 amino acid modification as compared to SEQ ID NO: 4. In some embodiments, the amino acid sequence has 2 amino acid modifications as compared to SEQ ID NO: 4. In some embodiments, the amino acid sequence has 3 amino acid modifications as compared to SEQ ID NO: 4. In some embodiments, the amino acid sequence has 4 amino acid modifications as compared to SEQ ID NO: 4. In some embodiments, the amino acid sequence has 5 amino acid modifications as compared to SEQ ID NO: 4. In some embodiments, the amino acid sequence has 6 amino acid modifications as compared to SEQ ID NO: 4. In some embodiments, the amino acid sequence has 7 amino acid modifications as compared to SEQ ID NO: 4. In some embodiments, the amino acid sequence has 8 amino acid modifications as compared to SEQ ID NO: 4. In some embodiments, the amino acid sequence has 9 amino acid modifications as compared to SEQ ID NO: 4. In some embodiments, the amino acid sequence has 10 amino acid modifications as compared to SEQ ID NO: 4. In some embodiments, the amino acid sequence has 11 amino acid modifications as compared to SEQ ID NO: 4. In some embodiments, the amino acid sequence has 12 amino acid modifications as compared to SEQ ID NO: 4. In some embodiments, the amino acid sequence has 13 amino acid modifications as compared to SEQ ID NO: 4. In some embodiments, the amino acid sequence has 14 amino acid modifications as compared to SEQ ID NO: 4. In some embodiments, the amino acid sequence has 15 amino acid modifications as compared to SEQ ID NO: 4. In some embodiments, the amino acid sequence has 16 amino acid modifications as compared to SEQ ID NO: 4. In some embodiments, the amino acid sequence has 17 amino acid modifications as compared to SEQ ID NO: 4. In some embodiments, the amino acid sequence has 18 amino acid modifications as compared to SEQ ID NO: 4. In some embodiments, the amino acid sequence has 19 amino acid modifications as compared to SEQ ID NO: 4. In some embodiments, the amino acid sequence has 20 amino acid modifications as compared to SEQ ID NO: 4. In some embodiments, the amino acid sequence has between one to twenty amino acids deleted at the C-terminus as compared to SEQ ID NO: 4. In some embodiments, the amino acid sequence has between ten and sixteen amino acids deleted at the C-terminus as compared to SEQ ID NO: 4. In some embodiments, the amino acid sequence with one to twenty amino acids (e.g., 10-16 amino acids) deleted at the C-terminus as compared to SEQ ID NO: 4 further comprises one to twenty amino acid modifications as described herein.

In some embodiments, the recombinant polypeptide comprises an amino acid sequence identical to SEQ ID NO: 1 with one to twenty amino acid substitutions, wherein the amino acid substitutions are located in SEQ ID NO: 1 at positions selected from 49, 51, 52, 53, 65, 67, 68, 69, 111, 113, 122, 124, 125, 126, 164, 165, 166, 167, 170, 214, 215, 217, 219, 229, 231, 233, 235, 268, 269, 270, 282, 283, 284, 285, 286, and 293. In some embodiments, the recombinant polypeptide comprises an amino acid sequence identical to SEQ ID NO: 1 with one to twenty amino acids deleted from the C-terminus and one to twenty amino acid substitutions, wherein the amino acid substitutions are located in SEQ ID NO: 1 at positions selected from 49, 51, 52, 53, 65, 67, 68, 69, 111, 113, 122, 124, 125, 126, 164, 165, 166, 167, 170, 214, 215, 217, 219, 229, 231, 233, 235, 268, 269, 270, 282, 283, 284, 285, 286, and 293.

In some embodiments, the recombinant polypeptide comprises an amino acid sequence identical to SEQ ID NO: 2 with one to twenty amino acid substitutions, wherein the amino acid substitutions are located in SEQ ID NO: 2 at positions selected from 38, 39, 40, 41, 42, 43, 44, 55, 57, 58, 59, 101, 103, 111, 112, 114, 115, 116, 117, 155, 156, 157, 160, 167, 169, 204, 205, 207, 209, 219, 221, 223, 224, 225, 258, 259, 260, 272, 273, 274, 275, 276, and 283. In some embodiments, the recombinant polypeptide comprises an amino acid sequence identical to SEQ ID NO: 2 with one to three amino acid substitutions, wherein the amino acid substitutions are located in SEQ ID NO: 2 at positions selected from 116, 205, and 260. In some embodiments, the recombinant polypeptide comprises an amino acid sequence identical to SEQ ID NO: 2 with one to twenty amino acid substitutions, wherein the amino acid substitutions comprise one or more substitutions located in SEQ ID NO: 2 at positions selected from 116, 205, and 260. In some embodiments, the remaining substitutions of the one to twenty substitutions are at the positions in SEQ ID NO: 2 identified above.

In some embodiments, the recombinant polypeptide comprises an amino acid sequence identical to SEQ ID NO: 2 with one to twenty amino acids deleted at the C-terminus and one to twenty amino acid substitutions, wherein the amino acid substitutions are located in SEQ ID NO: 2 at positions selected from 38, 39, 40, 41, 42, 43, 44, 55, 57, 58, 59, 101, 103, 111, 112, 114, 115, 116, 117, 155, 156, 157, 160, 167, 169, 204, 205, 207, 209, 219, 221, 223, 224, 225, 258, 259, 260, 272, 273, 274, 275, 276, and 283. In some embodiments, the recombinant polypeptide comprises an amino acid sequence identical to SEQ ID NO: 2 with one to twenty amino acids deleted at the C-terminus and one to three amino acid substitutions, wherein the amino acid substitutions are located in SEQ ID NO: 2 at positions selected from 116, 205, and 260. In some embodiments, the recombinant polypeptide comprises an amino acid sequence identical to SEQ ID NO: 2 with one to twenty amino acids deleted at the C-terminus and one to twenty amino acid substitutions, wherein the amino acid substitutions comprise one or more substitutions located in SEQ ID NO: 2 at positions selected from 116, 205, and 260. In some embodiments, the remaining substitutions of the one to twenty substitutions are at the positions in SEQ ID NO: 2 identified above.

In some embodiments, the recombinant polypeptide comprises an amino acid sequence identical to SEQ ID NO: 3 with one to twenty amino acid substitutions, wherein the amino acid substitutions are located in SEQ ID NO: 3 at positions selected from 108, 109, 110, 111, 112, 113, 114, 125, 127, 128, 129, 171, 173, 181, 182, 184, 185, 186, 187, 225, 226, 227, 230, 237, 239, 274, 275, 277, 279, 289, 291, 293, 294, 295, 328, 329, 330, 342, 343, 344, 345, 346, 353. In some embodiments, the recombinant polypeptide comprises an amino acid sequence identical to SEQ ID NO: 3 with one to three amino acid substitutions, wherein the amino acid substitutions are located in SEQ ID NO: 2 at positions selected from 186, 275, and 330. In some embodiments, the recombinant polypeptide comprises an amino acid sequence identical to SEQ ID NO: 3 with one to three amino acid substitutions, wherein the amino acid substitutions comprise one or more substitutions located in SEQ ID NO: 2 at positions selected from 186, 275, and 330. In some embodiments, the remaining substitutions of the one to twenty substitutions are at the positions in SEQ ID NO: 3 identified above.

In some embodiments, the recombinant polypeptide comprises an amino acid sequence identical to SEQ ID NO: 3 with one to twenty amino acid deleted at the C-terminus and one to twenty amino acid substitutions, wherein the amino acid substitutions are located in SEQ ID NO: 3 at positions selected from 108, 109, 110, 111, 112, 113, 114, 125, 127, 128, 129, 171, 173, 181, 182, 184, 185, 186, 187, 225, 226, 227, 230, 237, 239, 274, 275, 277, 279, 289, 291, 293, 294, 295, 328, 329, 330, 342, 343, 344, 345, 346, 353. In some embodiments, the recombinant polypeptide comprises an amino acid sequence identical to SEQ ID NO: 3 with one to twenty amino acids deleted at the C-terminus and one to three amino acid substitutions, wherein the amino acid substitutions are located in SEQ ID NO: 3 at positions selected from 186, 275, and 330. In some embodiments, the recombinant polypeptide comprises an amino acid sequence identical to SEQ ID NO: 3 with one to twenty amino acids deleted at the C-terminus and one to twenty amino acid substitutions, wherein the amino acid substitutions comprise one or more substitutions located in SEQ ID NO: 3 at positions selected from 186, 275, and 330. In some embodiments, the remaining substitutions of the one to twenty substitutions are at the positions in SEQ ID NO: 3 identified above.

In some embodiments, the recombinant polypeptide comprises an amino acid sequence identical to SEQ ID NO: 4 with one to twenty amino acid substitutions, wherein the amino acid substitutions are located in SEQ ID NO: 4 at positions selected from 38, 39, 40, 41, 42, 43, 44, 55, 57, 58, 59, 101, 103, 111, 112, 114, 115, 116, 117, 155, 156, 157, 160, 167, 169, 204, 205, 207, 209, 219, 221, 223, 224, 225, 258, 259, 260, 272, 273, 274, 275, 276, and 283. In some embodiments, the recombinant polypeptide comprises an amino acid sequence identical to SEQ ID NO: 4 with one to three amino acid substitutions, wherein the amino acid substitutions are located in SEQ ID NO: 2 at positions selected from 116, 205, and 260. In some embodiments, the recombinant polypeptide comprises an amino acid sequence identical to SEQ ID NO: 4 with one to three amino acid substitutions, wherein the amino acid substitutions comprise one or more substitutions located in SEQ ID NO: 2 at positions selected from 116, 205, and 260. In some embodiments, the remaining substitutions of the one to twenty substitutions are at the positions in SEQ ID NO: 4 identified above.

In some embodiments, the recombinant polypeptide comprises an amino acid sequence identical to SEQ ID NO: 4 with one to twenty amino acids deleted at the C-terminus and one to twenty amino acid substitutions, wherein the amino acid substitutions are located in SEQ ID NO: 4 at positions selected from 38, 39, 40, 41, 42, 43, 44, 55, 57, 58, 59, 101, 103, 111, 112, 114, 115, 116, 117, 155, 156, 157, 160, 167, 169, 204, 205, 207, 209, 219, 221, 223, 224, 225, 258, 259, 260, 272, 273, 274, 275, 276, and 283. In some embodiments, the recombinant polypeptide comprises an amino acid sequence identical to SEQ ID NO: 4 with one to twenty amino acids deleted at the C-terminus and one to three amino acid substitutions, wherein the amino acid substitutions are located in SEQ ID NO: 4 at positions selected from 116, 205, and 260. In some embodiments, the recombinant polypeptide comprises an amino acid sequence identical to SEQ ID NO: 4 with one to twenty amino acids deleted at the C-terminus and one to twenty amino acid substitutions, wherein the amino acid substitutions comprise one or more substitutions located in SEQ ID NO: 4 at positions selected from 116, 205, and 260. In some embodiments, the remaining substitutions of the one to twenty substitutions are at the positions in SEQ ID NO: 4 identified above.

In some embodiments, the recombinant polypeptide comprises an amino acid sequence identical to SEQ ID NO: 1 with one to twenty amino acid substitutions, wherein the amino acid substitutions are located in SEQ ID NO: 1 at positions selected from 49, 51, 52, 53, 65, 67, 68, 69, 111, 113, 122, 124, 125, 126, 164, 165, 166, 167, 170, 214, 215, 217, 219, 229, 231, 233, 235, 268, 269, 270, 282, 283, 284, 285, 286, and 293. In some embodiments, the recombinant polypeptide comprises an amino acid sequence identical to SEQ ID NO: 1 with one to twenty amino acids deleted from the C-terminus and one to twenty amino acid substitutions, wherein the amino acid substitutions are located in SEQ ID NO: 1 at positions selected from 49, 51, 52, 53, 65, 67, 68, 69, 111, 113, 122, 124, 125, 126, 164, 165, 166, 167, 170, 214, 215, 217, 219, 229, 231, 233, 235, 268, 269, 270, 282, 283, 284, 285, 286, 292, 293, 294, 295, 296, 297, and 298.

In some embodiments, the recombinant polypeptide comprises an amino acid sequence identical to SEQ ID NO: 2 with one to twenty amino acid substitutions, wherein the amino acid substitutions are located in SEQ ID NO: 2 at positions selected from 38, 39, 40, 41, 42, 43, 44, 55, 57, 58, 59, 101, 103, 111, 112, 114, 115, 116, 117, 155, 156, 157, 160, 167, 169, 204, 205, 207, 209, 219, 221, 223, 224, 225, 258, 259, 260, 272, 273, 274, 275, 276, 282, 283, 284, 285, 286, 287, and 288. In some embodiments, the recombinant polypeptide comprises an amino acid sequence identical to SEQ ID NO: 2 with one to three amino acid substitutions, wherein the amino acid substitutions are located in SEQ ID NO: 2 at positions selected from 116, 205, and 260. In some embodiments, the recombinant polypeptide comprises an amino acid sequence identical to SEQ ID NO: 2 with one to twenty amino acid substitutions, wherein the amino acid substitutions comprise one or more substitutions located in SEQ ID NO: 2 at positions selected from 116, 156, 205, 223, 225, 260, 276, 282, 283, 284. In some embodiments, the remaining substitutions of the one to twenty substitutions are at the positions in SEQ ID NO: 2 identified above.

In some embodiments, the recombinant polypeptide comprises an amino acid sequence identical to SEQ ID NO: 2 with one to twenty amino acids deleted at the C-terminus and one to twenty amino acid substitutions, wherein the amino acid substitutions are located in SEQ ID NO: 2 at positions selected from 38, 39, 40, 41, 42, 43, 44, 55, 57, 58, 59, 101, 103, 111, 112, 114, 115, 116, 117, 155, 156, 157, 160, 167, 169, 204, 205, 207, 209, 219, 221, 223, 224, 225, 258, 259, 260, 272, 273, 274, 275, 276, 282, 283, 284, 285, 286, 287, and 288. In some embodiments, the recombinant polypeptide comprises an amino acid sequence identical to SEQ ID NO: 2 with one to twenty amino acids deleted at the C-terminus and one to three amino acid substitutions, wherein the amino acid substitutions are located in SEQ ID NO: 2 at positions selected from 116, 205, and 260. In some embodiments, the recombinant polypeptide comprises an amino acid sequence identical to SEQ ID NO: 2 with one to twenty amino acids deleted at the C-terminus and one to twenty amino acid substitutions, wherein the amino acid substitutions comprise one or more substitutions located in SEQ ID NO: 2 at positions selected from 116, 156, 205, 223, 225, 260, 276, 282, 283, and 284. In some embodiments, the remaining substitutions of the one to twenty substitutions are at the positions in SEQ ID NO: 2 identified above.

In some embodiments, the recombinant polypeptide comprises an amino acid sequence identical to SEQ ID NO: 3 with one to twenty amino acid substitutions, wherein the amino acid substitutions are located in SEQ ID NO: 3 at positions selected from 108, 109, 110, 111, 112, 113, 114, 125, 127, 128, 129, 171, 173, 181, 182, 184, 185, 186, 187, 225, 226, 227, 230, 237, 239, 274, 275, 277, 279, 289, 291, 293, 294, 295, 328, 329, 330, 342, 343, 344, 345, 346, 352, 353, 354, 355, 356, 357 and 358. In some embodiments, the recombinant polypeptide comprises an amino acid sequence identical to SEQ ID NO: 3 with one to three amino acid substitutions, wherein the amino acid substitutions are located in SEQ ID NO: 3 at positions selected from 186, 275, and 330. In some embodiments, the recombinant polypeptide comprises an amino acid sequence identical to SEQ ID NO: 3 with one to three amino acid substitutions, wherein the amino acid substitutions comprise one or more substitutions located in SEQ ID NO: 2 at positions selected from 186, 275, and 330. In some embodiments, the recombinant polypeptide comprises an amino acid sequence identical to SEQ ID NO: 3 with one to three amino acid substitutions and between 10-16 amino acids deleted from the C-terminus, wherein the amino acid substitutions comprise one or more substitutions located in SEQ ID NO: 2 at positions selected from 186, 275, and 330. In some embodiments, the remaining substitutions of the one to twenty substitutions are at the positions in SEQ ID NO: 3 identified above.

In some embodiments, the recombinant polypeptide comprises an amino acid sequence identical to SEQ ID NO: 3 with a substitution at position 186. In some embodiments, the recombinant polypeptide comprises an amino acid sequence identical to SEQ ID NO: 3 with a substitution at position 275. In some embodiments, the recombinant polypeptide comprises an amino acid sequence identical to SEQ ID NO: 3 with a substitution at position 330. In some embodiments, the recombinant polypeptide comprises an amino acid sequence at least 95% identical to SEQ ID NO: 3 with a substitution at position 186. In some embodiments, the recombinant polypeptide comprises an amino acid sequence at least 95% identical to SEQ ID NO: 3 with a substitution at position 275. In some embodiments, the recombinant polypeptide comprises an amino acid sequence at least 95% identical to SEQ ID NO: 3 with a substitution at position 330. In some embodiments, the recombinant polypeptide comprises an amino acid sequence at least 99% identical to SEQ ID NO: 3 with a substitution at position 186. In some embodiments, the recombinant polypeptide comprises an amino acid sequence at least 99% identical to SEQ ID NO: 3 with a substitution at position 275. In some embodiments, the recombinant polypeptide comprises an amino acid sequence at least 99% identical to SEQ ID NO: 3 with a substitution at position 330.

In some embodiments, the recombinant polypeptide comprises an amino acid sequence identical to SEQ ID NO: 3 with one to twenty amino acid deleted at the C-terminus and one to twenty amino acid substitutions, wherein the amino acid substitutions are located in SEQ ID NO: 3 at positions selected from 108, 109, 110, 111, 112, 113, 114, 125, 127, 128, 129, 171, 173, 181, 182, 184, 185, 186, 187, 225, 226, 227, 230, 237, 239, 274, 275, 277, 279, 289, 291, 293, 294, 295, 328, 329, 330, 342, 343, 344, 345, 346, 352, 353, 354, 355, 356, 357 and 358. In some embodiments, the recombinant polypeptide comprises an amino acid sequence identical to SEQ ID NO: 3 with one to twenty (e.g., 10-16) amino acids deleted at the C-terminus and one to three amino acid substitutions, wherein the amino acid substitutions are located in SEQ ID NO: 3 at positions selected from 186, 275, and 330. In some embodiments, the recombinant polypeptide comprises an amino acid sequence identical to SEQ ID NO: 3 with one to twenty (e.g., 10-16) amino acids deleted at the C-terminus and one to twenty amino acid substitutions, wherein the amino acid substitutions comprise one or more substitutions located in SEQ ID NO: 3 at positions selected from 186, 275, and 330. In some embodiments, the remaining substitutions of the one to twenty substitutions are at the positions in SEQ ID NO: 3 identified above.

In some embodiments, the recombinant polypeptide comprises an amino acid sequence identical to SEQ ID NO: 3 with one to twenty (e.g., 10-16) amino acids deleted at the C-terminus and with a substitution at position 186. In some embodiments, the recombinant polypeptide comprises an amino acid sequence identical to SEQ ID NO: 3 with one to twenty amino acids deleted at the C-terminus and with a substitution at position 275. In some embodiments, the recombinant polypeptide comprises an amino acid sequence identical to SEQ ID NO: 3 with one to twenty amino acids deleted at the C-terminus and with a substitution at position 330. In some embodiments, the recombinant polypeptide comprises an amino acid sequence at least 95% identical to SEQ ID NO: 3 with one to twenty amino acids deleted at the C-terminus and with a substitution at position 186. In some embodiments, the recombinant polypeptide comprises an amino acid sequence at least 95% identical to SEQ ID NO: 3 with one to twenty amino acids deleted at the C-terminus and with a substitution at position 275. In some embodiments, the recombinant polypeptide comprises an amino acid sequence at least 95% identical to SEQ ID NO: 3 with one to twenty amino acids deleted at the C-terminus and with a substitution at position 330. In some embodiments, the recombinant polypeptide comprises an amino acid sequence at least 99% identical to SEQ ID NO: 3 with one to twenty amino acids deleted at the C-terminus and with a substitution at position 186. In some embodiments, the recombinant polypeptide comprises an amino acid sequence at least 99% identical to SEQ ID NO: 3 with one to twenty amino acids deleted at the C-terminus and with a substitution at position 275. In some embodiments, the recombinant polypeptide comprises an amino acid sequence at least 99% identical to SEQ ID NO: 3 with one to twenty amino acids deleted at the C-terminus and with a substitution at position 330.

In some embodiments, the recombinant polypeptide comprises an amino acid sequence identical to SEQ ID NO: 4 with one to twenty amino acid substitutions, wherein the amino acid substitutions are located in SEQ ID NO: 4 at positions selected from 38, 39, 40, 41, 42, 43, 44, 55, 57, 58, 59, 101, 103, 111, 112, 114, 115, 116, 117, 155, 156, 157, 160, 167, 169, 204, 205, 207, 209, 219, 221, 223, 224, 225, 258, 259, 260, 272, 273, 274, 275, 276, 282, 283, 284, 285, 286, 287, and 288. In some embodiments, the recombinant polypeptide comprises an amino acid sequence identical to SEQ ID NO: 4 with one to three amino acid substitutions, wherein the amino acid substitutions are located in SEQ ID NO: 2 at positions selected from 116, 205, and 260. In some embodiments, the recombinant polypeptide comprises an amino acid sequence identical to SEQ ID NO: 4 with one to three amino acid substitutions, wherein the amino acid substitutions comprise one or more substitutions located in SEQ ID NO: 2 at positions selected from 116, 156, 205, 223, 225, 260, 276, 282, 283, and 284. In some embodiments, the remaining substitutions of the one to twenty substitutions are at the positions in SEQ ID NO: 4 identified above.

In some embodiments, the recombinant polypeptide comprises an amino acid sequence identical to SEQ ID NO: 4 with one to twenty amino acids deleted at the C-terminus and one to twenty amino acid substitutions, wherein the amino acid substitutions are located in SEQ ID NO: 4 at positions selected from 38, 39, 40, 41, 42, 43, 44, 55, 57, 58, 59, 101, 103, 111, 112, 114, 115, 116, 117, 155, 156, 157, 160, 167, 169, 204, 205, 207, 209, 219, 221, 223, 224, 225, 258, 259, 260, 272, 273, 274, 275, 276, 282, 283, 284, 285, 286, 287, and 288. In some embodiments, the recombinant polypeptide comprises an amino acid sequence identical to SEQ ID NO: 4 with one to twenty amino acids deleted at the C-terminus and one to three amino acid substitutions, wherein the amino acid substitutions are located in SEQ ID NO: 4 at positions selected from 116, 205, and 260. In some embodiments, the recombinant polypeptide comprises an amino acid sequence identical to SEQ ID NO: 4 with one to twenty amino acids deleted at the C-terminus and one to twenty amino acid substitutions, wherein the amino acid substitutions comprise one or more substitutions located in SEQ ID NO: 4 at positions selected from 116, 156, 205, 223, 225, 260, 276, 282, 283, and 284. In some embodiments, the remaining substitutions of the one to twenty substitutions are at the positions in SEQ ID NO: 4 identified above.

In some embodiments, the recombinant polypeptide comprises an amino acid sequence identical to SEQ ID NO: 1, 2, 3, or 4 with one to twenty amino acid substitutions, and ten to sixteen amino acids deleted from the C-terminus. In some embodiments, the recombinant polypeptide comprises an amino acid sequence 90% identical to a sequence selected from SEQ ID NOs: 23-79 and 82-88. In some embodiments, the recombinant polypeptide comprises an amino acid sequence 91% identical to a sequence selected from SEQ ID NOs: 23-79 and 82-88. In some embodiments, the recombinant polypeptide comprises an amino acid sequence 92% identical to a sequence selected from SEQ ID NOs: 23-79 and 82-88. In some embodiments, the recombinant polypeptide comprises an amino acid sequence 93% identical to a sequence selected from SEQ ID NOs: 23-79 and 82-88. In some embodiments, the recombinant polypeptide comprises an amino acid sequence 94% identical to a sequence selected from SEQ ID NOs: 23-79 and 82-88. In some embodiments, the recombinant polypeptide comprises an amino acid sequence 95% identical to a sequence selected from SEQ ID NOs: 23-79 and 82-88. In some embodiments, the recombinant polypeptide comprises an amino acid sequence 96% identical to a sequence selected from SEQ ID NOs: 23-79 and 82-88. In some embodiments, the recombinant polypeptide comprises an amino acid sequence 97% identical to a sequence selected from SEQ ID NOs: 23-79 and 82-88. In some embodiments, the recombinant polypeptide comprises an amino acid sequence 98% identical to a sequence selected from SEQ ID NOs: 23-79 and 82-88. In some embodiments, the recombinant polypeptide comprises an amino acid sequence 99% identical to a sequence selected from SEQ ID NOs: 23-79 and 82-88. In some embodiments, the recombinant polypeptide comprises an amino acid sequence 99.5% identical to a sequence selected from SEQ ID NOs: 23-79 and 82-88. In some embodiments, the recombinant polypeptide comprises an amino acid sequence 99.9% identical to a sequence selected from SEQ ID NOs: 23-79 and 82-88. In some embodiments, the recombinant polypeptide comprises an amino acid sequence identical to a sequence selected from SEQ ID NOs: 23-79 and 82-88. In some embodiments, the sequence selected from SEQ ID NOs: 23-79 and 82-88 is SEQ ID NOs: 29-36, 43, 56, 67, 69, 70, or 74.

In some embodiments, the recombinant polypeptide comprises a fusion domain. In some embodiments, the fusion domain is a detectable tag or a moiety to improve expression in one or more expression systems, or to improve purification (e.g., affinity tag purification). Well-known examples of such fusion domains include, but are not limited to, polyhistidine (e.g., 6×His) (SEQ ID NO:10), Glu-Glu, glutathione S transferase (GST), thioredoxin, protein A, protein G, biotin, and an immunoglobulin heavy chain constant region (Fc), maltose binding protein (MBP), which are particularly useful for isolation of the fusion proteins by affinity chromatography. For the purpose of affinity purification, relevant matrices for affinity chromatography, such as glutathione-, amylase-, and nickel- or cobalt-conjugated resins are used. Fusion domains also include “epitope tags,” which are usually short peptide sequences for which a specific antibody is available. Well known epitope tags for which specific monoclonal antibodies are readily available include FLAG, influenza virus haemagglutinin (HA), His, and c-myc tags. An exemplary His tag has the sequence HHHHHH (SEQ ID NO: 10), and an exemplary c-myc tag has the sequence EQKLISEEDL (SEQ ID NO: 11). It is recognized that any such tags or fusions may be appended to either end of the recombinant polypeptide.

In some cases, the fusion domains have a protease cleavage site, such as for Factor Xa, cysteine protease (e.g., TEV protease), or Thrombin, which allows the relevant protease to partially digest the fusion proteins and thereby liberate the recombinant proteins therefrom. In some embodiments, the fusion domain or recombinant polypeptide comprises a TEV cleavage domain. The liberated proteins can then be isolated from the fusion domain by subsequent chromatographic separation. In some embodiments, the fusion domain targets the protein to a specific compartment of the cell such as the ER, vacuole, Golgi, peroxisome, lipid body (e.g., oleosome), or targets secretion of the protein from the cell into the outer membrane, periplasmic space or the culture media. In certain embodiments, the recombinant polypeptides may contain one or more modifications that are capable of stabilizing the polypeptides.

In some embodiments, the recombinant polypeptide comprises one or more of a histidine tag sequence, TEV cleavage sequence, and a glycine at the C-termini. In some embodiments, the recombinant polypeptide comprises a histidine tag sequence, TEV cleavage sequence, and an addition of a glycine at the C-termini (e.g., a fusion domain comprising or consisting of a histidine tag sequence, TEV cleavage sequence, and an addition of a glycine at the C-termini).

In some embodiments, the recombinant polypeptide is capable of converting olivetolic acid (OA) and geranyl diphosphate (GPP) to one or more products comprising cannabigerolic acid (CBGA). In some embodiments, the recombinant polypeptide is capable of producing CBGA in a cell free system, in a yeast cell, in a bacterial cell, in an algae cell, or in a plant cell. In some embodiments, the one or more products comprise at least 10%, 15%, 20%, 25%, 30%, 35%, 40%, 45%, 50%, 55%, 60%, 65%, 70%, 75%, 80%, 85%, 90%, 95%, or substantially 100% CBGA. In some embodiments, at least about 50% of the one or more products is CBGA. In some embodiments, more than about 90% of the one or more products is CBGA. In some embodiments, the recombinant polypeptide has a rate of formation of cannabigerolic acid (CBGA) from olivetolic acid (OA) and geranyl diphosphate (GPP) that is greater than the rate of formation of CBGA from OA and GPP by NphB under the same conditions. In some embodiments, the rate of formation of CBGA from OA and GPP is at least 1.1-fold, 1.2-fold, 1.3-fold, 1.4-fold, 1.5-fold, 1.6-fold, 1.7-fold, 1.8-fold, 1.9-fold, 2-fold, 2.5-fold, 5-fold, 10-fold, or more as compared to the rate of formation of CBGA from OA and GPP by NphB under the same conditions.

In some embodiments, the recombinant polypeptide is capable of converting olivetolic acid (OA) and farnesyl pyrophosphate (FPP) to one or more cannabinoids, cannabinoid derivatives or cannabinoid analogues. In some embodiments, the recombinant polypeptide is capable of producing cannabinoids, cannabinoid derivatives or cannabinoid analogues in a cell free system, in a yeast cell, in a bacterial cell, in an algae cell, or in a plant cell. In some embodiments, the activity of the recombinant polypeptide for converting OA and FPP to one or more cannabinoids, cannabinoid derivatives or cannabinoid analogues is at least about 10%, 15%, 20%, 25%, 30%, 35%, 40%, 45%, 50%, 55%, 60%, 65%, 70%, 75%, 80%, 85%, 90%, 95%, or substantially 100% of the activity of the recombinant polypeptide for converting OA and GPP to one or more cannabinoids, cannabinoid derivatives or cannabinoid analogues.

In some embodiments, the recombinant polypeptide has a rate of formation of CBGA from olivetolic acid (OA) and geranyl diphosphate (GPP) that is greater than the rate of formation of CBGA from OA and GPP by a polypeptide consisting of SEQ ID NO: 4 under the same conditions. In some embodiments, the recombinant polypeptide has a rate of formation of O-CBGA from OA and GPP that is greater than the rate of formation of O-CBGA from OA and GPP by a polypeptide consisting of SEQ ID NO: 4 under the same conditions. In some embodiments, recombinant polypeptide has a rate of formation of F-CBGA from OA and GPP that is greater than the rate of formation of F-CBGA from OA and GPP by a polypeptide consisting of SEQ ID NO: 4 under the same conditions. As used herein and in some embodiments, “greater than” is at least 1.1-fold, 1.2-fold, 1.3-fold, 1.4-fold, 1.5-fold, 1.6-fold, 1.7-fold, 1.8-fold, 1.9-fold, 2-fold, 2.5-fold, 5-fold, 10-fold, or more as compared to the relevant control.

In some embodiments, the recombinant polypeptide has a rate of formation of cannabigerovarinic acid (CBGVA) from divarinic acid (DVA) and geranyl diphosphate (GPP) that is greater than the rate of formation of CBGVA from DVA and GPP by a polypeptide consisting of SEQ ID NO: 4 under the same conditions. In some embodiments, the recombinant polypeptide has a rate of formation of O-CBGVA from DVA and GPP that is greater than the rate of formation of O-CBGVA from DVA and GPP by a polypeptide consisting of SEQ ID NO: 4 under the same conditions. In some embodiments, the recombinant polypeptide has a rate of formation of F-CBGVA from DVA and GPP that is greater than the rate of formation of F-CBGVA from DVA and GPP by a polypeptide consisting of SEQ ID NO: 4 under the same conditions. As used herein and in some embodiments, “greater than” is at least 1.1-fold, 1.2-fold, 1.3-fold, 1.4-fold, 1.5-fold, 1.6-fold, 1.7-fold, 1.8-fold, 1.9-fold, 2-fold, 2.5-fold, 5-fold, 10-fold, or more as compared to the relevant control.

In some embodiments, the recombinant polypeptide has a rate of formation of CBGA from OA and GPP that is at least 1.5-fold greater (e.g., 1.5-fold, 1.6-fold, 1.7-fold, 1.8-fold, 1.9-fold, 2-fold, 2.5-fold, 5-fold, 10-fold, or more) than the rate of formation of CBGA from OA and GPP by a polypeptide consisting of SEQ ID NO: 4 under the same conditions.

Cannabinoids, cannabinoid derivatives and cannabinoid analogues as recited herein are not limited. In some embodiments, cannabinoids may include, but are not limited to, cannabichromene (CBC) type (e.g. cannabichromenic acid), cannabigerol (CBG) type (e.g. cannabigerolic acid), cannabidiol (CBD) type (e.g. cannabidiolic acid), Δ⁹-trans-tetrahydrocannabinol (Δ⁹-THC) type (e.g. Δ⁹-tetrahydrocannabinolic acid), Δ⁸-trans-tetrahydrocannabinol (Δ⁸-THC) type, cannabicyclol (CBL) type, cannabielsoin (CBE) type, cannabinol (CBN) type, cannabinodiol (CBND) type, cannabitriol (CBT) type, cannabigerolic acid (CBGA), cannabigerolic acid monomethylether (CBGAM), cannabigerol (CBG), cannabigerol monomethylether (CBGM), cannabigerovarinic acid (CBGVA), cannabigerovarin (CBGV), cannabichromenic acid (CBCA), cannabichromene (CBC), cannabichromevarinic acid (CBCVA), cannabichromevarin (CBCV), cannabidiolic acid (CBDA), cannabidiol (CBD), cannabidiol monomethylether (CBDM), cannabidiol-C₄ (CBD-C₄), cannabidivarinic acid (CBDVA), cannabidivarin (CBDV), cannabidiorcol (CBD-C₁), Δ⁹-tetrahydrocannabinolic acid A (THCA-A), Δ⁹-tetrahydrocannabinolic acid B (THCA-B), Δ⁹-tetrahydrocannabinol (THC), A9-tetrahydrocannabinolic acid-C₄ (THCA-C₄), Δ⁹-tetrahydrocannabinol-C₄ (THC-C₄), Δ⁹-tetrahydrocannabivarinic acid (THCVA), Δ⁹-tetrahydrocannabivarin (THCV), Δ⁹-tetrahydrocannabiorcolic acid (THCA-C₁), Δ⁹-tetrahydrocannabiorcol (THC-C₁), Δ⁷-cis-iso-tetrahydrocannabivarin, Δ⁸-tetrahydrocannabinolic acid (Δ⁸-THCA), Δ⁸-tetrahydrocannabinol (Δ⁸-THC), cannabicyclolic acid (CBLA), cannabicyclol (CBL), cannabicyclovarin (CBLV), cannabielsoic acid A (CBEA-A), cannabielsoic acid B (CBEA-B), cannabielsoin (CBE), cannabielsoinic acid, cannabicitranic acid, cannabinolic acid (CBNA), cannabinol (CBN), cannabinol methylether (CBNM), cannabinol-C₄, (CBN-C₄), cannabivarin (CBV), cannabinol-C₂ (CNB-C₂), cannabiorcol (CBN-C₁), cannabinodiol (CBND), cannabinodivarin (CBVD), cannabitriol (CBT), 10-ethyoxy-9-hydroxy-delta-6a-tetrahydrocannabinol, 8,9-dihydroxyl-delta-6a-tetrahydrocannabinol, cannabitriolvarin (CBTVE), dehydrocannabifuran (DCBF), cannabifuran (CBF), cannabichromanon (CBCN), cannabicitran (CBT), 10-oxo-delta-6a-tetrahydrocannabinol (OTHC), delta-9-cis-tetrahydrocannabinol (cis-THC), 3,4,5,6-tetrahydro-7-hydroxy-alpha-alpha-2-trimethyl-9-n-propyl-2,6-methano-2H-1-benzoxocin-5-methanol (OH-iso-HHCV), cannabiripsol (CBR), and trihydroxy-delta-9-tetrahydrocannabinol (triOH-THC).

In some embodiments, the recombinant polypeptide is capable of converting divarinic acid (DVA) and GPP to one or more cannabinoids, cannabinoid derivatives or cannabinoid analogues. The cannabinoids are not limited and may be any disclosed herein. In some embodiments, the recombinant polypeptide is capable of producing cannabinoids, cannabinoid derivatives or cannabinoid analogues in a cell free system, in a yeast cell, in a bacterial cell, in an algae cell, or in a plant cell. In some embodiments, the activity of the recombinant polypeptide for converting DVA and FPP to one or more cannabinoids, cannabinoid derivatives or cannabinoid analogues is at least about 10%, 15%, 20%, 25%, 30%, 35%, 40%, 45%, 50%, 55%, 60%, 65%, 70%, 75%, 80%, 85%, 90%, 95%, or substantially 100% of the activity of the recombinant polypeptide for converting OA and GPP to one or more cannabinoids, cannabinoid derivatives or cannabinoid analogues.

### Cells Comprising Recombinant Proteins

Some aspects of the present disclosure are related to a cell comprising an exogenous nucleotide sequence coding for the recombinant polypeptides described herein. The cell is not limited and may be any suitable cell for expression. In some embodiments, the cell may be a microorganism or a plant. In some embodiments, the microorganism is a bacteria (e.g., E. coli), an algae, or a yeast. In some embodiments, the yeast is an oleaginous yeast (e.g., a Yarrowia lipolytica strain). In some embodiments, the bacteria is Escherichia coli.

Suitable cells may include, but are not limited to, Pichia pastoris, Pichia finlandica, Pichia trehalophila, Pichia koclamae, Pichia membranaefaciens, Pichia opuntiae, Pichia thermotolerans, Pichia salictaria, Pichia guercuum, Pichia pijperi, Pichia stiptis, Pichia methanolica, Pichia sp., Saccharomyces cerevisiae, Saccharomyces sp., Hansenula polymorpha (now known as Pichia angusta), Kluyveromyces sp., Kluyveromyces lactis, Kluyveromyces marxianus, Schizosaccharomyces pompe, Dekkera bruxellensis, Arxula adeninivorans, Candida albicans, Aspergillus nidulans, Aspergillus niger, Aspergillus oryzae, Trichoderma reesei, Chrysosporium lucknowense, Fusarium sp., Fusarium gramineum, Fusarium venenatum, Neurospora crassa, Chlamydomonas reinhardtii, Yarrowia lipolytica and the like. In some embodiments, the cell is a protease-deficient strain of Saccharomyces cerevisiae. In some embodiments, the cell is a eukaryotic cell other than a plant cell. In some embodiments, the cell is a plant cell. In some embodiments, the cell is a plant cell, where the plant cell is one that does not normally produce a cannabinoid, a cannabinoid derivative or analogue, a cannabinoid precursor, or a cannabinoid precursor derivative or analogue. In some embodiments, the cell is Saccharomyces cerevisiae. In some embodiments, the cell disclosed herein is cultured in vitro.

In some embodiments, the cell is a prokaryotic cell. Suitable prokaryotic cells may include, but are not limited to, any of a variety of laboratory strains of Escherichia coli, Lactobacillus sp., Salmonella sp., Shigella sp., and the like. See, e.g., Carrier et al, (1992) J. Immunol. 148:1176-1181; U.S. Pat. No. 6,447,784; and Sizemore et al. (1995) Science 270:299-302. Examples of Salmonella strains which can be employed may include, but are not limited to, Salmonella typhi and S. typhimurium. Suitable Shigella strains may include, but are not limited to, Shigella flexneri, Shigella sonnei, and Shigella disenteriae. Typically, the laboratory strain is one that is non-pathogenic. Non-limiting examples of other suitable bacteria may include, but are not limited to, Bacillus subtilis, Pseudomonas putida, Pseudomonas aeruginosa, Pseudomonas mevalonii, Rhodobacter sphaeroides, Rhodobacter capsulatus, Rhodospirillum rubrum, Rhodococcus sp., and the like.

An expression vector or vectors can be constructed to include exogenous nucleotide sequences coding for the recombinant polypeptides described herein operably linked to expression control sequences functional in the cell. Expression vectors applicable include, for example, plasmids, phage vectors, viral vectors, episomes and artificial chromosomes, including vectors and selection sequences or markers operable for stable integration into a host chromosome. Additionally, the expression vectors can include one or more selectable marker genes and appropriate expression control sequences. Selectable marker genes also can be included that, for example, provide resistance to antibiotics or toxins, complement auxotrophic deficiencies, or supply critical nutrients not in the culture media. Expression control sequences can include constitutive and inducible promoters, transcription enhancers, transcription terminators, and the like which are well known in the art. When two or more exogenous encoding nucleic acids are to be co-expressed, both nucleic acids can be inserted, for example, into a single expression vector or in separate expression vectors. For single vector expression, the encoding nucleic acids can be operationally linked to one common expression control sequence or linked to different expression control sequences, such as one inducible promoter and one constitutive promoter. The transformation of exogenous nucleic acid sequences can be confirmed using methods well known in the art. Such methods include, for example, nucleic acid analysis such as Northern blots or polymerase chain reaction (PCR) amplification of mRNA, or immunoblotting for expression of gene products, or other suitable analytical methods to test the expression of an introduced nucleic acid sequence or its corresponding gene product. It is understood by those skilled in the art that the exogenous nucleic acid is expressed in a sufficient amount to produce the desired product, and it is further understood that expression levels can be optimized to obtain sufficient expression using methods well known in the art and as disclosed herein.

The term “exogenous” is intended to mean that the referenced molecule or the referenced activity is introduced into the cell. The molecule can be introduced, for example, by introduction of an encoding nucleic acid into the host genetic material such as by integration into a host chromosome or as non-chromosomal genetic material such as a plasmid. Therefore, the term as it is used in reference to expression of an encoding nucleic acid refers to introduction of the encoding nucleic acid in an expressible form into the cell. When used in reference to a biosynthetic activity, the term refers to an activity that is introduced into the host. The source can be, for example, a homologous or heterologous encoding nucleic acid that expresses the referenced activity following introduction into the cell. Therefore, the term “endogenous” refers to a referenced molecule or activity that is present in the cell. Similarly, the term when used in reference to expression of an encoding nucleic acid refers to expression of an encoding nucleic acid contained within the microbial organism. The term “heterologous” refers to a molecule or activity derived from a source other than the referenced species whereas “homologous” refers to a molecule or activity derived from the host microbial organism. Accordingly, exogenous expression of an encoding nucleic acid can utilize either or both a heterologous or homologous encoding nucleic acid.

In some embodiments, the cell comprises an exogenous nucleotide sequence coding for a recombinant polypeptide comprising an amino acid sequence having at least 70% identity to SEQ ID NO: 1, 2, 3, or 4. In some embodiments, the cell comprises an exogenous nucleotide sequence coding for a recombinant polypeptide comprising an amino acid sequence having at least 75%, 80%, 85%, 90%, 95%, 99%, 99.5%, or 99.9% identity to SEQ ID NO: 1, 2, 3, or 4. In some embodiments, the cell comprises an exogenous nucleotide sequence coding for a recombinant polypeptide disclosed herein, wherein the exogenous nucleotide sequence comprises a sequence having at least 70% identity to SEQ ID NO: 16, 17, 18, or 19, or a codon degenerate nucleotide sequence thereof. In some embodiments, the cell comprises an exogenous nucleotide sequence coding for a recombinant polypeptide disclosed herein, wherein the exogenous nucleotide sequence comprises a sequence having at least 75%, 80%, 85%, 90%, 95%, 99%, 99.5%, or 99.9% identity to SEQ ID NO: 16, 17, 18, or 19, or a codon degenerate nucleotide sequence thereof. In some embodiments, the cell comprises an exogenous nucleotide sequence coding for a recombinant polypeptide disclosed herein, wherein the exogenous nucleotide sequence comprises SEQ ID NO: 16, 17, 18, or 19, or a codon degenerate nucleotide sequence thereof. In some embodiments, the cell comprises an exogenous nucleotide sequence coding for any recombinant polypeptide disclosed herein.

In some embodiments, the cell comprises an exogenous nucleotide sequence coding for a recombinant polypeptide comprising an amino acid sequence having at least 90% identity to SEQ ID NO: 5. In some embodiments, the cell comprises an exogenous nucleotide sequence coding for a recombinant polypeptide comprising an amino acid sequence having at least 75%, 80%, 85%, 90%, 95%, 99%, 99.5%, or 99.9% identity to SEQ ID NO: 5.

In some embodiments, the cell comprises an exogenous nucleotide sequence coding for a recombinant polypeptide comprising an amino acid sequence with at least one amino acid modification as compared to SEQ ID NO: 1, 3, or 4. In some embodiments, the cell comprises an exogenous nucleotide sequence coding for a recombinant polypeptide comprising an amino acid sequence with 1-20 amino acid modifications as compared to SEQ ID NO: 1, 2, 3, or 4. In some embodiments, the recombinant polypeptide further comprises a fusion domain. The fusion domain is not limited and may be any fusion domain disclosed herein. In some embodiments, the fusion domain is a domain useful for affinity chromatography. In some embodiments, the fusion domain targets the protein to a specific compartment of the cell such as the ER, vacuole, Golgi, peroxisome, lipid body (e.g., oleosome), or targets secretion of the protein from the cell into the outer membrane, periplasmic space or the culture media.

In some embodiments, the cell comprises an exogenous nucleotide sequence coding for a recombinant polypeptide comprising an amino acid sequence identical to SEQ ID NO: 1 with one to twenty amino acid substitutions and, optionally, one to twenty amino acids deleted from the C-terminus, wherein the amino acid substitutions are located in SEQ ID NO: 1 at positions selected from 49, 51, 52, 53, 65, 67, 68, 69, 111, 113, 122, 124, 125, 126, 164, 165, 166, 167, 170, 214, 215, 217, 219, 229, 231, 233, 235, 268, 269, 270, 282, 283, 284, 285, 286, 292, 293, 294, 295, 296, 297, and 298.

In some embodiments, the cell comprises an exogenous nucleotide sequence coding for a recombinant polypeptide comprising an amino acid sequence identical to SEQ ID NO: 2 with one to twenty amino acid substitutions and, optionally, one to twenty amino acids deleted from the C-terminus, wherein the amino acid substitutions are located in SEQ ID NO: 2 at positions selected from 38, 39, 40, 41, 42, 43, 44, 55, 57, 58, 59, 101, 103, 111, 112, 114, 115, 116, 117, 155, 156, 157, 160, 167, 169, 204, 205, 207, 209, 219, 221, 223, 224, 225, 258, 259, 260, 272, 273, 274, 275, 276, 282, 283, 284, 285, 286, 287, and 288.

In some embodiments, the cell comprises an exogenous nucleotide sequence coding for a recombinant polypeptide comprising an amino acid sequence identical to SEQ ID NO: 3 with one to twenty amino acid substitutions and, optionally, one to twenty amino acids deleted from the C-terminus, wherein the amino acid substitutions are located in SEQ ID NO: 3 at positions selected from 108, 109, 110, 111, 112, 113, 114, 125, 127, 128, 129, 171, 173, 181, 182, 184, 185, 186, 187, 225, 226, 227, 230, 237, 239, 274, 275, 277, 279, 289, 291, 293, 294, 295, 328, 329, 330, 342, 343, 344, 345, 346, 352, 353, 354, 355, 356, 357, and 358.

In some embodiments, the cell comprises an exogenous nucleotide sequence coding for a recombinant polypeptide comprising an amino acid sequence identical to SEQ ID NO: 4 with one to twenty amino acid substitutions and, optionally, one to twenty amino acids deleted from the C-terminus, wherein the amino acid substitutions are located in SEQ ID NO: 4 at positions selected from 38, 39, 40, 41, 42, 43, 44, 55, 57, 58, 59, 101, 103, 111, 112, 114, 115, 116, 117, 155, 156, 157, 160, 167, 169, 204, 205, 207, 209, 219, 221, 223, 224, 225, 258, 259, 260, 272, 273, 274, 275, 276, 282, 283, 284, 285, 286, 287, and 288.

In some embodiments, the recombinant polypeptide comprises an amino acid sequence identical to SEQ ID NO: 1, 2, 3, or 4 with one to twenty amino acid substitutions, and ten to sixteen amino acids deleted from the C-terminus. In some embodiments, the recombinant polypeptide comprises an amino acid sequence 90% identical to a sequence selected from SEQ ID NOs: 23-79 and 82-88. In some embodiments, the recombinant polypeptide comprises an amino acid sequence identical to a sequence selected from SEQ ID NOs: 23-79 and 82-88. In some embodiments, the sequence is selected from SEQ ID NOs: 29-36, 43, 56, 67, 69, 70, and 74.

In some embodiments, the recombinant polypeptide comprises an amino acid sequence at least 70% identical to SEQ ID NO: 23. In some embodiments, the recombinant polypeptide comprises an amino acid sequence at least 80% identical to SEQ ID NO: 23. In some embodiments, the recombinant polypeptide comprises an amino acid sequence at least 85% identical to SEQ ID NO: 23. In some embodiments, the recombinant polypeptide comprises an amino acid sequence at least 90% identical to SEQ ID NO: 23. In some embodiments, the recombinant polypeptide comprises an amino acid sequence at least 91% identical to SEQ ID NO: 23. In some embodiments, the recombinant polypeptide comprises an amino acid sequence at least 92% identical to SEQ ID NO: 23. In some embodiments, the recombinant polypeptide comprises an amino acid sequence at least 93% identical to SEQ ID NO: 23. In some embodiments, the recombinant polypeptide comprises an amino acid sequence at least 940% identical to SEQ ID NO: 23. In some embodiments, the recombinant polypeptide comprises an amino acid sequence at least 95% identical to SEQ ID NO: 23. In some embodiments, the recombinant polypeptide comprises an amino acid sequence at least 96% identical to SEQ ID NO: 23. In some embodiments, the recombinant polypeptide comprises an amino acid sequence at least 97% identical to SEQ ID NO: 23. In some embodiments, the recombinant polypeptide comprises an amino acid sequence at least 98% identical to SEQ ID NO: 23. In some embodiments, the recombinant polypeptide comprises an amino acid sequence at least 99% identical to SEQ ID NO: 23. In some embodiments, the recombinant polypeptide comprises an amino acid sequence identical to SEQ ID NO: 23. In some embodiments, the recombinant sequence does not comprise the n-terminal methionine. In some embodiments, the recombinant sequence does not comprise the n-terminal his tag sequence. In some embodiments, the recombinant sequence does not comprise the n-terminal methionine or the n-terminal his tag sequence.

In some embodiments, the recombinant polypeptide comprises an amino acid sequence at least 70% identical to SEQ ID NO: 24. In some embodiments, the recombinant polypeptide comprises an amino acid sequence at least 80% identical to SEQ ID NO: 24. In some embodiments, the recombinant polypeptide comprises an amino acid sequence at least 85% identical to SEQ ID NO: 24. In some embodiments, the recombinant polypeptide comprises an amino acid sequence at least 90% identical to SEQ ID NO: 24. In some embodiments, the recombinant polypeptide comprises an amino acid sequence at least 91% identical to SEQ ID NO: 24. In some embodiments, the recombinant polypeptide comprises an amino acid sequence at least 92% identical to SEQ ID NO: 24. In some embodiments, the recombinant polypeptide comprises an amino acid sequence at least 93% identical to SEQ ID NO: 24. In some embodiments, the recombinant polypeptide comprises an amino acid sequence at least 940% identical to SEQ ID NO: 24. In some embodiments, the recombinant polypeptide comprises an amino acid sequence at least 95% identical to SEQ ID NO: 24. In some embodiments, the recombinant polypeptide comprises an amino acid sequence at least 96% identical to SEQ ID NO: 24. In some embodiments, the recombinant polypeptide comprises an amino acid sequence at least 97% identical to SEQ ID NO: 24. In some embodiments, the recombinant polypeptide comprises an amino acid sequence at least 98% identical to SEQ ID NO: 24. In some embodiments, the recombinant polypeptide comprises an amino acid sequence at least 99% identical to SEQ ID NO: 24. In some embodiments, the recombinant polypeptide comprises an amino acid sequence identical to SEQ ID NO: 24. In some embodiments, the recombinant sequence does not comprise the n-terminal methionine. In some embodiments, the recombinant sequence does not comprise the n-terminal his tag sequence. In some embodiments, the recombinant sequence does not comprise the n-terminal methionine or the n-terminal his tag sequence.

In some embodiments, the recombinant polypeptide comprises an amino acid sequence at least 70% identical to SEQ ID NO: 25. In some embodiments, the recombinant polypeptide comprises an amino acid sequence at least 80% identical to SEQ ID NO: 25. In some embodiments, the recombinant polypeptide comprises an amino acid sequence at least 85% identical to SEQ ID NO: 25. In some embodiments, the recombinant polypeptide comprises an amino acid sequence at least 90% identical to SEQ ID NO: 25. In some embodiments, the recombinant polypeptide comprises an amino acid sequence at least 91% identical to SEQ ID NO: 25. In some embodiments, the recombinant polypeptide comprises an amino acid sequence at least 92% identical to SEQ ID NO: 25. In some embodiments, the recombinant polypeptide comprises an amino acid sequence at least 93% identical to SEQ ID NO: 25. In some embodiments, the recombinant polypeptide comprises an amino acid sequence at least 940% identical to SEQ ID NO: 25. In some embodiments, the recombinant polypeptide comprises an amino acid sequence at least 95% identical to SEQ ID NO: 25. In some embodiments, the recombinant polypeptide comprises an amino acid sequence at least 96% identical to SEQ ID NO: 25. In some embodiments, the recombinant polypeptide comprises an amino acid sequence at least 97% identical to SEQ ID NO: 25. In some embodiments, the recombinant polypeptide comprises an amino acid sequence at least 98% identical to SEQ ID NO: 25. In some embodiments, the recombinant polypeptide comprises an amino acid sequence at least 99% identical to SEQ ID NO: 25. In some embodiments, the recombinant polypeptide comprises an amino acid sequence identical to SEQ ID NO: 25. In some embodiments, the recombinant sequence does not comprise the n-terminal methionine. In some embodiments, the recombinant sequence does not comprise the n-terminal his tag sequence. In some embodiments, the recombinant sequence does not comprise the n-terminal methionine or the n-terminal his tag sequence.

In some embodiments, the recombinant polypeptide comprises an amino acid sequence at least 70% identical to SEQ ID NO: 26. In some embodiments, the recombinant polypeptide comprises an amino acid sequence at least 80% identical to SEQ ID NO: 26. In some embodiments, the recombinant polypeptide comprises an amino acid sequence at least 85% identical to SEQ ID NO: 26. In some embodiments, the recombinant polypeptide comprises an amino acid sequence at least 90% identical to SEQ ID NO: 26. In some embodiments, the recombinant polypeptide comprises an amino acid sequence at least 91% identical to SEQ ID NO: 26. In some embodiments, the recombinant polypeptide comprises an amino acid sequence at least 92% identical to SEQ ID NO: 26. In some embodiments, the recombinant polypeptide comprises an amino acid sequence at least 93% identical to SEQ ID NO: 26. In some embodiments, the recombinant polypeptide comprises an amino acid sequence at least 940% identical to SEQ ID NO: 26. In some embodiments, the recombinant polypeptide comprises an amino acid sequence at least 95% identical to SEQ ID NO: 26. In some embodiments, the recombinant polypeptide comprises an amino acid sequence at least 96% identical to SEQ ID NO: 26. In some embodiments, the recombinant polypeptide comprises an amino acid sequence at least 97% identical to SEQ ID NO: 26. In some embodiments, the recombinant polypeptide comprises an amino acid sequence at least 98% identical to SEQ ID NO: 26. In some embodiments, the recombinant polypeptide comprises an amino acid sequence at least 99% identical to SEQ ID NO: 26. In some embodiments, the recombinant polypeptide comprises an amino acid sequence identical to SEQ ID NO: 26. In some embodiments, the recombinant sequence does not comprise the n-terminal methionine. In some embodiments, the recombinant sequence does not comprise the n-terminal his tag sequence. In some embodiments, the recombinant sequence does not comprise the n-terminal methionine or the n-terminal his tag sequence.

In some embodiments, the recombinant polypeptide comprises an amino acid sequence at least 70% identical to SEQ ID NO: 27. In some embodiments, the recombinant polypeptide comprises an amino acid sequence at least 80% identical to SEQ ID NO: 27. In some embodiments, the recombinant polypeptide comprises an amino acid sequence at least 85% identical to SEQ ID NO: 27. In some embodiments, the recombinant polypeptide comprises an amino acid sequence at least 90% identical to SEQ ID NO: 27. In some embodiments, the recombinant polypeptide comprises an amino acid sequence at least 91% identical to SEQ ID NO: 27. In some embodiments, the recombinant polypeptide comprises an amino acid sequence at least 92% identical to SEQ ID NO: 27. In some embodiments, the recombinant polypeptide comprises an amino acid sequence at least 93% identical to SEQ ID NO: 27. In some embodiments, the recombinant polypeptide comprises an amino acid sequence at least 940% identical to SEQ ID NO: 27. In some embodiments, the recombinant polypeptide comprises an amino acid sequence at least 95% identical to SEQ ID NO: 27. In some embodiments, the recombinant polypeptide comprises an amino acid sequence at least 96% identical to SEQ ID NO: 27. In some embodiments, the recombinant polypeptide comprises an amino acid sequence at least 97% identical to SEQ ID NO: 27. In some embodiments, the recombinant polypeptide comprises an amino acid sequence at least 98% identical to SEQ ID NO: 27. In some embodiments, the recombinant polypeptide comprises an amino acid sequence at least 99% identical to SEQ ID NO: 27. In some embodiments, the recombinant polypeptide comprises an amino acid sequence identical to SEQ ID NO: 27. In some embodiments, the recombinant sequence does not comprise the n-terminal methionine. In some embodiments, the recombinant sequence does not comprise the n-terminal his tag sequence. In some embodiments, the recombinant sequence does not comprise the n-terminal methionine or the n-terminal his tag sequence.

In some embodiments, the recombinant polypeptide comprises an amino acid sequence at least 70% identical to SEQ ID NO: 28. In some embodiments, the recombinant polypeptide comprises an amino acid sequence at least 80% identical to SEQ ID NO: 28. In some embodiments, the recombinant polypeptide comprises an amino acid sequence at least 85% identical to SEQ ID NO: 28. In some embodiments, the recombinant polypeptide comprises an amino acid sequence at least 90% identical to SEQ ID NO: 28. In some embodiments, the recombinant polypeptide comprises an amino acid sequence at least 91% identical to SEQ ID NO: 28. In some embodiments, the recombinant polypeptide comprises an amino acid sequence at least 92% identical to SEQ ID NO: 28. In some embodiments, the recombinant polypeptide comprises an amino acid sequence at least 93% identical to SEQ ID NO: 28. In some embodiments, the recombinant polypeptide comprises an amino acid sequence at least 940% identical to SEQ ID NO: 28. In some embodiments, the recombinant polypeptide comprises an amino acid sequence at least 95% identical to SEQ ID NO: 28. In some embodiments, the recombinant polypeptide comprises an amino acid sequence at least 96% identical to SEQ ID NO: 28. In some embodiments, the recombinant polypeptide comprises an amino acid sequence at least 97% identical to SEQ ID NO: 28. In some embodiments, the recombinant polypeptide comprises an amino acid sequence at least 98% identical to SEQ ID NO: 28. In some embodiments, the recombinant polypeptide comprises an amino acid sequence at least 99% identical to SEQ ID NO: 28. In some embodiments, the recombinant polypeptide comprises an amino acid sequence identical to SEQ ID NO: 28. In some embodiments, the recombinant sequence does not comprise the n-terminal methionine. In some embodiments, the recombinant sequence does not comprise the n-terminal his tag sequence. In some embodiments, the recombinant sequence does not comprise the n-terminal methionine or the n-terminal his tag sequence.

In some embodiments, the recombinant polypeptide comprises an amino acid sequence at least 70% identical to SEQ ID NO: 29. In some embodiments, the recombinant polypeptide comprises an amino acid sequence at least 80% identical to SEQ ID NO: 29. In some embodiments, the recombinant polypeptide comprises an amino acid sequence at least 85% identical to SEQ ID NO: 29. In some embodiments, the recombinant polypeptide comprises an amino acid sequence at least 90% identical to SEQ ID NO: 29. In some embodiments, the recombinant polypeptide comprises an amino acid sequence at least 91% identical to SEQ ID NO: 29. In some embodiments, the recombinant polypeptide comprises an amino acid sequence at least 92% identical to SEQ ID NO: 29. In some embodiments, the recombinant polypeptide comprises an amino acid sequence at least 93% identical to SEQ ID NO: 29. In some embodiments, the recombinant polypeptide comprises an amino acid sequence at least 940% identical to SEQ ID NO: 29. In some embodiments, the recombinant polypeptide comprises an amino acid sequence at least 95% identical to SEQ ID NO: 29. In some embodiments, the recombinant polypeptide comprises an amino acid sequence at least 96% identical to SEQ ID NO: 29. In some embodiments, the recombinant polypeptide comprises an amino acid sequence at least 97% identical to SEQ ID NO: 29. In some embodiments, the recombinant polypeptide comprises an amino acid sequence at least 98% identical to SEQ ID NO: 29. In some embodiments, the recombinant polypeptide comprises an amino acid sequence at least 99% identical to SEQ ID NO: 29. In some embodiments, the recombinant polypeptide comprises an amino acid sequence identical to SEQ ID NO: 29. In some embodiments, the recombinant sequence does not comprise the n-terminal methionine. In some embodiments, the recombinant sequence does not comprise the n-terminal his tag sequence. In some embodiments, the recombinant sequence does not comprise the n-terminal methionine or the n-terminal his tag sequence.

In some embodiments, the recombinant polypeptide comprises an amino acid sequence at least 70% identical to SEQ ID NO: 30. In some embodiments, the recombinant polypeptide comprises an amino acid sequence at least 80% identical to SEQ ID NO: 30. In some embodiments, the recombinant polypeptide comprises an amino acid sequence at least 85% identical to SEQ ID NO: 30. In some embodiments, the recombinant polypeptide comprises an amino acid sequence at least 90% identical to SEQ ID NO: 30. In some embodiments, the recombinant polypeptide comprises an amino acid sequence at least 91% identical to SEQ ID NO: 30. In some embodiments, the recombinant polypeptide comprises an amino acid sequence at least 92% identical to SEQ ID NO: 30. In some embodiments, the recombinant polypeptide comprises an amino acid sequence at least 93% identical to SEQ ID NO: 30. In some embodiments, the recombinant polypeptide comprises an amino acid sequence at least 940% identical to SEQ ID NO: 30. In some embodiments, the recombinant polypeptide comprises an amino acid sequence at least 95% identical to SEQ ID NO: 30. In some embodiments, the recombinant polypeptide comprises an amino acid sequence at least 96% identical to SEQ ID NO: 30. In some embodiments, the recombinant polypeptide comprises an amino acid sequence at least 97% identical to SEQ ID NO: 30. In some embodiments, the recombinant polypeptide comprises an amino acid sequence at least 98% identical to SEQ ID NO: 30. In some embodiments, the recombinant polypeptide comprises an amino acid sequence at least 99% identical to SEQ ID NO: 30. In some embodiments, the recombinant polypeptide comprises an amino acid sequence identical to SEQ ID NO: 30. In some embodiments, the recombinant sequence does not comprise the n-terminal methionine. In some embodiments, the recombinant sequence does not comprise the n-terminal his tag sequence. In some embodiments, the recombinant sequence does not comprise the n-terminal methionine or the n-terminal his tag sequence.

In some embodiments, the recombinant polypeptide comprises an amino acid sequence at least 70% identical to SEQ ID NO: 31. In some embodiments, the recombinant polypeptide comprises an amino acid sequence at least 80% identical to SEQ ID NO: 31. In some embodiments, the recombinant polypeptide comprises an amino acid sequence at least 85% identical to SEQ ID NO: 31. In some embodiments, the recombinant polypeptide comprises an amino acid sequence at least 90% identical to SEQ ID NO: 31. In some embodiments, the recombinant polypeptide comprises an amino acid sequence at least 91% identical to SEQ ID NO: 31. In some embodiments, the recombinant polypeptide comprises an amino acid sequence at least 92% identical to SEQ ID NO: 31. In some embodiments, the recombinant polypeptide comprises an amino acid sequence at least 93% identical to SEQ ID NO: 31. In some embodiments, the recombinant polypeptide comprises an amino acid sequence at least 940% identical to SEQ ID NO: 31. In some embodiments, the recombinant polypeptide comprises an amino acid sequence at least 95% identical to SEQ ID NO: 31. In some embodiments, the recombinant polypeptide comprises an amino acid sequence at least 96% identical to SEQ ID NO: 31. In some embodiments, the recombinant polypeptide comprises an amino acid sequence at least 97% identical to SEQ ID NO: 31. In some embodiments, the recombinant polypeptide comprises an amino acid sequence at least 98% identical to SEQ ID NO: 31. In some embodiments, the recombinant polypeptide comprises an amino acid sequence at least 99% identical to SEQ ID NO: 31. In some embodiments, the recombinant polypeptide comprises an amino acid sequence identical to SEQ ID NO: 31. In some embodiments, the recombinant sequence does not comprise the n-terminal methionine. In some embodiments, the recombinant sequence does not comprise the n-terminal his tag sequence. In some embodiments, the recombinant sequence does not comprise the n-terminal methionine or the n-terminal his tag sequence.

In some embodiments, the recombinant polypeptide comprises an amino acid sequence at least 70% identical to SEQ ID NO: 32. In some embodiments, the recombinant polypeptide comprises an amino acid sequence at least 80% identical to SEQ ID NO: 32. In some embodiments, the recombinant polypeptide comprises an amino acid sequence at least 85% identical to SEQ ID NO: 32. In some embodiments, the recombinant polypeptide comprises an amino acid sequence at least 90% identical to SEQ ID NO: 32. In some embodiments, the recombinant polypeptide comprises an amino acid sequence at least 91% identical to SEQ ID NO: 32. In some embodiments, the recombinant polypeptide comprises an amino acid sequence at least 92% identical to SEQ ID NO: 32. In some embodiments, the recombinant polypeptide comprises an amino acid sequence at least 93% identical to SEQ ID NO: 32. In some embodiments, the recombinant polypeptide comprises an amino acid sequence at least 940% identical to SEQ ID NO: 32. In some embodiments, the recombinant polypeptide comprises an amino acid sequence at least 95% identical to SEQ ID NO: 32. In some embodiments, the recombinant polypeptide comprises an amino acid sequence at least 96% identical to SEQ ID NO: 32. In some embodiments, the recombinant polypeptide comprises an amino acid sequence at least 97% identical to SEQ ID NO: 32. In some embodiments, the recombinant polypeptide comprises an amino acid sequence at least 98% identical to SEQ ID NO: 32. In some embodiments, the recombinant polypeptide comprises an amino acid sequence at least 99% identical to SEQ ID NO: 32. In some embodiments, the recombinant polypeptide comprises an amino acid sequence identical to SEQ ID NO: 32. In some embodiments, the recombinant sequence does not comprise the n-terminal methionine. In some embodiments, the recombinant sequence does not comprise the n-terminal his tag sequence. In some embodiments, the recombinant sequence does not comprise the n-terminal methionine or the n-terminal his tag sequence.

In some embodiments, the recombinant polypeptide comprises an amino acid sequence at least 70% identical to SEQ ID NO: 33. In some embodiments, the recombinant polypeptide comprises an amino acid sequence at least 80% identical to SEQ ID NO: 33. In some embodiments, the recombinant polypeptide comprises an amino acid sequence at least 85% identical to SEQ ID NO: 33. In some embodiments, the recombinant polypeptide comprises an amino acid sequence at least 90% identical to SEQ ID NO: 33. In some embodiments, the recombinant polypeptide comprises an amino acid sequence at least 91% identical to SEQ ID NO: 33. In some embodiments, the recombinant polypeptide comprises an amino acid sequence at least 92% identical to SEQ ID NO: 33. In some embodiments, the recombinant polypeptide comprises an amino acid sequence at least 93% identical to SEQ ID NO: 33. In some embodiments, the recombinant polypeptide comprises an amino acid sequence at least 940% identical to SEQ ID NO: 33. In some embodiments, the recombinant polypeptide comprises an amino acid sequence at least 95% identical to SEQ ID NO: 33. In some embodiments, the recombinant polypeptide comprises an amino acid sequence at least 96% identical to SEQ ID NO: 33. In some embodiments, the recombinant polypeptide comprises an amino acid sequence at least 97% identical to SEQ ID NO: 33. In some embodiments, the recombinant polypeptide comprises an amino acid sequence at least 98% identical to SEQ ID NO: 33. In some embodiments, the recombinant polypeptide comprises an amino acid sequence at least 99% identical to SEQ ID NO: 33. In some embodiments, the recombinant polypeptide comprises an amino acid sequence identical to SEQ ID NO: 33. In some embodiments, the recombinant sequence does not comprise the n-terminal methionine. In some embodiments, the recombinant sequence does not comprise the n-terminal his tag sequence. In some embodiments, the recombinant sequence does not comprise the n-terminal methionine or the n-terminal his tag sequence.

In some embodiments, the recombinant polypeptide comprises an amino acid sequence at least 70% identical to SEQ ID NO: 34. In some embodiments, the recombinant polypeptide comprises an amino acid sequence at least 80% identical to SEQ ID NO: 34. In some embodiments, the recombinant polypeptide comprises an amino acid sequence at least 85% identical to SEQ ID NO: 34. In some embodiments, the recombinant polypeptide comprises an amino acid sequence at least 90% identical to SEQ ID NO: 34. In some embodiments, the recombinant polypeptide comprises an amino acid sequence at least 91% identical to SEQ ID NO: 34. In some embodiments, the recombinant polypeptide comprises an amino acid sequence at least 92% identical to SEQ ID NO: 34. In some embodiments, the recombinant polypeptide comprises an amino acid sequence at least 93% identical to SEQ ID NO: 34. In some embodiments, the recombinant polypeptide comprises an amino acid sequence at least 940% identical to SEQ ID NO: 34. In some embodiments, the recombinant polypeptide comprises an amino acid sequence at least 95% identical to SEQ ID NO: 34. In some embodiments, the recombinant polypeptide comprises an amino acid sequence at least 96% identical to SEQ ID NO: 34. In some embodiments, the recombinant polypeptide comprises an amino acid sequence at least 97% identical to SEQ ID NO: 34. In some embodiments, the recombinant polypeptide comprises an amino acid sequence at least 98% identical to SEQ ID NO: 34. In some embodiments, the recombinant polypeptide comprises an amino acid sequence at least 99% identical to SEQ ID NO: 34. In some embodiments, the recombinant polypeptide comprises an amino acid sequence identical to SEQ ID NO: 34. In some embodiments, the recombinant sequence does not comprise the n-terminal methionine. In some embodiments, the recombinant sequence does not comprise the n-terminal his tag sequence. In some embodiments, the recombinant sequence does not comprise the n-terminal methionine or the n-terminal his tag sequence.

In some embodiments, the recombinant polypeptide comprises an amino acid sequence at least 70% identical to SEQ ID NO: 35. In some embodiments, the recombinant polypeptide comprises an amino acid sequence at least 80% identical to SEQ ID NO: 35. In some embodiments, the recombinant polypeptide comprises an amino acid sequence at least 85% identical to SEQ ID NO: 35. In some embodiments, the recombinant polypeptide comprises an amino acid sequence at least 90% identical to SEQ ID NO: 35. In some embodiments, the recombinant polypeptide comprises an amino acid sequence at least 91% identical to SEQ ID NO: 35. In some embodiments, the recombinant polypeptide comprises an amino acid sequence at least 92% identical to SEQ ID NO: 35. In some embodiments, the recombinant polypeptide comprises an amino acid sequence at least 93% identical to SEQ ID NO: 35. In some embodiments, the recombinant polypeptide comprises an amino acid sequence at least 940% identical to SEQ ID NO: 35. In some embodiments, the recombinant polypeptide comprises an amino acid sequence at least 95% identical to SEQ ID NO: 35. In some embodiments, the recombinant polypeptide comprises an amino acid sequence at least 96% identical to SEQ ID NO: 35. In some embodiments, the recombinant polypeptide comprises an amino acid sequence at least 97% identical to SEQ ID NO: 35. In some embodiments, the recombinant polypeptide comprises an amino acid sequence at least 98% identical to SEQ ID NO: 35. In some embodiments, the recombinant polypeptide comprises an amino acid sequence at least 99% identical to SEQ ID NO: 35. In some embodiments, the recombinant polypeptide comprises an amino acid sequence identical to SEQ ID NO: 35. In some embodiments, the recombinant sequence does not comprise the n-terminal methionine. In some embodiments, the recombinant sequence does not comprise the n-terminal his tag sequence. In some embodiments, the recombinant sequence does not comprise the n-terminal methionine or the n-terminal his tag sequence.

In some embodiments, the recombinant polypeptide comprises an amino acid sequence at least 70% identical to SEQ ID NO: 36. In some embodiments, the recombinant polypeptide comprises an amino acid sequence at least 80% identical to SEQ ID NO: 36. In some embodiments, the recombinant polypeptide comprises an amino acid sequence at least 85% identical to SEQ ID NO: 36. In some embodiments, the recombinant polypeptide comprises an amino acid sequence at least 90% identical to SEQ ID NO: 36. In some embodiments, the recombinant polypeptide comprises an amino acid sequence at least 91% identical to SEQ ID NO: 36. In some embodiments, the recombinant polypeptide comprises an amino acid sequence at least 92% identical to SEQ ID NO: 36. In some embodiments, the recombinant polypeptide comprises an amino acid sequence at least 93% identical to SEQ ID NO: 36. In some embodiments, the recombinant polypeptide comprises an amino acid sequence at least 940% identical to SEQ ID NO: 36. In some embodiments, the recombinant polypeptide comprises an amino acid sequence at least 95% identical to SEQ ID NO: 36. In some embodiments, the recombinant polypeptide comprises an amino acid sequence at least 96% identical to SEQ ID NO: 36. In some embodiments, the recombinant polypeptide comprises an amino acid sequence at least 97% identical to SEQ ID NO: 36. In some embodiments, the recombinant polypeptide comprises an amino acid sequence at least 98% identical to SEQ ID NO: 36. In some embodiments, the recombinant polypeptide comprises an amino acid sequence at least 99% identical to SEQ ID NO: 36. In some embodiments, the recombinant polypeptide comprises an amino acid sequence identical to SEQ ID NO: 36. In some embodiments, the recombinant sequence does not comprise the n-terminal methionine. In some embodiments, the recombinant sequence does not comprise the n-terminal his tag sequence. In some embodiments, the recombinant sequence does not comprise the n-terminal methionine or the n-terminal his tag sequence.

In some embodiments, the recombinant polypeptide comprises an amino acid sequence at least 70% identical to SEQ ID NO: 37. In some embodiments, the recombinant polypeptide comprises an amino acid sequence at least 80% identical to SEQ ID NO: 37. In some embodiments, the recombinant polypeptide comprises an amino acid sequence at least 85% identical to SEQ ID NO: 37. In some embodiments, the recombinant polypeptide comprises an amino acid sequence at least 90% identical to SEQ ID NO: 37. In some embodiments, the recombinant polypeptide comprises an amino acid sequence at least 91% identical to SEQ ID NO: 37. In some embodiments, the recombinant polypeptide comprises an amino acid sequence at least 92% identical to SEQ ID NO: 37. In some embodiments, the recombinant polypeptide comprises an amino acid sequence at least 93% identical to SEQ ID NO: 37. In some embodiments, the recombinant polypeptide comprises an amino acid sequence at least 940% identical to SEQ ID NO: 37. In some embodiments, the recombinant polypeptide comprises an amino acid sequence at least 95% identical to SEQ ID NO: 37. In some embodiments, the recombinant polypeptide comprises an amino acid sequence at least 96% identical to SEQ ID NO: 37. In some embodiments, the recombinant polypeptide comprises an amino acid sequence at least 97% identical to SEQ ID NO: 37. In some embodiments, the recombinant polypeptide comprises an amino acid sequence at least 98% identical to SEQ ID NO: 37. In some embodiments, the recombinant polypeptide comprises an amino acid sequence at least 99% identical to SEQ ID NO: 37. In some embodiments, the recombinant polypeptide comprises an amino acid sequence identical to SEQ ID NO: 37. In some embodiments, the recombinant sequence does not comprise the n-terminal methionine. In some embodiments, the recombinant sequence does not comprise the n-terminal his tag sequence. In some embodiments, the recombinant sequence does not comprise the n-terminal methionine or the n-terminal his tag sequence.

In some embodiments, the recombinant polypeptide comprises an amino acid sequence at least 70% identical to SEQ ID NO: 38. In some embodiments, the recombinant polypeptide comprises an amino acid sequence at least 80% identical to SEQ ID NO: 38. In some embodiments, the recombinant polypeptide comprises an amino acid sequence at least 85% identical to SEQ ID NO: 38. In some embodiments, the recombinant polypeptide comprises an amino acid sequence at least 90% identical to SEQ ID NO: 38. In some embodiments, the recombinant polypeptide comprises an amino acid sequence at least 91% identical to SEQ ID NO: 38. In some embodiments, the recombinant polypeptide comprises an amino acid sequence at least 92% identical to SEQ ID NO: 38. In some embodiments, the recombinant polypeptide comprises an amino acid sequence at least 93% identical to SEQ ID NO: 38. In some embodiments, the recombinant polypeptide comprises an amino acid sequence at least 940% identical to SEQ ID NO: 38. In some embodiments, the recombinant polypeptide comprises an amino acid sequence at least 95% identical to SEQ ID NO: 38. In some embodiments, the recombinant polypeptide comprises an amino acid sequence at least 96% identical to SEQ ID NO: 38. In some embodiments, the recombinant polypeptide comprises an amino acid sequence at least 97% identical to SEQ ID NO: 38. In some embodiments, the recombinant polypeptide comprises an amino acid sequence at least 98% identical to SEQ ID NO: 38. In some embodiments, the recombinant polypeptide comprises an amino acid sequence at least 99% identical to SEQ ID NO: 38. In some embodiments, the recombinant polypeptide comprises an amino acid sequence identical to SEQ ID NO: 38. In some embodiments, the recombinant sequence does not comprise the n-terminal methionine. In some embodiments, the recombinant sequence does not comprise the n-terminal his tag sequence. In some embodiments, the recombinant sequence does not comprise the n-terminal methionine or the n-terminal his tag sequence.

In some embodiments, the recombinant polypeptide comprises an amino acid sequence at least 70% identical to SEQ ID NO: 39. In some embodiments, the recombinant polypeptide comprises an amino acid sequence at least 80% identical to SEQ ID NO: 39. In some embodiments, the recombinant polypeptide comprises an amino acid sequence at least 85% identical to SEQ ID NO: 39. In some embodiments, the recombinant polypeptide comprises an amino acid sequence at least 90% identical to SEQ ID NO: 39. In some embodiments, the recombinant polypeptide comprises an amino acid sequence at least 91% identical to SEQ ID NO: 39. In some embodiments, the recombinant polypeptide comprises an amino acid sequence at least 92% identical to SEQ ID NO: 39. In some embodiments, the recombinant polypeptide comprises an amino acid sequence at least 93% identical to SEQ ID NO: 39. In some embodiments, the recombinant polypeptide comprises an amino acid sequence at least 940% identical to SEQ ID NO: 39. In some embodiments, the recombinant polypeptide comprises an amino acid sequence at least 95% identical to SEQ ID NO: 39. In some embodiments, the recombinant polypeptide comprises an amino acid sequence at least 96% identical to SEQ ID NO: 39. In some embodiments, the recombinant polypeptide comprises an amino acid sequence at least 97% identical to SEQ ID NO: 39. In some embodiments, the recombinant polypeptide comprises an amino acid sequence at least 98% identical to SEQ ID NO: 39. In some embodiments, the recombinant polypeptide comprises an amino acid sequence at least 99% identical to SEQ ID NO: 39. In some embodiments, the recombinant polypeptide comprises an amino acid sequence identical to SEQ ID NO: 39. In some embodiments, the recombinant sequence does not comprise the n-terminal methionine. In some embodiments, the recombinant sequence does not comprise the n-terminal his tag sequence. In some embodiments, the recombinant sequence does not comprise the n-terminal methionine or the n-terminal his tag sequence.

In some embodiments, the recombinant polypeptide comprises an amino acid sequence at least 70% identical to SEQ ID NO: 40. In some embodiments, the recombinant polypeptide comprises an amino acid sequence at least 80% identical to SEQ ID NO: 40. In some embodiments, the recombinant polypeptide comprises an amino acid sequence at least 85% identical to SEQ ID NO: 40. In some embodiments, the recombinant polypeptide comprises an amino acid sequence at least 90% identical to SEQ ID NO: 40. In some embodiments, the recombinant polypeptide comprises an amino acid sequence at least 91% identical to SEQ ID NO: 40. In some embodiments, the recombinant polypeptide comprises an amino acid sequence at least 92% identical to SEQ ID NO: 40. In some embodiments, the recombinant polypeptide comprises an amino acid sequence at least 93% identical to SEQ ID NO: 40. In some embodiments, the recombinant polypeptide comprises an amino acid sequence at least 940% identical to SEQ ID NO: 40. In some embodiments, the recombinant polypeptide comprises an amino acid sequence at least 95% identical to SEQ ID NO: 40. In some embodiments, the recombinant polypeptide comprises an amino acid sequence at least 96% identical to SEQ ID NO: 40. In some embodiments, the recombinant polypeptide comprises an amino acid sequence at least 97% identical to SEQ ID NO: 40. In some embodiments, the recombinant polypeptide comprises an amino acid sequence at least 98% identical to SEQ ID NO: 40. In some embodiments, the recombinant polypeptide comprises an amino acid sequence at least 99% identical to SEQ ID NO: 40. In some embodiments, the recombinant polypeptide comprises an amino acid sequence identical to SEQ ID NO: 40. In some embodiments, the recombinant sequence does not comprise the n-terminal methionine. In some embodiments, the recombinant sequence does not comprise the n-terminal his tag sequence. In some embodiments, the recombinant sequence does not comprise the n-terminal methionine or the n-terminal his tag sequence.

In some embodiments, the recombinant polypeptide comprises an amino acid sequence at least 70% identical to SEQ ID NO: 41. In some embodiments, the recombinant polypeptide comprises an amino acid sequence at least 80% identical to SEQ ID NO: 41. In some embodiments, the recombinant polypeptide comprises an amino acid sequence at least 85% identical to SEQ ID NO: 41. In some embodiments, the recombinant polypeptide comprises an amino acid sequence at least 90% identical to SEQ ID NO: 41. In some embodiments, the recombinant polypeptide comprises an amino acid sequence at least 91% identical to SEQ ID NO: 41. In some embodiments, the recombinant polypeptide comprises an amino acid sequence at least 92% identical to SEQ ID NO: 41. In some embodiments, the recombinant polypeptide comprises an amino acid sequence at least 93% identical to SEQ ID NO: 41. In some embodiments, the recombinant polypeptide comprises an amino acid sequence at least 940% identical to SEQ ID NO: 41. In some embodiments, the recombinant polypeptide comprises an amino acid sequence at least 95% identical to SEQ ID NO: 41. In some embodiments, the recombinant polypeptide comprises an amino acid sequence at least 96% identical to SEQ ID NO: 41. In some embodiments, the recombinant polypeptide comprises an amino acid sequence at least 97% identical to SEQ ID NO: 41. In some embodiments, the recombinant polypeptide comprises an amino acid sequence at least 98% identical to SEQ ID NO: 41. In some embodiments, the recombinant polypeptide comprises an amino acid sequence at least 99% identical to SEQ ID NO: 41. In some embodiments, the recombinant polypeptide comprises an amino acid sequence identical to SEQ ID NO: 41. In some embodiments, the recombinant sequence does not comprise the n-terminal methionine. In some embodiments, the recombinant sequence does not comprise the n-terminal his tag sequence. In some embodiments, the recombinant sequence does not comprise the n-terminal methionine or the n-terminal his tag sequence.

In some embodiments, the recombinant polypeptide comprises an amino acid sequence at least 70% identical to SEQ ID NO: 42. In some embodiments, the recombinant polypeptide comprises an amino acid sequence at least 80% identical to SEQ ID NO: 42. In some embodiments, the recombinant polypeptide comprises an amino acid sequence at least 85% identical to SEQ ID NO: 42. In some embodiments, the recombinant polypeptide comprises an amino acid sequence at least 90% identical to SEQ ID NO: 42. In some embodiments, the recombinant polypeptide comprises an amino acid sequence at least 91% identical to SEQ ID NO: 42. In some embodiments, the recombinant polypeptide comprises an amino acid sequence at least 92% identical to SEQ ID NO: 42. In some embodiments, the recombinant polypeptide comprises an amino acid sequence at least 93% identical to SEQ ID NO: 42. In some embodiments, the recombinant polypeptide comprises an amino acid sequence at least 940% identical to SEQ ID NO: 42. In some embodiments, the recombinant polypeptide comprises an amino acid sequence at least 95% identical to SEQ ID NO: 42. In some embodiments, the recombinant polypeptide comprises an amino acid sequence at least 96% identical to SEQ ID NO: 42. In some embodiments, the recombinant polypeptide comprises an amino acid sequence at least 97% identical to SEQ ID NO: 42. In some embodiments, the recombinant polypeptide comprises an amino acid sequence at least 98% identical to SEQ ID NO: 42. In some embodiments, the recombinant polypeptide comprises an amino acid sequence at least 99% identical to SEQ ID NO: 42. In some embodiments, the recombinant polypeptide comprises an amino acid sequence identical to SEQ ID NO: 42. In some embodiments, the recombinant sequence does not comprise the n-terminal methionine. In some embodiments, the recombinant sequence does not comprise the n-terminal his tag sequence. In some embodiments, the recombinant sequence does not comprise the n-terminal methionine or the n-terminal his tag sequence.

In some embodiments, the recombinant polypeptide comprises an amino acid sequence at least 70% identical to SEQ ID NO: 43. In some embodiments, the recombinant polypeptide comprises an amino acid sequence at least 80% identical to SEQ ID NO: 43. In some embodiments, the recombinant polypeptide comprises an amino acid sequence at least 85% identical to SEQ ID NO: 43. In some embodiments, the recombinant polypeptide comprises an amino acid sequence at least 90% identical to SEQ ID NO: 43. In some embodiments, the recombinant polypeptide comprises an amino acid sequence at least 91% identical to SEQ ID NO: 43. In some embodiments, the recombinant polypeptide comprises an amino acid sequence at least 92% identical to SEQ ID NO: 43. In some embodiments, the recombinant polypeptide comprises an amino acid sequence at least 93% identical to SEQ ID NO: 43. In some embodiments, the recombinant polypeptide comprises an amino acid sequence at least 940% identical to SEQ ID NO: 43. In some embodiments, the recombinant polypeptide comprises an amino acid sequence at least 95% identical to SEQ ID NO: 43. In some embodiments, the recombinant polypeptide comprises an amino acid sequence at least 96% identical to SEQ ID NO: 43. In some embodiments, the recombinant polypeptide comprises an amino acid sequence at least 97% identical to SEQ ID NO: 43. In some embodiments, the recombinant polypeptide comprises an amino acid sequence at least 98% identical to SEQ ID NO: 43. In some embodiments, the recombinant polypeptide comprises an amino acid sequence at least 99% identical to SEQ ID NO: 43. In some embodiments, the recombinant polypeptide comprises an amino acid sequence identical to SEQ ID NO: 43. In some embodiments, the recombinant sequence does not comprise the n-terminal methionine. In some embodiments, the recombinant sequence does not comprise the n-terminal his tag sequence. In some embodiments, the recombinant sequence does not comprise the n-terminal methionine or the n-terminal his tag sequence.

In some embodiments, the recombinant polypeptide comprises an amino acid sequence at least 70% identical to SEQ ID NO: 44. In some embodiments, the recombinant polypeptide comprises an amino acid sequence at least 80% identical to SEQ ID NO: 44. In some embodiments, the recombinant polypeptide comprises an amino acid sequence at least 85% identical to SEQ ID NO: 44. In some embodiments, the recombinant polypeptide comprises an amino acid sequence at least 90% identical to SEQ ID NO: 44. In some embodiments, the recombinant polypeptide comprises an amino acid sequence at least 91% identical to SEQ ID NO: 44. In some embodiments, the recombinant polypeptide comprises an amino acid sequence at least 92% identical to SEQ ID NO: 44. In some embodiments, the recombinant polypeptide comprises an amino acid sequence at least 93% identical to SEQ ID NO: 44. In some embodiments, the recombinant polypeptide comprises an amino acid sequence at least 940% identical to SEQ ID NO: 44. In some embodiments, the recombinant polypeptide comprises an amino acid sequence at least 95% identical to SEQ ID NO: 44. In some embodiments, the recombinant polypeptide comprises an amino acid sequence at least 96% identical to SEQ ID NO: 44. In some embodiments, the recombinant polypeptide comprises an amino acid sequence at least 97% identical to SEQ ID NO: 44. In some embodiments, the recombinant polypeptide comprises an amino acid sequence at least 98% identical to SEQ ID NO: 44. In some embodiments, the recombinant polypeptide comprises an amino acid sequence at least 99% identical to SEQ ID NO: 44. In some embodiments, the recombinant polypeptide comprises an amino acid sequence identical to SEQ ID NO: 44. In some embodiments, the recombinant sequence does not comprise the n-terminal methionine. In some embodiments, the recombinant sequence does not comprise the n-terminal his tag sequence. In some embodiments, the recombinant sequence does not comprise the n-terminal methionine or the n-terminal his tag sequence.

In some embodiments, the recombinant polypeptide comprises an amino acid sequence at least 70% identical to SEQ ID NO: 45. In some embodiments, the recombinant polypeptide comprises an amino acid sequence at least 80% identical to SEQ ID NO: 45. In some embodiments, the recombinant polypeptide comprises an amino acid sequence at least 85% identical to SEQ ID NO: 45. In some embodiments, the recombinant polypeptide comprises an amino acid sequence at least 90% identical to SEQ ID NO: 45. In some embodiments, the recombinant polypeptide comprises an amino acid sequence at least 91% identical to SEQ ID NO: 45. In some embodiments, the recombinant polypeptide comprises an amino acid sequence at least 92% identical to SEQ ID NO: 45. In some embodiments, the recombinant polypeptide comprises an amino acid sequence at least 93% identical to SEQ ID NO: 45. In some embodiments, the recombinant polypeptide comprises an amino acid sequence at least 940% identical to SEQ ID NO: 45. In some embodiments, the recombinant polypeptide comprises an amino acid sequence at least 95% identical to SEQ ID NO: 45. In some embodiments, the recombinant polypeptide comprises an amino acid sequence at least 96% identical to SEQ ID NO: 45. In some embodiments, the recombinant polypeptide comprises an amino acid sequence at least 97% identical to SEQ ID NO: 45. In some embodiments, the recombinant polypeptide comprises an amino acid sequence at least 98% identical to SEQ ID NO: 45. In some embodiments, the recombinant polypeptide comprises an amino acid sequence at least 99% identical to SEQ ID NO: 45. In some embodiments, the recombinant polypeptide comprises an amino acid sequence identical to SEQ ID NO: 45. In some embodiments, the recombinant sequence does not comprise the n-terminal methionine. In some embodiments, the recombinant sequence does not comprise the n-terminal his tag sequence. In some embodiments, the recombinant sequence does not comprise the n-terminal methionine or the n-terminal his tag sequence.

In some embodiments, the recombinant polypeptide comprises an amino acid sequence at least 70% identical to SEQ ID NO: 46. In some embodiments, the recombinant polypeptide comprises an amino acid sequence at least 80% identical to SEQ ID NO: 46. In some embodiments, the recombinant polypeptide comprises an amino acid sequence at least 85% identical to SEQ ID NO: 46. In some embodiments, the recombinant polypeptide comprises an amino acid sequence at least 90% identical to SEQ ID NO: 46. In some embodiments, the recombinant polypeptide comprises an amino acid sequence at least 91% identical to SEQ ID NO: 46. In some embodiments, the recombinant polypeptide comprises an amino acid sequence at least 92% identical to SEQ ID NO: 46. In some embodiments, the recombinant polypeptide comprises an amino acid sequence at least 93% identical to SEQ ID NO: 46. In some embodiments, the recombinant polypeptide comprises an amino acid sequence at least 940% identical to SEQ ID NO: 46. In some embodiments, the recombinant polypeptide comprises an amino acid sequence at least 95% identical to SEQ ID NO: 46. In some embodiments, the recombinant polypeptide comprises an amino acid sequence at least 96% identical to SEQ ID NO: 46. In some embodiments, the recombinant polypeptide comprises an amino acid sequence at least 97% identical to SEQ ID NO: 46. In some embodiments, the recombinant polypeptide comprises an amino acid sequence at least 98% identical to SEQ ID NO: 46. In some embodiments, the recombinant polypeptide comprises an amino acid sequence at least 99% identical to SEQ ID NO: 46. In some embodiments, the recombinant polypeptide comprises an amino acid sequence identical to SEQ ID NO: 46. In some embodiments, the recombinant sequence does not comprise the n-terminal methionine. In some embodiments, the recombinant sequence does not comprise the n-terminal his tag sequence. In some embodiments, the recombinant sequence does not comprise the n-terminal methionine or the n-terminal his tag sequence.

In some embodiments, the recombinant polypeptide comprises an amino acid sequence at least 70% identical to SEQ ID NO: 47. In some embodiments, the recombinant polypeptide comprises an amino acid sequence at least 80% identical to SEQ ID NO: 47. In some embodiments, the recombinant polypeptide comprises an amino acid sequence at least 85% identical to SEQ ID NO: 47. In some embodiments, the recombinant polypeptide comprises an amino acid sequence at least 90% identical to SEQ ID NO: 47. In some embodiments, the recombinant polypeptide comprises an amino acid sequence at least 91% identical to SEQ ID NO: 47. In some embodiments, the recombinant polypeptide comprises an amino acid sequence at least 92% identical to SEQ ID NO: 47. In some embodiments, the recombinant polypeptide comprises an amino acid sequence at least 93% identical to SEQ ID NO: 47. In some embodiments, the recombinant polypeptide comprises an amino acid sequence at least 940% identical to SEQ ID NO: 47. In some embodiments, the recombinant polypeptide comprises an amino acid sequence at least 95% identical to SEQ ID NO: 47. In some embodiments, the recombinant polypeptide comprises an amino acid sequence at least 96% identical to SEQ ID NO: 47. In some embodiments, the recombinant polypeptide comprises an amino acid sequence at least 97% identical to SEQ ID NO: 47. In some embodiments, the recombinant polypeptide comprises an amino acid sequence at least 98% identical to SEQ ID NO: 47. In some embodiments, the recombinant polypeptide comprises an amino acid sequence at least 99% identical to SEQ ID NO: 47. In some embodiments, the recombinant polypeptide comprises an amino acid sequence identical to SEQ ID NO: 47. In some embodiments, the recombinant sequence does not comprise the n-terminal methionine. In some embodiments, the recombinant sequence does not comprise the n-terminal his tag sequence. In some embodiments, the recombinant sequence does not comprise the n-terminal methionine or the n-terminal his tag sequence.

In some embodiments, the recombinant polypeptide comprises an amino acid sequence at least 70% identical to SEQ ID NO: 48. In some embodiments, the recombinant polypeptide comprises an amino acid sequence at least 80% identical to SEQ ID NO: 48. In some embodiments, the recombinant polypeptide comprises an amino acid sequence at least 85% identical to SEQ ID NO: 48. In some embodiments, the recombinant polypeptide comprises an amino acid sequence at least 90% identical to SEQ ID NO: 48. In some embodiments, the recombinant polypeptide comprises an amino acid sequence at least 91% identical to SEQ ID NO: 48. In some embodiments, the recombinant polypeptide comprises an amino acid sequence at least 92% identical to SEQ ID NO: 48. In some embodiments, the recombinant polypeptide comprises an amino acid sequence at least 93% identical to SEQ ID NO: 48. In some embodiments, the recombinant polypeptide comprises an amino acid sequence at least 940% identical to SEQ ID NO: 48. In some embodiments, the recombinant polypeptide comprises an amino acid sequence at least 95% identical to SEQ ID NO: 48. In some embodiments, the recombinant polypeptide comprises an amino acid sequence at least 96% identical to SEQ ID NO: 48. In some embodiments, the recombinant polypeptide comprises an amino acid sequence at least 97% identical to SEQ ID NO: 48. In some embodiments, the recombinant polypeptide comprises an amino acid sequence at least 98% identical to SEQ ID NO: 48. In some embodiments, the recombinant polypeptide comprises an amino acid sequence at least 99% identical to SEQ ID NO: 48. In some embodiments, the recombinant polypeptide comprises an amino acid sequence identical to SEQ ID NO: 48. In some embodiments, the recombinant sequence does not comprise the n-terminal methionine. In some embodiments, the recombinant sequence does not comprise the n-terminal his tag sequence. In some embodiments, the recombinant sequence does not comprise the n-terminal methionine or the n-terminal his tag sequence.

In some embodiments, the recombinant polypeptide comprises an amino acid sequence at least 70% identical to SEQ ID NO: 49. In some embodiments, the recombinant polypeptide comprises an amino acid sequence at least 80% identical to SEQ ID NO: 49. In some embodiments, the recombinant polypeptide comprises an amino acid sequence at least 85% identical to SEQ ID NO: 49. In some embodiments, the recombinant polypeptide comprises an amino acid sequence at least 90% identical to SEQ ID NO: 49. In some embodiments, the recombinant polypeptide comprises an amino acid sequence at least 91% identical to SEQ ID NO: 49. In some embodiments, the recombinant polypeptide comprises an amino acid sequence at least 92% identical to SEQ ID NO: 49. In some embodiments, the recombinant polypeptide comprises an amino acid sequence at least 93% identical to SEQ ID NO: 49. In some embodiments, the recombinant polypeptide comprises an amino acid sequence at least 940% identical to SEQ ID NO: 49. In some embodiments, the recombinant polypeptide comprises an amino acid sequence at least 95% identical to SEQ ID NO: 49. In some embodiments, the recombinant polypeptide comprises an amino acid sequence at least 96% identical to SEQ ID NO: 49. In some embodiments, the recombinant polypeptide comprises an amino acid sequence at least 97% identical to SEQ ID NO: 49. In some embodiments, the recombinant polypeptide comprises an amino acid sequence at least 98% identical to SEQ ID NO: 49. In some embodiments, the recombinant polypeptide comprises an amino acid sequence at least 99% identical to SEQ ID NO: 49. In some embodiments, the recombinant polypeptide comprises an amino acid sequence identical to SEQ ID NO: 49. In some embodiments, the recombinant sequence does not comprise the n-terminal methionine. In some embodiments, the recombinant sequence does not comprise the n-terminal his tag sequence. In some embodiments, the recombinant sequence does not comprise the n-terminal methionine or the n-terminal his tag sequence.

In some embodiments, the recombinant polypeptide comprises an amino acid sequence at least 70% identical to SEQ ID NO: 50. In some embodiments, the recombinant polypeptide comprises an amino acid sequence at least 80% identical to SEQ ID NO: 50. In some embodiments, the recombinant polypeptide comprises an amino acid sequence at least 85% identical to SEQ ID NO: 50. In some embodiments, the recombinant polypeptide comprises an amino acid sequence at least 90% identical to SEQ ID NO: 50. In some embodiments, the recombinant polypeptide comprises an amino acid sequence at least 91% identical to SEQ ID NO: 50. In some embodiments, the recombinant polypeptide comprises an amino acid sequence at least 92% identical to SEQ ID NO: 50. In some embodiments, the recombinant polypeptide comprises an amino acid sequence at least 93% identical to SEQ ID NO: 50. In some embodiments, the recombinant polypeptide comprises an amino acid sequence at least 940% identical to SEQ ID NO: 50. In some embodiments, the recombinant polypeptide comprises an amino acid sequence at least 95% identical to SEQ ID NO: 50. In some embodiments, the recombinant polypeptide comprises an amino acid sequence at least 96% identical to SEQ ID NO: 50. In some embodiments, the recombinant polypeptide comprises an amino acid sequence at least 97% identical to SEQ ID NO: 50. In some embodiments, the recombinant polypeptide comprises an amino acid sequence at least 98% identical to SEQ ID NO: 50. In some embodiments, the recombinant polypeptide comprises an amino acid sequence at least 99% identical to SEQ ID NO: 50. In some embodiments, the recombinant polypeptide comprises an amino acid sequence identical to SEQ ID NO: 50. In some embodiments, the recombinant sequence does not comprise the n-terminal methionine. In some embodiments, the recombinant sequence does not comprise the n-terminal his tag sequence. In some embodiments, the recombinant sequence does not comprise the n-terminal methionine or the n-terminal his tag sequence.

In some embodiments, the recombinant polypeptide comprises an amino acid sequence at least 70% identical to SEQ ID NO: 51. In some embodiments, the recombinant polypeptide comprises an amino acid sequence at least 80% identical to SEQ ID NO: 51. In some embodiments, the recombinant polypeptide comprises an amino acid sequence at least 85% identical to SEQ ID NO: 51. In some embodiments, the recombinant polypeptide comprises an amino acid sequence at least 90% identical to SEQ ID NO: 51. In some embodiments, the recombinant polypeptide comprises an amino acid sequence at least 91% identical to SEQ ID NO: 51. In some embodiments, the recombinant polypeptide comprises an amino acid sequence at least 92% identical to SEQ ID NO: 51. In some embodiments, the recombinant polypeptide comprises an amino acid sequence at least 93% identical to SEQ ID NO: 51. In some embodiments, the recombinant polypeptide comprises an amino acid sequence at least 940% identical to SEQ ID NO: 51. In some embodiments, the recombinant polypeptide comprises an amino acid sequence at least 95% identical to SEQ ID NO: 51. In some embodiments, the recombinant polypeptide comprises an amino acid sequence at least 96% identical to SEQ ID NO: 51. In some embodiments, the recombinant polypeptide comprises an amino acid sequence at least 97% identical to SEQ ID NO: 51. In some embodiments, the recombinant polypeptide comprises an amino acid sequence at least 98% identical to SEQ ID NO: 51. In some embodiments, the recombinant polypeptide comprises an amino acid sequence at least 99% identical to SEQ ID NO: 51. In some embodiments, the recombinant polypeptide comprises an amino acid sequence identical to SEQ ID NO: 51. In some embodiments, the recombinant sequence does not comprise the n-terminal methionine. In some embodiments, the recombinant sequence does not comprise the n-terminal his tag sequence. In some embodiments, the recombinant sequence does not comprise the n-terminal methionine or the n-terminal his tag sequence.

In some embodiments, the recombinant polypeptide comprises an amino acid sequence at least 70% identical to SEQ ID NO: 52. In some embodiments, the recombinant polypeptide comprises an amino acid sequence at least 80% identical to SEQ ID NO: 52. In some embodiments, the recombinant polypeptide comprises an amino acid sequence at least 85% identical to SEQ ID NO: 52. In some embodiments, the recombinant polypeptide comprises an amino acid sequence at least 90% identical to SEQ ID NO: 52. In some embodiments, the recombinant polypeptide comprises an amino acid sequence at least 91% identical to SEQ ID NO: 52. In some embodiments, the recombinant polypeptide comprises an amino acid sequence at least 92% identical to SEQ ID NO: 52. In some embodiments, the recombinant polypeptide comprises an amino acid sequence at least 93% identical to SEQ ID NO: 52. In some embodiments, the recombinant polypeptide comprises an amino acid sequence at least 940% identical to SEQ ID NO: 52. In some embodiments, the recombinant polypeptide comprises an amino acid sequence at least 95% identical to SEQ ID NO: 52. In some embodiments, the recombinant polypeptide comprises an amino acid sequence at least 96% identical to SEQ ID NO: 52. In some embodiments, the recombinant polypeptide comprises an amino acid sequence at least 97% identical to SEQ ID NO: 52. In some embodiments, the recombinant polypeptide comprises an amino acid sequence at least 98% identical to SEQ ID NO: 52. In some embodiments, the recombinant polypeptide comprises an amino acid sequence at least 99% identical to SEQ ID NO: 52. In some embodiments, the recombinant polypeptide comprises an amino acid sequence identical to SEQ ID NO: 52. In some embodiments, the recombinant sequence does not comprise the n-terminal methionine. In some embodiments, the recombinant sequence does not comprise the n-terminal his tag sequence. In some embodiments, the recombinant sequence does not comprise the n-terminal methionine or the n-terminal his tag sequence.

In some embodiments, the recombinant polypeptide comprises an amino acid sequence at least 70% identical to SEQ ID NO: 53. In some embodiments, the recombinant polypeptide comprises an amino acid sequence at least 80% identical to SEQ ID NO: 53. In some embodiments, the recombinant polypeptide comprises an amino acid sequence at least 85% identical to SEQ ID NO: 53. In some embodiments, the recombinant polypeptide comprises an amino acid sequence at least 90% identical to SEQ ID NO: 53. In some embodiments, the recombinant polypeptide comprises an amino acid sequence at least 91% identical to SEQ ID NO: 53. In some embodiments, the recombinant polypeptide comprises an amino acid sequence at least 92% identical to SEQ ID NO: 53. In some embodiments, the recombinant polypeptide comprises an amino acid sequence at least 93% identical to SEQ ID NO: 53. In some embodiments, the recombinant polypeptide comprises an amino acid sequence at least 940% identical to SEQ ID NO: 53. In some embodiments, the recombinant polypeptide comprises an amino acid sequence at least 95% identical to SEQ ID NO: 53. In some embodiments, the recombinant polypeptide comprises an amino acid sequence at least 96% identical to SEQ ID NO: 53. In some embodiments, the recombinant polypeptide comprises an amino acid sequence at least 97% identical to SEQ ID NO: 53. In some embodiments, the recombinant polypeptide comprises an amino acid sequence at least 98% identical to SEQ ID NO: 53. In some embodiments, the recombinant polypeptide comprises an amino acid sequence at least 99% identical to SEQ ID NO: 53. In some embodiments, the recombinant polypeptide comprises an amino acid sequence identical to SEQ ID NO: 53. In some embodiments, the recombinant sequence does not comprise the n-terminal methionine. In some embodiments, the recombinant sequence does not comprise the n-terminal his tag sequence. In some embodiments, the recombinant sequence does not comprise the n-terminal methionine or the n-terminal his tag sequence.

In some embodiments, the recombinant polypeptide comprises an amino acid sequence at least 70% identical to SEQ ID NO: 54. In some embodiments, the recombinant polypeptide comprises an amino acid sequence at least 80% identical to SEQ ID NO: 54. In some embodiments, the recombinant polypeptide comprises an amino acid sequence at least 85% identical to SEQ ID NO: 54. In some embodiments, the recombinant polypeptide comprises an amino acid sequence at least 90% identical to SEQ ID NO: 54. In some embodiments, the recombinant polypeptide comprises an amino acid sequence at least 91% identical to SEQ ID NO: 54. In some embodiments, the recombinant polypeptide comprises an amino acid sequence at least 92% identical to SEQ ID NO: 54. In some embodiments, the recombinant polypeptide comprises an amino acid sequence at least 93% identical to SEQ ID NO: 54. In some embodiments, the recombinant polypeptide comprises an amino acid sequence at least 940% identical to SEQ ID NO: 54. In some embodiments, the recombinant polypeptide comprises an amino acid sequence at least 95% identical to SEQ ID NO: 54. In some embodiments, the recombinant polypeptide comprises an amino acid sequence at least 96% identical to SEQ ID NO: 54. In some embodiments, the recombinant polypeptide comprises an amino acid sequence at least 97% identical to SEQ ID NO: 54. In some embodiments, the recombinant polypeptide comprises an amino acid sequence at least 98% identical to SEQ ID NO: 54. In some embodiments, the recombinant polypeptide comprises an amino acid sequence at least 99% identical to SEQ ID NO: 54. In some embodiments, the recombinant polypeptide comprises an amino acid sequence identical to SEQ ID NO: 54. In some embodiments, the recombinant sequence does not comprise the n-terminal methionine. In some embodiments, the recombinant sequence does not comprise the n-terminal his tag sequence. In some embodiments, the recombinant sequence does not comprise the n-terminal methionine or the n-terminal his tag sequence.

In some embodiments, the recombinant polypeptide comprises an amino acid sequence at least 70% identical to SEQ ID NO: 55. In some embodiments, the recombinant polypeptide comprises an amino acid sequence at least 80% identical to SEQ ID NO: 55. In some embodiments, the recombinant polypeptide comprises an amino acid sequence at least 85% identical to SEQ ID NO: 55. In some embodiments, the recombinant polypeptide comprises an amino acid sequence at least 90% identical to SEQ ID NO: 55. In some embodiments, the recombinant polypeptide comprises an amino acid sequence at least 91% identical to SEQ ID NO: 55. In some embodiments, the recombinant polypeptide comprises an amino acid sequence at least 92% identical to SEQ ID NO: 55. In some embodiments, the recombinant polypeptide comprises an amino acid sequence at least 93% identical to SEQ ID NO: 55. In some embodiments, the recombinant polypeptide comprises an amino acid sequence at least 940% identical to SEQ ID NO: 55. In some embodiments, the recombinant polypeptide comprises an amino acid sequence at least 95% identical to SEQ ID NO: 55. In some embodiments, the recombinant polypeptide comprises an amino acid sequence at least 96% identical to SEQ ID NO: 55. In some embodiments, the recombinant polypeptide comprises an amino acid sequence at least 97% identical to SEQ ID NO: 55. In some embodiments, the recombinant polypeptide comprises an amino acid sequence at least 98% identical to SEQ ID NO: 55. In some embodiments, the recombinant polypeptide comprises an amino acid sequence at least 99% identical to SEQ ID NO: 55. In some embodiments, the recombinant polypeptide comprises an amino acid sequence identical to SEQ ID NO: 55. In some embodiments, the recombinant sequence does not comprise the n-terminal methionine. In some embodiments, the recombinant sequence does not comprise the n-terminal his tag sequence. In some embodiments, the recombinant sequence does not comprise the n-terminal methionine or the n-terminal his tag sequence.

In some embodiments, the recombinant polypeptide comprises an amino acid sequence at least 70% identical to SEQ ID NO: 56. In some embodiments, the recombinant polypeptide comprises an amino acid sequence at least 80% identical to SEQ ID NO: 56. In some embodiments, the recombinant polypeptide comprises an amino acid sequence at least 85% identical to SEQ ID NO: 56. In some embodiments, the recombinant polypeptide comprises an amino acid sequence at least 90% identical to SEQ ID NO: 56. In some embodiments, the recombinant polypeptide comprises an amino acid sequence at least 91% identical to SEQ ID NO: 56. In some embodiments, the recombinant polypeptide comprises an amino acid sequence at least 92% identical to SEQ ID NO: 56. In some embodiments, the recombinant polypeptide comprises an amino acid sequence at least 93% identical to SEQ ID NO: 56. In some embodiments, the recombinant polypeptide comprises an amino acid sequence at least 940% identical to SEQ ID NO: 56. In some embodiments, the recombinant polypeptide comprises an amino acid sequence at least 95% identical to SEQ ID NO: 56. In some embodiments, the recombinant polypeptide comprises an amino acid sequence at least 96% identical to SEQ ID NO: 56. In some embodiments, the recombinant polypeptide comprises an amino acid sequence at least 97% identical to SEQ ID NO: 56. In some embodiments, the recombinant polypeptide comprises an amino acid sequence at least 98% identical to SEQ ID NO: 56. In some embodiments, the recombinant polypeptide comprises an amino acid sequence at least 99% identical to SEQ ID NO: 56. In some embodiments, the recombinant polypeptide comprises an amino acid sequence identical to SEQ ID NO: 56. In some embodiments, the recombinant sequence does not comprise the n-terminal methionine. In some embodiments, the recombinant sequence does not comprise the n-terminal his tag sequence. In some embodiments, the recombinant sequence does not comprise the n-terminal methionine or the n-terminal his tag sequence.

In some embodiments, the recombinant polypeptide comprises an amino acid sequence at least 70% identical to SEQ ID NO: 57. In some embodiments, the recombinant polypeptide comprises an amino acid sequence at least 80% identical to SEQ ID NO: 57. In some embodiments, the recombinant polypeptide comprises an amino acid sequence at least 85% identical to SEQ ID NO: 57. In some embodiments, the recombinant polypeptide comprises an amino acid sequence at least 90% identical to SEQ ID NO: 57. In some embodiments, the recombinant polypeptide comprises an amino acid sequence at least 91% identical to SEQ ID NO: 57. In some embodiments, the recombinant polypeptide comprises an amino acid sequence at least 92% identical to SEQ ID NO: 57. In some embodiments, the recombinant polypeptide comprises an amino acid sequence at least 93% identical to SEQ ID NO: 57. In some embodiments, the recombinant polypeptide comprises an amino acid sequence at least 940% identical to SEQ ID NO: 57. In some embodiments, the recombinant polypeptide comprises an amino acid sequence at least 95% identical to SEQ ID NO: 57. In some embodiments, the recombinant polypeptide comprises an amino acid sequence at least 96% identical to SEQ ID NO: 57. In some embodiments, the recombinant polypeptide comprises an amino acid sequence at least 97% identical to SEQ ID NO: 57. In some embodiments, the recombinant polypeptide comprises an amino acid sequence at least 98% identical to SEQ ID NO: 57. In some embodiments, the recombinant polypeptide comprises an amino acid sequence at least 99% identical to SEQ ID NO: 57. In some embodiments, the recombinant polypeptide comprises an amino acid sequence identical to SEQ ID NO: 57. In some embodiments, the recombinant sequence does not comprise the n-terminal methionine. In some embodiments, the recombinant sequence does not comprise the n-terminal his tag sequence. In some embodiments, the recombinant sequence does not comprise the n-terminal methionine or the n-terminal his tag sequence.

In some embodiments, the recombinant polypeptide comprises an amino acid sequence at least 70% identical to SEQ ID NO: 58. In some embodiments, the recombinant polypeptide comprises an amino acid sequence at least 80% identical to SEQ ID NO: 58. In some embodiments, the recombinant polypeptide comprises an amino acid sequence at least 85% identical to SEQ ID NO: 58. In some embodiments, the recombinant polypeptide comprises an amino acid sequence at least 90% identical to SEQ ID NO: 58. In some embodiments, the recombinant polypeptide comprises an amino acid sequence at least 91% identical to SEQ ID NO: 58. In some embodiments, the recombinant polypeptide comprises an amino acid sequence at least 92% identical to SEQ ID NO: 58. In some embodiments, the recombinant polypeptide comprises an amino acid sequence at least 93% identical to SEQ ID NO: 58. In some embodiments, the recombinant polypeptide comprises an amino acid sequence at least 940% identical to SEQ ID NO: 58. In some embodiments, the recombinant polypeptide comprises an amino acid sequence at least 95% identical to SEQ ID NO: 58. In some embodiments, the recombinant polypeptide comprises an amino acid sequence at least 96% identical to SEQ ID NO: 58. In some embodiments, the recombinant polypeptide comprises an amino acid sequence at least 97% identical to SEQ ID NO: 58. In some embodiments, the recombinant polypeptide comprises an amino acid sequence at least 98% identical to SEQ ID NO: 58. In some embodiments, the recombinant polypeptide comprises an amino acid sequence at least 99% identical to SEQ ID NO: 58. In some embodiments, the recombinant polypeptide comprises an amino acid sequence identical to SEQ ID NO: 58. In some embodiments, the recombinant sequence does not comprise the n-terminal methionine. In some embodiments, the recombinant sequence does not comprise the n-terminal his tag sequence. In some embodiments, the recombinant sequence does not comprise the n-terminal methionine or the n-terminal his tag sequence.

In some embodiments, the recombinant polypeptide comprises an amino acid sequence at least 70% identical to SEQ ID NO: 59. In some embodiments, the recombinant polypeptide comprises an amino acid sequence at least 80% identical to SEQ ID NO: 59. In some embodiments, the recombinant polypeptide comprises an amino acid sequence at least 85% identical to SEQ ID NO: 59. In some embodiments, the recombinant polypeptide comprises an amino acid sequence at least 90% identical to SEQ ID NO: 59. In some embodiments, the recombinant polypeptide comprises an amino acid sequence at least 91% identical to SEQ ID NO: 59. In some embodiments, the recombinant polypeptide comprises an amino acid sequence at least 92% identical to SEQ ID NO: 59. In some embodiments, the recombinant polypeptide comprises an amino acid sequence at least 93% identical to SEQ ID NO: 59. In some embodiments, the recombinant polypeptide comprises an amino acid sequence at least 940% identical to SEQ ID NO: 59. In some embodiments, the recombinant polypeptide comprises an amino acid sequence at least 95% identical to SEQ ID NO: 59. In some embodiments, the recombinant polypeptide comprises an amino acid sequence at least 96% identical to SEQ ID NO: 59. In some embodiments, the recombinant polypeptide comprises an amino acid sequence at least 97% identical to SEQ ID NO: 59. In some embodiments, the recombinant polypeptide comprises an amino acid sequence at least 98% identical to SEQ ID NO: 59. In some embodiments, the recombinant polypeptide comprises an amino acid sequence at least 99% identical to SEQ ID NO: 59. In some embodiments, the recombinant polypeptide comprises an amino acid sequence identical to SEQ ID NO: 59. In some embodiments, the recombinant sequence does not comprise the n-terminal methionine. In some embodiments, the recombinant sequence does not comprise the n-terminal his tag sequence. In some embodiments, the recombinant sequence does not comprise the n-terminal methionine or the n-terminal his tag sequence.

In some embodiments, the recombinant polypeptide comprises an amino acid sequence at least 70% identical to SEQ ID NO: 60. In some embodiments, the recombinant polypeptide comprises an amino acid sequence at least 80% identical to SEQ ID NO: 60. In some embodiments, the recombinant polypeptide comprises an amino acid sequence at least 85% identical to SEQ ID NO: 60. In some embodiments, the recombinant polypeptide comprises an amino acid sequence at least 90% identical to SEQ ID NO: 60. In some embodiments, the recombinant polypeptide comprises an amino acid sequence at least 91% identical to SEQ ID NO: 60. In some embodiments, the recombinant polypeptide comprises an amino acid sequence at least 92% identical to SEQ ID NO: 60. In some embodiments, the recombinant polypeptide comprises an amino acid sequence at least 93% identical to SEQ ID NO: 60. In some embodiments, the recombinant polypeptide comprises an amino acid sequence at least 940% identical to SEQ ID NO: 60. In some embodiments, the recombinant polypeptide comprises an amino acid sequence at least 95% identical to SEQ ID NO: 60. In some embodiments, the recombinant polypeptide comprises an amino acid sequence at least 96% identical to SEQ ID NO: 60. In some embodiments, the recombinant polypeptide comprises an amino acid sequence at least 97% identical to SEQ ID NO: 60. In some embodiments, the recombinant polypeptide comprises an amino acid sequence at least 98% identical to SEQ ID NO: 60. In some embodiments, the recombinant polypeptide comprises an amino acid sequence at least 99% identical to SEQ ID NO: 60. In some embodiments, the recombinant polypeptide comprises an amino acid sequence identical to SEQ ID NO: 60. In some embodiments, the recombinant sequence does not comprise the n-terminal methionine. In some embodiments, the recombinant sequence does not comprise the n-terminal his tag sequence. In some embodiments, the recombinant sequence does not comprise the n-terminal methionine or the n-terminal his tag sequence.

In some embodiments, the recombinant polypeptide comprises an amino acid sequence at least 70% identical to SEQ ID NO: 61. In some embodiments, the recombinant polypeptide comprises an amino acid sequence at least 80% identical to SEQ ID NO: 61. In some embodiments, the recombinant polypeptide comprises an amino acid sequence at least 85% identical to SEQ ID NO: 61. In some embodiments, the recombinant polypeptide comprises an amino acid sequence at least 90% identical to SEQ ID NO: 61. In some embodiments, the recombinant polypeptide comprises an amino acid sequence at least 91% identical to SEQ ID NO: 61. In some embodiments, the recombinant polypeptide comprises an amino acid sequence at least 92% identical to SEQ ID NO: 61. In some embodiments, the recombinant polypeptide comprises an amino acid sequence at least 93% identical to SEQ ID NO: 61. In some embodiments, the recombinant polypeptide comprises an amino acid sequence at least 940% identical to SEQ ID NO: 61. In some embodiments, the recombinant polypeptide comprises an amino acid sequence at least 95% identical to SEQ ID NO: 61. In some embodiments, the recombinant polypeptide comprises an amino acid sequence at least 96% identical to SEQ ID NO: 61. In some embodiments, the recombinant polypeptide comprises an amino acid sequence at least 97% identical to SEQ ID NO: 61. In some embodiments, the recombinant polypeptide comprises an amino acid sequence at least 98% identical to SEQ ID NO: 61. In some embodiments, the recombinant polypeptide comprises an amino acid sequence at least 99% identical to SEQ ID NO: 61. In some embodiments, the recombinant polypeptide comprises an amino acid sequence identical to SEQ ID NO: 61. In some embodiments, the recombinant sequence does not comprise the n-terminal methionine. In some embodiments, the recombinant sequence does not comprise the n-terminal his tag sequence. In some embodiments, the recombinant sequence does not comprise the n-terminal methionine or the n-terminal his tag sequence.

In some embodiments, the recombinant polypeptide comprises an amino acid sequence at least 70% identical to SEQ ID NO: 62. In some embodiments, the recombinant polypeptide comprises an amino acid sequence at least 80% identical to SEQ ID NO: 62. In some embodiments, the recombinant polypeptide comprises an amino acid sequence at least 85% identical to SEQ ID NO: 62. In some embodiments, the recombinant polypeptide comprises an amino acid sequence at least 90% identical to SEQ ID NO: 62. In some embodiments, the recombinant polypeptide comprises an amino acid sequence at least 91% identical to SEQ ID NO: 62. In some embodiments, the recombinant polypeptide comprises an amino acid sequence at least 92% identical to SEQ ID NO: 62. In some embodiments, the recombinant polypeptide comprises an amino acid sequence at least 93% identical to SEQ ID NO: 62. In some embodiments, the recombinant polypeptide comprises an amino acid sequence at least 940% identical to SEQ ID NO: 62. In some embodiments, the recombinant polypeptide comprises an amino acid sequence at least 95% identical to SEQ ID NO: 62. In some embodiments, the recombinant polypeptide comprises an amino acid sequence at least 96% identical to SEQ ID NO: 62. In some embodiments, the recombinant polypeptide comprises an amino acid sequence at least 97% identical to SEQ ID NO: 62. In some embodiments, the recombinant polypeptide comprises an amino acid sequence at least 98% identical to SEQ ID NO: 62. In some embodiments, the recombinant polypeptide comprises an amino acid sequence at least 99% identical to SEQ ID NO: 62. In some embodiments, the recombinant polypeptide comprises an amino acid sequence identical to SEQ ID NO: 62. In some embodiments, the recombinant sequence does not comprise the n-terminal methionine. In some embodiments, the recombinant sequence does not comprise the n-terminal his tag sequence. In some embodiments, the recombinant sequence does not comprise the n-terminal methionine or the n-terminal his tag sequence.

In some embodiments, the recombinant polypeptide comprises an amino acid sequence at least 70% identical to SEQ ID NO: 63. In some embodiments, the recombinant polypeptide comprises an amino acid sequence at least 80% identical to SEQ ID NO: 63. In some embodiments, the recombinant polypeptide comprises an amino acid sequence at least 85% identical to SEQ ID NO: 63. In some embodiments, the recombinant polypeptide comprises an amino acid sequence at least 90% identical to SEQ ID NO: 63. In some embodiments, the recombinant polypeptide comprises an amino acid sequence at least 91% identical to SEQ ID NO: 63. In some embodiments, the recombinant polypeptide comprises an amino acid sequence at least 92% identical to SEQ ID NO: 63. In some embodiments, the recombinant polypeptide comprises an amino acid sequence at least 93% identical to SEQ ID NO: 63. In some embodiments, the recombinant polypeptide comprises an amino acid sequence at least 940% identical to SEQ ID NO: 63. In some embodiments, the recombinant polypeptide comprises an amino acid sequence at least 95% identical to SEQ ID NO: 63. In some embodiments, the recombinant polypeptide comprises an amino acid sequence at least 96% identical to SEQ ID NO: 63. In some embodiments, the recombinant polypeptide comprises an amino acid sequence at least 97% identical to SEQ ID NO: 63. In some embodiments, the recombinant polypeptide comprises an amino acid sequence at least 98% identical to SEQ ID NO: 63. In some embodiments, the recombinant polypeptide comprises an amino acid sequence at least 99% identical to SEQ ID NO: 63. In some embodiments, the recombinant polypeptide comprises an amino acid sequence identical to SEQ ID NO: 63. In some embodiments, the recombinant sequence does not comprise the n-terminal methionine. In some embodiments, the recombinant sequence does not comprise the n-terminal his tag sequence. In some embodiments, the recombinant sequence does not comprise the n-terminal methionine or the n-terminal his tag sequence.

In some embodiments, the recombinant polypeptide comprises an amino acid sequence at least 70% identical to SEQ ID NO: 64. In some embodiments, the recombinant polypeptide comprises an amino acid sequence at least 80% identical to SEQ ID NO: 64. In some embodiments, the recombinant polypeptide comprises an amino acid sequence at least 85% identical to SEQ ID NO: 64. In some embodiments, the recombinant polypeptide comprises an amino acid sequence at least 90% identical to SEQ ID NO: 64. In some embodiments, the recombinant polypeptide comprises an amino acid sequence at least 91% identical to SEQ ID NO: 64. In some embodiments, the recombinant polypeptide comprises an amino acid sequence at least 92% identical to SEQ ID NO: 64. In some embodiments, the recombinant polypeptide comprises an amino acid sequence at least 93% identical to SEQ ID NO: 64. In some embodiments, the recombinant polypeptide comprises an amino acid sequence at least 940% identical to SEQ ID NO: 64. In some embodiments, the recombinant polypeptide comprises an amino acid sequence at least 95% identical to SEQ ID NO: 64. In some embodiments, the recombinant polypeptide comprises an amino acid sequence at least 96% identical to SEQ ID NO: 64. In some embodiments, the recombinant polypeptide comprises an amino acid sequence at least 97% identical to SEQ ID NO: 64. In some embodiments, the recombinant polypeptide comprises an amino acid sequence at least 98% identical to SEQ ID NO: 64. In some embodiments, the recombinant polypeptide comprises an amino acid sequence at least 99% identical to SEQ ID NO: 64. In some embodiments, the recombinant polypeptide comprises an amino acid sequence identical to SEQ ID NO: 64. In some embodiments, the recombinant sequence does not comprise the n-terminal methionine. In some embodiments, the recombinant sequence does not comprise the n-terminal his tag sequence. In some embodiments, the recombinant sequence does not comprise the n-terminal methionine or the n-terminal his tag sequence.

In some embodiments, the recombinant polypeptide comprises an amino acid sequence at least 70% identical to SEQ ID NO: 65. In some embodiments, the recombinant polypeptide comprises an amino acid sequence at least 80% identical to SEQ ID NO: 65. In some embodiments, the recombinant polypeptide comprises an amino acid sequence at least 85% identical to SEQ ID NO: 65. In some embodiments, the recombinant polypeptide comprises an amino acid sequence at least 90% identical to SEQ ID NO: 65. In some embodiments, the recombinant polypeptide comprises an amino acid sequence at least 91% identical to SEQ ID NO: 65. In some embodiments, the recombinant polypeptide comprises an amino acid sequence at least 92% identical to SEQ ID NO: 65. In some embodiments, the recombinant polypeptide comprises an amino acid sequence at least 93% identical to SEQ ID NO: 65. In some embodiments, the recombinant polypeptide comprises an amino acid sequence at least 940% identical to SEQ ID NO: 65. In some embodiments, the recombinant polypeptide comprises an amino acid sequence at least 95% identical to SEQ ID NO: 65. In some embodiments, the recombinant polypeptide comprises an amino acid sequence at least 96% identical to SEQ ID NO: 65. In some embodiments, the recombinant polypeptide comprises an amino acid sequence at least 97% identical to SEQ ID NO: 65. In some embodiments, the recombinant polypeptide comprises an amino acid sequence at least 98% identical to SEQ ID NO: 65. In some embodiments, the recombinant polypeptide comprises an amino acid sequence at least 99% identical to SEQ ID NO: 65. In some embodiments, the recombinant polypeptide comprises an amino acid sequence identical to SEQ ID NO: 65. In some embodiments, the recombinant sequence does not comprise the n-terminal methionine. In some embodiments, the recombinant sequence does not comprise the n-terminal his tag sequence. In some embodiments, the recombinant sequence does not comprise the n-terminal methionine or the n-terminal his tag sequence.

In some embodiments, the recombinant polypeptide comprises an amino acid sequence at least 70% identical to SEQ ID NO: 66. In some embodiments, the recombinant polypeptide comprises an amino acid sequence at least 80% identical to SEQ ID NO: 66. In some embodiments, the recombinant polypeptide comprises an amino acid sequence at least 85% identical to SEQ ID NO: 66. In some embodiments, the recombinant polypeptide comprises an amino acid sequence at least 90% identical to SEQ ID NO: 66. In some embodiments, the recombinant polypeptide comprises an amino acid sequence at least 91% identical to SEQ ID NO: 66. In some embodiments, the recombinant polypeptide comprises an amino acid sequence at least 92% identical to SEQ ID NO: 66. In some embodiments, the recombinant polypeptide comprises an amino acid sequence at least 93% identical to SEQ ID NO: 66. In some embodiments, the recombinant polypeptide comprises an amino acid sequence at least 940% identical to SEQ ID NO: 66. In some embodiments, the recombinant polypeptide comprises an amino acid sequence at least 95% identical to SEQ ID NO: 66. In some embodiments, the recombinant polypeptide comprises an amino acid sequence at least 96% identical to SEQ ID NO: 66. In some embodiments, the recombinant polypeptide comprises an amino acid sequence at least 97% identical to SEQ ID NO: 66. In some embodiments, the recombinant polypeptide comprises an amino acid sequence at least 98% identical to SEQ ID NO: 66. In some embodiments, the recombinant polypeptide comprises an amino acid sequence at least 99% identical to SEQ ID NO: 66. In some embodiments, the recombinant polypeptide comprises an amino acid sequence identical to SEQ ID NO: 66. In some embodiments, the recombinant sequence does not comprise the n-terminal methionine. In some embodiments, the recombinant sequence does not comprise the n-terminal his tag sequence. In some embodiments, the recombinant sequence does not comprise the n-terminal methionine or the n-terminal his tag sequence.

In some embodiments, the recombinant polypeptide comprises an amino acid sequence at least 70% identical to SEQ ID NO: 67. In some embodiments, the recombinant polypeptide comprises an amino acid sequence at least 80% identical to SEQ ID NO: 67. In some embodiments, the recombinant polypeptide comprises an amino acid sequence at least 85% identical to SEQ ID NO: 67. In some embodiments, the recombinant polypeptide comprises an amino acid sequence at least 90% identical to SEQ ID NO: 67. In some embodiments, the recombinant polypeptide comprises an amino acid sequence at least 91% identical to SEQ ID NO: 67. In some embodiments, the recombinant polypeptide comprises an amino acid sequence at least 92% identical to SEQ ID NO: 67. In some embodiments, the recombinant polypeptide comprises an amino acid sequence at least 93% identical to SEQ ID NO: 67. In some embodiments, the recombinant polypeptide comprises an amino acid sequence at least 940% identical to SEQ ID NO: 67. In some embodiments, the recombinant polypeptide comprises an amino acid sequence at least 95% identical to SEQ ID NO: 67. In some embodiments, the recombinant polypeptide comprises an amino acid sequence at least 96% identical to SEQ ID NO: 67. In some embodiments, the recombinant polypeptide comprises an amino acid sequence at least 97% identical to SEQ ID NO: 67. In some embodiments, the recombinant polypeptide comprises an amino acid sequence at least 98% identical to SEQ ID NO: 67. In some embodiments, the recombinant polypeptide comprises an amino acid sequence at least 99% identical to SEQ ID NO: 67. In some embodiments, the recombinant polypeptide comprises an amino acid sequence identical to SEQ ID NO: 67. In some embodiments, the recombinant sequence does not comprise the n-terminal methionine. In some embodiments, the recombinant sequence does not comprise the n-terminal his tag sequence. In some embodiments, the recombinant sequence does not comprise the n-terminal methionine or the n-terminal his tag sequence.

In some embodiments, the recombinant polypeptide comprises an amino acid sequence at least 70% identical to SEQ ID NO: 68. In some embodiments, the recombinant polypeptide comprises an amino acid sequence at least 80% identical to SEQ ID NO: 68. In some embodiments, the recombinant polypeptide comprises an amino acid sequence at least 85% identical to SEQ ID NO: 68. In some embodiments, the recombinant polypeptide comprises an amino acid sequence at least 90% identical to SEQ ID NO: 68. In some embodiments, the recombinant polypeptide comprises an amino acid sequence at least 91% identical to SEQ ID NO: 68. In some embodiments, the recombinant polypeptide comprises an amino acid sequence at least 92% identical to SEQ ID NO: 68. In some embodiments, the recombinant polypeptide comprises an amino acid sequence at least 93% identical to SEQ ID NO: 68. In some embodiments, the recombinant polypeptide comprises an amino acid sequence at least 940% identical to SEQ ID NO: 68. In some embodiments, the recombinant polypeptide comprises an amino acid sequence at least 95% identical to SEQ ID NO: 68. In some embodiments, the recombinant polypeptide comprises an amino acid sequence at least 96% identical to SEQ ID NO: 68. In some embodiments, the recombinant polypeptide comprises an amino acid sequence at least 97% identical to SEQ ID NO: 68. In some embodiments, the recombinant polypeptide comprises an amino acid sequence at least 98% identical to SEQ ID NO: 68. In some embodiments, the recombinant polypeptide comprises an amino acid sequence at least 99% identical to SEQ ID NO: 68. In some embodiments, the recombinant polypeptide comprises an amino acid sequence identical to SEQ ID NO: 68. In some embodiments, the recombinant sequence does not comprise the n-terminal methionine. In some embodiments, the recombinant sequence does not comprise the n-terminal his tag sequence. In some embodiments, the recombinant sequence does not comprise the n-terminal methionine or the n-terminal his tag sequence.

In some embodiments, the recombinant polypeptide comprises an amino acid sequence at least 70% identical to SEQ ID NO: 69. In some embodiments, the recombinant polypeptide comprises an amino acid sequence at least 80% identical to SEQ ID NO: 69. In some embodiments, the recombinant polypeptide comprises an amino acid sequence at least 85% identical to SEQ ID NO: 69. In some embodiments, the recombinant polypeptide comprises an amino acid sequence at least 90% identical to SEQ ID NO: 69. In some embodiments, the recombinant polypeptide comprises an amino acid sequence at least 91% identical to SEQ ID NO: 69. In some embodiments, the recombinant polypeptide comprises an amino acid sequence at least 92% identical to SEQ ID NO: 69. In some embodiments, the recombinant polypeptide comprises an amino acid sequence at least 93% identical to SEQ ID NO: 69. In some embodiments, the recombinant polypeptide comprises an amino acid sequence at least 940% identical to SEQ ID NO: 69. In some embodiments, the recombinant polypeptide comprises an amino acid sequence at least 95% identical to SEQ ID NO: 69. In some embodiments, the recombinant polypeptide comprises an amino acid sequence at least 96% identical to SEQ ID NO: 69. In some embodiments, the recombinant polypeptide comprises an amino acid sequence at least 97% identical to SEQ ID NO: 69. In some embodiments, the recombinant polypeptide comprises an amino acid sequence at least 98% identical to SEQ ID NO: 69. In some embodiments, the recombinant polypeptide comprises an amino acid sequence at least 99% identical to SEQ ID NO: 69. In some embodiments, the recombinant polypeptide comprises an amino acid sequence identical to SEQ ID NO: 69. In some embodiments, the recombinant sequence does not comprise the n-terminal methionine. In some embodiments, the recombinant sequence does not comprise the n-terminal his tag sequence. In some embodiments, the recombinant sequence does not comprise the n-terminal methionine or the n-terminal his tag sequence.

In some embodiments, the recombinant polypeptide comprises an amino acid sequence at least 70% identical to SEQ ID NO: 70. In some embodiments, the recombinant polypeptide comprises an amino acid sequence at least 80% identical to SEQ ID NO: 70. In some embodiments, the recombinant polypeptide comprises an amino acid sequence at least 85% identical to SEQ ID NO: 70. In some embodiments, the recombinant polypeptide comprises an amino acid sequence at least 90% identical to SEQ ID NO: 70. In some embodiments, the recombinant polypeptide comprises an amino acid sequence at least 91% identical to SEQ ID NO: 70. In some embodiments, the recombinant polypeptide comprises an amino acid sequence at least 92% identical to SEQ ID NO: 70. In some embodiments, the recombinant polypeptide comprises an amino acid sequence at least 93% identical to SEQ ID NO: 70. In some embodiments, the recombinant polypeptide comprises an amino acid sequence at least 940% identical to SEQ ID NO: 70. In some embodiments, the recombinant polypeptide comprises an amino acid sequence at least 95% identical to SEQ ID NO: 70. In some embodiments, the recombinant polypeptide comprises an amino acid sequence at least 96% identical to SEQ ID NO: 70. In some embodiments, the recombinant polypeptide comprises an amino acid sequence at least 97% identical to SEQ ID NO: 70. In some embodiments, the recombinant polypeptide comprises an amino acid sequence at least 98% identical to SEQ ID NO: 70. In some embodiments, the recombinant polypeptide comprises an amino acid sequence at least 99% identical to SEQ ID NO: 70. In some embodiments, the recombinant polypeptide comprises an amino acid sequence identical to SEQ ID NO: 70. In some embodiments, the recombinant sequence does not comprise the n-terminal methionine. In some embodiments, the recombinant sequence does not comprise the n-terminal his tag sequence. In some embodiments, the recombinant sequence does not comprise the n-terminal methionine or the n-terminal his tag sequence.

In some embodiments, the recombinant polypeptide comprises an amino acid sequence at least 70% identical to SEQ ID NO: 71. In some embodiments, the recombinant polypeptide comprises an amino acid sequence at least 80% identical to SEQ ID NO: 71. In some embodiments, the recombinant polypeptide comprises an amino acid sequence at least 85% identical to SEQ ID NO: 71. In some embodiments, the recombinant polypeptide comprises an amino acid sequence at least 90% identical to SEQ ID NO: 71. In some embodiments, the recombinant polypeptide comprises an amino acid sequence at least 91% identical to SEQ ID NO: 71. In some embodiments, the recombinant polypeptide comprises an amino acid sequence at least 92% identical to SEQ ID NO: 71. In some embodiments, the recombinant polypeptide comprises an amino acid sequence at least 93% identical to SEQ ID NO: 71. In some embodiments, the recombinant polypeptide comprises an amino acid sequence at least 940% identical to SEQ ID NO: 71. In some embodiments, the recombinant polypeptide comprises an amino acid sequence at least 95% identical to SEQ ID NO: 71. In some embodiments, the recombinant polypeptide comprises an amino acid sequence at least 96% identical to SEQ ID NO: 71. In some embodiments, the recombinant polypeptide comprises an amino acid sequence at least 97% identical to SEQ ID NO: 71. In some embodiments, the recombinant polypeptide comprises an amino acid sequence at least 98% identical to SEQ ID NO: 71. In some embodiments, the recombinant polypeptide comprises an amino acid sequence at least 99% identical to SEQ ID NO: 71. In some embodiments, the recombinant polypeptide comprises an amino acid sequence identical to SEQ ID NO: 71. In some embodiments, the recombinant sequence does not comprise the n-terminal methionine. In some embodiments, the recombinant sequence does not comprise the n-terminal his tag sequence. In some embodiments, the recombinant sequence does not comprise the n-terminal methionine or the n-terminal his tag sequence.

In some embodiments, the recombinant polypeptide comprises an amino acid sequence at least 70% identical to SEQ ID NO: 72. In some embodiments, the recombinant polypeptide comprises an amino acid sequence at least 80% identical to SEQ ID NO: 72. In some embodiments, the recombinant polypeptide comprises an amino acid sequence at least 85% identical to SEQ ID NO: 72. In some embodiments, the recombinant polypeptide comprises an amino acid sequence at least 90% identical to SEQ ID NO: 72. In some embodiments, the recombinant polypeptide comprises an amino acid sequence at least 91% identical to SEQ ID NO: 72. In some embodiments, the recombinant polypeptide comprises an amino acid sequence at least 92% identical to SEQ ID NO: 72. In some embodiments, the recombinant polypeptide comprises an amino acid sequence at least 93% identical to SEQ ID NO: 72. In some embodiments, the recombinant polypeptide comprises an amino acid sequence at least 940% identical to SEQ ID NO: 72. In some embodiments, the recombinant polypeptide comprises an amino acid sequence at least 95% identical to SEQ ID NO: 72. In some embodiments, the recombinant polypeptide comprises an amino acid sequence at least 96% identical to SEQ ID NO: 72. In some embodiments, the recombinant polypeptide comprises an amino acid sequence at least 97% identical to SEQ ID NO: 72. In some embodiments, the recombinant polypeptide comprises an amino acid sequence at least 98% identical to SEQ ID NO: 72. In some embodiments, the recombinant polypeptide comprises an amino acid sequence at least 99% identical to SEQ ID NO: 72. In some embodiments, the recombinant polypeptide comprises an amino acid sequence identical to SEQ ID NO: 72. In some embodiments, the recombinant sequence does not comprise the n-terminal methionine. In some embodiments, the recombinant sequence does not comprise the n-terminal his tag sequence. In some embodiments, the recombinant sequence does not comprise the n-terminal methionine or the n-terminal his tag sequence.

In some embodiments, the recombinant polypeptide comprises an amino acid sequence at least 70% identical to SEQ ID NO: 73. In some embodiments, the recombinant polypeptide comprises an amino acid sequence at least 80% identical to SEQ ID NO: 73. In some embodiments, the recombinant polypeptide comprises an amino acid sequence at least 85% identical to SEQ ID NO: 73. In some embodiments, the recombinant polypeptide comprises an amino acid sequence at least 90% identical to SEQ ID NO: 73. In some embodiments, the recombinant polypeptide comprises an amino acid sequence at least 91% identical to SEQ ID NO: 73. In some embodiments, the recombinant polypeptide comprises an amino acid sequence at least 92% identical to SEQ ID NO: 73. In some embodiments, the recombinant polypeptide comprises an amino acid sequence at least 93% identical to SEQ ID NO: 73. In some embodiments, the recombinant polypeptide comprises an amino acid sequence at least 940% identical to SEQ ID NO: 73. In some embodiments, the recombinant polypeptide comprises an amino acid sequence at least 95% identical to SEQ ID NO: 73. In some embodiments, the recombinant polypeptide comprises an amino acid sequence at least 96% identical to SEQ ID NO: 73. In some embodiments, the recombinant polypeptide comprises an amino acid sequence at least 97% identical to SEQ ID NO: 73. In some embodiments, the recombinant polypeptide comprises an amino acid sequence at least 98% identical to SEQ ID NO: 73. In some embodiments, the recombinant polypeptide comprises an amino acid sequence at least 99% identical to SEQ ID NO: 73. In some embodiments, the recombinant polypeptide comprises an amino acid sequence identical to SEQ ID NO: 73. In some embodiments, the recombinant sequence does not comprise the n-terminal methionine. In some embodiments, the recombinant sequence does not comprise the n-terminal his tag sequence. In some embodiments, the recombinant sequence does not comprise the n-terminal methionine or the n-terminal his tag sequence.

In some embodiments, the recombinant polypeptide comprises an amino acid sequence at least 70% identical to SEQ ID NO: 74. In some embodiments, the recombinant polypeptide comprises an amino acid sequence at least 80% identical to SEQ ID NO: 74. In some embodiments, the recombinant polypeptide comprises an amino acid sequence at least 85% identical to SEQ ID NO: 74. In some embodiments, the recombinant polypeptide comprises an amino acid sequence at least 90% identical to SEQ ID NO: 74. In some embodiments, the recombinant polypeptide comprises an amino acid sequence at least 91% identical to SEQ ID NO: 74. In some embodiments, the recombinant polypeptide comprises an amino acid sequence at least 92% identical to SEQ ID NO: 74. In some embodiments, the recombinant polypeptide comprises an amino acid sequence at least 93% identical to SEQ ID NO: 74. In some embodiments, the recombinant polypeptide comprises an amino acid sequence at least 940% identical to SEQ ID NO: 74. In some embodiments, the recombinant polypeptide comprises an amino acid sequence at least 95% identical to SEQ ID NO: 74. In some embodiments, the recombinant polypeptide comprises an amino acid sequence at least 96% identical to SEQ ID NO: 74. In some embodiments, the recombinant polypeptide comprises an amino acid sequence at least 97% identical to SEQ ID NO: 74. In some embodiments, the recombinant polypeptide comprises an amino acid sequence at least 98% identical to SEQ ID NO: 74. In some embodiments, the recombinant polypeptide comprises an amino acid sequence at least 99% identical to SEQ ID NO: 74. In some embodiments, the recombinant polypeptide comprises an amino acid sequence identical to SEQ ID NO: 74. In some embodiments, the recombinant sequence does not comprise the n-terminal methionine. In some embodiments, the recombinant sequence does not comprise the n-terminal his tag sequence. In some embodiments, the recombinant sequence does not comprise the n-terminal methionine or the n-terminal his tag sequence.

In some embodiments, the recombinant polypeptide comprises an amino acid sequence at least 70% identical to SEQ ID NO: 75. In some embodiments, the recombinant polypeptide comprises an amino acid sequence at least 80% identical to SEQ ID NO: 75. In some embodiments, the recombinant polypeptide comprises an amino acid sequence at least 85% identical to SEQ ID NO: 75. In some embodiments, the recombinant polypeptide comprises an amino acid sequence at least 90% identical to SEQ ID NO: 75. In some embodiments, the recombinant polypeptide comprises an amino acid sequence at least 91% identical to SEQ ID NO: 75. In some embodiments, the recombinant polypeptide comprises an amino acid sequence at least 92% identical to SEQ ID NO: 75. In some embodiments, the recombinant polypeptide comprises an amino acid sequence at least 93% identical to SEQ ID NO: 75. In some embodiments, the recombinant polypeptide comprises an amino acid sequence at least 940% identical to SEQ ID NO: 75. In some embodiments, the recombinant polypeptide comprises an amino acid sequence at least 95% identical to SEQ ID NO: 75. In some embodiments, the recombinant polypeptide comprises an amino acid sequence at least 96% identical to SEQ ID NO: 75. In some embodiments, the recombinant polypeptide comprises an amino acid sequence at least 97% identical to SEQ ID NO: 75. In some embodiments, the recombinant polypeptide comprises an amino acid sequence at least 98% identical to SEQ ID NO: 75. In some embodiments, the recombinant polypeptide comprises an amino acid sequence at least 99% identical to SEQ ID NO: 75. In some embodiments, the recombinant polypeptide comprises an amino acid sequence identical to SEQ ID NO: 75. In some embodiments, the recombinant sequence does not comprise the n-terminal methionine. In some embodiments, the recombinant sequence does not comprise the n-terminal his tag sequence. In some embodiments, the recombinant sequence does not comprise the n-terminal methionine or the n-terminal his tag sequence.

In some embodiments, the recombinant polypeptide comprises an amino acid sequence at least 70% identical to SEQ ID NO: 76. In some embodiments, the recombinant polypeptide comprises an amino acid sequence at least 80% identical to SEQ ID NO: 76. In some embodiments, the recombinant polypeptide comprises an amino acid sequence at least 85% identical to SEQ ID NO: 76. In some embodiments, the recombinant polypeptide comprises an amino acid sequence at least 90% identical to SEQ ID NO: 76. In some embodiments, the recombinant polypeptide comprises an amino acid sequence at least 91% identical to SEQ ID NO: 76. In some embodiments, the recombinant polypeptide comprises an amino acid sequence at least 92% identical to SEQ ID NO: 76. In some embodiments, the recombinant polypeptide comprises an amino acid sequence at least 93% identical to SEQ ID NO: 76. In some embodiments, the recombinant polypeptide comprises an amino acid sequence at least 940% identical to SEQ ID NO: 76. In some embodiments, the recombinant polypeptide comprises an amino acid sequence at least 95% identical to SEQ ID NO: 76. In some embodiments, the recombinant polypeptide comprises an amino acid sequence at least 96% identical to SEQ ID NO: 76. In some embodiments, the recombinant polypeptide comprises an amino acid sequence at least 97% identical to SEQ ID NO: 76. In some embodiments, the recombinant polypeptide comprises an amino acid sequence at least 98% identical to SEQ ID NO: 76. In some embodiments, the recombinant polypeptide comprises an amino acid sequence at least 99% identical to SEQ ID NO: 76. In some embodiments, the recombinant polypeptide comprises an amino acid sequence identical to SEQ ID NO: 76. In some embodiments, the recombinant sequence does not comprise the n-terminal methionine. In some embodiments, the recombinant sequence does not comprise the n-terminal his tag sequence. In some embodiments, the recombinant sequence does not comprise the n-terminal methionine or the n-terminal his tag sequence.

In some embodiments, the recombinant polypeptide comprises an amino acid sequence at least 70% identical to SEQ ID NO: 77. In some embodiments, the recombinant polypeptide comprises an amino acid sequence at least 80% identical to SEQ ID NO: 77. In some embodiments, the recombinant polypeptide comprises an amino acid sequence at least 85% identical to SEQ ID NO: 77. In some embodiments, the recombinant polypeptide comprises an amino acid sequence at least 90% identical to SEQ ID NO: 77. In some embodiments, the recombinant polypeptide comprises an amino acid sequence at least 91% identical to SEQ ID NO: 77. In some embodiments, the recombinant polypeptide comprises an amino acid sequence at least 92% identical to SEQ ID NO: 77. In some embodiments, the recombinant polypeptide comprises an amino acid sequence at least 93% identical to SEQ ID NO: 77. In some embodiments, the recombinant polypeptide comprises an amino acid sequence at least 940% identical to SEQ ID NO: 77. In some embodiments, the recombinant polypeptide comprises an amino acid sequence at least 95% identical to SEQ ID NO: 77. In some embodiments, the recombinant polypeptide comprises an amino acid sequence at least 96% identical to SEQ ID NO: 77. In some embodiments, the recombinant polypeptide comprises an amino acid sequence at least 97% identical to SEQ ID NO: 77. In some embodiments, the recombinant polypeptide comprises an amino acid sequence at least 98% identical to SEQ ID NO: 77. In some embodiments, the recombinant polypeptide comprises an amino acid sequence at least 99% identical to SEQ ID NO: 77. In some embodiments, the recombinant polypeptide comprises an amino acid sequence identical to SEQ ID NO: 77. In some embodiments, the recombinant sequence does not comprise the n-terminal methionine. In some embodiments, the recombinant sequence does not comprise the n-terminal his tag sequence. In some embodiments, the recombinant sequence does not comprise the n-terminal methionine or the n-terminal his tag sequence.

In some embodiments, the recombinant polypeptide comprises an amino acid sequence at least 70% identical to SEQ ID NO: 78. In some embodiments, the recombinant polypeptide comprises an amino acid sequence at least 80% identical to SEQ ID NO: 78. In some embodiments, the recombinant polypeptide comprises an amino acid sequence at least 85% identical to SEQ ID NO: 78. In some embodiments, the recombinant polypeptide comprises an amino acid sequence at least 90% identical to SEQ ID NO: 78. In some embodiments, the recombinant polypeptide comprises an amino acid sequence at least 91% identical to SEQ ID NO: 78. In some embodiments, the recombinant polypeptide comprises an amino acid sequence at least 92% identical to SEQ ID NO: 78. In some embodiments, the recombinant polypeptide comprises an amino acid sequence at least 93% identical to SEQ ID NO: 78. In some embodiments, the recombinant polypeptide comprises an amino acid sequence at least 940% identical to SEQ ID NO: 78. In some embodiments, the recombinant polypeptide comprises an amino acid sequence at least 95% identical to SEQ ID NO: 78. In some embodiments, the recombinant polypeptide comprises an amino acid sequence at least 96% identical to SEQ ID NO: 78. In some embodiments, the recombinant polypeptide comprises an amino acid sequence at least 97% identical to SEQ ID NO: 78. In some embodiments, the recombinant polypeptide comprises an amino acid sequence at least 98% identical to SEQ ID NO: 78. In some embodiments, the recombinant polypeptide comprises an amino acid sequence at least 99% identical to SEQ ID NO: 78. In some embodiments, the recombinant polypeptide comprises an amino acid sequence identical to SEQ ID NO: 78. In some embodiments, the recombinant sequence does not comprise the n-terminal methionine. In some embodiments, the recombinant sequence does not comprise the n-terminal his tag sequence. In some embodiments, the recombinant sequence does not comprise the n-terminal methionine or the n-terminal his tag sequence.

In some embodiments, the recombinant polypeptide comprises an amino acid sequence at least 70% identical to SEQ ID NO: 79. In some embodiments, the recombinant polypeptide comprises an amino acid sequence at least 80% identical to SEQ ID NO: 79. In some embodiments, the recombinant polypeptide comprises an amino acid sequence at least 85% identical to SEQ ID NO: 79. In some embodiments, the recombinant polypeptide comprises an amino acid sequence at least 90% identical to SEQ ID NO: 79. In some embodiments, the recombinant polypeptide comprises an amino acid sequence at least 91% identical to SEQ ID NO: 79. In some embodiments, the recombinant polypeptide comprises an amino acid sequence at least 92% identical to SEQ ID NO: 79. In some embodiments, the recombinant polypeptide comprises an amino acid sequence at least 93% identical to SEQ ID NO: 79. In some embodiments, the recombinant polypeptide comprises an amino acid sequence at least 940% identical to SEQ ID NO: 79. In some embodiments, the recombinant polypeptide comprises an amino acid sequence at least 95% identical to SEQ ID NO: 79. In some embodiments, the recombinant polypeptide comprises an amino acid sequence at least 96% identical to SEQ ID NO: 79. In some embodiments, the recombinant polypeptide comprises an amino acid sequence at least 97% identical to SEQ ID NO: 79. In some embodiments, the recombinant polypeptide comprises an amino acid sequence at least 98% identical to SEQ ID NO: 79. In some embodiments, the recombinant polypeptide comprises an amino acid sequence at least 99% identical to SEQ ID NO: 79. In some embodiments, the recombinant polypeptide comprises an amino acid sequence identical to SEQ ID NO: 79. In some embodiments, the recombinant sequence does not comprise the n-terminal methionine. In some embodiments, the recombinant sequence does not comprise the n-terminal his tag sequence. In some embodiments, the recombinant sequence does not comprise the n-terminal methionine or the n-terminal his tag sequence.

In some embodiments, the recombinant polypeptide comprises an amino acid sequence at least 70% identical to SEQ ID NO: 82. In some embodiments, the recombinant polypeptide comprises an amino acid sequence at least 80% identical to SEQ ID NO: 82. In some embodiments, the recombinant polypeptide comprises an amino acid sequence at least 85% identical to SEQ ID NO: 82. In some embodiments, the recombinant polypeptide comprises an amino acid sequence at least 90% identical to SEQ ID NO: 82. In some embodiments, the recombinant polypeptide comprises an amino acid sequence at least 91% identical to SEQ ID NO: 82. In some embodiments, the recombinant polypeptide comprises an amino acid sequence at least 92% identical to SEQ ID NO: 82. In some embodiments, the recombinant polypeptide comprises an amino acid sequence at least 93% identical to SEQ ID NO: 82. In some embodiments, the recombinant polypeptide comprises an amino acid sequence at least 940% identical to SEQ ID NO: 82. In some embodiments, the recombinant polypeptide comprises an amino acid sequence at least 95% identical to SEQ ID NO: 82. In some embodiments, the recombinant polypeptide comprises an amino acid sequence at least 96% identical to SEQ ID NO: 82. In some embodiments, the recombinant polypeptide comprises an amino acid sequence at least 97% identical to SEQ ID NO: 82. In some embodiments, the recombinant polypeptide comprises an amino acid sequence at least 98% identical to SEQ ID NO: 82. In some embodiments, the recombinant polypeptide comprises an amino acid sequence at least 99% identical to SEQ ID NO: 82. In some embodiments, the recombinant polypeptide comprises an amino acid sequence identical to SEQ ID NO: 82. In some embodiments, the recombinant sequence does not comprise the n-terminal methionine. In some embodiments, the recombinant sequence does not comprise the n-terminal his tag sequence. In some embodiments, the recombinant sequence does not comprise the n-terminal methionine or the n-terminal his tag sequence.

In some embodiments, the recombinant polypeptide comprises an amino acid sequence at least 70% identical to SEQ ID NO: 83. In some embodiments, the recombinant polypeptide comprises an amino acid sequence at least 80% identical to SEQ ID NO: 83. In some embodiments, the recombinant polypeptide comprises an amino acid sequence at least 85% identical to SEQ ID NO: 83. In some embodiments, the recombinant polypeptide comprises an amino acid sequence at least 90% identical to SEQ ID NO: 83. In some embodiments, the recombinant polypeptide comprises an amino acid sequence at least 91% identical to SEQ ID NO: 83. In some embodiments, the recombinant polypeptide comprises an amino acid sequence at least 92% identical to SEQ ID NO: 83. In some embodiments, the recombinant polypeptide comprises an amino acid sequence at least 93% identical to SEQ ID NO: 83. In some embodiments, the recombinant polypeptide comprises an amino acid sequence at least 940% identical to SEQ ID NO: 83. In some embodiments, the recombinant polypeptide comprises an amino acid sequence at least 95% identical to SEQ ID NO: 83. In some embodiments, the recombinant polypeptide comprises an amino acid sequence at least 96% identical to SEQ ID NO: 83. In some embodiments, the recombinant polypeptide comprises an amino acid sequence at least 97% identical to SEQ ID NO: 83. In some embodiments, the recombinant polypeptide comprises an amino acid sequence at least 98% identical to SEQ ID NO: 83. In some embodiments, the recombinant polypeptide comprises an amino acid sequence at least 99% identical to SEQ ID NO: 83. In some embodiments, the recombinant polypeptide comprises an amino acid sequence identical to SEQ ID NO: 83. In some embodiments, the recombinant sequence does not comprise the n-terminal methionine. In some embodiments, the recombinant sequence does not comprise the n-terminal his tag sequence. In some embodiments, the recombinant sequence does not comprise the n-terminal methionine or the n-terminal his tag sequence.

In some embodiments, the recombinant polypeptide comprises an amino acid sequence at least 70% identical to SEQ ID NO: 84. In some embodiments, the recombinant polypeptide comprises an amino acid sequence at least 80% identical to SEQ ID NO: 84. In some embodiments, the recombinant polypeptide comprises an amino acid sequence at least 85% identical to SEQ ID NO: 84. In some embodiments, the recombinant polypeptide comprises an amino acid sequence at least 90% identical to SEQ ID NO: 84. In some embodiments, the recombinant polypeptide comprises an amino acid sequence at least 91% identical to SEQ ID NO: 84. In some embodiments, the recombinant polypeptide comprises an amino acid sequence at least 92% identical to SEQ ID NO: 84. In some embodiments, the recombinant polypeptide comprises an amino acid sequence at least 93% identical to SEQ ID NO: 84. In some embodiments, the recombinant polypeptide comprises an amino acid sequence at least 940% identical to SEQ ID NO: 84. In some embodiments, the recombinant polypeptide comprises an amino acid sequence at least 95% identical to SEQ ID NO: 84. In some embodiments, the recombinant polypeptide comprises an amino acid sequence at least 96% identical to SEQ ID NO: 84. In some embodiments, the recombinant polypeptide comprises an amino acid sequence at least 97% identical to SEQ ID NO: 84. In some embodiments, the recombinant polypeptide comprises an amino acid sequence at least 98% identical to SEQ ID NO: 84. In some embodiments, the recombinant polypeptide comprises an amino acid sequence at least 99% identical to SEQ ID NO: 84. In some embodiments, the recombinant polypeptide comprises an amino acid sequence identical to SEQ ID NO: 84. In some embodiments, the recombinant sequence does not comprise the n-terminal methionine. In some embodiments, the recombinant sequence does not comprise the n-terminal his tag sequence. In some embodiments, the recombinant sequence does not comprise the n-terminal methionine or the n-terminal his tag sequence.

In some embodiments, the recombinant polypeptide comprises an amino acid sequence at least 70% identical to SEQ ID NO: 85. In some embodiments, the recombinant polypeptide comprises an amino acid sequence at least 80% identical to SEQ ID NO: 85. In some embodiments, the recombinant polypeptide comprises an amino acid sequence at least 85% identical to SEQ ID NO: 85. In some embodiments, the recombinant polypeptide comprises an amino acid sequence at least 90% identical to SEQ ID NO: 85. In some embodiments, the recombinant polypeptide comprises an amino acid sequence at least 91% identical to SEQ ID NO: 85. In some embodiments, the recombinant polypeptide comprises an amino acid sequence at least 92% identical to SEQ ID NO: 85. In some embodiments, the recombinant polypeptide comprises an amino acid sequence at least 93% identical to SEQ ID NO: 85. In some embodiments, the recombinant polypeptide comprises an amino acid sequence at least 940% identical to SEQ ID NO: 85. In some embodiments, the recombinant polypeptide comprises an amino acid sequence at least 95% identical to SEQ ID NO: 85. In some embodiments, the recombinant polypeptide comprises an amino acid sequence at least 96% identical to SEQ ID NO: 85. In some embodiments, the recombinant polypeptide comprises an amino acid sequence at least 97% identical to SEQ ID NO: 85. In some embodiments, the recombinant polypeptide comprises an amino acid sequence at least 98% identical to SEQ ID NO: 85. In some embodiments, the recombinant polypeptide comprises an amino acid sequence at least 99% identical to SEQ ID NO: 85. In some embodiments, the recombinant polypeptide comprises an amino acid sequence identical to SEQ ID NO: 85. In some embodiments, the recombinant sequence does not comprise the n-terminal methionine. In some embodiments, the recombinant sequence does not comprise the n-terminal his tag sequence. In some embodiments, the recombinant sequence does not comprise the n-terminal methionine or the n-terminal his tag sequence.

In some embodiments, the recombinant polypeptide comprises an amino acid sequence at least 70% identical to SEQ ID NO: 86. In some embodiments, the recombinant polypeptide comprises an amino acid sequence at least 80% identical to SEQ ID NO: 86. In some embodiments, the recombinant polypeptide comprises an amino acid sequence at least 85% identical to SEQ ID NO: 86. In some embodiments, the recombinant polypeptide comprises an amino acid sequence at least 90% identical to SEQ ID NO: 86. In some embodiments, the recombinant polypeptide comprises an amino acid sequence at least 91% identical to SEQ ID NO: 86. In some embodiments, the recombinant polypeptide comprises an amino acid sequence at least 92% identical to SEQ ID NO: 86. In some embodiments, the recombinant polypeptide comprises an amino acid sequence at least 93% identical to SEQ ID NO: 86. In some embodiments, the recombinant polypeptide comprises an amino acid sequence at least 940% identical to SEQ ID NO: 86. In some embodiments, the recombinant polypeptide comprises an amino acid sequence at least 95% identical to SEQ ID NO: 86. In some embodiments, the recombinant polypeptide comprises an amino acid sequence at least 96% identical to SEQ ID NO: 86. In some embodiments, the recombinant polypeptide comprises an amino acid sequence at least 97% identical to SEQ ID NO: 86. In some embodiments, the recombinant polypeptide comprises an amino acid sequence at least 98% identical to SEQ ID NO: 86. In some embodiments, the recombinant polypeptide comprises an amino acid sequence at least 99% identical to SEQ ID NO: 86. In some embodiments, the recombinant polypeptide comprises an amino acid sequence identical to SEQ ID NO: 86. In some embodiments, the recombinant sequence does not comprise the n-terminal methionine. In some embodiments, the recombinant sequence does not comprise the n-terminal his tag sequence. In some embodiments, the recombinant sequence does not comprise the n-terminal methionine or the n-terminal his tag sequence.

In some embodiments, the recombinant polypeptide comprises an amino acid sequence at least 70% identical to SEQ ID NO: 87. In some embodiments, the recombinant polypeptide comprises an amino acid sequence at least 80% identical to SEQ ID NO: 87. In some embodiments, the recombinant polypeptide comprises an amino acid sequence at least 85% identical to SEQ ID NO: 87. In some embodiments, the recombinant polypeptide comprises an amino acid sequence at least 90% identical to SEQ ID NO: 87. In some embodiments, the recombinant polypeptide comprises an amino acid sequence at least 91% identical to SEQ ID NO: 87. In some embodiments, the recombinant polypeptide comprises an amino acid sequence at least 92% identical to SEQ ID NO: 87. In some embodiments, the recombinant polypeptide comprises an amino acid sequence at least 93% identical to SEQ ID NO: 87. In some embodiments, the recombinant polypeptide comprises an amino acid sequence at least 940% identical to SEQ ID NO: 87. In some embodiments, the recombinant polypeptide comprises an amino acid sequence at least 95% identical to SEQ ID NO: 87. In some embodiments, the recombinant polypeptide comprises an amino acid sequence at least 96% identical to SEQ ID NO: 87. In some embodiments, the recombinant polypeptide comprises an amino acid sequence at least 97% identical to SEQ ID NO: 87. In some embodiments, the recombinant polypeptide comprises an amino acid sequence at least 98% identical to SEQ ID NO: 87. In some embodiments, the recombinant polypeptide comprises an amino acid sequence at least 99% identical to SEQ ID NO: 87. In some embodiments, the recombinant polypeptide comprises an amino acid sequence identical to SEQ ID NO: 87. In some embodiments, the recombinant sequence does not comprise the n-terminal methionine. In some embodiments, the recombinant sequence does not comprise the n-terminal his tag sequence. In some embodiments, the recombinant sequence does not comprise the n-terminal methionine or the n-terminal his tag sequence.

In some embodiments, the recombinant polypeptide comprises an amino acid sequence at least 70% identical to SEQ ID NO: 88. In some embodiments, the recombinant polypeptide comprises an amino acid sequence at least 80% identical to SEQ ID NO: 88. In some embodiments, the recombinant polypeptide comprises an amino acid sequence at least 85% identical to SEQ ID NO: 88. In some embodiments, the recombinant polypeptide comprises an amino acid sequence at least 90% identical to SEQ ID NO: 88. In some embodiments, the recombinant polypeptide comprises an amino acid sequence at least 91% identical to SEQ ID NO: 88. In some embodiments, the recombinant polypeptide comprises an amino acid sequence at least 92% identical to SEQ ID NO: 88. In some embodiments, the recombinant polypeptide comprises an amino acid sequence at least 93% identical to SEQ ID NO: 88. In some embodiments, the recombinant polypeptide comprises an amino acid sequence at least 940% identical to SEQ ID NO: 88. In some embodiments, the recombinant polypeptide comprises an amino acid sequence at least 95% identical to SEQ ID NO: 88. In some embodiments, the recombinant polypeptide comprises an amino acid sequence at least 96% identical to SEQ ID NO: 88. In some embodiments, the recombinant polypeptide comprises an amino acid sequence at least 97% identical to SEQ ID NO: 88. In some embodiments, the recombinant polypeptide comprises an amino acid sequence at least 98% identical to SEQ ID NO: 88. In some embodiments, the recombinant polypeptide comprises an amino acid sequence at least 99% identical to SEQ ID NO: 88. In some embodiments, the recombinant polypeptide comprises an amino acid sequence identical to SEQ ID NO: 88. In some embodiments, the recombinant sequence does not comprise the n-terminal methionine. In some embodiments, the recombinant sequence does not comprise the n-terminal his tag sequence. In some embodiments, the recombinant sequence does not comprise the n-terminal methionine or the n-terminal his tag sequence.

In some embodiments, the recombinant polypeptide converts olivetolic acid (OA) and geranyl diphosphate (GPP) to one or more products comprising cannabigerolic acid (CBGA). In some embodiments, at least about 50% of the one or more products is CBGA. In some embodiments, more than about 90% of the one or more products is CBGA. In some embodiments, the recombinant polypeptide has a rate of formation of cannabigerolic acid (CBGA) from olivetolic acid (OA) and geranyl diphosphate (GPP) that is greater than the rate of formation of CBGA from OA and GPP by NphB under the same conditions.

In some embodiments, the recombinant polypeptide converts olivetolic acid (OA) and farnesyl pyrophosphate (FPP) to one or more cannabinoid, cannabinoid derivatives, or cannabinoid analogues. In some embodiments, the recombinant polypeptide converts divarinic acid (DVA) and geranyl diphosphate (GPP) to CBGVA or one or more cannabinoids, cannabinoid derivatives, or cannabinoid analogues. The cannabinoids are not limited and may be any cannabinoid disclosed herein.

In some embodiments, the recombinant polypeptide has a rate of formation of CBGA from olivetolic acid (OA) and geranyl diphosphate (GPP) that is greater than the rate of formation of CBGA from OA and GPP by a polypeptide consisting of SEQ ID NO: 4 under the same conditions. In some embodiments, the recombinant polypeptide has a rate of formation of O-CBGA from OA and GPP that is greater than the rate of formation of O-CBGA from OA and GPP by a polypeptide consisting of SEQ ID NO: 4 under the same conditions. In some embodiments, recombinant polypeptide has a rate of formation of F-CBGA from OA and GPP that is greater than the rate of formation of F-CBGA from OA and GPP by a polypeptide consisting of SEQ ID NO: 4 under the same conditions.

In some embodiments, the recombinant polypeptide has a rate of formation of cannabigerovarinic acid (CBGVA) from divarinic acid (DVA) and geranyl diphosphate (GPP) that is greater than the rate of formation of CBGVA from DVA and GPP by a polypeptide consisting of SEQ ID NO: 4 under the same conditions. In some embodiments, the recombinant polypeptide has a rate of formation of O-CBGVA from DVA and GPP that is greater than the rate of formation of O-CBGVA from DVA and GPP by a polypeptide consisting of SEQ ID NO: 4 under the same conditions. In some embodiments, the recombinant polypeptide has a rate of formation of F-CBGVA from DVA and GPP that is greater than the rate of formation of F-CBGVA from DVA and GPP by a polypeptide consisting of SEQ ID NO: 4 under the same conditions.

In some embodiments, the recombinant polypeptide has a rate of formation of CBGA from OA and GPP that is at least 1.5-fold greater than the rate of formation of CBGA from OA and GPP by a polypeptide consisting of SEQ ID NO: 4 under the same conditions.

In some embodiments, the cell described herein comprises one or more additional metabolic pathway transgene(s). In some embodiments, the cell comprises an olivetolic acid pathway. In some embodiments, the olivetolic acid pathway comprises a polyketide cyclase. In some embodiments, an exogenous nucleotide codes for the polyketide cyclase. In some embodiments, the olivetolic acid pathway comprises polyketide synthase/olivetol synthase (condensation of hexanoyl coenzyme A (CoA) and 3× malonyl CoAs). In some embodiments, the cell comprises a geranyl pyrophosphate (GPP) pathway. In some embodiments, the GPP pathway comprises geranyl pyrophosphate synthase. In some embodiments, an exogenous nucleotide codes for the geranyl pyrophosphate synthase. In some embodiments, the cell comprises a farnesyl pyrophosphate (FPP) pathway. In some embodiments, the FPP pathway comprises a farnesyl pyrophosphate synthase. In some embodiments, the farnesyl pyrophosphate synthase is a mutant form. In some embodiments, the mutant farnesyl pyrophosphate synthase is described in (Jian G-Z, et al Metabolic Engineering, 2017, 41, 57, incorporated herein). In some embodiments, an exogenous nucleotide codes for the farnesyl pyrophosphate synthase. In some embodiments, the cell comprises a divarinic acid (DVA) pathway. In some embodiments, the DVA pathway comprises divarinic acid synthase. In some embodiments, an exogenous nucleotide codes for the divarinic acid synthase. In some embodiments, the cell comprises a mevalonate pathway. In some embodiments, the cell expresses HMG-COA reductase. In some embodiments, an endogenous mevalonate pathway of the cell has been manipulated to reduce or increase production of mevalonate, isopentyl pyrophosphate (IPP) or dimethylallyl pyrophosphate (DMAP), geranyl pyrophosphate (GPP) or farnesyl pyrophosphate (FPP). In some embodiments, the cell comprises a polyketide cyclase that produces OA, DVA, and/or derivatives thereof. In some embodiments, the cell comprises a polyketide synthase that produces a tetraketide substrate of the polyketide cyclase. In some embodiments, the cell comprises a polytetide synthase that can directly form OA and derivatives from acetyl-CoA or hexanoyl-CoA and malonyl-CoA.

In some embodiments, the cell is capable of producing a cannabinoid, a cannabinoid derivative, or cannabinoid analogue. The cannabinoids are not limited and may be any cannabinoid described herein. In some embodiments, the cannabinoid is selected from tetrahydrocannabinol, cannabidiol, cannabigerol, or an acid or analogue thereof.

In some embodiments, production of the cannabinoid by the cell is under control of a constitutional or inducible promoter. The promoter is not limited and may be any suitable promoter known in the art.

Some aspects of the present disclosure are directed to a composition comprising a cannabinoid, cannabinoid derivative, or cannabinoid analogue produced by a cell disclosed herein. In some embodiments, the composition further comprises a cell as described herein. In some embodiments, the composition comprises purified or isolated cannabinoid, cannabinoid derivative, or cannabinoid analogue produced by a cell disclosed herein. In some embodiments, the composition comprises cannabigerolic acid, tetrahydrocannabinol, cannabidiol, cannabigerol, or an acid, derivative, or an analogue thereof.

### Methods of Producing Cannabinoids

Some aspects of the present disclosure are directed to a method of producing a cannabinoid or an acid, derivative, or analogue thereof, the method comprising providing a cell as described herein comprising an exogenous nucleotide sequence coding for a recombinant polypeptide as described herein, and culturing the cell to produce the cannabinoid, cannabinoid derivative, or cannabinoid analogue thereof. In some embodiments, the cell expresses one or more of OA, GPP, FPP, and MVA. In some embodiments, the cell expresses OA and FPP. In some embodiments, the cell expresses OA and GPP. In some embodiments, the cell expresses MVA and GPP. In some embodiments, one or more of OA, GPP, FPP, and MVA is provided in a culture medium for use by the cell.

Depending on the cell, the appropriate culture medium may be used. For example, descriptions of various culture media may be found in “Manual of Methods for General Bacteriology” of the American Society for Bacteriology (Washington D.C., USA, 1981). As used here, “medium” as it relates to the growth source refers to the starting medium be it in a solid or liquid form. “Cultured medium”, on the other hand and as used here refers to medium (e.g. liquid medium) containing microbes that have been fermentatively grown and can include other cellular biomass. The medium generally includes one or more carbon sources, nitrogen sources, inorganic salts, vitamins and/or trace elements.

Exemplary carbon sources include sugar carbons such as sucrose, glucose, galactose, fructose, mannose, isomaltose, xylose, pannose, maltose, arabinose, cellobiose and 3-, 4-, or 5-oligomers thereof. Other carbon sources include alcohol carbon sources such as methanol, ethanol, glycerol. Other carbon sources include acid and esters such as acetate, formate, fatty acids having four to twenty-two carbon atoms or fatty acid esters thereof. Other carbon sources can include renewal feedstocks and biomass. Exemplary renewal feedstocks include cellulosic biomass, hemicellulosic biomass and lignin feedstocks. Mixed carbon sources can also be used, such as a fatty acid and a sugar as described herein.

The culture conditions can include, for example, liquid culture procedures as well as fermentation and other large-scale culture procedures. Useful yields of the products can be obtained under aerobic culture conditions. An exemplary growth condition for achieving, one or more cannabinoid products includes aerobic culture or fermentation conditions. In certain embodiments, the microbial organism can be sustained, cultured or fermented under aerobic conditions.

Substantially aerobic conditions include, for example, a culture, batch fermentation or continuous fermentation such that the dissolved oxygen concentration in the medium remains between 5% and 100% of saturation. The percent of dissolved oxygen can be maintained by, for example, sparging air, pure oxygen or a mixture of air and oxygen.

The culture conditions can be scaled up and grown continuously for manufacturing cannabinoid product. Exemplary growth procedures include, for example, fed-batch fermentation and batch separation; fed-batch fermentation and continuous separation, or continuous fermentation and continuous separation. All of these processes are well known in the art. Fermentation procedures are particularly useful for the biosynthetic production of commercial quantities of cannabinoid product. Generally, and as with non-continuous culture procedures, the continuous and/or near-continuous production of cannabinoid product will include culturing a cannabinoid producing organism on sufficient nutrients and medium to sustain and/or nearly sustain growth in an exponential phase. Continuo us culture under such conditions can include, for example, 1 day, 2, 3, 4, 5, 6 or 7 days or more. Additionally, continuous culture can include 1 week, 2, 3, 4 or 5 or more weeks and up to several months. Alternatively, the desired microorganism can be cultured for hours, if suitable for a particular application. It is to be understood that the continuous and/or near-continuous culture conditions also can include all time intervals in between these exemplary periods. It is further understood that the time of culturing the microbial organism is for a sufficient period of time to produce a sufficient amount of product for a desired purpose.

Fermentation procedures are well known in the art. Briefly, fermentation for the biosynthetic production of cannabinoid product can be utilized in, for example, fed-batch fermentation and batch separation; fed-batch fermentation and continuous separation, or continuous fermentation and continuous separation. Examples of batch and continuous fermentation procedures are well known in the art.

In some embodiments, the method comprises providing a cell as described herein comprising an exogenous nucleotide sequence coding for a recombinant polypeptide comprising an amino acid sequence having at least 70% identity to SEQ ID NO: 1, 2, 3, or 4 and culturing the cell to produce the cannabinoid, cannabinoid derivative, or cannabinoid analogue thereof. In some embodiments, the method comprises providing a cell as described herein comprising an exogenous nucleotide sequence coding for a recombinant polypeptide comprising an amino acid sequence having at least 90% identity to SEQ ID NO: 5 and culturing the cell to produce the cannabinoid or analogue thereof. In some embodiments, the amino acid sequence comprises at least one amino acid substitution as compared to SEQ ID NO: 1, 3, or 4. In some embodiments, the recombinant polypeptide further comprises a fusion domain as described herein.

In some embodiments, the expressed recombinant polypeptide comprises an amino acid sequence identical to SEQ ID NO: 1 with one to twenty amino acid substitutions and, optionally, one to twenty amino acids deleted from the C-terminus, wherein the amino acid substitutions are located in SEQ ID NO: 1 at positions selected from 49, 51, 52, 53, 65, 67, 68, 69, 111, 113, 122, 124, 125, 126, 164, 165, 166, 167, 170, 214, 215, 217, 219, 229, 231, 233, 235, 268, 269, 270, 282, 283, 284, 285, 286, 292, 293, 294, 295, 296, A297, and 298.

In some embodiments, the expressed recombinant polypeptide comprises an amino acid sequence identical to SEQ ID NO: 2 with one to twenty amino acid substitutions and, optionally, one to twenty amino acids deleted from the C-terminus, wherein the amino acid substitutions are located in SEQ ID NO: 2 at positions selected from 38, 39, 40, 41, 42, 43, 44, 55, 57, 58, 59, 101, 103, 111, 112, 114, 115, 116, 117, 155, 156, 157, 160, 167, 169, 204, 205, 207, 209, 219, 221, 223, 224, 225, 258, 259, 260, 272, 273, 274, 275, 276, 282, 283, 284, 285, 286, 287, and 288. In some embodiments, the recombinant polypeptide comprises an amino acid sequence identical to SEQ ID NO: 2 with one to three amino acid substitutions, wherein the amino acid substitutions are located in SEQ ID NO: 2 at positions selected from 116, 205, and 260. In some embodiments, the recombinant polypeptide comprises an amino acid sequence identical to SEQ ID NO: 2 with one to twenty amino acid substitutions, wherein the amino acid substitutions comprise one or more substitutions located in SEQ ID NO: 2 at positions selected from 116, 156, 205, 223, 225, 260, 276, 282, 283, and 284. In some embodiments, the remaining substitutions of the one to twenty substitutions are at the positions in SEQ ID NO: 2 identified above. In some embodiments, the recombinant protein comprising amino acid modifications identified above further comprising one to twenty amino acid deletions at the C-terminus as compared to SEQ ID NO: 2.

In some embodiments, the expressed recombinant polypeptide comprises an amino acid sequence identical to SEQ ID NO: 3 with one to twenty amino acid substitutions and, optionally, one to twenty amino acids deleted from the C-terminus, wherein the amino acid substitutions are located in SEQ ID NO: 3 at positions selected from 108, 109, 110, 111, 112, 113, 114, 125, 127, 128, 129, 171, 173, 181, 182, 184, 185, 186, 187, 225, 226, 227, 230, 237, 239, 274, 275, 277, 279, 289, 291, 293, 294, 295, 328, 329, 330, 342, 343, 344, 345, 346, 352, 353, 354, 355, 356, 357 and 358. In some embodiments, the recombinant polypeptide comprises an amino acid sequence identical to SEQ ID NO: 3 with one to three amino acid substitutions, wherein the amino acid substitutions are located in SEQ ID NO: 3 at positions selected from 186, 275, and 330. In some embodiments, the recombinant polypeptide comprises an amino acid sequence identical to SEQ ID NO: 3 with one to three amino acid substitutions, wherein the amino acid substitutions comprise one or more substitutions located in SEQ ID NO: 3 at positions selected from 186, 275, and 330. In some embodiments, the remaining substitutions of the one to twenty substitutions are at the positions in SEQ ID NO: 3 identified above. In some embodiments, the recombinant protein comprising amino acid modifications identified above further comprising one to twenty amino acid deletions at the C-terminus as compared to SEQ ID NO: 3.

In some embodiments, the expressed recombinant polypeptide comprises an amino acid sequence identical to SEQ ID NO: 4 with one to twenty amino acid substitutions and, optionally, one to twenty amino acids deleted from the C-terminus, wherein the amino acid substitutions are located in SEQ ID NO: 4 at positions selected from 38, 39, 40, 41, 42, 43, 44, 55, 57, 58, 59, 101, 103, 111, 112, 114, 115, 116, 117, 155, 156, 157, 160, 167, 169, 204, 205, 207, 209, 219, 221, 223, 224, 225, 258, 259, 260, 272, 273, 274, 275, 276, 282, 283, 284, 285, 286, 287, and 288. In some embodiments, the recombinant polypeptide comprises an amino acid sequence identical to SEQ ID NO: 4 with one to three amino acid substitutions, wherein the amino acid substitutions are located in SEQ ID NO: 4 at positions selected from 116, 205, and 260. In some embodiments, the recombinant polypeptide comprises an amino acid sequence identical to SEQ ID NO: 4 with one to three amino acid substitutions, wherein the amino acid substitutions comprise one or more substitutions located in SEQ ID NO: 4 at positions selected from 116, 156, 205, 223, 225, 260, 276, 282, 283, 284. In some embodiments, the remaining substitutions of the one to twenty substitutions are at the positions in SEQ ID NO: 4 identified above. In some embodiments, the recombinant protein comprising amino acid modifications identified above further comprising one to twenty amino acid deletions at the C-terminus as compared to SEQ ID NO: 4.

In some embodiments, the expressed recombinant polypeptide is capable of converting olivetolic acid (OA) and geranyl diphosphate (GPP) to one or more products comprising cannabigerolic acid (CBGA). In some embodiments, the one or more products comprise at least 10%, 15%, 20%, 25%, 30%, 35%, 40%, 45%, 50%, 55%, 60%, 65%, 70%, 75%, 80%, 85%, 90%, 95%, or substantially 100% CBGA. In some embodiments, at least about 50% of the one or more products is CBGA. In some embodiments, more than about 90% of the one or more products is CBGA. In some embodiments, the expressed recombinant polypeptide has a rate of formation of cannabigerolic acid (CBGA) from olivetolic acid (OA) and geranyl diphosphate (GPP) that is greater than the rate of formation of CBGA from OA and GPP by NphB under the same conditions. In some embodiments, the rate of formation of CBGA from OA and GPP is at least 1.1-fold, 1.2-fold, 1.3-fold, 1.4-fold, 1.5-fold, 1.6-fold, 1.7-fold, 1.8-fold, 1.9-fold, 2-fold, 2.5-fold, 5-fold, 10-fold, or more as compared to the rate of formation of CBGA from OA and GPP by NphB under the same conditions.

In some embodiments, the expressed recombinant polypeptide is capable of converting olivetolic acid (OA) and farnesyl pyrophosphate (FPP) to one or more cannabinoids, cannabinoid derivatives or cannabinoid analogues. In some embodiments, the activity of the recombinant polypeptide for converting OA and FPP to one or more cannabinoids, cannabinoid derivatives or cannabinoid analogues is at least about 10%, 15%, 20%, 25%, 30%, 35%, 40%, 45%, 50%, 55%, 60%, 65%, 70%, 75%, 80%, 85%, 90%, 95%, or substantially 100% of the activity of the recombinant polypeptide for converting OA and GPP to one or more cannabinoids, cannabinoid derivatives or cannabinoid analogues.

The cannabinoids, cannabinoid derivatives and cannabinoid analogues produced by the methods disclosed herein are not limited and may be any disclosed cannabinoid. In some embodiments, the cannabinoids, cannabinoid derivatives and cannabinoid analogues are selected from cannabigerolic acid, tetrahydrocannabinolic acid, tetrahydrocannabinol, cannabidiolic acid, cannabidiol, cannabigerol, cannabichromenic acid, cannabichromene, or an acid or derivative or analogue thereof.

In some embodiments, the methods further comprise a step of purifying or isolating the cannabinoids, derivatives or analogues thereof from the culture. Methods of isolation are not limited and may be any suitable method known in the art. Purification methods include, for example, extraction procedures as well as methods that include continuous liquid-liquid extraction, pervaporation, evaporation, filtration, membrane filtration (including reverse osmosis, nanofiltration, ultrafiltration, and microfiltration), membrane filtration with diafiltration, membrane separation, reverse osmosis, electrodialysis, distillation, extractive distillation, reactive distillation, azeotropic distillation, crystallization and recrystallization, centrifugation, extractive filtration, ion exchange chromatography, size exclusion chromatography, adsorption chromatography, carbon adsorption, hydrogenation, and ultrafiltration or centrifugal partition chromatography (CPC).

In some embodiments, the cells are grown in stirred tank fermenters with feed supplementation (sugars with or without organic acids) where the dissolved oxygen, temperature, and pH are be controlled according to the optimal growth and production process. In some embodiments, aqueous non-miscible organic solvents are supplemented to dissolve added organic acids or extract the cannabinoid products as they are being synthesized. In some embodiments, these solvents may include, but are not limited to, isopropyl myristate (IPM), diisobutyl adipate, decane, dodecane, hexadecane or anther organic solvent with log P>5. The later number (log P) is defined as the log of a compound's partition between water and octanol and is a standard parameter of a compound's hydrophobicity (the larger the log P the less soluble in water). Depending on the fermentation process, the products can be isolated and purified using different methods.

If no organic cosolvent is used and the targeted cannabinoid(s) is being secreted to the culture supernatant, different methods can be applied. In one embodiment, an aqueous miscible organic solvent (ethanol, acetonitrile, etc.) is added to dissolve the products. In some embodiments, a simple filtration, ultrafiltration or centrifugation can remove the cells and the aqueous media evaporated to dryness or to a small volume from which the cannabinoid product will precipitate or crystalize. Alternatively, the cell supernatant can be extracted with an aqueous immiscible organic solvent (ethyl acetate, heptane, decane, etc.) to extract the cannabinoids. Evaporation of the organic solvent and a possible recrystallization will produce pure cannabinoid. If the cannabinoid products are not secreted to the media and are trapped inside the cell, different methods for their extraction and purification can be utilized. In some embodiments, cells are disrupted using mechanical methods or by suspension in appropriate lysis buffers from which the cannabinoids can be extracted with an organic aqueous immiscible solvent (ethyl acetate, hexane, decane, methylene chloride, etc.). In other embodiments, cells may be suspended in an organic solvent (ethanol, methanol, methylene chloride, etc.) that extracts the cannabinoids from the cells.

In some embodiments, an organic solvent is required during growth that is separated at the end of the fermentation. Back extraction with basic aqueous solvent or a different organic solvent with low boiling point and high polarity (ethanol, acetonitrile, etc.) will remove the cannabinoids. Isolation can then involve a simple pH shift if water is used, or an evaporation if organic solvents are used. In both cases, a recrystallization step may be required at the end to improve purity of the product.

### Further Embodiments of the Disclosure

1. A recombinant polypeptide comprising an amino acid sequence with at least 70% identity to SEQ ID NO: 1, 2, 3, or 4, wherein the amino acid sequence comprises at least one amino acid modification as compared to SEQ ID NO: 1, 3, or 4.

2. A recombinant polypeptide comprising an amino acid sequence with at least 90% identity to SEQ ID NO: 5, wherein the amino acid sequence comprises at least one amino acid modification as compared to SEQ ID NO: 1, 2, 3, or 4.

3. The recombinant polypeptide of items 1-2, further comprises a histidine tag sequence.

4. The recombinant polypeptide of items 1-3 comprising an amino acid sequence identical to SEQ ID NO: 1 with one to twenty amino acid substitutions, wherein the amino acid substitutions are located in SEQ ID NO: 1 at positions selected from 49, 51, 52, 53, 65, 67, 68, 69, 111, 113, 122, 124, 125, 126, 164, 165, 166, 167, 170, 214, 215, 217, 219, 229, 231, 233, 235, 268, 269, 270, 282, 283, 284, 285, 286, and 293.

5. The recombinant polypeptide of items 1-3 comprising an amino acid sequence identical to SEQ ID NO: 2 with one to twenty amino acid substitutions, wherein the amino acid substitutions are located in SEQ ID NO: 2 at positions selected from 38, 39, 40, 41, 42, 43, 44, 55, 57, 58, 59, 101, 103, 111, 112, 114, 115, 116, 117, 155, 156, 157, 160, 167, 169, 204, 205, 207, 209, 219, 221, 223, 224, 225, 258, 259, 260, 272, 273, 274, 275, 276, and 283.

6. The recombinant polypeptide of items 1-3 comprising an amino acid sequence identical to SEQ ID NO: 3 with one to twenty amino acid substitutions, wherein the amino acid substitutions are located in SEQ ID NO: 3 at positions selected from 108, 109, 110, 111, 112, 113, 114, 125, 127, 128, 129, 171, 173, 181, 182, 184, 185, 186, 187, 225, 226, 227, 230, 237, 239, 274, 275, 277, 279, 289, 291, 293, 294, 295, 328, 329, 330, 342, 343, 344, 345, 346, 353.

7. The recombinant polypeptide of items 1-3 comprising an amino acid sequence identical to SEQ ID NO: 4 with one to twenty amino acid substitutions, wherein the amino acid substitutions are located in SEQ ID NO: 4 at positions selected from 38, 39, 40, 41, 42, 43, 44, 55, 57, 58, 59, 101, 103, 111, 112, 114, 115, 116, 117, 155, 156, 157, 160, 167, 169, 204, 205, 207, 209, 219, 221, 223, 224, 225, 258, 259, 260, 272, 273, 274, 275, 276, and 283.

8. The recombinant polypeptide of items 1-7, wherein the recombinant polypeptide converts olivetolic acid (OA) and geranyl diphosphate (GPP) to one or more products comprising cannabigerolic acid (CBGA).

9. The recombinant polypeptide of item 8, wherein at least about 50% of the one or more products is CBGA.

10. The recombinant polypeptide of items 1-9, wherein the recombinant polypeptide has a rate of formation of cannabigerolic acid (CBGA) from olivetolic acid (OA) and geranyl diphosphate (GPP) that is greater than the rate of formation of CBGA from OA and GPP by NphB under the same conditions.

11. The recombinant polypeptide of items 1-10, wherein the recombinant polypeptide converts olivetolic acid (OA) and farnesyl pyrophosphate (FPP) to one or more cannabinoids, cannabinoid derivatives, or cannabinoid analogues.

12. The recombinant polypeptide of items 1-11, wherein the recombinant polypeptide converts divarinic acid (DVA) and geranyl diphosphate (GPP) to one or more cannabinoids, cannabinoid derivatives, or cannabinoid analogues.

13. A cell comprising an exogenous nucleotide sequence coding for the recombinant polypeptide of items 1-12.

14. The cell of item 13, wherein the cell is a bacteria, an algae, a yeast, or a plant cell.

15. The cell of item 14, wherein the yeast is an oleaginous yeast.

16. The cell of item 14, wherein the bacteria is Escherichia coli.

17. A cell comprising an exogenous nucleotide sequence coding for a recombinant polypeptide comprising an amino acid sequence having at least 70% identity to SEQ ID NO: 1, 2, 3, or 4.

18. A cell comprising an exogenous nucleotide sequence coding for a recombinant polypeptide comprising an amino acid sequence having at least 90% identity to SEQ ID NO: 5.

19. The cell of item 17 or 18, wherein the amino acid sequence comprises at least one amino acid modification as compared to SEQ ID NO: 1, 3, or 4.

20. The cell of items 18-19, wherein the recombinant polypeptide comprises a histidine tag sequence.

21. The cell of items 18-20 comprising an exogenous nucleotide sequence coding for a recombinant polypeptide comprising an amino acid sequence identical to SEQ ID NO: 1 with one to twenty amino acid substitutions, wherein the amino acid substitutions are located in SEQ ID NO: 1 at positions selected from 49, 51, 52, 53, 65, 67, 68, 69, 111, 113, 122, 124, 125, 126, 164, 165, 166, 167, 170, 214, 215, 217, 219, 229, 231, 233, 235, 268, 269, 270, 282, 283, 284, 285, 286, and 293.

22. The cell of items 18-20 comprising an exogenous nucleotide sequence coding for a recombinant polypeptide comprising an amino acid sequence identical to SEQ ID NO: 2 with one to twenty amino acid substitutions, wherein the amino acid substitutions are located in SEQ ID NO: 2 at positions selected from 38, 39, 40, 41, 42, 43, 44, 55, 57, 58, 59, 101, 103, 111, 112, 114, 115, 116, 117, 155, 156, 157, 160, 167, 169, 204, 205, 207, 209, 219, 221, 223, 224, 225, 258, 259, 260, 272, 273, 274, 275, 276, and 283.

23. The cell of items 18-20 comprising an exogenous nucleotide sequence coding for a recombinant polypeptide comprising an amino acid sequence identical to SEQ ID NO: 3 with one to twenty amino acid substitutions, wherein the amino acid substitutions are located in SEQ ID NO: 3 at positions selected from 108, 109, 110, 111, 112, 113, 114, 125, 127, 128, 129, 171, 173, 181, 182, 184, 185, 186, 187, 225, 226, 227, 230, 237, 239, 274, 275, 277, 279, 289, 291, 293, 294, 295, 328, 329, 330, 342, 343, 344, 345, 346, 353.

24. The cell of items 18-20 comprising an exogenous nucleotide sequence coding for a recombinant polypeptide comprising an amino acid sequence identical to SEQ ID NO: 4 with one to twenty amino acid substitutions, wherein the amino acid substitutions are located in SEQ ID NO: 4 at positions selected from 38, 39, 40, 41, 42, 43, 44, 55, 57, 58, 59, 101, 103, 111, 112, 114, 115, 116, 117, 155, 156, 157, 160, 167, 169, 204, 205, 207, 209, 219, 221, 223, 224, 225, 258, 259, 260, 272, 273, 274, 275, 276, and 283.

25. The cell of items 17-24, wherein the recombinant polypeptide is capable of converting olivetolic acid (OA) and geranyl diphosphate (GPP) to one or more products comprising cannabigerolic acid (CBGA).

26. The cell of item 25, wherein at least about 50% of the one or more products is CBGA.

27. The cell of items 17-26, wherein the recombinant polypeptide has a rate of formation of cannabigerolic acid (CBGA) from olivetolic acid (OA) and geranyl diphosphate (GPP) that is greater than the rate of formation of CBGA from OA and GPP by NphB under the same conditions.

28. The cell of items 17-27, wherein the recombinant polypeptide is capable of converting olivetolic acid (OA) and farnesyl pyrophosphate (FPP) to one or more cannabinoid, cannabinoid derivatives, or cannabinoid analogues.

29. The cell of items 17-28, wherein the recombinant polypeptide is capable of converting divarinic acid (DVA) and geranyl diphosphate (GPP) to one or more cannabinoid, cannabinoid derivatives, or cannabinoid analogues.

30. The cell of items 17-29, wherein the cell comprises an olivetolic acid pathway.

31. The cell of item 30, wherein the olivetolic acid pathway comprises a polyketide cyclase.

32. The cell of item 31, wherein an exogenous nucleotide codes for the polyketide cyclase.

33. The cell of items 17-32, wherein the cell comprises a geranyl pyrophosphate (GPP) pathway.

34. The cell of item 33, wherein the GPP pathway comprises geranyl pyrophosphate synthase.

35. The cell of item 34, wherein an exogenous nucleotide codes for the geranyl pyrophosphate synthase.

36. The cell of items 17-35, wherein the cell comprises a farnesyl pyrophosphate (FPP) pathway.

37. The cell of item 36, wherein the FPP pathway comprises a farnesyl pyrophosphate synthase.

38. The cell of item 37, wherein an exogenous nucleotide codes for the farnesyl pyrophosphate synthase.

39. The cell of items 17-38, wherein the cell comprises a divarinic acid (DVA) pathway.

40. The cell of item 39, wherein the DVA pathway comprises divarinic acid synthase.

41. The cell of item 40, wherein an exogenous nucleotide codes for the divarinic acid synthase.

42. The cell of items 17-41, wherein the cell is capable of producing a cannabinoid selected from tetrahydrocannabinol, cannabidiol, cannabigerol, or an acid, derivative, or analogue thereof.

43. The cell of item 42, wherein production of the cannabinoid is under control of an inducible promoter.

44. The cell of items 17-43, wherein the cell is a bacteria, an algae, or a yeast.

45. The cell of item 44, wherein the yeast is an oleaginous yeast.

46. The cell of item 44, wherein the bacteria is Escherichia coli.

47. A composition comprising cannabigerolic acid, tetrahydrocannabinol, cannabidiol, cannabigerol, or an acid, derivative, or an analogue thereof produced by the cell of items 17-46.

48. A method of producing a cannabinoid or an acid, derivative, or analogue thereof, the method comprising providing a cell comprising an exogenous nucleotide sequence coding for a recombinant polypeptide comprising an amino acid sequence having at least 70% identity to SEQ ID NO: 1, 2, 3, or 4 and culturing the cell to produce the cannabinoid or acid, derivative, or analogue thereof.

49. A method of producing a cannabinoid or an acid, derivative, or analogue thereof, the method comprising providing a cell comprising an exogenous nucleotide sequence coding for a recombinant polypeptide comprising an amino acid sequence having at least 90% identity to SEQ ID NO: 5 and culturing the cell to produce the cannabinoid or an acid, derivative, or analogue thereof.

50. The method of items 48-49, wherein the amino acid sequence comprises at least one amino acid modification as compared to SEQ ID NO: 1, 3, or 4.

51. The method of items 48-50, wherein the recombinant polypeptide further comprises a histidine tag sequence.

52. The method of items 48-51 wherein the amino acid sequence is identical to SEQ ID NO: 1 with one to twenty amino acid substitutions, wherein the amino acid substitutions are located in SEQ ID NO: 1 at positions selected from 49, 51, 52, 53, 65, 67, 68, 69, 111, 113, 122, 124, 125, 126, 164, 165, 166, 167, 170, 214, 215, 217, 219, 229, 231, 233, 235, 268, 269, 270, 282, 283, 284, 285, 286, and 293.

53. The method of items 48-51 wherein the amino acid sequence is identical to SEQ ID NO: 2 with one to twenty amino acid substitutions, wherein the amino acid substitutions are located in SEQ ID NO: 2 at positions selected from 38, 39, 40, 41, 42, 43, 44, 55, 57, 58, 59, 101, 103, 111, 112, 114, 115, 116, 117, 155, 156, 157, 160, 167, 169, 204, 205, 207, 209, 219, 221, 223, 224, 225, 258, 259, 260, 272, 273, 274, 275, 276, and 283.

54. The method of items 48-51 wherein the amino acid sequence is identical to SEQ ID NO: 3 with one to twenty amino acid substitutions, wherein the amino acid substitutions are located in SEQ ID NO: 3 at positions selected from 108, 109, 110, 111, 112, 113, 114, 125, 127, 128, 129, 171, 173, 181, 182, 184, 185, 186, 187, 225, 226, 227, 230, 237, 239, 274, 275, 277, 279, 289, 291, 293, 294, 295, 328, 329, 330, 342, 343, 344, 345, 346, 353.

55. The method of items 48-51 wherein the amino acid sequence is identical to SEQ ID NO: 4 with one to twenty amino acid substitutions, wherein the amino acid substitutions are located in SEQ ID NO: 4 at positions selected from 38, 39, 40, 41, 42, 43, 44, 55, 57, 58, 59, 101, 103, 111, 112, 114, 115, 116, 117, 155, 156, 157, 160, 167, 169, 204, 205, 207, 209, 219, 221, 223, 224, 225, 258, 259, 260, 272, 273, 274, 275, 276, and 283.

56. The method of items 48-55, wherein the recombinant polypeptide converts olivetolic acid (OA) and geranyl diphosphate (GPP) to one or more products comprising cannabigerolic acid (CBGA).

57. The method of item 56, wherein at least about 50% of the one or more products is CBGA.

58. The method of item 57, wherein the recombinant polypeptide has a rate of formation of cannabigerolic acid (CBGA) from olivetolic acid (OA) and geranyl diphosphate (GPP) that is greater than the rate of formation of CBGA from OA and GPP by NphB under the same conditions.

59. The method of items 48-58, wherein the recombinant polypeptide converts olivetolic acid (OA) and farnesyl pyrophosphate (FPP) to one or more cannabinoid, cannabinoid derivatives, or cannabinoid analogues.

60. The method of items 48-58, wherein the cell comprises an olivetolic acid pathway.

61. The method of item 60, wherein the olivetolic acid pathway comprises a polyketide cyclase.

62. The method of item 61, wherein an exogenous nucleotide codes for the polyketide cyclase.

63. The method of items 48-62, wherein the cell comprises a geranyl pyrophosphate (GPP) pathway.

64. The method of item 63, wherein the GPP pathway comprises geranyl pyrophosphate synthase.

65. The method of item 64, wherein an exogenous nucleotide codes for the geranyl pyrophosphate synthase.

66. The method of items 48-65, wherein the cell comprises a farnesyl pyrophosphate (FPP) pathway.

67. The method of item 66, wherein the FPP pathway comprises a farnesyl pyrophosphate synthase.

68. The method of item 67, wherein an exogenous nucleotide codes for the farnesyl pyrophosphate synthase.

69. The method of items 48-68, wherein the cell comprises a divarinic acid (DVA) pathway.

70. The cell of item 69, wherein the DVA pathway comprises divarinic acid synthase.

71. The cell of item 70, wherein an exogenous nucleotide codes for the divarinic acid synthase.

72. The method of items 48-71, wherein the cannabinoid or analogue thereof is selected from cannabigerolic acid, tetrahydrocannabinol, cannabidiol, cannabigerol, or an acid, derivative or analogue thereof.

73. The method of item 72, wherein production of the cannabinoid or acid, derivative or analogue thereof is under control of an inducible promoter.

74. The method of items 48-73, wherein the cell is a bacteria, an algae, or a yeast.

75. The method of item 74, wherein the yeast is an oleaginous yeast.

76. The method of item 74, wherein the bacteria is Escherichia coli.

77. The method of items 48-76, further comprising a step of purifying or isolating the cannabinoid or derivative or analogue thereof from the culture.

Specific examples of certain aspects of the inventions disclosed herein are set forth below in the Examples.

One skilled in the art readily appreciates that the present invention is well adapted to carry out the objects and obtain the ends and advantages mentioned, as well as those inherent therein. The details of the description and the examples herein are representative of certain embodiments, are exemplary, and are not intended as limitations on the scope of the invention. Modifications therein and other uses will occur to those skilled in the art. These modifications are encompassed within the spirit of the invention. It will be readily apparent to a person skilled in the art that varying substitutions and modifications may be made to the invention disclosed herein without departing from the scope and spirit of the invention.

The articles “a” and “an” as used herein in the specification and in the claims, unless clearly indicated to the contrary, should be understood to include the plural referents. Claims or descriptions that include “or” between one or more members of a group are considered satisfied if one, more than one, or all of the group members are present in, employed in, or otherwise relevant to a given product or process unless indicated to the contrary or otherwise evident from the context. The invention includes embodiments in which exactly one member of the group is present in, employed in, or otherwise relevant to a given product or process. The invention also includes embodiments in which more than one, or all of the group members are present in, employed in, or otherwise relevant to a given product or process. Furthermore, it is to be understood that the invention provides all variations, combinations, and permutations in which one or more limitations, elements, clauses, descriptive terms, etc., from one or more of the listed claims is introduced into another claim dependent on the same base claim (or, as relevant, any other claim) unless otherwise indicated or unless it would be evident to one of ordinary skill in the art that a contradiction or inconsistency would arise. It is contemplated that all embodiments described herein are applicable to all different aspects of the invention where appropriate. It is also contemplated that any of the embodiments or aspects can be freely combined with one or more other such embodiments or aspects whenever appropriate. Where elements are presented as lists, e.g., in Markush group or similar format, it is to be understood that each subgroup of the elements is also disclosed, and any element(s) can be removed from the group. It should be understood that, in general, where the invention, or aspects of the invention, is/are referred to as comprising particular elements, features, etc., certain embodiments of the invention or aspects of the invention consist, or consist essentially of, such elements, features, etc. For purposes of simplicity those embodiments have not in every case been specifically set forth in so many words herein. It should also be understood that any embodiment or aspect of the invention can be explicitly excluded from the claims, regardless of whether the specific exclusion is recited in the specification. For example, any one or more nucleic acids, polypeptides, cells, species or types of organism, disorders, subjects, or combinations thereof, can be excluded.

Where the claims or description relate to a composition of matter, e.g., a nucleic acid, polypeptide, or cell, it is to be understood that methods of making or using the composition of matter according to any of the methods disclosed herein, and methods of using the composition of matter for any of the purposes disclosed herein are aspects of the invention, unless otherwise indicated or unless it would be evident to one of ordinary skill in the art that a contradiction or inconsistency would arise. Where the claims or description relate to a method, e.g., it is to be understood that methods of making compositions useful for performing the method, and products produced according to the method, are aspects of the invention, unless otherwise indicated or unless it would be evident to one of ordinary skill in the art that a contradiction or inconsistency would arise.

Where ranges are given herein, the invention includes embodiments in which the endpoints are included, embodiments in which both endpoints are excluded, and embodiments in which one endpoint is included and the other is excluded. It should be assumed that both endpoints are included unless indicated otherwise. Furthermore, it is to be understood that unless otherwise indicated or otherwise evident from the context and understanding of one of ordinary skill in the art, values that are expressed as ranges can assume any specific value or subrange within the stated ranges in different embodiments of the invention, to the tenth of the unit of the lower limit of the range, unless the context clearly dictates otherwise. It is also understood that where a series of numerical values is stated herein, the invention includes embodiments that relate analogously to any intervening value or range defined by any two values in the series, and that the lowest value may be taken as a minimum and the greatest value may be taken as a maximum. Numerical values, as used herein, include values expressed as percentages. For any embodiment of the invention in which a numerical value is prefaced by “about” or “approximately”, the invention includes an embodiment in which the exact value is recited. For any embodiment of the invention in which a numerical value is not prefaced by “about” or “approximately”, the invention includes an embodiment in which the value is prefaced by “about” or “approximately”. “Approximately” or “about” generally includes numbers that fall within a range of 1% or in some embodiments within a range of 5% of a number or in some embodiments within a range of 10% of a number in either direction (greater than or less than the number) unless otherwise stated or otherwise evident from the context (except where such number would impermissibly exceed 100% of a possible value). It should be understood that, unless clearly indicated to the contrary, in any methods claimed herein that include more than one act, the order of the acts of the method is not necessarily limited to the order in which the acts of the method are recited, but the invention includes embodiments in which the order is so limited. It should also be understood that unless otherwise indicated or evident from the context, any product or composition described herein may be considered “isolated”.

### EXAMPLES

### Example 1—Identification and Cloning of Soluble Prenyltransferases

### Enzyme Discovery Strategy.

The strategy taken to identify soluble aromatic prenyltransferases (APT) with putative OA activity relied on three general approaches. The first approach involved identifying and selecting sequence homologs of NphB (the only known microbial prenyltransferase with this activity). The second relied on a literature search of enzymes that use GPP as the prenyl donor (many prenyltransferases use DMAPP or FPP) for transfer to aromatic substrates. The third utilized artificial intelligence methods to identify potential enzymes with activity on OA and GPP. After this analysis, a total of 89 new enzymes were identified and cloned. NphB and a couple of its mutants with reported increased activity and selectivity were also cloned and used as benchmark for comparison in some assays.

### Cloning Methods, Vectors and Strains

Genes for each APT enzyme were optimized for expression, synthesized (SGI-DNA), and cloned into the pM264-c vector (ATUM). Genes were sequenced verified and then subcloned into the pD441-NHT expression vector (ATUM) with N-terminal His tag and TEV protease cleavage site under control of the T5 promoter. Plasmids were transformed into chemically competent E. coli BL21 (DE3) cells (NEB), plated on LB agar plates with 50 μg/mL kanamycin, and grown overnight at 37° C. Colony PCR was run to verify gene fragment insertion and positive colonies were used to start overnight cultures in liquid LB media with 50 μg/mL kanamycin. Cultures were grown overnight at 37° C. and then diluted with sterile-filtered glycerol to create stocks containing 25% glycerol, which were stored at −80° C.

### Example 2—Growth and High Throughput Screening of Enzymes

Glycerol stocks containing each APT plasmid (and two strains containing plasmid vector only) were used to inoculate 10 mL of TB media with 50 μg/mL kanamycin in sterile Falcon tubes. The cultures were grown at 37° C. with 200 rpm shaking until reaching an OD600 of 0.8-0.9. At this point, the tubes were transferred to a shaker at room temperature for 45 min, after which they were induced with 0.25 mM IPTG. After 16 hours at room temperature with 120 rpm shaking, 2 mL of each culture was transferred to a well of a deep 96-well plate and centrifuged at 4750 rpm for 15 min. The clarified supernatant was decanted and an additional 2 mL of the same culture was added to each well. After centrifugation and decanting of liquid, the cell pellets (from 4 mL total culture) were stored at −80° C.

The plate containing the frozen cell pellets was removed from the freezer, and 0.5 mL of lysis buffer (B-PER (Thermo Scientific) with 5 mM MgCl₂, 0.1 mg/mL lysozyme, and 2 μL/mL DNase I (TURBO DNase ThermoFisher) was added to each well. The pellets were thawed and resuspend in this solution by mixing with a pipette. The plate was sealed and shaken at room temperature for 10 min before it was centrifuged at 4750 rpm for 20 min at 4° C. to precipitate the pellets of the lysed cells. Using a multichannel pipette, 0.2 mL of each lysate was mixed with 0.2 mL of reaction buffer (100 mM HEPES pH 7.4 with 100 mM NaCl, 10 mM MgCl₂, 3 mM OA, and 2 mM GPP). Another plate was prepared using the same method except with the addition of FPP instead of GPP as a substrate. The plate was sealed and incubated in a shaker oven at 33° C. with shaking at 200 rpm. After 12 h, 0.2 mL of each reaction was transferred to a new plate and mixed with 0.4 mL of acetonitrile containing 0.1% formic acid. The plate was centrifuged at 4750 rpm for 15 min to precipitate proteins and salts and 0.3 mL of each well was transferred to a clean plate that was sealed and analyzed for products by HPLC using UV (DAD detector) and MS (qToF) detection.

### HPLC Methods and Analysis

Column: 2.1×100 mm COSMOCORE C18 (Nacalai USA, Inc.)
Buffers
   Buffer A: Water, 10 mM ammonium formate, 0.1% formic acid
   Buffer B: Acetonitrile, 0.1% formic acid
Flow rate: 0.3 mL/min
Column temperature: 40 Celsius
Injection volume: 2 uL

Method:

| Time | Buffer B |
|---|---|
| 0.0 | 50% |
| 0.5 | 50% |
| 1.0 | 80% |
| 4.0 | 90% |
| 6.0 | 90% |
| 6.1 | 50% |
| 8.0 | 50% |

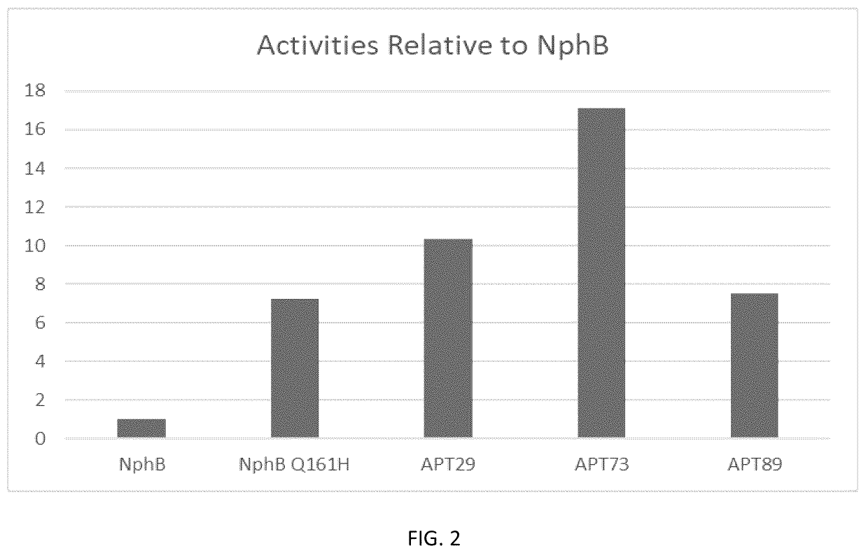

Using this method, CBGA elutes in 3.85 min and OA at 1.32 min. This method also identified several byproducts. In this screening, NphB and one of its best reported mutants, NphB Q161R, were included as positive controls. Under these conditions, the expression of each APT in the lysate varied significantly, with some APTs having no visible protein and others showing clear overexpression as evaluated by protein gels. The enzymes were later purified and more accurate comparisons were made (FIG. 2).

### Alternate HPLC Methods and Analysis

Column: 2.1×50 mm COSMOCORE PBr (Nacalai USA, Inc.)
Buffers
   Buffer A: Water, 0.1% formic acid
   Buffer B: Acetonitrile, 0.1% formic acid
Flow rate: 0.4 mL/min
Column temperature: 50 Celsius
Injection volume: 1 uL

Method:

| Time | Buffer B |
|---|---|
| 0.0 | 5% |
| 4.3 | 70% |
| 8.3 | 90% |
| 8.6 | 5% |
| 10 | 5% |

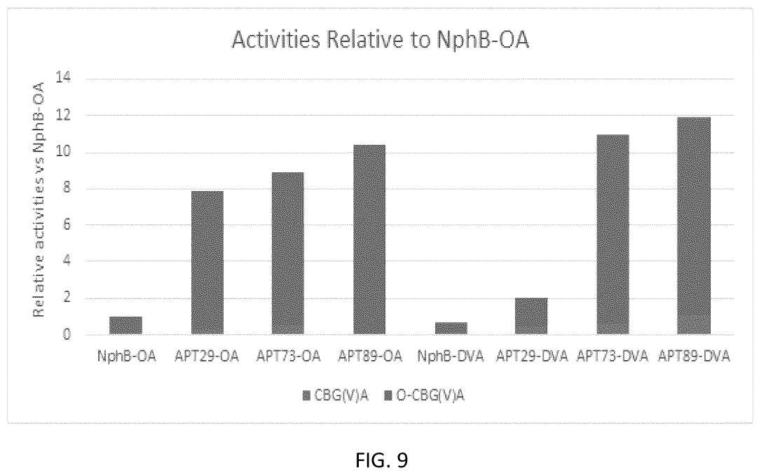

Using this alternate method, CBGA elutes in 5.13 min, CBGVA elutes at 4.63 min, O-CBGA elutes at 5.24 min, O-CBGVA elutes at 4.73 min, F-CBGA elutes at 6.38 min and F-CBGVA elutes at 5.9 min and OA at 2.91 min and Divarinic acid at 2.05 min. Activities relative to NphB with OA were assessed (See FIG. 9).

### Example 3—Purification and Activity Characterization of APTs

In order to compare the enzyme's activities more accurately, larger cultures of the best hits and controls from the first screen were grown and the enzymes were purified according to the following protocol. Glycerol stocks of each recombinant strain were used to inoculate 2 mL of LB with 50 μg/mL kanamycin. After overnight growth at 37° C., 0.5 mL was used to inoculate 100 mL of TB (with 50 μg/mL kanamycin). The cultures were grown at 37° C. with 250 rpm shaking until an OD600 of approximately 0.8-1. At this point, the cultures were transferred to a shaker at room temperature for 30 min, after which they were induced with 0.25 mM IPTG. After 16 h at 150 rpm shaking, the cells were pelleted by centrifugation

Cell pellets were resuspended in 10 mL lysis buffer (B-PER with 5 mM MgCl₂, 100 μg/mL lysozyme, and 2 μL/mL DNaseI (TURBO DNase ThermoFisher)). After incubation at room temperature for 10 min, the cell debris were removed by centrifugation at 4750 rpm for 10 min at 4° C. Lysates were loaded in pre-equilibrated cobalt spin columns (ThermoFisher, HisPur Cobalt spin column, 1 mL) and tagged proteins were purified according to manufacturer's protocol. The eluted proteins were exchanged into the final buffer (TrisHCl 25 mM, 5 mM MgCl₂, 150 mM NaCl 10% v/v glycerol) using Amicon Ultra 15 centrifugal filters (10 kDa MWCO). Proteins can be stored at −20° C. for at least a week.

### Characterization of APTs

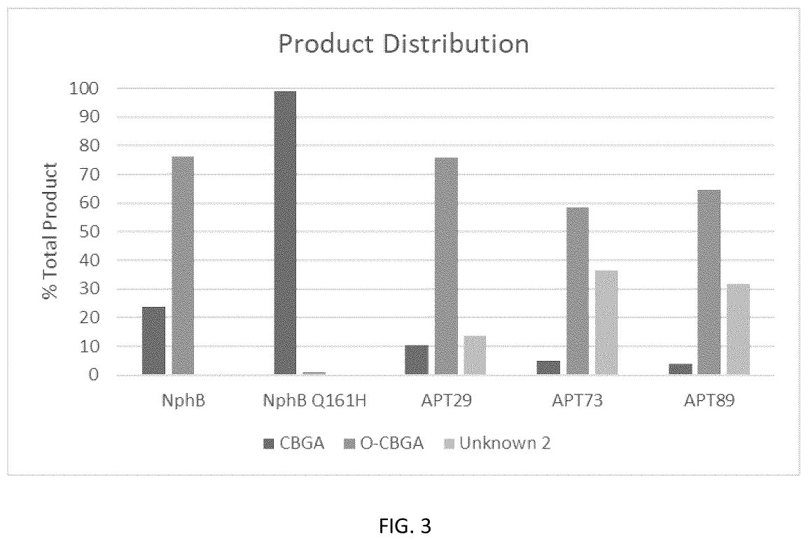

Small scale reactions were then prepared using purified enzymes as follows. The purified enzymes were normalized to the same concentration before adding to the reaction. In a 96 well plate 0.15 mL of purified enzyme (0.1 to 0.2 mg/mL final concentration) was mixed with 0.3 mL of reaction buffer (75 mM HEPES, 75 mM NaCl, 5 mM MgCl₂, 1.6 mM OA and 1.2 mM GPP, pH 7.4). The reaction was shaken at 33° C. After 1, 2, and 4 h, 0.1 mL samples were removed, mixed with 0.2 mL acetonitrile containing 0.1% formic acid, and centrifuged to remove salts and protein. Clarified solution (0.2 mL) from each reaction was removed and analyzed by HPLC using the method described earlier. The relative activity (accounting all products made in each reaction) compared to NphB is shown FIG. 3.

### Product Analysis:

All products shown in FIGS. 2-3 were analyzed by MS (qToF Agilent 6520) to confirm that the peaks at the same retention time for each sample gave the same product and verify the production of CBGA.

Authentic CBGA elutes at 3.85 min and has the same MS fingerprint as all samples that contain this peak. The major fragments M/Z=383.2164 (CBGA-Mg), 361.2319 (M+H), 343.2225 (CBGA-H2O).

The second major peak at 4.05 min is also produced by NphB and has been reported to be prenylation at the 2-OH of the olivetol ring. The major fragments are M/Z=383.2164 (CBGA-Mg), 361.2319 (M+H) but also M/Z=237.1097 which is indicative of the CH2-O-CBGA obtained from fragmenting the GPP side chain.

The product at 5.65 min, Unknown 2, has a peak at M/Z=497.3573 which is indicative of double prenylated OA, M/Z=361.22336 (single prenylated OA) and 237.1094 indicative of C2-OH prenylation.

Products with FPP

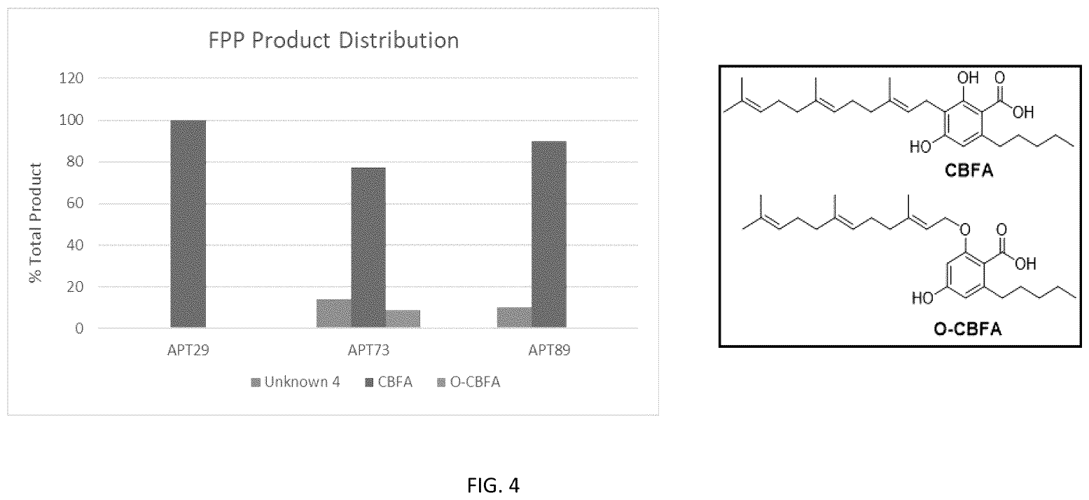

Enzymes APT29, APT73, and APT89 all yielded products using OA and FPP as substrates. The activity with these substrates was about 10% of their activity using OA and GPP. MS analysis of the products formed in these reactions strongly suggest that analogous products to the ones made using GPP are produced as shown in FIG. 4.

Products with Divarinic Acid

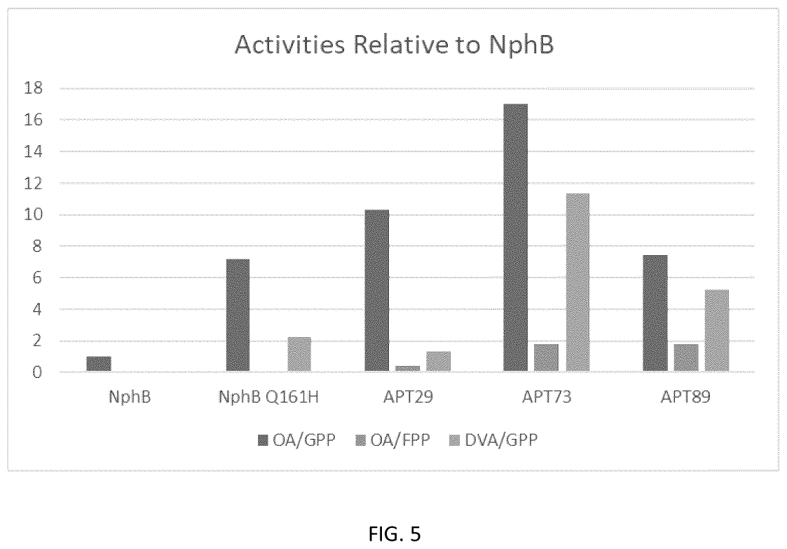

APT29, APT73, and APT89 can also accept olivetolic acid analogs as substrates as shown by their reactivity with divarinic acid (2,4 dihydroxy-6-propyl-benzoic acid: DVA) and GPP. A summary of the activity profile of all enzymes with different substrates is shown in FIG. 5.

### Alternate Characterization of APTs

Small scale reactions were also prepared using purified enzymes as follows. The purified enzymes were normalized to the same concentration before adding to the reaction. In a 96 well plate 0.3 mL of purified enzyme 1 or 5 μM final concentration) was mixed with 0.9 mL of reaction buffer (100 mM HEPES, 75 mM NaCl, 5 mM MgCl₂, 1.3 mM OA or DVA and 1.3 mM GPP, pH 7.4). The reaction was incubated at 33° C. with 250 rpm shaking. At time points between 0 and 180 min, 0.1 mL samples were removed, mixed with 0.2 mL acetonitrile containing 0.2% formic acid and 0.5 mg/mL pentylbenzoate and then centrifuged to remove salts and protein. Clarified solution (0.2 mL) from each reaction was removed and analyzed by HPLC using the method described earlier. Products were plotted vs time and slopes were determined from the linear portion of the plots and then normalized to the total product (CBGA and O-CBGA) of NphB, which was set to 1, as shown in FIG. 9. After review, it was determined that the activity for APT89 shown in FIG. 9 is accurate while the activity of APT89 shown in FIG. 2 is incorrect due to an experimental error.

### Alternate Product Analysis:

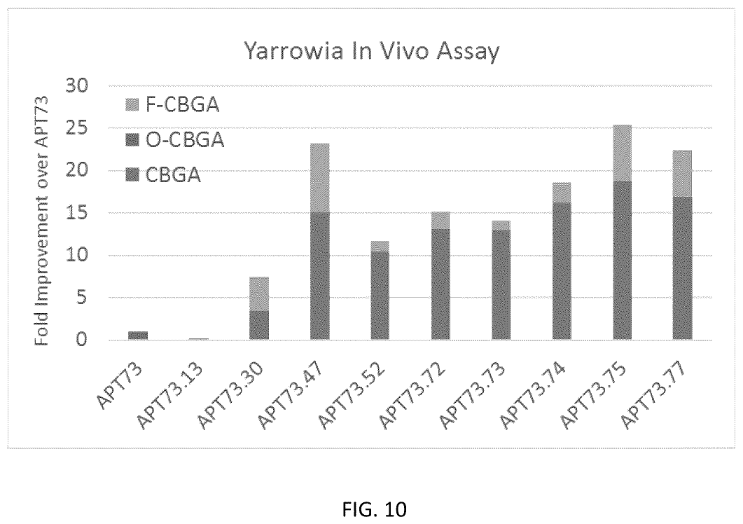

All products shown in FIGS. 9 and 10 were analyzed by MS (qToF Agilent 6520) to confirm that the peaks at the same retention time for each sample gave the same product and verify the production of CBGA.

Authentic CBGA elutes at 5.13 min and has the same MS fingerprint as all samples that contain this peak. The major fragments M/Z=383.2164 (CBGA-Mg), 361.2319 (M+H), 343.2225 (CBGA-H2O).

The second major peak at 5.24 min is also produced by NphB and has been reported to be prenylation at the 2-OH of the olivetol ring. The major fragments are M/Z=383.2164 (CBGA-Mg), 361.2319 (M+H) but also M/Z=237.1097 which is indicative of the CH2=O-CBGA obtained from fragmenting the GPP side chain.

Products with FPP

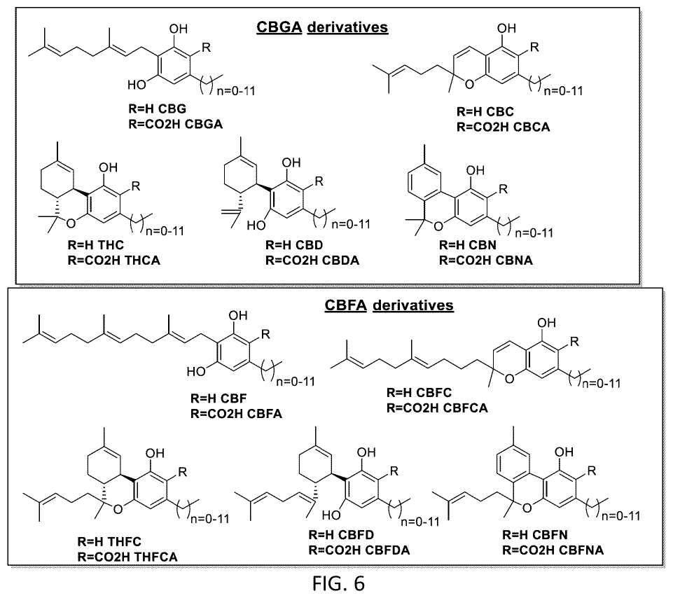

Enzymes APT29, APT73, and APT89 all yielded products using OA and FPP as substrates. The activity with these substrates was about 10% of their activity using OA and GPP. MS analysis of the products formed in these reactions strongly suggest that these are analogous products to the ones made using GPP (CBGA and O-CBGA analogues of FPP). The activity and selectivity of certain mutants in the presence of varying amounts of FPP and GPP with OA is described in FIG. 10. The activity with FPP and OA is lower for all enzymes, however, the CBFA derivatives shown in FIG. 6 can be accessed with the enzymes disclosed herein and their mutants.

Products with Divarinic Acid

APT29, APT73, and APT89 can also accept olivetolic acid analogs as substrates as shown by their reactivity with divarinic acid (2,4 dihydroxy-6-propyl-benzoic acid: DVA) and GPP. A summary of the activity profile of these enzymes with different substrates (OA or DVA) is shown in FIG. 9.

### Enzyme Classification and Homology

As described above, APT29, APT73, and APT89 have high activity using OA and GPP but lower selectivity toward CBGA formation. However, APT29, APT73, and APT89 enzymes do produce CBGA and so can be assigned as CBGA producing enzymes (whose selectivity and specific activity will be improved by engineering). Of note, APT89 is a truncated version of APT88, wherein the first 70 AA were removed. APT88 was not successfully expressed in E. coli, but is expected to have the same activity as APT89. A table listing the relative sequence identities shared by the enzymes is shown below.

| | APT29 | APT89 | APT73 |
|---|---|---|---|
| APT29 | | 71.0 | 69.4 |
| APT89 | 71.0 | | 95.6 |
| APT73 | 69.4 | 95.6 | |

Note that there is no other enzyme in the public domain that shares more than 53% sequence identity with APT29, APT73, APT88 or APT89: this is a close homology group. In addition, as shown herein, the activity and selectivity of the enzymes in this group is very similar to each other.

### Example 4—Modeling and Mutatgenesis of APT29, APT73, and APT89

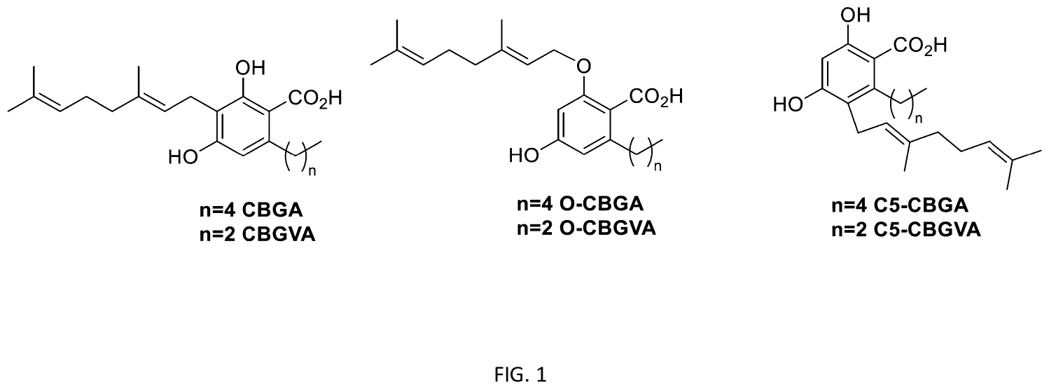

All APT enzymes described herein can utilize GPP and OA as substrates but produce O-CBGA as a major product (See FIGS. 1 and 9). As a result, protein engineering can be used to increase both the selectivity and the specific activity towards CBGA formation. The engineering approach detailed herein begins by creating structural models. A variety of commercial and free software packages are available to create structure models using crystal structures of homologous proteins as templates. The selection of the template structures used in the homology modelling process of APT29, APT73, and APT89 considered three important factors: i) sequence identity between the template enzyme(s) and the target enzyme(s) [only those with >30% sequence identity were used]; ii) the atomic resolution at which the template enzyme(s) were solved; and iii) The percent of sequence coverage between the target enzyme and the template enzyme(s) (i.e., differences in the length of the enzymes). Using this approach, 8 to 10 templates (depending on software) were used to generate the homology models. All enzymes were different prenyltransferases. The homology models were evaluated for accuracy using specific software (MolProbity) that showed significant refinement of the structures was required. This was likely due to the low sequence identity between the template structures used in modeling and the sequences of APT29, APT73, and APT89 (˜30-40%). The structure refinement and correction were achieved using secondary software that can energy minimize the protein structure. This relaxes the force on the atoms in the initial model of the protein structure, which ultimately refines the model of the protein structure. As a result, the structural quality of the homology model is significantly improved compared to the initial model (using MolProbity analyses as a comparison).

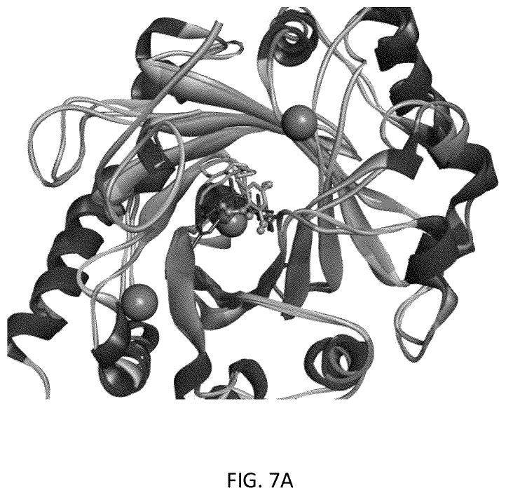

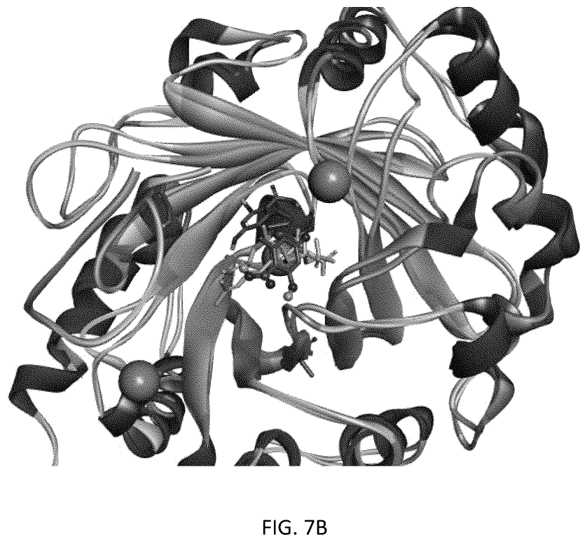

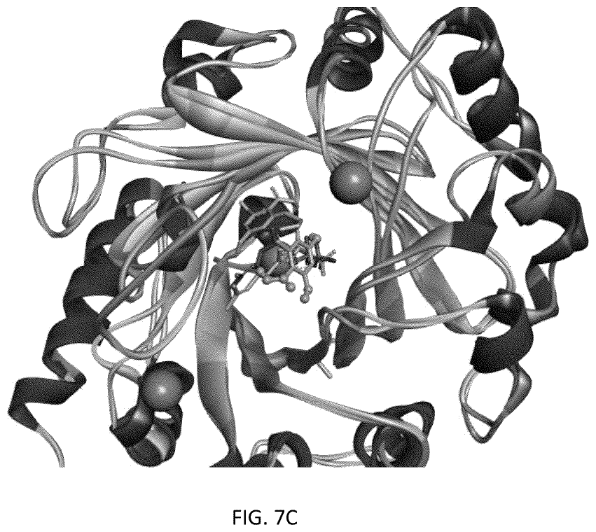

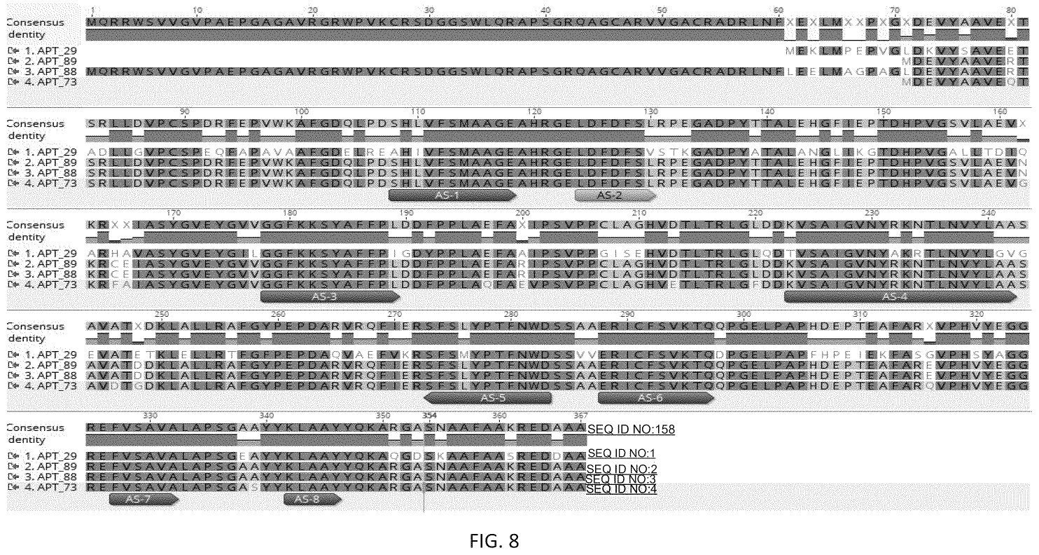

Finally, the OA and GPP substrates were docked in the active site using a different software package. The top two (of a number of possible orientations) docking poses for OA and GPP were selected based on calculated binding energy and the orientation in the active site that brings OA and GPP into the proper position for the reaction. After this modeling exercise was completed, amino acids in the active site that are 5 Å from each substrate were identified and were selected for mutagenesis. FIGS. 7A-7C show the structural alignment of APT29 and APT73 models and the two positions of OA and GPP bound in the active site. In yellow, the amino acids that are 5 Å away from any of these substrates are also highlighted.

Not surprisingly, the structural models of APT29, APT73, and APT89 are very similar (APT89 is not shown because it is essentially identical to APT73). The approach used to improve the activity/selectivity of APT29, APT73, and APT89 was the mutagenesis of one or a combination of 2 to 20 (double, triple, quadruple, etc. mutation combinations) of the amino acids highlighted in FIG. 7C in yellow. Mutagenesis at the same positions, but likely with different mutations, will improve the activity and selectivity of these enzymes towards CBGA derivatives or analogues coming from the reaction of OA analogs (such as DVA) and GPP and/or FPP. Additional mutations outside the highlighted region may also be introduced to improve other required enzyme properties such as stability, expression, etc.

The approach for mutagenesis will follow three steps. In the first step, site saturation mutagenesis (SSM) is performed at each residue in the active site (FIG. 7C), one position at a time. After initial screening, mutants with improved properties (activity or selectivity) will be used as templates to perform a second round of SSM. This process will be repeated multiple times until high activity and selectivity are achieved. In a parallel approach, the screening results of the first and second round will be used to create a sequence-function model using appropriate Artificial Intelligence (AI) software. The later will then predict mutants with combinations of mutations with improved activity that will be synthesized and tested. This process of AI predicted mutations will be iteratively repeated until optimal activity and selectivity are achieved.

The exact amino acid positions of FIG. 7C in APT29, APT73, and APT89 are shown below in bold/underline and specified by amino acid and position. In addition to active site amino acids, it was identified that the C-terminus amino acids may play a role in activity and/or expression of the protein. For this reason, the amino acids at the C-terminus are also included as important for activity and as a targets for potential mutagenesis.

>APT29
(SEQ ID NO: 1)
MEKLMPEPVGLDKVYSAVEETADLLGVPCSPEQFAPAVAAFGDELREAHI
VFSMAAGEAHRGELDFDFSVSTKGADPYATALANGLIKGTDHPVGALLTD
IQARHAVASYGVEYGILGGFKKSYAFFPIGDYPPLAEFAAIPSVPPGISE
HVDTLTRLGLQDTVSAIGVNYAKRTLNVYLGVGEVATETKLELLRTFGFP
EPDAQVAEFVKRSFSMYPTFNWDSSVVERICFSVKTQDPGELPAPFHPEI
EKFASGVPHSYAGGREFVSAVALAPSGEAYYKLAAYYQKAQGDSKAAFAA
SREDDAA

### Positions Around Active Site:

H49, V51, F52, S53, D65, D67, F68, S69, G111, E113, K122, Y124, A125, F126, V164, S165, A166, 1167, N170, F214, S215, Y217, T219, R229, C231, S233, K235, V268, S269, A270, K282, L283, A284, A285, Y286, G292, D293, S294, K295, A296, A297, F298

>APT73
(SEQ ID NO: 4)
MDEVYAAVEQTSRLLDVPCSPDRFEPVWKAFGDQLPDSHLVFSMAAGEAH
RGELDFDFSLRPEGADPYTTALEHGFIEPTDHPVGSVLAEVGKRFAIASY
GVEYGVVGGFKKSYAFFPLDDFPPLAQFAEVPSVPPCLAGHVETLTRLGF
DDKVSAIGVNYRKNTLNVYLAASAVDTGDKLALLRAFGYPEPDARVRQFI
ERSFSLYPTFNWDSSAAERICFSVKTQQPGELPAPHDEPTEAFARQVPHV
YEGGREFVSAVALAPSGASYYKLAAYYQKARGASNAAFAAKREDAAA

### Positions Around Active Site

S38, H39, L40, V41, F42, S43, M44, D55, D57, F58, S59, G101, E103, K111, K112, Y114, A115, F116, F117, S155, A156, 1157, N160, N167, Y169, F204, S205, Y207, T209, R219, C221, S223, V224, K225, V258, S259, A260, K272, L273, A274, A275, Y276, G282, A283 S284, N285, A286, A287, F288

>APT89
(SEQ ID NO: 2)
MDEVYAAVERTSRLLDVPCSPDRFEPVWKAFGDQLPDSHLVFSMAAYGVG
EAHRGELDFDFSLRPEGADPYTTALEHGFIEPTDHPVGSVLAEVNKRCEI
ASEYGVVGGFKKSYAFFPLDDFPPLAEFARIPSVPPCLAGHVDTLTRLGL
DDKVSAIGVNYRKNTLNVYLAASAVATDDKLALLRAFGYPEPDARVRQFI
ERSFSLYPTFNWDSSAAERICFSVKTQQPGELPAPHDEPTEAFAREVPHV
YEGGREFVSAVALAPSGAAYYKLAAYYQKARGASNAAFAAKREDAAA

### Positions in the Active Site are Identical to APT_73

S38, H39, L40, V41, F42, S43, M44, D55, D57, F58, S59, G101, E103, K111, K112, Y114, A115, F116, F117, S155, A156, 1157, N160, N167, Y169, F204, S205, Y207, T209, R219, C221, S223, V224, K225, V258, S259, A260, K272, L273, A274, A275, Y276, G282, A283 S284, N285, A286, A287, F288

>APT88
(SEQ ID NO: 3)
MQRRWSVVGVPAEPGAGAVRGRWPVKCRSDGGSWLQRAPSGRQAGCARVV
GACRADRLNFLEELMAGPAGLDEVYAAVERTSRLLDVPCSPDRFEPVWKA
FGDQLPDSHLVFSMAAGEAHRGELDFDFSLRPEGADPYTTALEHGFIEPT
DHPVGSVLAEVNKRCEIASYGVEYGVVGGFKKSYAFFPLDDFPPLAEFAR
IPSVPPCLAGHVDTLTRLGLDDKVSAIGVNYRKNTLNVYLAASAVATDDK
LALLRAFGYPEPDARVRQFIERSFSLYPTFNWDSSAAERICFSVKTQQPG
ELPAPHDEPTEAFAREVPHVYEGGREFVSAVALAPSGAAYYKLAAYYQKA
RGASNAAFAAKREDAAA

### Example 5—APT29, APT73, and APT89 Expression in E. coli

As discussed above, APT29, APT73, and APT89 were expressed in E. Coli with an N-terminal His tag. The expressed proteins including the His tag are as follows:

APT29-Expressed-Sequence
(SEQ ID NO: 7)
MKHHHHHHGTSENLYFQGMEKLMPEPVGLDKVYSAVEETADLLGVPCSPE
QFAPAVAAFGDELREAHIVFSMAAGEAHRGELDFDFSVSTKGADPYATAL
ANGLIKGTDHPVGALLTDIQARHAVASYGVEYGILGGFKKSYAFFPIGDY
PPLAEFAAIPSVPPGISEHVDTLTRLGLQDTVSAIGVNYAKRTLNVYLGV
GEVATETKLELLRTFGFPEPDAQVAEFVKRSFSMYPTFNWDSSVVERICF
SVKTQDPGELPAPFHPEIEKFASGVPHSYAGGREFVSAVALAPSGEAYYK
LAAYYQKAQGDSKAAFAASREDDAAG
>APT73-Expressed-Sequence
(SEQ ID NO: 8)
MKHHHHHHGTSENLYFQGMDEVYAAVEQTSRLLDVPCSPDRFEPVWKAFG
DQLPDSHLVFSMAAGEAHRGELDFDFSLRPEGADPYTTALEHGFIEPTDH
PVGSVLAEVGKRFAIASYGVEYGVVGGFKKSYAFFPLDDFPPLAQFAEVP
SVPPCLAGHVETLTRLGFDDKVSAIGVNYRKNTLNVYLAASAVDTGDKLA
LLRAFGYPEPDARVRQFIERSFSLYPTFNWDSSAAERICFSVKTQQPGEL
PAPHDEPTEAFARQVPHVYEGGREFVSAVALAPSGASYYKLAAYYQKARG
ASNAAFAAKREDAAAG
>APT89-Expressed-Sequence
(SEQ ID NO: 9)
MKHHHHHHGTSENLYFQGMDEVYAAVERTSRLLDVPCSPDRFEPVWKAFG
DQLPDSHLVFSMAAGEAHRGELDFDFSLRPEGADPYTTALEHGFIEPTDH
PVGSVLAEVNKRCEIASYGVEYGVVGGFKKSYAFFPLDDFPPLAEFARIP
SVPPCLAGHVDTLTRLGLDDKVSAIGVNYRKNTLNVYLAASAVATDDKLA
LLRAFGYPEPDARVRQFIERSFSLYPTFNWDSSAAERICFSVKTQQPGEL
PAPHDEPTEAFAREVPHVYEGGREFVSAVALAPSGAAYYKLAAYYQKARG
ASNAAFAAKREDAAAG

The nucleotide sequences used to express APT29, APT73, and APT89 with an N-terminal His tag in E. coli (SEQ ID NOS: 7-9) are as follows:

>APT29-Expressed-Sequence
(SEQ ID NO: 12)
ATGAAACATCACCACCACCACCATGGCACATCTGAAAACTTGTATTTCCA
GGGCATGGAGAAGCTGATGCCTGAACCTGTCGGTCTGGACAAGGTCTACTCTGCT
GTCGAGGAAACCGCTGATCTGCTTGGTGTTCCCTGTTCTCCTGAGCAGTTCGCTCC
TGCTGTTGCTGCTTTTGGTGATGAGCTTCGAGAGGCCCACATCGTCTTCTCTATGG
CTGCTGGTGAGGCTCATCGAGGTGAACTGGATTTCGACTTCTCCGTCTCCACCAA
GGGTGCTGATCCTTACGCTACTGCTCTGGCTAACGGTCTGATCAAGGGTACTGAC
CACCCTGTTGGTGCTCTGCTGACCGATATTCAGGCTCGACACGCTGTTGCCTCTTA
TGGTGTTGAGTACGGCATTCTGGGCGGCTTCAAGAAGTCTTACGCCTTCTTCCCCA
TCGGCGACTATCCTCCTTTGGCTGAGTTTGCCGCTATCCCCTCTGTTCCTCCTGGT
ATTTCTGAGCACGTCGACACTCTTACCCGACTTGGTCTTCAGGACACCGTCTCTGC
CATTGGCGTCAACTATGCTAAGCGAACCCTGAACGTCTACCTGGGTGTTGGTGAG
GTTGCTACTGAGACCAAGCTGGAGCTTCTGCGAACCTTCGGTTTTCCTGAGCCTG
ATGCTCAGGTTGCTGAGTTCGTCAAGCGATCCTTCTCCATGTACCCCACCTTCAAC
TGGGATTCCTCTGTCGTCGAGCGAATCTGCTTCTCCGTCAAGACCCAGGATCCTG
GTGAGTTACCTGCTCCTTTTCATCCCGAGATCGAGAAGTTCGCCTCTGGTGTTCCC
CACTCTTACGCTGGTGGTCGAGAGTTCGTTTCTGCTGTTGCTCTTGCTCCTTCTGG
TGAGGCTTACTACAAGCTGGCTGCCTACTACCAGAAGGCTCAGGGTGATTCTAAG
GCCGCTTTTGCCGCTTCTCGAGAGGATGATGCTGCCGGTTAG
>APT73-Expressed-Sequence
(SEQ ID NO: 13)
ATGAAACATCACCACCACCACCATGGCACATCTGAAAACTTGTATTTCCA
GGGCATGGACGAGGTGTATGCGGCAGTGGAACAGACTTCTCGACTTCTGGACGTT
CCCTGTTCTCCTGACCGATTTGAGCCCGTTTGGAAGGCCTTTGGTGATCAGCTGCC
TGATTCTCACCTGGTCTTCTCTATGGCTGCTGGTGAGGCTCATCGAGGTGAGCTGG
ACTTTGACTTCTCTCTTCGACCTGAGGGTGCTGATCCTTACACTACCGCTCTGGAG
CACGGCTTCATTGAGCCTACTGATCATCCTGTCGGTTCTGTCCTTGCTGAGGTTGG
TAAGCGATTCGCCATCGCCTCTTACGGTGTCGAATACGGTGTTGTCGGTGGCTTC
AAGAAGTCTTACGCCTTCTTCCCCCTGGACGACTTTCCCCCTCTTGCTCAGTTTGC
TGAGGTCCCCTCTGTTCCTCCTTGTTTAGCTGGTCACGTTGAGACCCTTACCCGAC
TGGGATTTGACGACAAGGTCTCGGCCATTGGCGTCAACTACCGAAAGAACACCCT
GAACGTCTACCTGGCTGCTTCTGCTGTTGACACTGGCGATAAGCTGGCTCTGCTTC
GAGCTTTCGGTTACCCTGAACCTGATGCCCGAGTTCGACAGTTCATCGAGCGATC
CTTCTCCCTTTACCCCACCTTCAACTGGGATTCTTCTGCCGCTGAGCGAATCTGCT
TCTCCGTCAAGACACAGCAACCTGGTGAGCTTCCTGCACCTCATGATGAGCCTAC
TGAGGCTTTCGCTCGACAGGTTCCTCACGTTTACGAAGGTGGTCGAGAGTTCGTC
TCTGCTGTTGCTCTTGCTCCTTCTGGTGCCTCCTACTACAAGCTTGCTGCCTACTAC
CAGAAGGCTAGAGGTGCCTCTAATGCCGCTTTTGCTGCCAAGCGAGAGGATGCTG
CTGCTGGTTAG
>APT89-Expressed-Sequence
(SEQ ID NO: 14)
ATGAAACATCACCACCACCACCATGGCACATCTGAAAACTTGTATTTCCA
GGGCATGGACGAGGTGTATGCGGCAGTGGAACGAACTTCTCGACTTCTGGACGTT
CCCTGTTCTCCTGACCGATTTGAGCCCGTTTGGAAGGCCTTTGGTGATCAGCTGCC
TGATTCTCACCTGGTCTTCTCTATGGCTGCTGGTGAGGCTCATCGAGGTGAGCTGG
ACTTTGACTTCTCTCTTCGACCTGAGGGTGCTGATCCTTACACTACCGCTCTGGAG
CACGGCTTCATTGAGCCTACTGATCATCCTGTCGGTTCTGTCCTTGCTGAGGTCAA
CAAGCGATGCGAGATCGCCTCTTACGGTGTCGAATACGGTGTCGTTGGTGGCTTC
AAGAAGTCCTACGCCTTCTTCCCTCTGGACGACTTTCCCCCTCTTGCTGAGTTTGC
CCGAATCCCCTCTGTTCCTCCTTGTTTAGCTGGTCACGTTGACACTCTGACCCGAC
TTGGTCTGGACGACAAGGTCTCTGCCATTGGCGTCAACTACCGAAAGAACACCCT
GAACGTCTACCTTGCTGCTTCTGCCGTTGCTACCGACGATAAGCTGGCTCTGCTTC
GAGCTTTCGGTTACCCTGAACCTGATGCTAGAGTGCGACAGTTCATCGAGCGATC
CTTCTCCCTGTACCCCACCTTCAACTGGGATTCTTCTGCTGCTGAGCGAATCTGCT
TCTCCGTCAAGACACAGCAGCCTGGTGAGCTTCCTGCACCTCATGATGAACCTAC
TGAGGCTTTCGCTCGAGAGGTTCCTCACGTTTACGAGGGTGGTCGAGAGTTCGTC
TCTGCTGTTGCTTTAGCTCCTTCTGGTGCCGCTTACTACAAGCTTGCTGCCTACTA
CCAGAAGGCCAGAGGTGCCTCTAATGCTGCTTTTGCTGCCAAGCGAGAGGATGCT
GCTGCTGGTTAG

Furthermore, a nucleotide sequence that can be used to express APT88 with an N-terminal His tag is as follows:

>APT88-Expressed-Sequence
(SEQ ID NO: 15)
ATGAAACATCACCACCACCACCATGGCACATCTGAAAACTTGTATTTCCA
GGGCATGCAGAGACGATGGAGTGTGGTGGGTGTTCCTGCTGAACCTGGTG
CTGGTGCTGTTAGAGGTAGATGGCCTGTTAAGTGTCGATCTGACGGTGGT
TCTTGGCTTCAGCGAGCTCCCTCTGGTAGACAAGCTGGTTGTGCTCGAGT
TGTTGGTGCTTGTCGAGCTGATCGACTGAACTTCCTGGAGGAACTGATGG
CTGGTCCTGCTGGTCTTGATGAGGTCTATGCTGCTGTTGAGCGAACCTCT
CGACTGCTGGATGTTCCCTGTTCTCCTGACCGATTTGAGCCCGTTTGGAA
GGCTTTTGGTGACCAGCTGCCCGATTCTCACCTGGTCTTCTCTATGGCTG
CTGGTGAGGCTCATAGAGGTGAACTGGACTTCGACTTCTCCCTTCGACCT
GAGGGTGCTGATCCTTACACTACCGCTCTGGAACACGGTTTCATTGAGCC
TACTGACCACCCTGTCGGTTCTGTCCTTGCTGAGGTCAACAAGCGATGCG
AGATCGCCTCTTATGGTGTTGAGTACGGTGTCGTCGGTGGCTTCAAGAAG
TCCTACGCCTTCTTCCCTCTGGACGACTTTCCTCCTTTGGCTGAGTTTGC
CCGAATCCCCTCTGTTCCTCCTTGTCTTGCTGGTCACGTTGACACTCTTA
CCCGACTTGGTCTGGACGACAAGGTCTCTGCCATTGGCGTCAACTACCGG
AAGAACACCCTGAACGTTTACCTTGCTGCCTCTGCTGTTGCTACTGACGA
CAAGCTGGCTCTGCTGCGAGCTTTCGGTTACCCTGAACCTGATGCTAGAG
TTCGACAGTTCATCGAGCGATCCTTCTCCCTGTACCCCACCTTCAACTGG
GATTCCTCTGCTGCTGAGCGAATCTGCTTCTCTGTCAAGACACAGCAGCC
TGGTGAGCTTCCTGCACCTCATGATGAGCCTACTGAGGCTTTCGCTCGAG
AAGTTCCTCACGTTTACGAGGGTGGTCGAGAGTTCGTCTCTGCTGTTGCT
CTTGCTCCTTCTGGTGCTGCTTACTACAAGCTTGCTGCCTACTACCAGAA
GGCCAGAGGTGCCTCTAATGCCGCTTTTGCCGCTAAGCGAGAAGATGCTG
CTGCTGGTTAG

Finally, nucleotide sequences for expressing APT29. APT73, APT88, and APT89 are as follows:

>APT29
(SEQ ID NO: 16)
ATGGAGAAGCTGATGCCTGAACCTGTCGGTCTGGACAAGGTCTACTCTGC
TGTCGAGGAAACCGCTGATCTGCTTGGTGTTCCCTGTTCTCCTGAGCAGTTCGCTC
CTGCTGTTGCTGCTTTTGGTGATGAGCTTCGAGAGGCCCACATCGTCTTCTCTATG
GCTGCTGGTGAGGCTCATCGAGGTGAACTGGATTTCGACTTCTCCGTCTCCACCA
AGGGTGCTGATCCTTACGCTACTGCTCTGGCTAACGGTCTGATCAAGGGTACTGA
CCACCCTGTTGGTGCTCTGCTGACCGATATTCAGGCTCGACACGCTGTTGCCTCTT
ATGGTGTTGAGTACGGCATTCTGGGCGGCTTCAAGAAGTCTTACGCCTTCTTCCCC
ATCGGCGACTATCCTCCTTTGGCTGAGTTTGCCGCTATCCCCTCTGTTCCTCCTGG
TATTTCTGAGCACGTCGACACTCTTACCCGACTTGGTCTTCAGGACACCGTCTCTG
CCATTGGCGTCAACTATGCTAAGCGAACCCTGAACGTCTACCTGGGTGTTGGTGA
GGTTGCTACTGAGACCAAGCTGGAGCTTCTGCGAACCTTCGGTTTTCCTGAGCCT
GATGCTCAGGTTGCTGAGTTCGTCAAGCGATCCTTCTCCATGTACCCCACCTTCAA
CTGGGATTCCTCTGTCGTCGAGCGAATCTGCTTCTCCGTCAAGACCCAGGATCCT
GGTGAGTTACCTGCTCCTTTTCATCCCGAGATCGAGAAGTTCGCCTCTGGTGTTCC
CCACTCTTACGCTGGTGGTCGAGAGTTCGTTTCTGCTGTTGCTCTTGCTCCTTCTG
GTGAGGCTTACTACAAGCTGGCTGCCTACTACCAGAAGGCTCAGGGTGATTCTAA
GGCCGCTTTTGCCGCTTCTCGAGAGGATGATGCTGCC
>APT73
(SEQ ID NO: 17)
ATGGACGAGGTGTATGCGGCAGTGGAACAGACTTCTCGACTTCTGGACGT
TCCCTGTTCTCCTGACCGATTTGAGCCCGTTTGGAAGGCCTTTGGTGATCAGCTGC
CTGATTCTCACCTGGTCTTCTCTATGGCTGCTGGTGAGGCTCATCGAGGTGAGCTG
GACTTTGACTTCTCTCTTCGACCTGAGGGTGCTGATCCTTACACTACCGCTCTGGA
GCACGGCTTCATTGAGCCTACTGATCATCCTGTCGGTTCTGTCCTTGCTGAGGTTG
GTAAGCGATTCGCCATCGCCTCTTACGGTGTCGAATACGGTGTTGTCGGTGGCTT
CAAGAAGTCTTACGCCTTCTTCCCCCTGGACGACTTTCCCCCTCTTGCTCAGTTTG
CTGAGGTCCCCTCTGTTCCTCCTTGTTTAGCTGGTCACGTTGAGACCCTTACCCGA
CTGGGATTTGACGACAAGGTCTCGGCCATTGGCGTCAACTACCGAAAGAACACCC
TGAACGTCTACCTGGCTGCTTCTGCTGTTGACACTGGCGATAAGCTGGCTCTGCTT
CGAGCTTTCGGTTACCCTGAACCTGATGCCCGAGTTCGACAGTTCATCGAGCGAT
CCTTCTCCCTTTACCCCACCTTCAACTGGGATTCTTCTGCCGCTGAGCGAATCTGC
TTCTCCGTCAAGACACAGCAACCTGGTGAGCTTCCTGCACCTCATGATGAGCCTA
CTGAGGCTTTCGCTCGACAGGTTCCTCACGTTTACGAAGGTGGTCGAGAGTTCGT
CTCTGCTGTTGCTCTTGCTCCTTCTGGTGCCTCCTACTACAAGCTTGCTGCCTACTA
CCAGAAGGCTAGAGGTGCCTCTAATGCCGCTTTTGCTGCCAAGCGAGAGGATGCT
GCTGCT
>APT88
(SEQ ID NO: 18)
ATGCAGAGACGATGGAGTGTGGTGGGTGTTCCTGCTGAACCTGGTGCTGG
TGCTGTTAGAGGTAGATGGCCTGTTAAGTGTCGATCTGACGGTGGTTCTTGGCTTC
AGCGAGCTCCCTCTGGTAGACAAGCTGGTTGTGCTCGAGTTGTTGGTGCTTGTCG
AGCTGATCGACTGAACTTCCTGGAGGAACTGATGGCTGGTCCTGCTGGTCTTGAT
GAGGTCTATGCTGCTGTTGAGCGAACCTCTCGACTGCTGGATGTTCCCTGTTCTCC
TGACCGATTTGAGCCCGTTTGGAAGGCTTTTGGTGACCAGCTGCCCGATTCTCAC
CTGGTCTTCTCTATGGCTGCTGGTGAGGCTCATAGAGGTGAACTGGACTTCGACT
TCTCCCTTCGACCTGAGGGTGCTGATCCTTACACTACCGCTCTGGAACACGGTTTC
ATTGAGCCTACTGACCACCCTGTCGGTTCTGTCCTTGCTGAGGTCAACAAGCGAT
GCGAGATCGCCTCTTATGGTGTTGAGTACGGTGTCGTCGGTGGCTTCAAGAAGTC
CTACGCCTTCTTCCCTCTGGACGACTTTCCTCCTTTGGCTGAGTTTGCCCGAATCC
CCTCTGTTCCTCCTTGTCTTGCTGGTCACGTTGACACTCTTACCCGACTTGGTCTG
GACGACAAGGTCTCTGCCATTGGCGTCAACTACCGGAAGAACACCCTGAACGTTT
ACCTTGCTGCCTCTGCTGTTGCTACTGACGACAAGCTGGCTCTGCTGCGAGCTTTC
GGTTACCCTGAACCTGATGCTAGAGTTCGACAGTTCATCGAGCGATCCTTCTCCCT
GTACCCCACCTTCAACTGGGATTCCTCTGCTGCTGAGCGAATCTGCTTCTCTGTCA
AGACACAGCAGCCTGGTGAGCTTCCTGCACCTCATGATGAGCCTACTGAGGCTTT
CGCTCGAGAAGTTCCTCACGTTTACGAGGGTGGTCGAGAGTTCGTCTCTGCTGTT
GCTCTTGCTCCTTCTGGTGCTGCTTACTACAAGCTTGCTGCCTACTACCAGAAGGC
CAGAGGTGCCTCTAATGCCGCTTTTGCCGCTAAGCGAGAAGATGCTGCTGCT
>APT89
(SEQ ID NO: 19)
ATGGACGAGGTGTATGCGGCAGTGGAACGAACTTCTCGACTTCTGGACGT
TCCCTGTTCTCCTGACCGATTTGAGCCCGTTTGGAAGGCCTTTGGTGATCAGCTGC
CTGATTCTCACCTGGTCTTCTCTATGGCTGCTGGTGAGGCTCATCGAGGTGAGCTG
GACTTTGACTTCTCTCTTCGACCTGAGGGTGCTGATCCTTACACTACCGCTCTGGA
GCACGGCTTCATTGAGCCTACTGATCATCCTGTCGGTTCTGTCCTTGCTGAGGTCA
ACAAGCGATGCGAGATCGCCTCTTACGGTGTCGAATACGGTGTCGTTGGTGGCTT
CAAGAAGTCCTACGCCTTCTTCCCTCTGGACGACTTTCCCCCTCTTGCTGAGTTTG
CCCGAATCCCCTCTGTTCCTCCTTGTTTAGCTGGTCACGTTGACACTCTGACCCGA
CTTGGTCTGGACGACAAGGTCTCTGCCATTGGCGTCAACTACCGAAAGAACACCC
TGAACGTCTACCTTGCTGCTTCTGCCGTTGCTACCGACGATAAGCTGGCTCTGCTT
CGAGCTTTCGGTTACCCTGAACCTGATGCTAGAGTGCGACAGTTCATCGAGCGAT
CCTTCTCCCTGTACCCCACCTTCAACTGGGATTCTTCTGCTGCTGAGCGAATCTGC
TTCTCCGTCAAGACACAGCAGCCTGGTGAGCTTCCTGCACCTCATGATGAACCTA
CTGAGGCTTTCGCTCGAGAGGTTCCTCACGTTTACGAGGGTGGTCGAGAGTTCGT
CTCTGCTGTTGCTTTAGCTCCTTCTGGTGCCGCTTACTACAAGCTTGCTGCCTACT
ACCAGAAGGCCAGAGGTGCCTCTAATGCTGCTTTTGCTGCCAAGCGAGAGGATGC
TGCTGCT

### Example 6—APT29, APT73, and APT89 Consensus Sequence

Consensus sequence for APT29. APT73, and APT89 with 0 to 65% amino acid bias showing as X (X means variation of amino acid in 50% or more in the alignment). This sequence does not change until biased for 75% or more where it creates too much variation in the sequence. In the current bias restrictions, the variation in each position is shown as X1-X6.

>APT29/73/89_Consensus_0_65_Restrict
(SEQ ID NO: 5)
MDEVYAAVEX1TSRLLDVPCSPDRFEPVWKAFGDQLPDSHLVFSMAAGEA
HRGELDFDFSLRPEGADPYTTALEHGFIEPTDHPVGSVLAEVX2KRX3AI
ASYGVEYGVVGGFKKSYAFFPLDDFPPLAEFAX4IPSVPPCLAGHVDTLT
RLGLDDKVSAIGVNYRKNTLNVYLAASAVATX5DKLALLRAFGYPEPDAR
VRQFIERSFSLYPTFNWDSSAAERICFSVKTQQPGELPAPHDEPTEAFAR
X6VPHVYEGGREFVSAVALAPSGAAYYKLAAYYQKARGASNAAFAAKRED
AAA

Consensus Sequence with the amino acids present in each position is shown below:

(SEQ ID NO: 6)
MDEVYAAVE(E, R, or Q)TSRLLDVPCSPDRFEPVWKAFGDQLPDSH
LVFSMAAGEAHRGELDFDFSLRPEGADPYTTALEHGFIEPTDHPVGSVLA
EV(Q, N, or G)KR(H, C, or F)AIASYGVEYGVVGGFKKSYAFF
PLDDFPPLAEFA(A, R, or E)IPSVPPCLAGHVDTLTRLGLDDKVSA
IGVNYRKNTLNVYLAASAVAT(E, D, or G)DKLALLRAFGYPEPDAR
VRQFIERSFSLYPTFNWDSSAAERICFSVKTQQPGELPAPHDEPTEAFAR
(G, E, or Q)VPHVYEGGREFVSAVALAPSGAAYYKLAAYYQKARGAS
NAAFAAKREDAAA

### Example 7—Mutagenesis Methods

Mutants at specific positions or combination of positions were made by CODEX DNA, Inc. (San Diego, CA) and provided as plasmid DNA for each mutant. Saturation mutagenesis at specified positions was also performed by CODEX DNA, Inc. and provided as pooled plasmid DNA containing all 20 amino acids at roughly equal amounts.

### Example 8—Screening in E. coli

Plasmids were transformed into 25 μL of chemically competent BL21 (DE3) cells from NEB, plated on LB agar plates with 50 μg/mL kanamycin, and grown overnight at 37° C. Colonies were each picked into 1 mL LB media with 50 μg/mL kanamycin in 96dw blocks and grown overnight at 33° C. with 250 rpm shaking. From each well of the overnight cultures, 250 μL was added to 250 μL 50% glycerol to create glycerol stock blocks that were stored at −80° C. Additionally, 20 μL from each well was used to inoculate 750 μL TB media with 50 μg/mL kanamycin in 96dw blocks. Blocks were grown for 3 h at 33° C. with 250 rpm shaking, after which they were induced by adding 250 μL TB media with 50 μg/mL kanamycin and IPTG to a final concentration of 1 mM and incubated overnight at 27° C. with 250 rpm shaking. Cultures were centrifuged at 4600×g for 10 min at 4° C., decanted, and stored at −80° C. for 30 min. Cell pellets were thawed at room temperature for 30 min and then 0.4 mL B-PER with 5 mM MgCl2, 0.5 mg/mL lysozyme and 2 μL/mL DNase was added to each well. Pellets were resuspended by vortexing for 1 min and then incubated for 20 min at 33° C. with 250 rpm shaking. To pellet cell debris, lysates were centrifuged at 4600×g for 10 min at 4° C. Olivetolic acid was dissolved in DMSO and then added to 100 mM HEPES pH 7.4 with 100 mM NaCl, 10 mM MgCl2 and 1.5 mM GPP (solubilized by sonication in a water bath for 30 min) to a concentration of 1.5% DMSO and 1.5 mM olivetolic acid. Lysate and reaction buffers were incubated at 33° C. before combination. Reactions were initiated by adding 100 μL lysate to 200 μL reaction buffer for a final concentration of 1% DMSO, 1 mM olivetolic acid, and 1 mM GPP. Reactions were incubated at 33° C. with 250 rpm shaking. After 30 min, reactions were quenched by addition of 200 μL 1:1 acetonitrile:water with 0.2% formic acid and 0.5 mg/mL pentyl-benzoic acid. Precipitates were pelleted by centrifuging at 4600×g for 10 min and then 200 μL was transferred to fresh plates, sealed, and analyzed via HPLC.

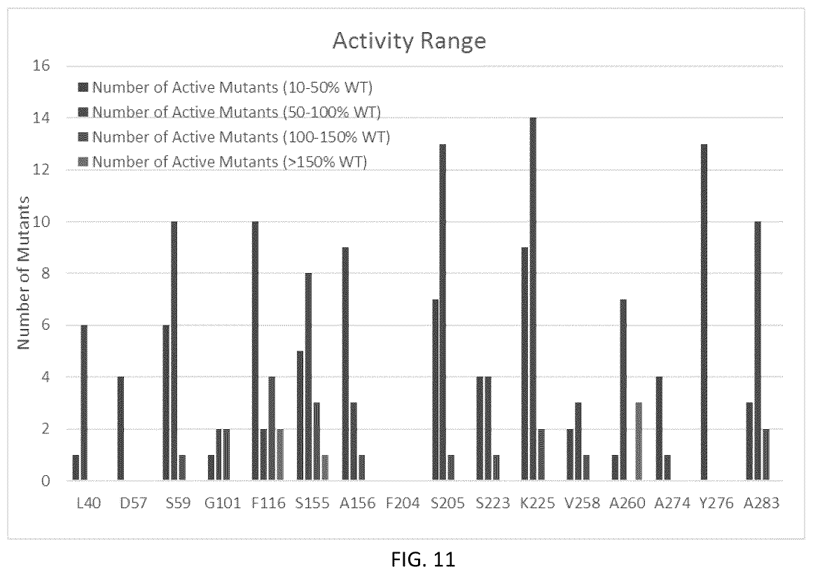

FIG. 11 represents the overall activity in selected positions around the active site after saturation mutagenesis and screening of APT73 (SEQ ID NO: 4). This graph shows that many mutations can be tolerated around the active site, and some alone can improve the enzyme's activity such as F116, S155, A260, while many others give mutants with the same or slightly higher activity than wild type, such as S59, A156, S205, S223, K225, V258 and A283. On the other hand, it was clearly shown that the enzyme can't tolerate mutations at L40, D57, G101, F204, A274. Some positions that showed neutral or reduced activity upon mutagenesis of WT sequence like Y276, were later shown that their modification can improve activity in combination with other mutations around the active site.

Based on the above results and further modeling analysis additional libraries were made and were screened. Selected mutants with improved activity and selectivity are shown in the following Table 1. All activities and selectivities in this Table are compared to APT73. For example, APT73 makes O-CBGA at 96% of total products in this lysate assay. Mutant APT73.1 (S205R) has 65% of the total activity of APT73 and makes CBGA as a single product.

TABLE 1
Screening of APT73 mutants in E. coli lysates. Activities and selectivities
of all enzymes were normalized to APT73 wild type, whose activity is set to 1

| Enzyme | Mutations | Trunc¹ | CBGA² | O-CBGA² |
|---|---|---|---|---|
| NphB | | | 0.00 | 0.01 |
| APT73 | | | 0.04 | 0.96 |
| APT73.F116C | F116C | | 0.11 | 2.28 |
| APT73.F116L | F116L | | 0.15 | 1.17 |
| APT73.F116A | F116A | | 0.11 | 1.38 |
| APT73.S155A | S155A | | 0.00 | 1.51 |
| APT73.A260S | A260S | | 0.01 | 2.24 |
| APT73.1 | S205R | | 0.65 | 0.00 |
| APT73.13 | S205R, G282R, A283C, S284L | Yes | 1.16 | 0.00 |
| APT73.14 | S205R, K225F, G282R, A283C, S284L | Yes | 1.99 | 0.00 |
| APT73.15 | S205R, S223A, G282R, A283C, S284L | Yes | 2.21 | 0.02 |
| APT73.16 | S205R, A260G, G282R, A283C, S284L | Yes | 1.27 | 0.00 |
| APT73.17 | S205R, Y276L, G282R, A283C, S284L | Yes | 1.81 | 0.00 |
| APT73.18 | S205R, Y276C, G282R, A283C, S284L | Yes | 1.73 | 0.00 |
| APT73.19 | S205R, Y276S, G282R, A283C, S284L | Yes | 1.87 | 0.00 |
| APT73.20 | S205R, G282R, A283P, S284L | Yes | 1.70 | 0.00 |
| APT73.13.Y276V | S205R, Y276V, G282R, A283C, S284L | Yes | 1.86 | 0.00 |
| APT73.13.C283A | S205R, G282R, S284L | Yes | 1.40 | 0.00 |
| APT73.13.C283G | S205R, G282R, A283G, S284L | Yes | 1.36 | 0.00 |
| APT73.21 | A156C, S205R, K225F, Y276C, G282R, A283C, | Yes | 2.62 | 0.00 |
| | S284L | | | |
| APT73.22 | S205R, K225F, A260G, Y276L, G282R, A283C, | Yes | 2.42 | 0.00 |
| | S284L | | | |
| APT73.23 | S205R, K225H, A260G, Y276L, G282R, A283C, | Yes | 2.88 | 0.00 |
| | S284L | | | |
| APT73.24 | S205R, K225R, A260G, Y276L, G282R, A283C, | Yes | 2.41 | 0.00 |
| | S284L | | | |
| APT73.25 | S205R, K225H, A260G, Y276V, G282R, A283C, | Yes | 2.34 | 0.00 |
| | S284L | | | |
| APT73.26 | S205R, S223A, K225R, Y276L, G282R, A283C, | Yes | 2.50 | 0.00 |
| | S284L | | | |
| APT73.27 | S205R, S223A, K225R, Y276C, G282R, A283C, | Yes | 2.62 | 0.00 |
| | S284L | | | |
| APT73.28 | S205R, S223A, K225F, Y276S, G282R, A283C, | Yes | 2.39 | 0.00 |
| | S284L | | | |
| APT73.29 | S205R, S223A, K225H, Y276V, G282R, A283C, | Yes | 2.74 | 0.00 |
| | S284L | | | |
| APT73.30 | S205R, S223A, K225H, A260G, Y276L, G282R, | Yes | 3.02 | 0.00 |
| | A283C, S284L | | | |
| APT73.31 | S205R, S223A, K225R, A260G, Y276V, G282R, | Yes | 2.94 | 0.00 |
| | A283C, S284L | | | |
| APT73.32 | S205R, S223A, Y276S, G282R, A283P, S284L | Yes | 3.32 | 0.00 |
| APT73.33 | S205R, S223A, Y276A, G282R, A283P, S284L | Yes | 3.09 | 0.00 |
| APT73.34 | S205R, S223A, Y276C, G282R, A283E, S284L | Yes | 3.14 | 0.00 |
| APT73.35 | S205R, S223A, Y276S, G282R, A283E, S284L | Yes | 3.61 | 0.00 |
| APT73.36 | S205R, S223A, A260G, Y276C, G282R, A283P, | Yes | 3.13 | 0.00 |
| | S284L | | | |
| APT73.37 | S205R, S223A, A260G, Y276V, G282R, A283P, | Yes | 4.20 | 0.00 |
| | S284L | | | |
| APT89 | | | 0.05 | 1.00 |
| APT89.F116L | F116L | | 0.27 | 1.80 |
| APT89.F116A | F116A | | 0.10 | 0.87 |
| APT89.S155A | S155A | | 0.04 | 1.46 |
| APT89.A260S | A260S | | 0.04 | 2.26 |
| APT89.6 | S205K | | 0.53 | 0.10 |

¹Refers to the removal of residues 285 onward from the C-terminus
²Product concentrations are normalized to the total amount produced (CBGA and O-CBGA) by APT73, which is set to 1.

These screening results clearly show that S205 was important in changing the enzyme's selectivity from prenylation in the 2-hydroxy position to the correct C₃ making CBGA or CBGVA as the major product. Other amino acids also contribute to the enzyme's activity include F116, A165, S223, A260, Y276, G282, A283 and S284. It as also clearly shown that equivalent to APT73 mutations in APT89 had a similar effect in enzyme's activity and selectivity. For example, S205R mutation also switched APT89's selectivity to CBGA.

Top mutants would be selected, sequenced, and re-screened in E. coli. The genes were also transferred in Yarrowia plasmids for screening (Example 9). Selected mutants were also purified from E. coli and their activity and selectivity properties were assessed (Table 4)

### Example 9: Screening Libraries in Yarrowia

Yarrowia screening using preselected mutants from E. coli screens or from mutant libraries directly transformed in Yarrowia was performed in 96 well plates. The Yarrowia strain has a genomic modification to increase flux towards GPP formation.

Plasmids were transformed into Yarrowia, plated on minimal media agar plates, and grown for 48 h at 30° C. Colonies were picked into 0.5 mL YNBD+CAA (6.71 g/L YNBD+Nitrogen, 5 g/L casamino acids, and 2% glucose) media with 100 mM MES pH 6.5 in 96w blocks. The blocks were grown for 48 h at 30° C. with 1000 rpm shaking. Then, 2 μL from each well of the pre-cultures was used to inoculate 0.5 mL YNBD+CAA media with 100 mM MES pH 6.5 and 2 mM olivetolic acid assay cultures which were grown at 30° C. with 1000 rpm shaking. After 24 h, an additional 2% glucose was added. After an additional 24 h (48 h total), assay cultures were quenched by addition of 200 μL 1:1 acetonitrile:water with 0.2% formic acid and 0.5 mg/mL pentyl-benzoic acid. Precipitates were pelleted by centrifuging at 4600×g for 10 min and then 200 μL was transferred to fresh plates, sealed, and analyzed via HPLC. Assay cultures with divarinic acid instead of olivetolic acid were handled in the same way but were grown for 96 h total (instead of 48 h), with 2% glucose added every 24 h. Results of selected mutants are shown in Tables 2 and 3 (for OA and DVA feeds respectively). Like before Table show the relative activity normalized to APT73, which under these plate screening conditions APT73 made about 12-15 μM of O-CBGA and 40 μM O-CBGVA.

TABLE 2
APT73 mutants. Product formation in Yarrowia plate screening with Olivetolic acid (OA) feeding. Activities
and selectivities of all enzymes were normalized to APT73 wild type, whose activity is set to 1

| Enzyme | Mutations | Trunc¹ | CBGA² | O-CBGA² | F-CBGA² |
|---|---|---|---|---|---|
| APT73 | | | 0.15 | 0.85 | 0.00 |
| APT73.13 | S205R, G282R, A283C, S284L | Yes | 0.20 | 0.00 | 0.00 |
| APT73.15 | S205R, S223A, G282R, A283C, S284L | Yes | 0.26 | 0.00 | 0.34 |
| APT73.16 | S205R, A260G, G282R, A283C, S284L | Yes | 0.15 | 0.00 | 0.00 |
| APT73.17 | S205R, Y276L, G282R, A283C, S284L | Yes | 0.48 | 0.00 | 1.03 |
| APT73.18 | S205R, Y276C, G282R, A283C, S284L | Yes | 0.10 | 0.00 | 0.00 |
| APT73.19 | S205R, Y276S, G282R, A283C, S284L | Yes | 0.33 | 0.00 | 0.00 |
| APT73.20 | S205R, G282R, A283P, S284L | Yes | 0.25 | 0.00 | 0.00 |
| APT73.21 | A156C, S205R, K225F, Y276C, G282R, A283C, S284L | Yes | 0.48 | 0.00 | 0.28 |
| APT73.22 | S205R, K225F, A260G, Y276L, G282R, A283C, S284L | Yes | 1.18 | 0.00 | 1.45 |
| APT73.23 | S205R, K225H, A260G, Y276L, G282R, A283C, S284L | Yes | 0.97 | 0.00 | 1.42 |
| APT73.24 | S205R, K225R, A260G, Y276L, G282R, A283C, S284L | Yes | 0.41 | 0.00 | 0.54 |
| APT73.25 | S205R, K225H, A260G, Y276V, G282R, A283C, S284L | Yes | 0.35 | 0.00 | 0.00 |
| APT73.26 | S205R, S223A, K225R, Y276L, G282R, A283C, S284L | Yes | 1.47 | 0.00 | 2.22 |
| APT73.27 | S205R, S223A, K225R, Y276C, G282R, A283C, S284L | Yes | 0.83 | 0.00 | 0.57 |
| APT73.28 | S205R, S223A, K225F, Y276S, G282R, A283C, S284L | Yes | 1.15 | 0.00 | 0.46 |
| APT73.29 | S205R, S223A, K225H, Y276V, G282R, A283C, S284L | Yes | 0.81 | 0.00 | 0.49 |
| APT73.30 | S205R, S223A, K225H, A260G, Y276L, G282R, A283C, | Yes | 3.59 | 0.00 | 3.89 |
| | S284L | | | | |
| APT73.31 | S205R, S223A, K225R, A260G, Y276V, G282R, A283C, | Yes | 0.75 | 0.00 | 0.36 |
| | S284L | | | | |
| APT73.32 | S205R, S223A, Y276S, G282R, A283P, S284L | Yes | 1.63 | 0.00 | 0.97 |
| APT73.33 | S205R, S223A, Y276A, G282R, A283P, S284L | Yes | 1.81 | 0.00 | 1.21 |
| APT73.34 | S205R, S223A, Y276C, G282R, A283E, S284L | Yes | 0.55 | 0.00 | 0.33 |
| APT73.35 | S205R, S223A, Y276S, G282R, A283E, S284L | Yes | 1.56 | 0.00 | 0.87 |
| APT73.36 | S205R, S223A, A260G, Y276C, G282R, A283P, S284L | Yes | 2.88 | 0.00 | 1.38 |
| APT73.37 | S205R, S223A, A260G, Y276V, G282R, A283P, S284L | Yes | 2.22 | 0.00 | 0.61 |
| APT73.44 | S205R, K225A, Y276L, G282R, A283C, S284L | Yes | 0.23 | 0.00 | 0.44 |
| APT73.45 | S205R, K225S, Y276C, G282R, A283C, S284L | Yes | 0.23 | 0.00 | 0.17 |
| APT73.46 | S205R, K225F, Y276L, G282R, A283C, S284L | Yes | 0.28 | 0.00 | 0.00 |
| APT73.47 | A156I, S205R, S223A, K225H, A260G, Y276L, G282R, | Yes | 14.97 | 0.00 | 8.19 |
| | A283C, S284L | | | | |
| APT73.49 | S205R, S223A, K225M, A260G, Y276L, G282R, A283C, | Yes | 4.88 | 0.00 | 5.56 |
| | S284L | | | | |
| APT73.52 | S205R, S223A, K225H, A260G, Y276E, G282R, A283C, | Yes | 10.41 | 0.00 | 1.31 |
| | S284L | | | | |
| APT73.53 | S205R, S223A, K225H, A260G, Y276H, G282R, A283C, | Yes | 5.58 | 0.00 | 9.06 |
| | S284L | | | | |
| APT73.54 | S205R, S223A, K225H, A260G, Y276M, G282R, A283C, | Yes | 3.22 | 0.00 | 3.78 |
| | S284L | | | | |
| APT73.58 | S205R, S223A, K225H, A260G, Y276L, G282R, A283K, | Yes | 5.01 | 0.00 | 5.17 |
| | S284L | | | | |
| APT73.59 | S205R, S223A, K225H, A260G, Y276L, G282R, A283M, | Yes | 2.56 | 0.00 | 3.24 |
| | S284L | | | | |
| APT73.64 | A156C, S205R, S223A, K225H, A260G, Y276L, G282R, | Yes | 11.24 | 0.00 | 8.61 |
| | A283C, S284L | | | | |
| APT73.72 | S205R, S223A, K225H, A260G, Y276E, A280P, G282R, | Yes | 13.05 | 0.00 | 2.12 |
| | A283C, S284L | | | | |
| APT73.73 | S205R, S223A, K225H, A260G, Y276E, A280E, G282R, | Yes | 12.98 | 0.00 | 1.15 |
| | A283C, S284L | | | | |
| APT73.74 | A1561, S205R, S223A, K225H, A260G, Y276L, G282R, | Yes³ | 16.13 | 0.00 | 2.40 |
| | A283C, S284L, N285H | | | | |
| APT73.75 | A1561, S205R, S223A, K225H, A260G, Y276L, G282R, | Yes³ | 18.68 | 0.00 | 6.66 |
| | A283C, S284L, N285G | | | | |
| APT73.77 | A156I, S205R, S223A, K225H, A260G, Y276L, G282R, | Yes³ | 16.89 | 0.00 | 5.48 |
| | A283C, S284L, N285D | | | | |
| APT89 | | | 0.00 | 1.76 | 0.00 |

¹Refers to the removal of residues 285 onward from the C-terminus
²Product concentrations are normalized to the total amount produced (CBGA, O-CBGA, and F-CBGA) by APT73, which is set to 1.
³Refers to removal of residues 286 onward from the C-terminus

TABLE 3
APT73 mutants. Product formation in Yarrowia plate screening with Divarinic acid (Div) feeding. Activities
and selectivities of all enzymes were normalized to APT73 wild type, whose activity is set to 1.

| | | | | O- | F- |
|---|---|---|---|---|---|
| Enzyme | Mutations | Trunc¹ | CBGVA² | CBGVA² | CBGVA² |
| APT73 | | | 0.00 | 1.00 | 0.00 |
| APT73.13 | S205R, G282R, A283C, S284L | Yes | 0.00 | 0.00 | 0.00 |
| APT73.15 | S205R, S223A, G282R, A283C, S284L | Yes | 0.21 | 0.00 | 0.15 |
| APT73.16 | S205R, A260G, G282R, A283C, S284L | Yes | 0.10 | 0.00 | 0.00 |
| APT73.17 | S205R, Y276L, G282R, A283C, S284L | Yes | 0.21 | 0.00 | 0.15 |
| APT73.18 | S205R, Y276C, G282R, A283C, S284L | Yes | 0.00 | 0.00 | 0.00 |
| APT73.19 | S205R, Y276S, G282R, A283C, S284L | Yes | 0.00 | 0.00 | 0.00 |
| APT73.20 | S205R, G282R, A283P, S284L | Yes | 0.13 | 0.00 | 0.00 |
| APT73.21 | A156C, S205R, K225F, Y276C, G282R, A283C, S284L | Yes | 0.00 | 0.00 | 0.00 |
| APT73.22 | S205R, K225F, A260G, Y276L, G282R, A283C, S284L | Yes | 0.10 | 0.00 | 0.00 |
| APT73.23 | S205R, K225H, A260G, Y276L, G282R, A283C, S284L | Yes | 0.13 | 0.00 | 0.12 |
| APT73.24 | S205R, K225R, A260G, Y276L, G282R, A283C, S284L | Yes | 0.13 | 0.00 | 0.12 |
| APT73.25 | S205R, K225H, A260G, Y276V, G282R, A283C, S284L | Yes | 0.11 | 0.00 | 0.00 |
| APT73.26 | S205R, S223A, K225R, Y276L, G282R, A283C, S284L | Yes | 0.24 | 0.00 | 0.24 |
| APT73.27 | S205R, S223A, K225R, Y276C, G282R, A283C, S284L | Yes | 0.15 | 0.00 | 0.11 |
| APT73.28 | S205R, S223A, K225F, Y276S, G282R, A283C, S284L | Yes | 0.12 | 0.00 | 0.10 |
| APT73.29 | S205R, S223A, K225H, Y276V, G282R, A283C, S284L | Yes | 0.34 | 0.00 | 0.33 |
| APT73.30 | S205R, S223A, K225H, A260G, Y276L, G282R, A283C, | Yes | 0.23 | 0.00 | 0.24 |
| | S284L | | | | |
| APT73.31 | S205R, S223A, K225R, A260G, Y276V, G282R, A283C, | Yes | 0.53 | 0.00 | 0.39 |
| | S284L | | | | |
| APT73.32 | S205R, S223A, Y276S, G282R, A283P, S284L | Yes | 0.22 | 0.00 | 0.20 |
| APT73.33 | S205R, S223A, Y276A, G282R, A283P, S284L | Yes | 0.21 | 0.00 | 0.14 |
| APT73.34 | S205R, S223A, Y276C, G282R, A283E, S284L | Yes | 0.15 | 0.00 | 0.13 |
| APT73.35 | S205R, S223A, Y276S, G282R, A283E, S284L | Yes | 0.40 | 0.00 | 0.33 |
| APT73.36 | S205R, S223A, A260G, Y276C, G282R, A283P, S284L | Yes | 0.55 | 0.00 | 0.35 |
| APT73.37 | S205R, S223A, A260G, Y276V, G282R, A283P, S284L | Yes | 0.91 | 0.00 | 0.52 |
| APT73.44 | S205R, K225A, Y276L, G282R, A283C, S284L | Yes | 0.15 | 0.00 | 0.00 |
| APT73.45 | S205R, K225S, Y276C, G282R, A283C, S284L | Yes | 0.48 | 0.00 | 0.00 |
| APT73.46 | S205R, K225F, Y276L, G282R, A283C, S284L | Yes | 0.00 | 0.00 | 0.00 |
| APT73.47 | A156I, S205R, S223A, K225H, A260G, Y276L, G282R, | Yes | 1.56 | 0.00 | 0.70 |
| | A283C, S284L | | | | |
| APT73.49 | S205R, S223A, K225M, A260G, Y276L, G282R, A283C, | Yes | 0.47 | 0.00 | 0.61 |
| | S284L | | | | |
| APT73.52 | S205R, S223A, K225H, A260G, Y276E, G282R, A283C, | Yes | 0.75 | 0.00 | 0.35 |
| | S284L | | | | |
| APT73.53 | S205R, S223A, K225H, A260G, Y276H, G282R, A283C, | Yes | 0.71 | 0.00 | 1.01 |
| | S284L | | | | |
| APT73.54 | S205R, S223A, K225H, A260G, Y276M, G282R, A283C, | Yes | 0.35 | 0.00 | 0.44 |
| | S284L | | | | |
| APT73.58 | S205R, S223A, K225H, A260G, Y276L, G282R, A283K, | Yes | 0.26 | 0.00 | 0.32 |
| | S284L | | | | |
| APT73.59 | S205R, S223A, K225H, A260G, Y276L, G282R, A283M, | Yes | 0.25 | 0.00 | 0.32 |
| | S284L | | | | |
| APT73.64 | A156C, S205R, S223A, K225H, A260G, Y276L, G282R, | Yes | 0.57 | 0.00 | 0.43 |
| | A283C, S284L | | | | |
| APT73.72 | S205R, S223A, K225H, A260G, Y276E, A280P, G282R, | Yes | 1.20 | 0.00 | 0.36 |
| | A283C, S284L | | | | |
| APT73.73 | S205R, S223A, K225H, A260G, Y276E, A280E, G282R, | Yes | 0.59 | 0.00 | 0.15 |
| | A283C, S284L | | | | |
| APT73.74 | A156I, S205R, S223A, K225H, A260G, Y276L, G282R, | Yes³ | 2.18 | 0.00 | 0.12 |
| | A283C, S284L, N285H | | | | |
| APT73.75 | A156I, S205R, S223A, K225H, A260G, Y276L, G282R, | Yes³ | 1.49 | 0.00 | 0.41 |
| | A283C, S284L, N285G | | | | |
| APT73.77 | A156I, S205R, S223A, K225H, A260G, Y276L, G282R, | Yes³ | 3.62 | 0.00 | 0.55 |
| | A283C, S284L, N285D | | | | |
| APT89 | | | 0.00 | 2.33 | 0.00 |

¹Refers to the removal of residues 285 onward from the C-terminus
²Product concentrations are normalized to the total amount produced (CBGVA, O-CBGVA, and F-CBGVA) by APT73, which is set to 1.
³Refers to removal of residues 286 onward from the C-terminus

The screening on Yarrowia may give somewhat different results for certain mutants in terms of activity and selectivity compared to E. coli. This is due to 1) the level of expression that may vary between strains and 2) the intracellular amount of OA or Div. As a result, enzyme screening in Yarrowia will identify enzymes with potentially lower Km for substrates (GPP, FPP, OA, Div) as well as better Kcat for their conversion to products. Furthermore, the selectivity of the enzyme towards GPP vs FPP is also assessed since both substrates are available in Yarrowia's cytosol. This screening represents a more accurate depiction of activity of mutants if this organism is used as a host strain for cannabinoid production, however advantaged enzymes should also improve products in other yeasts (i.e., Saccharomyces) or bacteria (E. coli).

Tables 2 and 3 screened the same enzymes for activity against OA and Div. The relative activity and selectivity trends were very similar for both compounds proving that the targeted positions that improve OA activity and selectivity will also work for Div, (the exact mutation maybe be different). For example, APT73.47 was the most active mutant for both OA and Div substrates. Most mutants can make FCBGA with some strongly preferring GPP to form CBGA (APT73.52) while others showing similar preference for GPP and FPP (i.e APT73.64) or more preference for FPP (i.e APT73.17, APT73.53). Thus, mutagenesis of APT73 (or APT29 and APT89) can create FPP selective enzymes for prenylation of both OA and Div to F-CBGA and F-CBGVA respectively.

### Example 10—Purification of Selected Mutants and Kinetic Characterization

Selected mutants from the previous screenings were cloned in E. coli vectors and were purified as described in Example 3.

TABLE 4
Kinetic characterization of selected APT73 and APT89
(purified enzymes) with OA and GPP as substrates

| | | | | | | % |
|---|---|---|---|---|---|---|
| | | | Kcat | Km | Km | CBGA |
| | | | (CBGA) | (OA) | (GPP) | of total |
| Enzyme | Mutations | Trunc¹ | sec⁻¹ | μM | μM | products |
| NphB | | | 0.000035² | 640 ± 0.08² | N.D. | 11.1 |
| APT73.13 | S205R, G282R, A283C, | Yes | 0.049 | 1105 ± 93 | 13.02 ± 4.25 | 99.4 |
| | S284L | | | | | |
| APT73.30 | S205R, S223A, K225H, | Yes | 0.113 | 501 ± 88 | N.D. | 100 |
| | A260G, Y276L, G282R, | | | | | |
| | A283C, S284L | | | | | |
| APT73.47 | A156I, S205R, S223A, | Yes | 0.175 | 205 ± 165 | N.D. | 100 |
| | K225H, A260G, Y276L, | | | | | |
| | G282R, A283C, S284L | | | | | |
| APT73.52 | S205R, S223A, K225H, | Yes | 0.08 | 101 ± 14 | N.D. | 100 |
| | A260G, Y276E, G282R, | | | | | |
| | A283C, S284L | | | | | |
| APT89 | | | 0.001 | 33 ± 4 | N.D. | 9.8 |

¹Refers to the removal of residues 285 onward from the C-terminus
²The NphB kinetic numbers are from literature (Valliere, MA, etal Nature Commun. 2019, 10, 565)

The results from the purified enzymes clearly show that the enzymes selected from the E. coli lysate and the Yarrowia whole cell screening identified enzymes with true improvements in activity and selectivity. Furthermore, equivalent mutations in APT73 and APT89 gave similar results as shown by comparing APT73.13 and APT89.2

### Example 11—Testing of C-Terminal Truncations

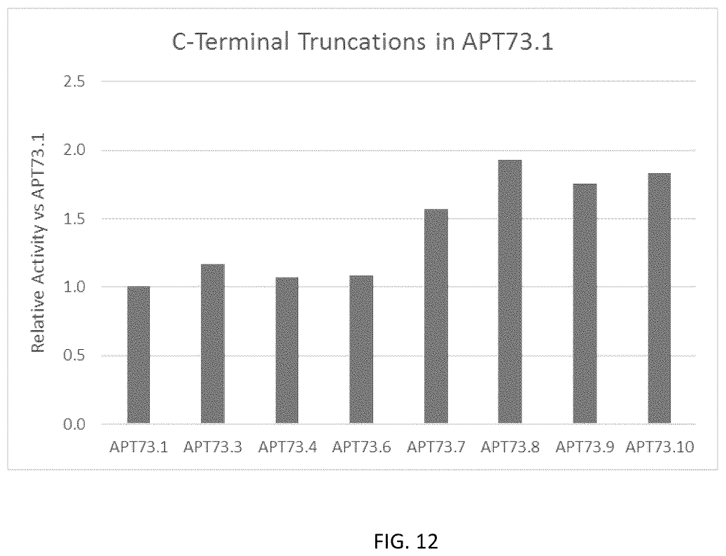

Extensive in silico modeling suggested that the enzyme's C-terminus may play a role in the activity and possibly even selectivity of the enzyme. For this reason, ten different truncations were made in APT73, the enzymes were expressed in E. coli and purified as described in Example 3. In Table 5 and FIG. 12, the relative activities of 7 truncated enzymes compared to the template, APT73.1 (S205R), are shown. All enzymes made CBGA as the major product (>99%).

TABLE 5
Relative activity of truncated APT73

| Enzyme | Residues Removed from C-terminal | CBGA¹ |
|---|---|---|
| APT73.1 | 0 | 1.00 |
| APT73.3 | 2 | 1.17 |
| APT73.4 | 4 | 1.07 |
| APT73.6 | 8 | 1.08 |
| APT73.7 | 10 | 1.57 |
| APT73.8 | 12 | 1.93 |
| APT73.9 | 14 | 1.75 |
| APT73.10 | 16 | 1.83 |

¹Product concentrations are normalized to the amount of CBGA produced by APT73.1, which is set to 1.

FIG. 12 shows CBGA production of C-terminal truncations in APT73.1 (data from Table 5). All enzymes produced CBGA as the major product (>99%). The data show that removing 2-8 residues from the C-terminus results in a small increase in CBGA production, while removing 10-16 residues results in a larger increase in CBGA production. DNA constructs for two additional truncated enzymes with 18 and 20 residues removed from the C-terminus were built, but these enzymes did not express, likely due to instability.

### Example 12: Selectivity of GPP Vs FPP of Selected APT73 and APT89 Mutants

The selectivity of enzymes in the presence of GPP, FPP or mixtures of FPP and GPP was evaluated using APT73, APT89 and selected mutants. The enzymes were expressed in E. coli and were purified as described in Example 3. Enzymes were incubated with OA (1 mM) and varying ratios of FPP/GPP and concentrations ranging from 0 to 0.5 mM GPP and FPP. For example, a 1/1 ratio of GPP/FPP contained 0.5 mM of each, a 2/1 ratio contained 0.5 mM GPP and 0.25 mM FPP, a 4/1 ratio contained 0.5 mM GPP and 0.125 mM FPP, etc.

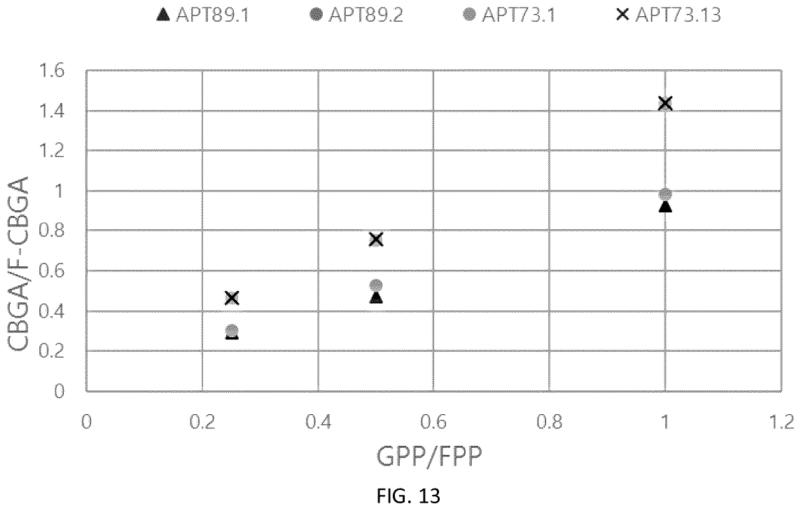

As clearly shown in FIG. 13, there is a linear dependence on the CBGA/FCBA product ratio to the supplied GPP/FPP ratio. The same mutation in either APT73 or APT89 had the same effect in each enzyme's activity and selectivity proving yet again that mutations between these templates are transferable (the same mutation has the same effect in either APT89 or APT73). Furthermore, as the enzymes are improved, the selectivity towards CBGA is increasing as clearly seen by comparing the product ratio of APT73.1, APT89.1 to APT73.13 and APT89.2 respectively. Removal of FCBGA when CBGA is the desired product and vice versa will require improvement of the enzyme's selectivity towards each substrate (FPP or GPP) as well as engineering of the cell to minimize the undesired substrate.

### Example 13—Making Cannabinoids Through Fermentation

The disclosed enzymes can be used in cell free reactions (in vitro) to produce CBGA and analogs by the feeding of the appropriate substrates, or can be introduced into a recombinant organism (yeast, bacteria, fungus, algae, or plant) to improve the flux towards CBGA or any of its analogs. These recombinant organisms will contain the appropriate genes to synthesize olivetolic acid or its analogs and a native or engineered mevalonate or MEP pathway to increase flux towards GPP or FPP. Olivetolic acid can be synthesized using the action of a polyketide or tetrakedtide synthase (TKS) followed by an OA-specific cyclase (OAC). These enzymes have been identified in Cannabis, but other enzymes with this activity can also be used.

In order to improve flux and increase the intracellular concentration of GPP, mutant farnesyl pyrophosphate synthases may be used as have been described in yeast (Jian G-Z, et al Metabolic Engineering, 2017, 41, 57) or GPP specific synthases can be introduced (Schmidt A, Gershenzon J. Phytochemistry, 2008, 69, 49). Other enzymes in the mevalonate pathway (for example HMG-CoA reductase) may need to be manipulated (truncated or mutated) or be overexpressed.

The formation of GPP/FPP and OA can occur when the organism is grown with simple carbon sources, such as glucose, sucrose, glycerol, or another simple or complex sugar mixture. External organic acids with carbon chains varying from 4 to more than 12 (in straight or branched chains) can also be supplemented during growth. These organic acids can be used as carbon sources for growth and for producing key intermediates such as butyric acid, hexanoic acid, octanoic acid. With supplementation, introduction of the appropriate acid-CoA synthase may be required to produce the corresponding organic acid-CoAs that can then be used by TKS and OAC to produce OA analogs. The organism can also express the appropriate synthase that cyclizes CBGA or any of its analogs to other cannabinoids as shown in FIG. 6.

The cells are grown in stirred tank fermenters with feed supplementation (sugars with or without organic acids) where the dissolved oxygen, temperature, and pH will be controlled according to the optimal growth and production process. Addition of aqueous non-miscible organic solvents to dissolve added organic acids or extract the cannabinoid products as they are being synthesized may also be required. These solvents may include, but are not limited to, isopropyl myristate (IPM), diisobutyl adipate, decane, dodecane, hexadecane or anther organic solvent with log P>5. Depending on the fermentation process, the products can be isolated and purified using different methods.

If no organic cosolvent is used and the targeted cannabinoid(s) is being secreted to the culture supernatant, different methods can be applied. In one, an aqueous miscible organic solvent (ethanol, acetonitrile, etc.) may be added to dissolve the products. A simple filtration, ultrafiltration or centrifugation will then remove the cells. The aqueous media can be evaporated to dryness or to a small volume from which the cannabinoid product will be precipitated or crystalized. Alternatively, the cell supernatant can be extracted with an aqueous immiscible organic solvent (ethyl acetate, heptane, decane, etc.) to extract the cannabinoids. Evaporation of the organic solvent and a possible recrystallization will produce pure cannabinoid. If the cannabinoid products are not secreted to the media and are trapped inside the cell, different methods for their extraction and purification may be required. In one method, cells will be disrupted using mechanical methods or by suspending in appropriate lysis buffers from which the cannabinoids can be extracted with an organic aqueous immiscible solvent (ethyl acetate, hexane, decane, methylene chloride, etc.). In a different method, cells may be suspended in an organic solvent (ethanol, methanol, methylene chloride, etc.) that extracts the cannabinoids from the cells.

If an organic solvent is required during growth, it will be separated at the end of the fermentation. Back extraction with basic aqueous solvent or a different organic solvent with low boiling point and high polarity (ethanol, acetonitrile, etc.) will remove the cannabinoids. Isolation can then involve a simple pH shift if water is used or an evaporation if organic solvents are used. In both cases, a recrystallization step may be required at the end to improve purity of the product.

Further Sequences

SEQ ID NO: 20 <NphB>
MKHHHHHHGTSENLYFQGMSEAADVERVYAAMEEAAGLLGVACA
RDKIYPLLSTFQDTLVEGGSVVVFSMASGRHSTELDFSISVPTSHGDPYATVVEKGLF
PATGHPVDDLLADTQKHLPVSMFAIDGEVTGGFKKTYAFFPTDNMPGVAELSAIPSM
PPAVAENAELFARYGLDKVQMTSMDYKKRQVNLYFSELSAQTLEAESVLALVRELG
LHVPNELGLKFCKRSFSVYPTLNWETGKIDRLCFAVISNDPTLVPSSDEGDIEKFHNY
ATKAPYAYVGEKRTLVYGLTLSPKEEYYKLGAYYHITDVQRGLLKAFDSLEDG
SEQ ID NO: 21 <APT29>
MKHHHHHHGTSENLYFQGMEKLMPEPVGLDKVYSAVEETADLLGV
PCSPEQFAPAVAAFGDELREAHIVESMAAGEAHRGELDFDFSVSTKGADPYATALAN
GLIKGTDHPVGALLTDIQARHAVASYGVEYGILGGFKKSYAFFPIGDYPPLAEFAAIPS
VPPGISEHVDTLTRLGLQDTVSAIGVNYAKRTLNVYLGVGEVATETKLELLRTFGFPE
PDAQVAEFVKRSFSMYPTFNWDSSVVERICFSVKTQDPGELPAPFHPEIEKFASGVPH
SYAGGREFVSAVALAPSGEAYYKLAAYYQKAQGDSKAAFAASREDDAAG
SEQ ID NO: 22 <APT73>
MKHHHHHHGTSENLYFQGMDEVYAAVEQTSRLLDVPCSPDRFEPV
WKAFGDQLPDSHLVFSMAAGEAHRGELDFDFSLRPEGADPYTTALEHGFIEPTDHPV
GSVLAEVGKRFAIASYGVEYGVVGGFKKSYAFFPLDDFPPLAQFAEVPSVPPCLAGH
VETLTRLGFDDKVSAIGVNYRKNTLNVYLAASAVDTGDKLALLRAFGYPEPDARVR
QFIERSFSLYPTFNWDSSAAERICFSVKTQQPGELPAPHDEPTEAFARQVPHVYEGGR
EFVSAVALAPSGASYYKLAAYYQKARGASNAAFAAKREDAAAG
SEQ ID NO: 23 <APT73.F116C>
MKHHHHHHGTSENLYFQGMDEVYAAVEQTSRLLDVPCSPDRFEPV
WKAFGDQLPDSHLVFSMAAGEAHRGELDFDFSLRPEGADPYTTALEHGFIEPTDHPV
GSVLAEVGKRFAIASYGVEYGVVGGFKKSYACFPLDDFPPLAQFAEVPSVPPCLAGH
VETLTRLGFDDKVSAIGVNYRKNTLNVYLAASAVDTGDKLALLRAFGYPEPDARVR
QFIERSFSLYPTFNWDSSAAERICFSVKTQQPGELPAPHDEPTEAFARQVPHVYEGGR
EFVSAVALAPSGASYYKLAAYYQKARGASNAAFAAKREDAAAG
SEQ ID NO: 24 <APT73.F116L>
MKHHHHHHGTSENLYFQGMDEVYAAVEQTSRLLDVPCSPDRFEPV
WKAFGDQLPDSHLVFSMAAGEAHRGELDFDFSLRPEGADPYTTALEHGFIEPTDHPV
GSVLAEVGKRFAIASYGVEYGVVGGFKKSYALFPLDDFPPLAQFAEVPSVPPCLAGH
VETLTRLGFDDKVSAIGVNYRKNTLNVYLAASAVDTGDKLALLRAFGYPEPDARVR
QFIERSFSLYPTFNWDSSAAERICFSVKTQQPGELPAPHDEPTEAFARQVPHVYEGGR
EFVSAVALAPSGASYYKLAAYYQKARGASNAAFAAKREDAAAG
SEQ ID NO: 25 <APT73.F116A>
MKHHHHHHGTSENLYFQGMDEVYAAVEQTSRLLDVPCSPDRFEPV
WKAFGDQLPDSHLVFSMAAGEAHRGELDFDFSLRPEGADPYTTALEHGFIEPTDHPV
GSVLAEVGKRFAIASYGVEYGVVGGFKKSYAAFPLDDFPPLAQFAEVPSVPPCLAGH
VETLTRLGFDDKVSAIGVNYRKNTLNVYLAASAVDTGDKLALLRAFGYPEPDARVR
QFIERSFSLYPTFNWDSSAAERICFSVKTQQPGELPAPHDEPTEAFARQVPHVYEGGR
EFVSAVALAPSGASYYKLAAYYQKARGASNAAFAAKREDAAAG
SEQ ID NO: 26 <APT73.S155A>
MKHHHHHHGTSENLYFQGMDEVYAAVEQTSRLLDVPCSPDRFEPV
WKAFGDQLPDSHLVFSMAAGEAHRGELDFDFSLRPEGADPYTTALEHGFIEPTDHPV
GSVLAEVGKRFAIASYGVEYGVVGGFKKSYAFFPLDDFPPLAQFAEVPSVPPCLAGH
VETLTRLGFDDKVAAIGVNYRKNTLNVYLAASAVDTGDKLALLRAFGYPEPDARVR
QFIERSFSLYPTFNWDSSAAERICFSVKTQQPGELPAPHDEPTEAFARQVPHVYEGGR
EFVSAVALAPSGASYYKLAAYYQKARGASNAAFAAKREDAAAG
SEQ ID NO: 27 <APT73.A260S>
MKHHHHHHGTSENLYFQGMDEVYAAVEQTSRLLDVPCSPDRFEPV
WKAFGDQLPDSHLVFSMAAGEAHRGELDFDFSLRPEGADPYTTALEHGFIEPTDHPV
GSVLAEVGKRFAIASYGVEYGVVGGFKKSYAFFPLDDFPPLAQFAEVPSVPPCLAGH
VETLTRLGFDDKVSAIGVNYRKNTLNVYLAASAVDTGDKLALLRAFGYPEPDARVR
QFIERSFSLYPTFNWDSSAAERICFSVKTQQPGELPAPHDEPTEAFARQVPHVYEGGR
EFVSSVALAPSGASYYKLAAYYQKARGASNAAFAAKREDAAAG
SEQ ID NO: 28 <APT73.1>
MKHHHHHHGTSENLYFQGMDEVYAAVEQTSRLLDVPCSPDRFEPV
WKAFGDQLPDSHLVFSMAAGEAHRGELDFDFSLRPEGADPYTTALEHGFIEPTDHPV
GSVLAEVGKRFAIASYGVEYGVVGGFKKSYAFFPLDDFPPLAQFAEVPSVPPCLAGH
VETLTRLGFDDKVSAIGVNYRKNTLNVYLAASAVDTGDKLALLRAFGYPEPDARVR
QFIERSFRLYPTFNWDSSAAERICFSVKTQQPGELPAPHDEPTEAFARQVPHVYEGGR
EFVSAVALAPSGASYYKLAAYYQKARGASNAAFAAKREDAAAG
SEQ ID NO: 29 <APT73.3>
MKHHHHHHGTSENLYFQGMDEVYAAVEQTSRLLDVPCSPDRFEPV
WKAFGDQLPDSHLVFSMAAGEAHRGELDFDFSLRPEGADPYTTALEHGFIEPTDHPV
GSVLAEVGKRFAIASYGVEYGVVGGFKKSYAFFPLDDFPPLAQFAEVPSVPPCLAGH
VETLTRLGFDDKVSAIGVNYRKNTLNVYLAASAVDTGDKLALLRAFGYPEPDARVR
QFIERSFRLYPTFNWDSSAAERICFSVKTQQPGELPAPHDEPTEAFARQVPHVYEGGR
EFVSAVALAPSGASYYKLAAYYQKARGASNAAFAAKREDAG
SEQ ID NO: 30 <APT73.4>
MKHHHHHHGTSENLYFQGMDEVYAAVEQTSRLLDVPCSPDRFEPV
WKAFGDQLPDSHLVFSMAAGEAHRGELDFDFSLRPEGADPYTTALEHGFIEPTDHPV
GSVLAEVGKRFAIASYGVEYGVVGGFKKSYAFFPLDDFPPLAQFAEVPSVPPCLAGH
VETLTRLGFDDKVSAIGVNYRKNTLNVYLAASAVDTGDKLALLRAFGYPEPDARVR
QFIERSFRLYPTFNWDSSAAERICFSVKTQQPGELPAPHDEPTEAFARQVPHVYEGGR
EFVSAVALAPSGASYYKLAAYYQKARGASNAAFAAKREG
SEQ ID NO: 31 <APT73.6>
MKHHHHHHGTSENLYFQGMDEVYAAVEQTSRLLDVPCSPDRFEPV
WKAFGDQLPDSHLVFSMAAGEAHRGELDFDFSLRPEGADPYTTALEHGFIEPTDHPV
GSVLAEVGKRFAIASYGVEYGVVGGFKKSYAFFPLDDFPPLAQFAEVPSVPPCLAGH
VETLTRLGFDDKVSAIGVNYRKNTLNVYLAASAVDTGDKLALLRAFGYPEPDARVR
QFIERSFRLYPTFNWDSSAAERICFSVKTQQPGELPAPHDEPTEAFARQVPHVYEGGR
EFVSAVALAPSGASYYKLAAYYQKARGASNAAFAG
SEQ ID NO: 32 <APT73.7>
MKHHHHHHGTSENLYFQGMDEVYAAVEQTSRLLDVPCSPDRFEPV
WKAFGDQLPDSHLVFSMAAGEAHRGELDFDFSLRPEGADPYTTALEHGFIEPTDHPV
GSVLAEVGKRFAIASYGVEYGVVGGFKKSYAFFPLDDFPPLAQFAEVPSVPPCLAGH
VETLTRLGFDDKVSAIGVNYRKNTLNVYLAASAVDTGDKLALLRAFGYPEPDARVR
QFIERSFRLYPTFNWDSSAAERICFSVKTQQPGELPAPHDEPTEAFARQVPHVYEGGR
EFVSAVALAPSGASYYKLAAYYQKARGASNAAG
SEQ ID NO: 33 <APT73.8>
MKHHHHHHGTSENLYFQGMDEVYAAVEQTSRLLDVPCSPDRFEPV
WKAFGDQLPDSHLVFSMAAGEAHRGELDFDFSLRPEGADPYTTALEHGFIEPTDHPV
GSVLAEVGKRFAIASYGVEYGVVGGFKKSYAFFPLDDFPPLAQFAEVPSVPPCLAGH
VETLTRLGFDDKVSAIGVNYRKNTLNVYLAASAVDTGDKLALLRAFGYPEPDARVR
QFIERSFRLYPTFNWDSSAAERICFSVKTQQPGELPAPHDEPTEAFARQVPHVYEGGR
EFVSAVALAPSGASYYKLAAYYQKARGASNG
SEQ ID NO: 34 <APT73.9>
MKHHHHHHGTSENLYFQGMDEVYAAVEQTSRLLDVPCSPDRFEPV
WKAFGDQLPDSHLVFSMAAGEAHRGELDFDFSLRPEGADPYTTALEHGFIEPTDHPV
GSVLAEVGKRFAIASYGVEYGVVGGFKKSYAFFPLDDFPPLAQFAEVPSVPPCLAGH
VETLTRLGFDDKVSAIGVNYRKNTLNVYLAASAVDTGDKLALLRAFGYPEPDARVR
QFIERSFRLYPTFNWDSSAAERICFSVKTQQPGELPAPHDEPTEAFARQVPHVYEGGR
EFVSAVALAPSGASYYKLAAYYQKARGAG
SEQ ID NO: 35 <APT73.10>
MKHHHHHHGTSENLYFQGMDEVYAAVEQTSRLLDVPCSPDRFEPV
WKAFGDQLPDSHLVFSMAAGEAHRGELDFDFSLRPEGADPYTTALEHGFIEPTDHPV
GSVLAEVGKRFAIASYGVEYGVVGGFKKSYAFFPLDDFPPLAQFAEVPSVPPCLAGH
VETLTRLGFDDKVSAIGVNYRKNTLNVYLAASAVDTGDKLALLRAFGYPEPDARVR
QFIERSFRLYPTFNWDSSAAERICFSVKTQQPGELPAPHDEPTEAFARQVPHVYEGGR
EFVSAVALAPSGASYYKLAAYYQKARG
SEQ ID NO: 36 <APT73.13>
MKHHHHHHGTSENLYFQGMDEVYAAVEQTSRLLDVPCSPDRFEPV
WKAFGDQLPDSHLVFSMAAGEAHRGELDFDFSLRPEGADPYTTALEHGFIEPTDHPV
GSVLAEVGKRFAIASYGVEYGVVGGFKKSYAFFPLDDFPPLAQFAEVPSVPPCLAGH
VETLTRLGFDDKVSAIGVNYRKNTLNVYLAASAVDTGDKLALLRAFGYPEPDARVR
QFIERSFRLYPTFNWDSSAAERICFSVKTQQPGELPAPHDEPTEAFARQVPHVYEGGR
EFVSAVALAPSGASYYKLAAYYQKARRCLG
SEQ ID NO: 37 <APT73.13.Y276V>
MKHHHHHHGTSENLYFQGMDEVYAAVEQTSRLLDVPCSPDRFEPV
WKAFGDQLPDSHLVFSMAAGEAHRGELDFDFSLRPEGADPYTTALEHGFIEPTDHPV
GSVLAEVGKRFAIASYGVEYGVVGGFKKSYAFFPLDDFPPLAQFAEVPSVPPCLAGH
VETLTRLGFDDKVSAIGVNYRKNTLNVYLAASAVDTGDKLALLRAFGYPEPDARVR
QFIERSFRLYPTFNWDSSAAERICFSVKTQQPGELPAPHDEPTEAFARQVPHVYEGGR
EFVSAVALAPSGASYYKLAAVYQKARRCLG
SEQ ID NO: 38 <APT73.13.C283A>
MKHHHHHHGTSENLYFQGMDEVYAAVEQTSRLLDVPCSPDRFEPV
WKAFGDQLPDSHLVFSMAAGEAHRGELDFDFSLRPEGADPYTTALEHGFIEPTDHPV
GSVLAEVGKRFAIASYGVEYGVVGGFKKSYAFFPLDDFPPLAQFAEVPSVPPCLAGH
VETLTRLGFDDKVSAIGVNYRKNTLNVYLAASAVDTGDKLALLRAFGYPEPDARVR
QFIERSFRLYPTFNWDSSAAERICFSVKTQQPGELPAPHDEPTEAFARQVPHVYEGGR
EFVSAVALAPSGASYYKLAAYYQKARRALG
SEQ ID NO: 39 <APT73.13.C283G>
MKHHHHHHGTSENLYFQGMDEVYAAVEQTSRLLDVPCSPDRFEPV
WKAFGDQLPDSHLVFSMAAGEAHRGELDFDFSLRPEGADPYTTALEHGFIEPTDHPV
GSVLAEVGKRFAIASYGVEYGVVGGFKKSYAFFPLDDFPPLAQFAEVPSVPPCLAGH
VETLTRLGFDDKVSAIGVNYRKNTLNVYLAASAVDTGDKLALLRAFGYPEPDARVR
QFIERSFRLYPTFNWDSSAAERICFSVKTQQPGELPAPHDEPTEAFARQVPHVYEGGR
EFVSAVALAPSGASYYKLAAYYQKARRGLG
SEQ ID NO: 40 <APT73.14>
MKHHHHHHGTSENLYFQGMDEVYAAVEQTSRLLDVPCSPDRFEPV
WKAFGDQLPDSHLVFSMAAGEAHRGELDFDFSLRPEGADPYTTALEHGFIEPTDHPV
GSVLAEVGKRFAIASYGVEYGVVGGFKKSYAFFPLDDFPPLAQFAEVPSVPPCLAGH
VETLTRLGFDDKVSAIGVNYRKNTLNVYLAASAVDTGDKLALLRAFGYPEPDARVR
QFIERSFRLYPTFNWDSSAAERICFSVKTQQPGELPAPHDEPTEAFARQVPHVYEGGR
EFVSAVALAPSGASYYKLAAYYQKARRCLG
SEQ ID NO: 41 <APT73.15>
MKHHHHHHGTSENLYFQGMDEVYAAVEQTSRLLDVPCSPDRFEPV
WKAFGDQLPDSHLVFSMAAGEAHRGELDFDFSLRPEGADPYTTALEHGFIEPTDHPV
GSVLAEVGKRFAIASYGVEYGVVGGFKKSYAFFPLDDFPPLAQFAEVPSVPPCLAGH
VETLTRLGFDDKVSAIGVNYRKNTLNVYLAASAVDTGDKLALLRAFGYPEPDARVR
QFIERSFRLYPTFNWDSSAAERICFAVKTQQPGELPAPHDEPTEAFARQVPHVYEGGR
EFVSAVALAPSGASYYKLAAYYQKARRCLG
SEQ ID NO: 42 <APT73.16>
MKHHHHHHGTSENLYFQGMDEVYAAVEQTSRLLDVPCSPDRFEPV
WKAFGDQLPDSHLVFSMAAGEAHRGELDFDFSLRPEGADPYTTALEHGFIEPTDHPV
GSVLAEVGKRFAIASYGVEYGVVGGFKKSYAFFPLDDFPPLAQFAEVPSVPPCLAGH
VETLTRLGFDDKVSAIGVNYRKNTLNVYLAASAVDTGDKLALLRAFGYPEPDARVR
QFIERSFRLYPTFNWDSSAAERICFSVKTQQPGELPAPHDEPTEAFARQVPHVYEGGR
EFVSGVALAPSGASYYKLAAYYQKARRCLG
SEQ ID NO: 43 <APT73.17>
MKHHHHHHGTSENLYFQGMDEVYAAVEQTSRLLDVPCSPDRFEPV
WKAFGDQLPDSHLVFSMAAGEAHRGELDFDFSLRPEGADPYTTALEHGFIEPTDHPV
GSVLAEVGKRFAIASYGVEYGVVGGFKKSYAFFPLDDFPPLAQFAEVPSVPPCLAGH
VETLTRLGFDDKVSAIGVNYRKNTLNVYLAASAVDTGDKLALLRAFGYPEPDARVR
QFIERSFRLYPTFNWDSSAAERICFSVKTQQPGELPAPHDEPTEAFARQVPHVYEGGR
EFVSAVALAPSGASYYKLAALYQKARRCLG
SEQ ID NO: 44 <APT73.18>
MKHHHHHHGTSENLYFQGMDEVYAAVEQTSRLLDVPCSPDRFEPV
WKAFGDQLPDSHLVFSMAAGEAHRGELDFDFSLRPEGADPYTTALEHGFIEPTDHPV
GSVLAEVGKRFAIASYGVEYGVVGGFKKSYAFFPLDDFPPLAQFAEVPSVPPCLAGH
VETLTRLGFDDKVSAIGVNYRKNTLNVYLAASAVDTGDKLALLRAFGYPEPDARVR
QFIERSFRLYPTFNWDSSAAERICFSVKTQQPGELPAPHDEPTEAFARQVPHVYEGGR
EFVSAVALAPSGASYYKLAACYQKARRCLG
SEQ ID NO: 45 <APT73.19>
MKHHHHHHGTSENLYFQGMDEVYAAVEQTSRLLDVPCSPDRFEPV
WKAFGDQLPDSHLVFSMAAGEAHRGELDFDFSLRPEGADPYTTALEHGFIEPTDHPV
GSVLAEVGKRFAIASYGVEYGVVGGFKKSYAFFPLDDFPPLAQFAEVPSVPPCLAGH
VETLTRLGFDDKVSAIGVNYRKNTLNVYLAASAVDTGDKLALLRAFGYPEPDARVR
QFIERSFRLYPTFNWDSSAAERICFSVKTQQPGELPAPHDEPTEAFARQVPHVYEGGR
EFVSAVALAPSGASYYKLAASYQKARRCLG
SEQ ID NO: 46 <APT73.20>
MKHHHHHHGTSENLYFQGMDEVYAAVEQTSRLLDVPCSPDRFEPV
WKAFGDQLPDSHLVFSMAAGEAHRGELDFDFSLRPEGADPYTTALEHGFIEPTDHPV
GSVLAEVGKRFAIASYGVEYGVVGGFKKSYAFFPLDDFPPLAQFAEVPSVPPCLAGH
VETLTRLGFDDKVSAIGVNYRKNTLNVYLAASAVDTGDKLALLRAFGYPEPDARVR
QFIERSFRLYPTFNWDSSAAERICFSVKTQQPGELPAPHDEPTEAFARQVPHVYEGGR
EFVSAVALAPSGASYYKLAAYYQKARRPLG
SEQ ID NO: 47 <APT73.21>
MKHHHHHHGTSENLYFQGMDEVYAAVEQTSRLLDVPCSPDRFEPV
WKAFGDQLPDSHLVFSMAAGEAHRGELDFDFSLRPEGADPYTTALEHGFIEPTDHPV
GSVLAEVGKRFAIASYGVEYGVVGGFKKSYAFFPLDDFPPLAQFAEVPSVPPCLAGH
VETLTRLGFDDKVSCIGVNYRKNTLNVYLAASAVDTGDKLALLRAFGYPEPDARVR
QFIERSFRLYPTFNWDSSAAERICFSVFTQQPGELPAPHDEPTEAFARQVPHVYEGGRE
FVSAVALAPSGASYYKLAALYQKARRCLG
SEQ ID NO: 48 <APT73.22>
MKHHHHHHGTSENLYFQGMDEVYAAVEQTSRLLDVPCSPDRFEPV
WKAFGDQLPDSHLVFSMAAGEAHRGELDFDFSLRPEGADPYTTALEHGFIEPTDHPV
GSVLAEVGKRFAIASYGVEYGVVGGFKKSYAFFPLDDFPPLAQFAEVPSVPPCLAGH
VETLTRLGFDDKVSAIGVNYRKNTLNVYLAASAVDTGDKLALLRAFGYPEPDARVR
QFIERSFRLYPTFNWDSSAAERICFSVFTQQPGELPAPHDEPTEAFARQVPHVYEGGRE
FVSGVALAPSGASYYKLAALYQKARRCLG
SEQ ID NO: 49 <APT73.23>
MKHHHHHHGTSENLYFQGMDEVYAAVEQTSRLLDVPCSPDRFEPV
WKAFGDQLPDSHLVFSMAAGEAHRGELDFDFSLRPEGADPYTTALEHGFIEPTDHPV
GSVLAEVGKRFAIASYGVEYGVVGGFKKSYAFFPLDDFPPLAQFAEVPSVPPCLAGH
VETLTRLGFDDKVSAIGVNYRKNTLNVYLAASAVDTGDKLALLRAFGYPEPDARVR
QFIERSFRLYPTFNWDSSAAERICFSVHTQQPGELPAPHDEPTEAFARQVPHVYEGGR
EFVSGVALAPSGASYYKLAALYQKARRCLG
SEQ ID NO: 50 <APT73.24>
MKHHHHHHGTSENLYFQGMDEVYAAVEQTSRLLDVPCSPDRFEPV
WKAFGDQLPDSHLVFSMAAGEAHRGELDFDFSLRPEGADPYTTALEHGFIEPTDHPV
GSVLAEVGKRFAIASYGVEYGVVGGFKKSYAFFPLDDFPPLAQFAEVPSVPPCLAGH
VETLTRLGFDDKVSAIGVNYRKNTLNVYLAASAVDTGDKLALLRAFGYPEPDARVR
QFIERSFRLYPTFNWDSSAAERICFSVRTQQPGELPAPHDEPTEAFARQVPHVYEGGR
EFVSGVALAPSGASYYKLAALYQKARRCLG
SEQ ID NO: 51 <APT73.25>
MKHHHHHHGTSENLYFQGMDEVYAAVEQTSRLLDVPCSPDRFEPV
WKAFGDQLPDSHLVFSMAAGEAHRGELDFDFSLRPEGADPYTTALEHGFIEPTDHPV
GSVLAEVGKRFAIASYGVEYGVVGGFKKSYAFFPLDDFPPLAQFAEVPSVPPCLAGH
VETLTRLGFDDKVSAIGVNYRKNTLNVYLAASAVDTGDKLALLRAFGYPEPDARVR
QFIERSFRLYPTFNWDSSAAERICFSVHTQQPGELPAPHDEPTEAFARQVPHVYEGGR
EFVSGVALAPSGASYYKLAAVYQKARRCLG
SEQ ID NO: 52 <APT73.26>
MKHHHHHHGTSENLYFQGMDEVYAAVEQTSRLLDVPCSPDRFEPV
WKAFGDQLPDSHLVFSMAAGEAHRGELDFDFSLRPEGADPYTTALEHGFIEPTDHPV
GSVLAEVGKRFAIASYGVEYGVVGGFKKSYAFFPLDDFPPLAQFAEVPSVPPCLAGH
VETLTRLGFDDKVSAIGVNYRKNTLNVYLAASAVDTGDKLALLRAFGYPEPDARVR
QFIERSFRLYPTFNWDSSAAERICFAVRTQQPGELPAPHDEPTEAFARQVPHVYEGGR
EFVSAVALAPSGASYYKLAALYQKARRCLG
SEQ ID NO: 53 <APT73.27>
MKHHHHHHGTSENLYFQGMDEVYAAVEQTSRLLDVPCSPDRFEPV
WKAFGDQLPDSHLVFSMAAGEAHRGELDFDFSLRPEGADPYTTALEHGFIEPTDHPV
GSVLAEVGKRFAIASYGVEYGVVGGFKKSYAFFPLDDFPPLAQFAEVPSVPPCLAGH
VETLTRLGFDDKVSAIGVNYRKNTLNVYLAASAVDTGDKLALLRAFGYPEPDARVR
QFIERSFRLYPTFNWDSSAAERICFAVRTQQPGELPAPHDEPTEAFARQVPHVYEGGR
EFVSAVALAPSGASYYKLAACYQKARRCLG
SEQ ID NO: 54 <APT73.28>
MKHHHHHHGTSENLYFQGMDEVYAAVEQTSRLLDVPCSPDRFEPV
WKAFGDQLPDSHLVFSMAAGEAHRGELDFDFSLRPEGADPYTTALEHGFIEPTDHPV
GSVLAEVGKRFAIASYGVEYGVVGGFKKSYAFFPLDDFPPLAQFAEVPSVPPCLAGH
VETLTRLGFDDKVSAIGVNYRKNTLNVYLAASAVDTGDKLALLRAFGYPEPDARVR
QFIERSFRLYPTFNWDSSAAERICFAVFTQQPGELPAPHDEPTEAFARQVPHVYEGGR
EFVSAVALAPSGASYYKLAASYQKARRCLG
SEQ ID NO: 55 <APT73.29>
MKHHHHHHGTSENLYFQGMDEVYAAVEQTSRLLDVPCSPDRFEPV
WKAFGDQLPDSHLVFSMAAGEAHRGELDFDFSLRPEGADPYTTALEHGFIEPTDHPV
GSVLAEVGKRFAIASYGVEYGVVGGFKKSYAFFPLDDFPPLAQFAEVPSVPPCLAGH
VETLTRLGFDDKVSAIGVNYRKNTLNVYLAASAVDTGDKLALLRAFGYPEPDARVR
QFIERSFRLYPTFNWDSSAAERICFAVHTQQPGELPAPHDEPTEAFARQVPHVYEGGR
EFVSAVALAPSGASYYKLAAVYQKARRCLG
SEQ ID NO: 56 <APT73.30>
MKHHHHHHGTSENLYFQGMDEVYAAVEQTSRLLDVPCSPDRFEPV
WKAFGDQLPDSHLVFSMAAGEAHRGELDFDFSLRPEGADPYTTALEHGFIEPTDHPV
GSVLAEVGKRFAIASYGVEYGVVGGFKKSYAFFPLDDFPPLAQFAEVPSVPPCLAGH
VETLTRLGFDDKVSAIGVNYRKNTLNVYLAASAVDTGDKLALLRAFGYPEPDARVR
QFIERSFRLYPTFNWDSSAAERICFAVHTQQPGELPAPHDEPTEAFARQVPHVYEGGR
EFVSGVALAPSGASYYKLAALYQKARRCLG
SEQ ID NO: 57 <APT73.31>
MKHHHHHHGTSENLYFQGMDEVYAAVEQTSRLLDVPCSPDRFEPV
WKAFGDQLPDSHLVFSMAAGEAHRGELDFDFSLRPEGADPYTTALEHGFIEPTDHPV
GSVLAEVGKRFAIASYGVEYGVVGGFKKSYAFFPLDDFPPLAQFAEVPSVPPCLAGH
VETLTRLGFDDKVSAIGVNYRKNTLNVYLAASAVDTGDKLALLRAFGYPEPDARVR
QFIERSFRLYPTFNWDSSAAERICFAVRTQQPGELPAPHDEPTEAFARQVPHVYEGGR
EFVSGVALAPSGASYYKLAAVYQKARRCLG
SEQ ID NO: 58 <APT73.32>
MKHHHHHHGTSENLYFQGMDEVYAAVEQTSRLLDVPCSPDRFEPV
WKAFGDQLPDSHLVFSMAAGEAHRGELDFDFSLRPEGADPYTTALEHGFIEPTDHPV
GSVLAEVGKRFAIASYGVEYGVVGGFKKSYAFFPLDDFPPLAQFAEVPSVPPCLAGH
VETLTRLGFDDKVSAIGVNYRKNTLNVYLAASAVDTGDKLALLRAFGYPEPDARVR
QFIERSFRLYPTFNWDSSAAERICFAVKTQQPGELPAPHDEPTEAFARQVPHVYEGGR
EFVSAVALAPSGASYYKLAASYQKARRPLG
SEQ ID NO: 59 <APT73.33>
MKHHHHHHGTSENLYFQGMDEVYAAVEQTSRLLDVPCSPDRFEPV
WKAFGDQLPDSHLVFSMAAGEAHRGELDFDFSLRPEGADPYTTALEHGFIEPTDHPV
GSVLAEVGKRFAIASYGVEYGVVGGFKKSYAFFPLDDFPPLAQFAEVPSVPPCLAGH
VETLTRLGFDDKVSAIGVNYRKNTLNVYLAASAVDTGDKLALLRAFGYPEPDARVR
QFIERSFRLYPTFNWDSSAAERICFAVKTQQPGELPAPHDEPTEAFARQVPHVYEGGR
EFVSAVALAPSGASYYKLAAAYQKARRPLG
SEQ ID NO: 60 <APT73.34>
MKHHHHHHGTSENLYFQGMDEVYAAVEQTSRLLDVPCSPDRFEPV
WKAFGDQLPDSHLVFSMAAGEAHRGELDFDFSLRPEGADPYTTALEHGFIEPTDHPV
GSVLAEVGKRFAIASYGVEYGVVGGFKKSYAFFPLDDFPPLAQFAEVPSVPPCLAGH
VETLTRLGFDDKVSAIGVNYRKNTLNVYLAASAVDTGDKLALLRAFGYPEPDARVR
QFIERSFRLYPTFNWDSSAAERICFAVKTQQPGELPAPHDEPTEAFARQVPHVYEGGR
EFVSAVALAPSGASYYKLAACYQKARRELG
SEQ ID NO: 61 <APT73.35>
MKHHHHHHGTSENLYFQGMDEVYAAVEQTSRLLDVPCSPDRFEPV
WKAFGDQLPDSHLVFSMAAGEAHRGELDFDFSLRPEGADPYTTALEHGFIEPTDHPV
GSVLAEVGKRFAIASYGVEYGVVGGFKKSYAFFPLDDFPPLAQFAEVPSVPPCLAGH
VETLTRLGFDDKVSAIGVNYRKNTLNVYLAASAVDTGDKLALLRAFGYPEPDARVR
QFIERSFRLYPTFNWDSSAAERICFAVKTQQPGELPAPHDEPTEAFARQVPHVYEGGR
EFVSAVALAPSGASYYKLAASYQKARRELG
SEQ ID NO: 62 <APT73.36>
MKHHHHHHGTSENLYFQGMDEVYAAVEQTSRLLDVPCSPDRFEPV
WKAFGDQLPDSHLVFSMAAGEAHRGELDFDFSLRPEGADPYTTALEHGFIEPTDHPV
GSVLAEVGKRFAIASYGVEYGVVGGFKKSYAFFPLDDFPPLAQFAEVPSVPPCLAGH
VETLTRLGFDDKVSAIGVNYRKNTLNVYLAASAVDTGDKLALLRAFGYPEPDARVR
QFIERSFRLYPTFNWDSSAAERICFAVKTQQPGELPAPHDEPTEAFARQVPHVYEGGR
EFVSGVALAPSGASYYKLAACYQKARRPLG
SEQ ID NO: 63 <APT73.37>
MKHHHHHHGTSENLYFQGMDEVYAAVEQTSRLLDVPCSPDRFEPV
WKAFGDQLPDSHLVFSMAAGEAHRGELDFDFSLRPEGADPYTTALEHGFIEPTDHPV
GSVLAEVGKRFAIASYGVEYGVVGGFKKSYAFFPLDDFPPLAQFAEVPSVPPCLAGH
VETLTRLGFDDKVSAIGVNYRKNTLNVYLAASAVDTGDKLALLRAFGYPEPDARVR
QFIERSFRLYPTFNWDSSAAERICFAVKTQQPGELPAPHDEPTEAFARQVPHVYEGGR
EFVSGVALAPSGASYYKLAAVYQKARRPLG
SEQ ID NO: 64 <APT73.44>
MKHHHHHHGTSENLYFQGMDEVYAAVEQTSRLLDVPCSPDRFEPV
WKAFGDQLPDSHLVFSMAAGEAHRGELDFDFSLRPEGADPYTTALEHGFIEPTDHPV
GSVLAEVGKRFAIASYGVEYGVVGGFKKSYAFFPLDDFPPLAQFAEVPSVPPCLAGH
VETLTRLGFDDKVSAIGVNYRKNTLNVYLAASAVDTGDKLALLRAFGYPEPDARVR
QFIERSFRLYPTFNWDSSAAERICFSVATQQPGELPAPHDEPTEAFARQVPHVYEGGR
EFVSAVALAPSGASYYKLAALYQKARRCLG
SEQ ID NO: 65 <APT73.45>
MKHHHHHHGTSENLYFQGMDEVYAAVEQTSRLLDVPCSPDRFEPV
WKAFGDQLPDSHLVFSMAAGEAHRGELDFDFSLRPEGADPYTTALEHGFIEPTDHPV
GSVLAEVGKRFAIASYGVEYGVVGGFKKSYAFFPLDDFPPLAQFAEVPSVPPCLAGH
VETLTRLGFDDKVSAIGVNYRKNTLNVYLAASAVDTGDKLALLRAFGYPEPDARVR
QFIERSFRLYPTFNWDSSAAERICFSVSTQQPGELPAPHDEPTEAFARQVPHVYEGGRE
FVSAVALAPSGASYYKLAACYQKARRCLG
SEQ ID NO: 66 <APT73.46>
MKHHHHHHGTSENLYFQGMDEVYAAVEQTSRLLDVPCSPDRFEPV
WKAFGDQLPDSHLVFSMAAGEAHRGELDFDFSLRPEGADPYTTALEHGFIEPTDHPV
GSVLAEVGKRFAIASYGVEYGVVGGFKKSYAFFPLDDFPPLAQFAEVPSVPPCLAGH
VETLTRLGFDDKVSAIGVNYRKNTLNVYLAASAVDTGDKLALLRAFGYPEPDARVR
QFIERSFRLYPTFNWDSSAAERICFSVFTQQPGELPAPHDEPTEAFARQVPHVYEGGRE
FVSAVALAPSGASYYKLAATYQKARRCLG
SEQ ID NO: 67 <APT73.47>
MKHHHHHHGTSENLYFQGMDEVYAAVEQTSRLLDVPCSPDRFEPV
WKAFGDQLPDSHLVFSMAAGEAHRGELDFDFSLRPEGADPYTTALEHGFIEPTDHPV
GSVLAEVGKRFAIASYGVEYGVVGGFKKSYAFFPLDDFPPLAQFAEVPSVPPCLAGH
VETLTRLGFDDKVSIIGVNYRKNTLNVYLAASAVDTGDKLALLRAFGYPEPDARVRQ
FIERSFRLYPTFNWDSSAAERICFAVHTQQPGELPAPHDEPTEAFARQVPHVYEGGRE
FVSGVALAPSGASYYKLAALYQKARRCLG
SEQ ID NO: 68 <APT73.49>
MKHHHHHHGTSENLYFQGMDEVYAAVEQTSRLLDVPCSPDRFEPV
WKAFGDQLPDSHLVFSMAAGEAHRGELDFDFSLRPEGADPYTTALEHGFIEPTDHPV
GSVLAEVGKRFAIASYGVEYGVVGGFKKSYAFFPLDDFPPLAQFAEVPSVPPCLAGH
VETLTRLGFDDKVSAIGVNYRKNTLNVYLAASAVDTGDKLALLRAFGYPEPDARVR
QFIERSFRLYPTFNWDSSAAERICFAVMTQQPGELPAPHDEPTEAFARQVPHVYEGGR
EFVSGVALAPSGASYYKLAALYQKARRCLG
SEQ ID NO: 69 <APT73.52>
MKHHHHHHGTSENLYFQGMDEVYAAVEQTSRLLDVPCSPDRFEPV
WKAFGDQLPDSHLVFSMAAGEAHRGELDFDFSLRPEGADPYTTALEHGFIEPTDHPV
GSVLAEVGKRFAIASYGVEYGVVGGFKKSYAFFPLDDFPPLAQFAEVPSVPPCLAGH
VETLTRLGFDDKVSAIGVNYRKNTLNVYLAASAVDTGDKLALLRAFGYPEPDARVR
QFIERSFRLYPTFNWDSSAAERICFAVHTQQPGELPAPHDEPTEAFARQVPHVYEGGR
EFVSGVALAPSGASYYKLAAEYQKARRCLG
SEQ ID NO: 70 <APT73.53>
MKHHHHHHGTSENLYFQGMDEVYAAVEQTSRLLDVPCSPDRFEPV
WKAFGDQLPDSHLVFSMAAGEAHRGELDFDFSLRPEGADPYTTALEHGFIEPTDHPV
GSVLAEVGKRFAIASYGVEYGVVGGFKKSYAFFPLDDFPPLAQFAEVPSVPPCLAGH
VETLTRLGFDDKVSAIGVNYRKNTLNVYLAASAVDTGDKLALLRAFGYPEPDARVR
QFIERSFRLYPTFNWDSSAAERICFAVHTQQPGELPAPHDEPTEAFARQVPHVYEGGR
EFVSGVALAPSGASYYKLAAHYQKARRCLG
SEQ ID NO: 71 <APT73.54>
MKHHHHHHGTSENLYFQGMDEVYAAVEQTSRLLDVPCSPDRFEPV
WKAFGDQLPDSHLVFSMAAGEAHRGELDFDFSLRPEGADPYTTALEHGFIEPTDHPV
GSVLAEVGKRFAIASYGVEYGVVGGFKKSYAFFPLDDFPPLAQFAEVPSVPPCLAGH
VETLTRLGFDDKVSAIGVNYRKNTLNVYLAASAVDTGDKLALLRAFGYPEPDARVR
QFIERSFRLYPTFNWDSSAAERICFAVHTQQPGELPAPHDEPTEAFARQVPHVYEGGR
EFVSGVALAPSGASYYKLAAMYQKARRCLG
SEQ ID NO: 72 <APT73.58>
MKHHHHHHGTSENLYFQGMDEVYAAVEQTSRLLDVPCSPDRFEPV
WKAFGDQLPDSHLVFSMAAGEAHRGELDFDFSLRPEGADPYTTALEHGFIEPTDHPV
GSVLAEVGKRFAIASYGVEYGVVGGFKKSYAFFPLDDFPPLAQFAEVPSVPPCLAGH
VETLTRLGFDDKVSAIGVNYRKNTLNVYLAASAVDTGDKLALLRAFGYPEPDARVR
QFIERSFRLYPTFNWDSSAAERICFAVHTQQPGELPAPHDEPTEAFARQVPHVYEGGR
EFVSGVALAPSGASYYKLAALYQKARRKLG
SEQ ID NO: 73 <APT73.59>
MKHHHHHHGTSENLYFQGMDEVYAAVEQTSRLLDVPCSPDRFEPV
WKAFGDQLPDSHLVFSMAAGEAHRGELDFDFSLRPEGADPYTTALEHGFIEPTDHPV
GSVLAEVGKRFAIASYGVEYGVVGGFKKSYAFFPLDDFPPLAQFAEVPSVPPCLAGH
VETLTRLGFDDKVSAIGVNYRKNTLNVYLAASAVDTGDKLALLRAFGYPEPDARVR
QFIERSFRLYPTFNWDSSAAERICFAVHTQQPGELPAPHDEPTEAFARQVPHVYEGGR
EFVSGVALAPSGASYYKLAALYQKARRMLG
SEQ ID NO: 74 <APT73.64>
MKHHHHHHGTSENLYFQGMDEVYAAVEQTSRLLDVPCSPDRFEPV
WKAFGDQLPDSHLVFSMAAGEAHRGELDFDFSLRPEGADPYTTALEHGFIEPTDHPV
GSVLAEVGKRFAIASYGVEYGVVGGFKKSYAFFPLDDFPPLAQFAEVPSVPPCLAGH
VETLTRLGFDDKVSCIGVNYRKNTLNVYLAASAVDTGDKLALLRAFGYPEPDARVR
QFIERSFRLYPTFNWDSSAAERICFAVHTQQPGELPAPHDEPTEAFARQVPHVYEGGR
EFVSGVALAPSGASYYKLAALYQKARRCLG
SEQ ID NO: 75 <APT73.72>
MKHHHHHHGTSENLYFQGMDEVYAAVEQTSRLLDVPCSPDRFEPV
WKAFGDQLPDSHLVFSMAAGEAHRGELDFDFSLRPEGADPYTTALEHGFIEPTDHPV
GSVLAEVGKRFAIASYGVEYGVVGGFKKSYAFFPLDDFPPLAQFAEVPSVPPCLAGH
VETLTRLGFDDKVSAIGVNYRKNTLNVYLAASAVDTGDKLALLRAFGYPEPDARVR
QFIERSFRLYPTFNWDSSAAERICFAVHTQQPGELPAPHDEPTEAFARQVPHVYEGGR
EFVSGVALAPSGASYYKLAAEYQKPRRCLG
SEQ ID NO: 76 <APT73.73>
MKHHHHHHGTSENLYFQGMDEVYAAVEQTSRLLDVPCSPDRFEPV
WKAFGDQLPDSHLVFSMAAGEAHRGELDFDFSLRPEGADPYTTALEHGFIEPTDHPV
GSVLAEVGKRFAIASYGVEYGVVGGFKKSYAFFPLDDFPPLAQFAEVPSVPPCLAGH
VETLTRLGFDDKVSAIGVNYRKNTLNVYLAASAVDTGDKLALLRAFGYPEPDARVR
QFIERSFRLYPTFNWDSSAAERICFAVHTQQPGELPAPHDEPTEAFARQVPHVYEGGR
EFVSGVALAPSGASYYKLAAEYQKERRCLG
SEQ ID NO: 77 <APT73.74>
MKHHHHHHGTSENLYFQGMDEVYAAVEQTSRLLDVPCSPDRFEPV
WKAFGDQLPDSHLVFSMAAGEAHRGELDFDFSLRPEGADPYTTALEHGFIEPTDHPV
GSVLAEVGKRFAIASYGVEYGVVGGFKKSYAFFPLDDFPPLAQFAEVPSVPPCLAGH
VETLTRLGFDDKVSIIGVNYRKNTLNVYLAASAVDTGDKLALLRAFGYPEPDARVRQ
FIERSFRLYPTFNWDSSAAERICFAVHTQQPGELPAPHDEPTEAFARQVPHVYEGGRE
FVSGVALAPSGASYYKLAALYQKARRCLHG
SEQ ID NO: 78 <APT73.75>
MKHHHHHHGTSENLYFQGMDEVYAAVEQTSRLLDVPCSPDRFEPV
WKAFGDQLPDSHLVFSMAAGEAHRGELDFDFSLRPEGADPYTTALEHGFIEPTDHPV
GSVLAEVGKRFAIASYGVEYGVVGGFKKSYAFFPLDDFPPLAQFAEVPSVPPCLAGH
VETLTRLGFDDKVSIIGVNYRKNTLNVYLAASAVDTGDKLALLRAFGYPEPDARVRQ
FIERSFRLYPTFNWDSSAAERICFAVHTQQPGELPAPHDEPTEAFARQVPHVYEGGRE
FVSGVALAPSGASYYKLAALYQKARRCLGG
SEQ ID NO: 79 <APT73.77>
MKHHHHHHGTSENLYFQGMDEVYAAVEQTSRLLDVPCSPDRFEPV
WKAFGDQLPDSHLVFSMAAGEAHRGELDFDFSLRPEGADPYTTALEHGFIEPTDHPV
GSVLAEVGKRFAIASYGVEYGVVGGFKKSYAFFPLDDFPPLAQFAEVPSVPPCLAGH
VETLTRLGFDDKVSIIGVNYRKNTLNVYLAASAVDTGDKLALLRAFGYPEPDARVRQ
FIERSFRLYPTFNWDSSAAERICFAVHTQQPGELPAPHDEPTEAFARQVPHVYEGGRE
FVSGVALAPSGASYYKLAALYQKARRCLDG
SEQ ID NO: 80 <APT88>
MKHHHHHHGTSENLYFQGMMQRRWSVVGVPAEPGAGAVRGRWPV
KCRSDGGSWLQRAPSGRQAGCARVVGACRADRLNFLEELMAGPAGLDEVYAAVER
TSRLLDVPCSPDRFEPVWKAFGDQLPDSHLVFSMAAGEAHRGELDFDFSLRPEGADP
YTTALEHGFIEPTDHPVGSVLAEVNKRCEIASYGVEYGVVGGFKKSYAFFPLDDFPPL
AEFARIPSVPPCLAGHVDTLTRLGLDDKVSAIGVNYRKNTLNVYLAASAVATDDKLA
LLRAFGYPEPDARVRQFIERSFSLYPTFNWDSSAAERICFSVKTQQPGELPAPHDEPTE
AFAREVPHVYEGGREFVSAVALAPSGAAYYKLAAYYQKARGASNAAFAAKREDAA
AG
SEQ ID NO: 81 <APT89>
MKHHHHHHGTSENLYFQGMDEVYAAVERTSRLLDVPCSPDRFEPV
WKAFGDQLPDSHLVFSMAAGEAHRGELDFDFSLRPEGADPYTTALEHGFIEPTDHPV
GSVLAEVNKRCEIASYGVEYGVVGGFKKSYAFFPLDDFPPLAEFARIPSVPPCLAGHV
DTLTRLGLDDKVSAIGVNYRKNTLNVYLAASAVATDDKLALLRAFGYPEPDARVRQ
FIERSFSLYPTFNWDSSAAERICFSVKTQQPGELPAPHDEPTEAFAREVPHVYEGGREF
VSAVALAPSGAAYYKLAAYYQKARGASNAAFAAKREDAAAG
SEQ ID NO: 82 <APT89.F116L>
MKHHHHHHGTSENLYFQGMDEVYAAVERTSRLLDVPCSPDRFEPV
WKAFGDQLPDSHLVFSMAAGEAHRGELDFDFSLRPEGADPYTTALEHGFIEPTDHPV
GSVLAEVNKRCEIASYGVEYGVVGGFKKSYALFPLDDFPPLAEFARIPSVPPCLAGHV
DTLTRLGLDDKVSAIGVNYRKNTLNVYLAASAVATDDKLALLRAFGYPEPDARVRQ
FIERSFSLYPTFNWDSSAAERICFSVKTQQPGELPAPHDEPTEAFAREVPHVYEGGREF
VSAVALAPSGAAYYKLAAYYQKARGASNAAFAAKREDAAAG
SEQ ID NO: 83 <APT89.F116A>
MKHHHHHHGTSENLYFQGMDEVYAAVERTSRLLDVPCSPDRFEPV
WKAFGDQLPDSHLVFSMAAGEAHRGELDFDFSLRPEGADPYTTALEHGFIEPTDHPV
GSVLAEVNKRCEIASYGVEYGVVGGFKKSYAAFPLDDFPPLAEFARIPSVPPCLAGHV
DTLTRLGLDDKVSAIGVNYRKNTLNVYLAASAVATDDKLALLRAFGYPEPDARVRQ
FIERSFSLYPTFNWDSSAAERICFSVKTQQPGELPAPHDEPTEAFAREVPHVYEGGREF
VSAVALAPSGAAYYKLAAYYQKARGASNAAFAAKREDAAAG
SEQ ID NO: 84 <APT89.S155A>
MKHHHHHHGTSENLYFQGMDEVYAAVERTSRLLDVPCSPDRFEPV
WKAFGDQLPDSHLVFSMAAGEAHRGELDFDFSLRPEGADPYTTALEHGFIEPTDHPV
GSVLAEVNKRCEIASYGVEYGVVGGFKKSYAFFPLDDFPPLAEFARIPSVPPCLAGHV
DTLTRLGLDDKVAAIGVNYRKNTLNVYLAASAVATDDKLALLRAFGYPEPDARVRQ
FIERSFSLYPTFNWDSSAAERICFSVKTQQPGELPAPHDEPTEAFAREVPHVYEGGREF
VSAVALAPSGAAYYKLAAYYQKARGASNAAFAAKREDAAAG
SEQ ID NO: 85 <APT89.A260S>
MKHHHHHHGTSENLYFQGMDEVYAAVERTSRLLDVPCSPDRFEPV
WKAFGDQLPDSHLVFSMAAGEAHRGELDFDFSLRPEGADPYTTALEHGFIEPTDHPV
GSVLAEVNKRCEIASYGVEYGVVGGFKKSYAFFPLDDFPPLAEFARIPSVPPCLAGHV
DTLTRLGLDDKVSAIGVNYRKNTLNVYLAASAVATDDKLALLRAFGYPEPDARVRQ
FIERSFSLYPTFNWDSSAAERICFSVKTQQPGELPAPHDEPTEAFAREVPHVYEGGREF
VSSVALAPSGAAYYKLAAYYQKARGASNAAFAAKREDAAAG
SEQ ID NO: 86 <APT89.1>
MKHHHHHHGTSENLYFQGMDEVYAAVERTSRLLDVPCSPDRFEPV
WKAFGDQLPDSHLVFSMAAGEAHRGELDFDFSLRPEGADPYTTALEHGFIEPTDHPV
GSVLAEVNKRCEIASYGVEYGVVGGFKKSYAFFPLDDFPPLAEFARIPSVPPCLAGHV
DTLTRLGLDDKVSAIGVNYRKNTLNVYLAASAVATDDKLALLRAFGYPEPDARVRQ
FIERSFRLYPTFNWDSSAAERICFSVKTQQPGELPAPHDEPTEAFAREVPHVYEGGREF
VSAVALAPSGAAYYKLAAYYQKARGASNAAFAAKREDAAAG
SEQ ID NO: 87 <APT89.2>
MKHHHHHHGTSENLYFQGMDEVYAAVERTSRLLDVPCSPDRFEPV
WKAFGDQLPDSHLVFSMAAGEAHRGELDFDFSLRPEGADPYTTALEHGFIEPTDHPV
GSVLAEVNKRCEIASYGVEYGVVGGFKKSYAFFPLDDFPPLAEFARIPSVPPCLAGHV
DTLTRLGLDDKVSAIGVNYRKNTLNVYLAASAVATDDKLALLRAFGYPEPDARVRQ
FIERSFRLYPTFNWDSSAAERICFSVKTQQPGELPAPHDEPTEAFAREVPHVYEGGREF
VSAVALAPSGAAYYKLAAYYQKARRCLG
SEQ ID NO: 88 <APT89.6>
MKHHHHHHGTSENLYFQGMDEVYAAVERTSRLLDVPCSPDRFEPV
WKAFGDQLPDSHLVFSMAAGEAHRGELDFDFSLRPEGADPYTTALEHGFIEPTDHPV
GSVLAEVNKRCEIASYGVEYGVVGGFKKSYAFFPLDDFPPLAEFARIPSVPPCLAGHV
DTLTRLGLDDKVSAIGVNYRKNTLNVYLAASAVATDDKLALLRAFGYPEPDARVRQ
FIERSFKLYPTFNWDSSAAERICFSVKTQQPGELPAPHDEPTEAFAREVPHVYEGGREF
VSAVALAPSGAAYYKLAAYYQKARGASNAAFAAKREDAAAG
SEQ ID NO: 89 <NphB.NTS>
ATGAAACATCACCACCACCACCATGGCACATCTGAAAACTTGTAT
TTCCAGGGCATGAGCGAAGCTGCAGATGTTGAACGCGTTTATGCAGCAATGGAA
GAAGCAGCAGGTTTACTGGGTGTTGCATGTGCACGCGATAAAATTTACCCTTTAC
TGAGCACCTTCCAGGATACCCTGGTTGAAGGTGGTAGCGTTGTGGTGTTCTCAAT
GGCAAGTGGTAGACATAGTACCGAACTGGATTTCAGCATTAGTGTTCCGACCAGC
CATGGTGATCCTTATGCAACCGTTGTGGAAAAAGGCCTGTTTCCTGCAACAGGTC
ATCCTGTTGATGATCTGTTAGCCGATACCCAGAAACATCTGCCTGTTAGCATGTTT
GCCATTGATGGCGAAGTTACAGGTGGCTTCAAGAAGACCTACGCCTTTTTTCCGA
CCGACAATATGCCTGGTGTTGCCGAATTAAGTGCAATTCCGTCTATGCCTCCTGC
AGTTGCAGAAAATGCCGAATTATTTGCCCGCTATGGCCTGGATAAAGTTCAGATG
ACCAGCATGGACTACAAGAAACGTCAGGTGAACCTGTATTTCAGCGAGCTGTCA
GCACAGACCTTAGAAGCAGAAAGCGTTTTAGCCTTAGTGCGTGAATTAGGTCTGC
ATGTGCCGAATGAACTGGGCCTGAAATTCTGCAAACGCTCATTTAGCGTTTATCC
GACACTGAACTGGGAAACCGGCAAAATTGACCGCCTGTGTTTTGCAGTGATCAGC
AATGATCCTACATTAGTTCCGAGCAGCGATGAGGGCGATATCGAGAAGTTCCACA
ATTATGCCACCAAAGCACCTTATGCATATGTGGGCGAAAAACGTACCCTGGTGTA
TGGTCTGACCTTAAGTCCGAAGGAAGAGTACTACAAATTAGGCGCCTACTATCAC
ATCACCGACGTTCAACGTGGTCTGTTAAAGGCCTTCGATAGCCTGGAAGATGGTT
AG
SEQ ID NO: 90 <APT29.NTS>
ATGAAACATCACCACCACCACCATGGCACATCTGAAAACTTGTAT
TTCCAGGGCATGGAGAAGCTGATGCCTGAACCTGTCGGTCTGGACAAGGTCTACT
CTGCTGTCGAGGAAACCGCTGATCTGCTTGGTGTTCCCTGTTCTCCTGAGCAGTTC
GCTCCTGCTGTTGCTGCTTTTGGTGATGAGCTTCGAGAGGCCCACATCGTCTTCTC
TATGGCTGCTGGTGAGGCTCATCGAGGTGAACTGGATTTCGACTTCTCCGTCTCC
ACCAAGGGTGCTGATCCTTACGCTACTGCTCTGGCTAACGGTCTGATCAAGGGTA
CTGACCACCCTGTTGGTGCTCTGCTGACCGATATTCAGGCTCGACACGCTGTTGCC
TCTTATGGTGTTGAGTACGGCATTCTGGGCGGCTTCAAGAAGTCTTACGCCTTCTT
CCCCATCGGCGACTATCCTCCTTTGGCTGAGTTTGCCGCTATCCCCTCTGTTCCTC
CTGGTATTTCTGAGCACGTCGACACTCTTACCCGACTTGGTCTTCAGGACACCGTC
TCTGCCATTGGCGTCAACTATGCTAAGCGAACCCTGAACGTCTACCTGGGTGTTG
GTGAGGTTGCTACTGAGACCAAGCTGGAGCTTCTGCGAACCTTCGGTTTTCCTGA
GCCTGATGCTCAGGTTGCTGAGTTCGTCAAGCGATCCTTCTCCATGTACCCCACCT
TCAACTGGGATTCCTCTGTCGTCGAGCGAATCTGCTTCTCCGTCAAGACCCAGGA
TCCTGGTGAGTTACCTGCTCCTTTTCATCCCGAGATCGAGAAGTTCGCCTCTGGTG
TTCCCCACTCTTACGCTGGTGGTCGAGAGTTCGTTTCTGCTGTTGCTCTTGCTCCTT
CTGGTGAGGCTTACTACAAGCTGGCTGCCTACTACCAGAAGGCTCAGGGTGATTC
TAAGGCCGCTTTTGCCGCTTCTCGAGAGGATGATGCTGCCGGTTAG
SEQ ID NO: 91 <APT73.NTS>
ATGAAACATCACCACCACCACCATGGCACATCTGAAAACTTGTAT
TTCCAGGGCATGGACGAGGTGTATGCGGCAGTGGAACAGACTTCTCGACTTCTGG
ACGTTCCCTGTTCTCCTGACCGATTTGAGCCCGTTTGGAAGGCCTTTGGTGATCAG
CTGCCTGATTCTCACCTGGTCTTCTCTATGGCTGCTGGTGAGGCTCATCGAGGTGA
GCTGGACTTTGACTTCTCTCTTCGACCTGAGGGTGCTGATCCTTACACTACCGCTC
TGGAGCACGGCTTCATTGAGCCTACTGATCATCCTGTCGGTTCTGTCCTTGCTGAG
GTTGGTAAGCGATTCGCCATCGCCTCTTACGGTGTCGAATACGGTGTTGTCGGTG
GCTTCAAGAAGTCTTACGCCTTCTTCCCCCTGGACGACTTTCCCCCTCTTGCTCAG
TTTGCTGAGGTCCCCTCTGTTCCTCCTTGTTTAGCTGGTCACGTTGAGACCCTTAC
CCGACTGGGATTTGACGACAAGGTCTCGGCCATTGGCGTCAACTACCGAAAGAA
CACCCTGAACGTCTACCTGGCTGCTTCTGCTGTTGACACTGGCGATAAGCTGGCT
CTGCTTCGAGCTTTCGGTTACCCTGAACCTGATGCCCGAGTTCGACAGTTCATCGA
GCGATCCTTCTCCCTTTACCCCACCTTCAACTGGGATTCTTCTGCCGCTGAGCGAA
TCTGCTTCTCCGTCAAGACACAGCAACCTGGTGAGCTTCCTGCACCTCATGATGA
GCCTACTGAGGCTTTCGCTCGACAGGTTCCTCACGTTTACGAAGGTGGTCGAGAG
TTCGTCTCTGCTGTTGCTCTTGCTCCTTCTGGTGCCTCCTACTACAAGCTTGCTGCC
TACTACCAGAAGGCTAGAGGTGCCTCTAATGCCGCTTTTGCTGCCAAGCGAGAGG
ATGCTGCTGCTGGTTAG
SEQ ID NO: 92 <APT73.F116C.NTS>
ATGAAACATCACCACCACCACCATGGCACATCTGAAAACTTGTAT
TTCCAGGGCATGGACGAGGTGTATGCGGCAGTGGAACAGACTTCTCGACTTCTGG
ACGTTCCCTGTTCTCCTGACCGATTTGAGCCCGTTTGGAAGGCCTTTGGTGATCAG
CTGCCTGATTCTCACCTGGTCTTCTCTATGGCTGCTGGTGAGGCTCATCGAGGTGA
GCTGGACTTTGACTTCTCTCTTCGACCTGAGGGTGCTGATCCTTACACTACCGCTC
TGGAGCACGGCTTCATTGAGCCTACTGATCATCCTGTCGGTTCTGTCCTTGCTGAG
GTTGGTAAGCGATTCGCCATCGCCTCTTACGGTGTCGAATACGGTGTTGTCGGTG
GCTTCAAGAAGTCTTACGCCTGTTTCCCCCTGGACGACTTTCCCCCTCTTGCTCAG
TTTGCTGAGGTCCCCTCTGTTCCTCCTTGTTTAGCTGGTCACGTTGAGACCCTTAC
CCGACTGGGATTTGACGACAAGGTCTCGGCCATTGGCGTCAACTACCGAAAGAA
CACCCTGAACGTCTACCTGGCTGCTTCTGCTGTTGACACTGGCGATAAGCTGGCT
CTGCTTCGAGCTTTCGGTTACCCTGAACCTGATGCCCGAGTTCGACAGTTCATCGA
GCGATCCTTCTCCCTTTACCCCACCTTCAACTGGGATTCTTCTGCCGCTGAGCGAA
TCTGCTTCTCCGTCAAGACACAGCAACCTGGTGAGCTTCCTGCACCTCATGATGA
GCCTACTGAGGCTTTCGCTCGACAGGTTCCTCACGTTTACGAAGGTGGTCGAGAG
TTCGTCTCTGCTGTTGCTCTTGCTCCTTCTGGTGCCTCCTACTACAAGCTTGCTGCC
TACTACCAGAAGGCTAGAGGTGCCTCTAATGCCGCTTTTGCTGCCAAGCGAGAGG
ATGCTGCTGCTGGTTAG
SEQ ID NO: 93 <APT73.F116L.NTS>
ATGAAACATCACCACCACCACCATGGCACATCTGAAAACTTGTAT
TTCCAGGGCATGGACGAGGTGTATGCGGCAGTGGAACAGACTTCTCGACTTCTGG
ACGTTCCCTGTTCTCCTGACCGATTTGAGCCCGTTTGGAAGGCCTTTGGTGATCAA
CTGCCTGATTCTCACCTGGTCTTCTCTATGGCTGCTGGTGAGGCTCATCGAGGTGA
GCTGGACTTTGACTTCTCTCTTCGACCTGAGGGTGCTGATCCTTACACTACCGCTC
TGGAGCACGGCTTCATTGAGCCTACTGATCATCCTGTCGGTTCTGTCCTTGCTGAG
GTTGGTAAGCGATTCGCCATCGCCTCTTACGGTGTCGAATACGGTGTTGTCGGTG
GCTTCAAGAAGTCTTACGCCCTTTTCCCCCTGGACGACTTTCCCCCTCTTGCTCAG
TTTGCTGAGGTCCCCTCTGTTCCTCCTTGTTTAGCTGGTCACGTTGAGACCCTTAC
CCGACTGGGATTTGACGACAAGGTCTCGGCCATTGGCGTCAACTACCGAAAGAA
CACCCTGAACGTCTACCTGGCTGCTTCTGCTGTTGACACTGGCGATAAGCTGGCT
CTGCTTCGAGCTTTCGGTTACCCTGAACCTGATGCCCGAGTTCGACAGTTCATCGA
GCGATCCTTCTCCCTTTACCCCACCTTCAACTGGGATTCTTCTGCCGCTGAGCGAA
TCTGCTTCTCCGTCAAGACACAGCAACCTGGTGAGCTTCCTGCACCTCATGATGA
GCCTACTGAGGCTTTCGCTCGACAGGTTCCTCACGTTTACGAAGGTGGTCGAGAG
TTCGTCTCTGCTGTTGCTCTTGCTCCTTCTGGTGCCTCCTACTACAAGCTTGCTGCC
TACTACCAGAAGGCTAGAGGTGCCTCTAATGCCGCTTTTGCTGCCAAGCGAGAGG
ATGCTGCTGCTGGTTAG
SEQ ID NO: 94 <APT73.F116A.NTS>
ATGAAACATCACCACCACCACCATGGCACATCTGAAAACTTGTAT
TTCCAGGGCATGGACGAGGTGTATGCGGCAGTGGAACAGACTTCTCGACTTCTGG
ACGTTCCCTGTTCTCCTGACCGATTTGAGCCCGTTTGGAAGGCCTTTGGTGATCAG
CTGCCTGATTCTCACCTGGTCTTCTCTATGGCTGCTGGTGAGGCTCATCGAGGTGA
GCTGGACTTTGACTTCTCTCTTCGACCTGAGGGTGCTGATCCTTACACTACCGCTC
TGGAGCACGGCTTCATTGAGCCTACTGATCATCCTGTCGGTTCTGTCCTTGCTGAG
GTTGGTAAGCGATTCGCCATCGCCTCTTACGGTGTCGAATACGGTGTTGTCGGTG
GCTTCAAGAAGTCTTACGCCGCGTTCCCCCTGGACGACTTTCCCCCTCTTGCTCAG
TTTGCTGAGGTCCCCTCTGTTCCTCCTTGTTTAGCTGGTCACGTTGAGACCCTTAC
CCGACTGGGATTTGACGACAAGGTCTCGGCCATTGGCGTCAACTACCGAAAGAA
CACCCTGAACGTCTACCTGGCTGCTTCTGCTGTTGACACTGGCGATAAGCTGGCT
CTGCTTCGAGCTTTCGGTTACCCTGAACCTGATGCCCGAGTTCGACAGTTCATCGA
GCGATCCTTCTCCCTTTACCCCACCTTCAACTGGGATTCTTCTGCCGCTGAGCGAA
TCTGCTTCTCCGTCAAGACACAGCAACCTGGTGAGCTTCCTGCACCTCATGATGA
GCCTACTGAGGCTTTCGCTCGACAGGTTCCTCACGTTTACGAAGGTGGTCGAGAG
TTCGTCTCTGCTGTTGCTCTTGCTCCTTCTGGTGCCTCCTACTACAAGCTTGCTGCC
TACTACCAGAAGGCTAGAGGTGCCTCTAATGCCGCTTTTGCTGCCAAGCGAGAGG
ATGCTGCTGCTGGTTAG
SEQ ID NO: 95 <APT73.S155A.NTS>
ATGAAACATCACCACCACCACCATGGCACATCTGAAAACTTGTAT
TTCCAGGGCATGGACGAGGTGTATGCGGCAGTGGAACAGACTTCTCGACTTCTGG
ACGTTCCCTGTTCTCCTGACCGATTTGAGCCCGTTTGGAAGGCCTTTGGTGATCAG
CTGCCTGATTCTCACCTGGTCTTCTCTATGGCTGCTGGTGAGGCTCATCGAGGTGA
GCTGGACTTTGACTTCTCTCTTCGACCTGAGGGTGCTGATCCTTACACTACCGCTC
TGGAGCACGGCTTCATTGAGCCTACTGATCATCCTGTCGGTTCTGTCCTTGCTGAG
GTTGGTAAGCGATTCGCCATCGCCTCTTACGGTGTCGAATACGGTGTTGTCGGTG
GCTTCAAGAAGTCTTACGCCTTCTTCCCCCTGGACGACTTTCCCCCTCTTGCTCAG
TTTGCTGAGGTCCCCTCTGTTCCTCCTTGTTTAGCTGGTCACGTTGAGACCCTTAC
CCGACTGGGATTTGACGACAAGGTCGCGGCCATTGGCGTCAACTACCGAAAGAA
CACCCTGAACGTCTACCTGGCTGCTTCTGCTGTTGACACTGGCGATAAGCTGGCT
CTGCTTCGAGCTTTCGGTTACCCTGAACCTGATGCCCGAGTTCGACAGTTCATCGA
GCGATCCTTCTCCCTTTACCCCACCTTCAACTGGGATTCTTCTGCCGCTGAGCGAA
TCTGCTTCTCCGTCAAGACACAGCAACCTGGTGAGCTTCCTGCACCTCATGATGA
GCCTACTGAGGCTTTCGCTCGACAGGTTCCTCACGTTTACGAAGGTGGTCGAGAG
TTCGTCTCTGCTGTTGCTCTTGCTCCTTCTGGTGCCTCCTACTACAAGCTTGCTGCC
TACTACCAGAAGGCTAGAGGTGCCTCTAATGCCGCTTTTGCTGCCAAGCGAGAGG
ATGCTGCTGCTGGTTAG
SEQ ID NO: 96 <APT73.A260S.NTS>
ATGAAACATCACCACCACCACCATGGCACATCTGAAAACTTGTAT
TTCCAGGGCATGGACGAGGTGTATGCGGCAGTGGAACAGACTTCTCGACTTCTGG
ACGTTCCCTGTTCTCCTGACCGATTTGAGCCCGTTTGGAAGGCCTTTGGTGATCAG
CTGCCTGATTCTCACCTGGTCTTCTCTATGGCTGCTGGTGAGGCTCATCGAGGTGA
GCTGGACTTTGACTTCTCTCTTCGACCTGAGGGTGCTGATCCTTACACTACCGCTC
TGGAGCACGGCTTCATTGAGCCTACTGATCATCCTGTCGGTTCTGTCCTTGCTGAG
GTTGGTAAGCGATTCGCCATCGCCTCTTACGGTGTCGAATACGGTGTTGTCGGTG
GCTTCAAGAAGTCTTACGCCTTCTTCCCCCTGGACGACTTTCCCCCTCTTGCTCAG
TTTGCTGAGGTCCCCTCTGTTCCTCCTTGTTTAGCTGGTCACGTTGAGACCCTTAC
CCGACTGGGATTTGACGACAAGGTCTCGGCCATTGGCGTCAACTACCGAAAGAA
CACCCTGAACGTCTACCTGGCTGCTTCTGCTGTTGACACTGGCGATAAGCTGGCT
CTGCTTCGAGCTTTCGGTTACCCTGAACCTGATGCCCGAGTTCGACAGTTCATCGA
GCGATCCTTCTCCCTTTACCCCACCTTCAACTGGGATTCTTCTGCCGCTGAGCGAA
TCTGCTTCTCCGTCAAGACACAGCAACCTGGTGAGCTTCCTGCACCTCATGATGA
GCCTACTGAGGCTTTCGCTCGACAGGTTCCTCACGTTTACGAAGGTGGTCGAGAG
TTCGTCTCTAGTGTTGCTCTTGCTCCTTCTGGTGCCTCCTACTACAAGCTTGCTGCC
TACTACCAGAAGGCTAGAGGTGCCTCTAATGCCGCTTTTGCTGCCAAGCGAGAGG
ATGCTGCTGCTGGTTAG
SEQ ID NO: 97 <APT73.1.NTS>
ATGAAACATCACCACCACCACCATGGCACATCTGAAAACTTGTAT
TTCCAGGGCATGGACGAGGTGTATGCGGCAGTGGAACAGACTTCTCGACTTCTGG
ACGTTCCCTGTTCTCCTGACCGATTTGAGCCCGTTTGGAAGGCCTTTGGTGATCAG
CTGCCTGATTCTCACCTGGTCTTCTCTATGGCTGCTGGTGAGGCTCATCGAGGTGA
GCTGGACTTTGACTTCTCTCTTCGACCTGAGGGTGCTGATCCTTACACTACCGCTC
TGGAGCACGGCTTCATTGAGCCTACTGATCATCCTGTCGGTTCTGTCCTTGCTGAG
GTTGGTAAGCGATTCGCCATCGCCTCTTACGGTGTCGAATACGGTGTTGTCGGTG
GCTTCAAGAAGTCTTACGCCTTCTTCCCCCTGGACGACTTTCCCCCTCTTGCTCAG
TTTGCTGAGGTCCCCTCTGTTCCTCCTTGTTTAGCTGGTCACGTTGAGACCCTTAC
CCGACTGGGATTTGACGACAAGGTCTCGGCCATTGGCGTCAACTACCGAAAGAA
CACCCTGAACGTCTACCTGGCTGCTTCTGCTGTTGACACTGGCGATAAGCTGGCT
CTGCTTCGAGCTTTCGGTTACCCTGAACCTGATGCCCGAGTTCGACAGTTCATCGA
GCGATCCTTCTCCCTTTACCCCACCTTCAACTGGGATTCTTCTGCCGCTGAGCGAA
TCTGCTTCTCCGTCAAGACACAGCAACCTGGTGAGCTTCCTGCACCTCATGATGA
GCCTACTGAGGCTTTCGCTCGACAGGTTCCTCACGTTTACGAAGGTGGTCGAGAG
TTCGTCTCTGCTGTTGCTCTTGCTCCTTCTGGTGCCTCCTACTACAAGCTTGCTGCC
TACTACCAGAAGGCTAGAGGTGCCTCTAATGCCGCTTTTGCTGCCAAGCGAGAGG
ATGCTGCTGCTGGTTAG
SEQ ID NO: 98 <APT73.3.NTS>
ATGAAACATCACCACCACCACCATGGCACATCTGAAAACTTGTAT
TTCCAGGGCATGGACGAGGTGTATGCGGCAGTGGAACAGACTTCTCGACTTCTGG
ACGTTCCCTGTTCTCCTGACCGATTTGAGCCCGTTTGGAAGGCCTTTGGTGATCAG
CTGCCTGATTCTCACCTGGTCTTCTCTATGGCTGCTGGTGAGGCTCATCGAGGTGA
GCTGGACTTTGACTTCTCTCTTCGACCTGAGGGTGCTGATCCTTACACTACCGCTC
TGGAGCACGGCTTCATTGAGCCTACTGATCATCCTGTCGGTTCTGTCCTTGCTGAG
GTTGGTAAGCGATTCGCCATCGCCTCTTACGGTGTCGAATACGGTGTTGTCGGTG
GCTTCAAGAAGTCTTACGCCTTCTTCCCCCTGGACGACTTTCCCCCTCTTGCTCAG
TTTGCTGAGGTCCCCTCTGTTCCTCCTTGTTTAGCTGGTCACGTTGAGACCCTTAC
CCGACTGGGATTTGACGACAAGGTCTCGGCCATTGGCGTCAACTACCGAAAGAA
CACCCTGAACGTCTACCTGGCTGCTTCTGCTGTTGACACTGGCGATAAGCTGGCT
CTGCTTCGAGCTTTCGGTTACCCTGAACCTGATGCCCGAGTTCGACAGTTCATCGA
GCGATCCTTCCGTCTTTACCCCACCTTCAACTGGGATTCTTCTGCCGCTGAGCGAA
TCTGCTTCTCCGTCAAGACACAGCAACCTGGTGAGCTTCCTGCACCTCATGATGA
GCCTACTGAGGCTTTCGCTCGACAGGTTCCTCACGTTTACGAAGGTGGTCGAGAG
TTCGTCTCTGCTGTTGCTCTTGCTCCTTCTGGTGCCTCCTACTACAAGCTTGCTGCC
TACTACCAGAAGGCTAGAGGTGCCTCTAATGCCGCTTTTGCTGCCAAGCGAGAGG
ATGCTGGTTAG
SEQ ID NO: 99 <APT73.4.NTS>
ATGAAACATCACCACCACCACCATGGCACATCTGAAAACTTGTAT
TTCCAGGGCATGGACGAGGTGTATGCGGCAGTGGAACAGACTTCTCGACTTCTGG
ACGTTCCCTGTTCTCCTGACCGATTTGAGCCCGTTTGGAAGGCCTTTGGTGATCAG
CTGCCTGATTCTCACCTGGTCTTCTCTATGGCTGCTGGTGAGGCTCATCGAGGTGA
GCTGGACTTTGACTTCTCTCTTCGACCTGAGGGTGCTGATCCTTACACTACCGCTC
TGGAGCACGGCTTCATTGAGCCTACTGATCATCCTGTCGGTTCTGTCCTTGCTGAG
GTTGGTAAGCGATTCGCCATCGCCTCTTACGGTGTCGAATACGGTGTTGTCGGTG
GCTTCAAGAAGTCTTACGCCTTCTTCCCCCTGGACGACTTTCCCCCTCTTGCTCAG
TTTGCTGAGGTCCCCTCTGTTCCTCCTTGTTTAGCTGGTCACGTTGAGACCCTTAC
CCGACTGGGATTTGACGACAAGGTCTCGGCCATTGGCGTCAACTACCGAAAGAA
CACCCTGAACGTCTACCTGGCTGCTTCTGCTGTTGACACTGGCGATAAGCTGGCT
CTGCTTCGAGCTTTCGGTTACCCTGAACCTGATGCCCGAGTTCGACAGTTCATCGA
GCGATCCTTCCGTCTTTACCCCACCTTCAACTGGGATTCTTCTGCCGCTGAGCGAA
TCTGCTTCTCCGTCAAGACACAGCAACCTGGTGAGCTTCCTGCACCTCATGATGA
GCCTACTGAGGCTTTCGCTCGACAGGTTCCTCACGTTTACGAAGGTGGTCGAGAG
TTCGTCTCTGCTGTTGCTCTTGCTCCTTCTGGTGCCTCCTACTACAAGCTTGCTGCC
TACTACCAGAAGGCTAGAGGTGCCTCTAATGCCGCTTTTGCTGCCAAGCGAGAGG
GTTAG
SEQ ID NO: 100 <APT73.6.NTS>
ATGAAACATCACCACCACCACCATGGCACATCTGAAAACTTGTAT
TTCCAGGGCATGGACGAGGTGTATGCGGCAGTGGAACAGACTTCTCGACTTCTGG
ACGTTCCCTGTTCTCCTGACCGATTTGAGCCCGTTTGGAAGGCCTTTGGTGATCAG
CTGCCTGATTCTCACCTGGTCTTCTCTATGGCTGCTGGTGAGGCTCATCGAGGTGA
GCTGGACTTTGACTTCTCTCTTCGACCTGAGGGTGCTGATCCTTACACTACCGCTC
TGGAGCACGGCTTCATTGAGCCTACTGATCATCCTGTCGGTTCTGTCCTTGCTGAG
GTTGGTAAGCGATTCGCCATCGCCTCTTACGGTGTCGAATACGGTGTTGTCGGTG
GCTTCAAGAAGTCTTACGCCTTCTTCCCCCTGGACGACTTTCCCCCTCTTGCTCAG
TTTGCTGAGGTCCCCTCTGTTCCTCCTTGTTTAGCTGGTCACGTTGAGACCCTTAC
CCGACTGGGATTTGACGACAAGGTCTCGGCCATTGGCGTCAACTACCGAAAGAA
CACCCTGAACGTCTACCTGGCTGCTTCTGCTGTTGACACTGGCGATAAGCTGGCT
CTGCTTCGAGCTTTCGGTTACCCTGAACCTGATGCCCGAGTTCGACAGTTCATCGA
GCGATCCTTCCGTCTTTACCCCACCTTCAACTGGGATTCTTCTGCCGCTGAGCGAA
TCTGCTTCTCCGTCAAGACACAGCAACCTGGTGAGCTTCCTGCACCTCATGATGA
GCCTACTGAGGCTTTCGCTCGACAGGTTCCTCACGTTTACGAAGGTGGTCGAGAG
TTCGTCTCTGCTGTTGCTCTTGCTCCTTCTGGTGCCTCCTACTACAAGCTTGCTGCC
TACTACCAGAAGGCTAGAGGTGCCTCTAATGCCGCTTTTGCTGGTTAG
SEQ ID NO: 101 <APT73.7.NTS>
ATGAAACATCACCACCACCACCATGGCACATCTGAAAACTTGTAT
TTCCAGGGCATGGACGAGGTGTATGCGGCAGTGGAACAGACTTCTCGACTTCTGG
ACGTTCCCTGTTCTCCTGACCGATTTGAGCCCGTTTGGAAGGCCTTTGGTGATCAG
CTGCCTGATTCTCACCTGGTCTTCTCTATGGCTGCTGGTGAGGCTCATCGAGGTGA
GCTGGACTTTGACTTCTCTCTTCGACCTGAGGGTGCTGATCCTTACACTACCGCTC
TGGAGCACGGCTTCATTGAGCCTACTGATCATCCTGTCGGTTCTGTCCTTGCTGAG
GTTGGTAAGCGATTCGCCATCGCCTCTTACGGTGTCGAATACGGTGTTGTCGGTG
GCTTCAAGAAGTCTTACGCCTTCTTCCCCCTGGACGACTTTCCCCCTCTTGCTCAG
TTTGCTGAGGTCCCCTCTGTTCCTCCTTGTTTAGCTGGTCACGTTGAGACCCTTAC
CCGACTGGGATTTGACGACAAGGTCTCGGCCATTGGCGTCAACTACCGAAAGAA
CACCCTGAACGTCTACCTGGCTGCTTCTGCTGTTGACACTGGCGATAAGCTGGCT
CTGCTTCGAGCTTTCGGTTACCCTGAACCTGATGCCCGAGTTCGACAGTTCATCGA
GCGATCCTTCCGTCTTTACCCCACCTTCAACTGGGATTCTTCTGCCGCTGAGCGAA
TCTGCTTCTCCGTCAAGACACAGCAACCTGGTGAGCTTCCTGCACCTCATGATGA
GCCTACTGAGGCTTTCGCTCGACAGGTTCCTCACGTTTACGAAGGTGGTCGAGAG
TTCGTCTCTGCTGTTGCTCTTGCTCCTTCTGGTGCCTCCTACTACAAGCTTGCTGCC
TACTACCAGAAGGCTAGAGGTGCCTCTAATGCCGCTGGTTAG
SEQ ID NO: 102 <APT73.8.NTS>
ATGAAACATCACCACCACCACCATGGCACATCTGAAAACTTGTAT
TTCCAGGGCATGGACGAGGTGTATGCGGCAGTGGAACAGACTTCTCGACTTCTGG
ACGTTCCCTGTTCTCCTGACCGATTTGAGCCCGTTTGGAAGGCCTTTGGTGATCAG
CTGCCTGATTCTCACCTGGTCTTCTCTATGGCTGCTGGTGAGGCTCATCGAGGTGA
GCTGGACTTTGACTTCTCTCTTCGACCTGAGGGTGCTGATCCTTACACTACCGCTC
TGGAGCACGGCTTCATTGAGCCTACTGATCATCCTGTCGGTTCTGTCCTTGCTGAG
GTTGGTAAGCGATTCGCCATCGCCTCTTACGGTGTCGAATACGGTGTTGTCGGTG
GCTTCAAGAAGTCTTACGCCTTCTTCCCCCTGGACGACTTTCCCCCTCTTGCTCAG
TTTGCTGAGGTCCCCTCTGTTCCTCCTTGTTTAGCTGGTCACGTTGAGACCCTTAC
CCGACTGGGATTTGACGACAAGGTCTCGGCCATTGGCGTCAACTACCGAAAGAA
CACCCTGAACGTCTACCTGGCTGCTTCTGCTGTTGACACTGGCGATAAGCTGGCT
CTGCTTCGAGCTTTCGGTTACCCTGAACCTGATGCCCGAGTTCGACAGTTCATCGA
GCGATCCTTCCGTCTTTACCCCACCTTCAACTGGGATTCTTCTGCCGCTGAGCGAA
TCTGCTTCTCCGTCAAGACACAGCAACCTGGTGAGCTTCCTGCACCTCATGATGA
GCCTACTGAGGCTTTCGCTCGACAGGTTCCTCACGTTTACGAAGGTGGTCGAGAG
TTCGTCTCTGCTGTTGCTCTTGCTCCTTCTGGTGCCTCCTACTACAAGCTTGCTGCC
TACTACCAGAAGGCTAGAGGTGCCTCTAATGGTTAG
SEQ ID NO: 103 <APT73.9.NTS>
ATGAAACATCACCACCACCACCATGGCACATCTGAAAACTTGTAT
TTCCAGGGCATGGACGAGGTGTATGCGGCAGTGGAACAGACTTCTCGACTTCTGG
ACGTTCCCTGTTCTCCTGACCGATTTGAGCCCGTTTGGAAGGCCTTTGGTGATCAG
CTGCCTGATTCTCACCTGGTCTTCTCTATGGCTGCTGGTGAGGCTCATCGAGGTGA
GCTGGACTTTGACTTCTCTCTTCGACCTGAGGGTGCTGATCCTTACACTACCGCTC
TGGAGCACGGCTTCATTGAGCCTACTGATCATCCTGTCGGTTCTGTCCTTGCTGAG
GTTGGTAAGCGATTCGCCATCGCCTCTTACGGTGTCGAATACGGTGTTGTCGGTG
GCTTCAAGAAGTCTTACGCCTTCTTCCCCCTGGACGACTTTCCCCCTCTTGCTCAG
TTTGCTGAGGTCCCCTCTGTTCCTCCTTGTTTAGCTGGTCACGTTGAGACCCTTAC
CCGACTGGGATTTGACGACAAGGTCTCGGCCATTGGCGTCAACTACCGAAAGAA
CACCCTGAACGTCTACCTGGCTGCTTCTGCTGTTGACACTGGCGATAAGCTGGCT
CTGCTTCGAGCTTTCGGTTACCCTGAACCTGATGCCCGAGTTCGACAGTTCATCGA
GCGATCCTTCCGTCTTTACCCCACCTTCAACTGGGATTCTTCTGCCGCTGAGCGAA
TCTGCTTCTCCGTCAAGACACAGCAACCTGGTGAGCTTCCTGCACCTCATGATGA
GCCTACTGAGGCTTTCGCTCGACAGGTTCCTCACGTTTACGAAGGTGGTCGAGAG
TTCGTCTCTGCTGTTGCTCTTGCTCCTTCTGGTGCCTCCTACTACAAGCTTGCTGCC
TACTACCAGAAGGCTAGAGGTGCCGGTTAG
SEQ ID NO: 104 <APT73.10.NTS>
ATGAAACATCACCACCACCACCATGGCACATCTGAAAACTTGTAT
TTCCAGGGCATGGACGAGGTGTATGCGGCAGTGGAACAGACTTCTCGACTTCTGG
ACGTTCCCTGTTCTCCTGACCGATTTGAGCCCGTTTGGAAGGCCTTTGGTGATCAG
CTGCCTGATTCTCACCTGGTCTTCTCTATGGCTGCTGGTGAGGCTCATCGAGGTGA
GCTGGACTTTGACTTCTCTCTTCGACCTGAGGGTGCTGATCCTTACACTACCGCTC
TGGAGCACGGCTTCATTGAGCCTACTGATCATCCTGTCGGTTCTGTCCTTGCTGAG
GTTGGTAAGCGATTCGCCATCGCCTCTTACGGTGTCGAATACGGTGTTGTCGGTG
GCTTCAAGAAGTCTTACGCCTTCTTCCCCCTGGACGACTTTCCCCCTCTTGCTCAG
TTTGCTGAGGTCCCCTCTGTTCCTCCTTGTTTAGCTGGTCACGTTGAGACCCTTAC
CCGACTGGGATTTGACGACAAGGTCTCGGCCATTGGCGTCAACTACCGAAAGAA
CACCCTGAACGTCTACCTGGCTGCTTCTGCTGTTGACACTGGCGATAAGCTGGCT
CTGCTTCGAGCTTTCGGTTACCCTGAACCTGATGCCCGAGTTCGACAGTTCATCGA
GCGATCCTTCCGTCTTTACCCCACCTTCAACTGGGATTCTTCTGCCGCTGAGCGAA
TCTGCTTCTCCGTCAAGACACAGCAACCTGGTGAGCTTCCTGCACCTCATGATGA
GCCTACTGAGGCTTTCGCTCGACAGGTTCCTCACGTTTACGAAGGTGGTCGAGAG
TTCGTCTCTGCTGTTGCTCTTGCTCCTTCTGGTGCCTCCTACTACAAGCTTGCTGCC
TACTACCAGAAGGCTAGAGGTTAG
SEQ ID NO: 105 <APT73.13.NTS>
ATGAAACATCACCACCACCACCATGGCACATCTGAAAACTTGTAT
TTCCAGGGCATGGACGAGGTGTATGCGGCAGTGGAACAGACTTCTCGACTTCTGG
ACGTTCCCTGTTCTCCTGACCGATTTGAGCCCGTTTGGAAGGCCTTTGGTGATCAG
CTGCCTGATTCTCACCTGGTCTTCTCTATGGCTGCTGGTGAGGCTCATCGAGGTGA
GCTGGACTTTGACTTCTCTCTTCGACCTGAGGGTGCTGATCCTTACACTACCGCTC
TGGAGCACGGCTTCATTGAGCCTACTGATCATCCTGTCGGTTCTGTCCTTGCTGAG
GTTGGTAAGCGATTCGCCATCGCCTCTTACGGTGTCGAATACGGTGTTGTCGGTG
GCTTCAAGAAGTCTTACGCCTTCTTCCCCCTGGACGACTTTCCCCCTCTTGCTCAG
TTTGCTGAGGTCCCCTCTGTTCCTCCTTGTTTAGCTGGTCACGTTGAGACCCTTAC
CCGACTGGGATTTGACGACAAGGTCTCGGCCATTGGCGTCAACTACCGAAAGAA
CACCCTGAACGTCTACCTGGCTGCTTCTGCTGTTGACACTGGCGATAAGCTGGCT
CTGCTTCGAGCTTTCGGTTACCCTGAACCTGATGCCCGAGTTCGACAGTTCATCGA
GCGATCCTTCCGTCTTTACCCCACCTTCAACTGGGATTCTTCTGCCGCTGAGCGAA
TCTGCTTCTCCGTCAAGACACAGCAACCTGGTGAGCTTCCTGCACCTCATGATGA
GCCTACTGAGGCTTTCGCTCGACAGGTTCCTCACGTTTACGAAGGTGGTCGAGAG
TTCGTCTCTGCTGTTGCTCTTGCTCCTTCTGGTGCCTCCTACTACAAGCTTGCTGCC
TACTACCAGAAGGCTAGAAGGTGCCTCGGTTAG
SEQ ID NO: 106 <APT73.13.Y276V.NTS>
ATGAAACATCACCACCACCACCATGGCACATCTGAAAACTTGTAT
TTCCAGGGCATGGACGAGGTGTATGCGGCAGTGGAACAGACTTCTCGACTTCTGG
ACGTTCCCTGTTCTCCTGACCGATTTGAGCCCGTTTGGAAGGCCTTTGGTGATCAG
CTGCCTGATTCTCACCTGGTCTTCTCTATGGCTGCTGGTGAGGCTCATCGAGGTGA
GCTGGACTTTGACTTCTCTCTTCGACCTGAGGGTGCTGATCCTTACACTACCGCTC
TGGAGCACGGCTTCATTGAGCCTACTGATCATCCTGTCGGTTCTGTCCTTGCTGAG
GTTGGTAAGCGATTCGCCATCGCCTCTTACGGTGTCGAATACGGTGTTGTCGGTG
GCTTCAAGAAGTCTTACGCCTTCTTCCCCCTGGACGACTTTCCCCCTCTTGCTCAG
TTTGCTGAGGTCCCCTCTGTTCCTCCTTGTTTAGCTGGTCACGTTGAGACCCTTAC
CCGACTGGGATTTGACGACAAGGTCTCGGCCATTGGCGTCAACTACCGAAAGAA
CACCCTGAACGTCTACCTGGCTGCTTCTGCTGTTGACACTGGCGATAAGCTGGCT
CTGCTTCGAGCTTTCGGTTACCCTGAACCTGATGCCCGAGTTCGACAGTTCATCGA
GCGATCCTTCCGTCTTTACCCCACCTTCAACTGGGATTCTTCTGCCGCTGAGCGAA
TCTGCTTCTCCGTCAAGACACAGCAACCTGGTGAGCTTCCTGCACCTCATGATGA
GCCTACTGAGGCTTTCGCTCGACAGGTTCCTCACGTTTACGAAGGTGGTCGAGAG
TTCGTCTCTGCTGTTGCTCTTGCTCCTTCTGGTGCCTCCTACTACAAGCTTGCTGCC
GTGTACCAGAAGGCTAGAAGGTGCCTCGGTTAG
SEQ ID NO: 107 <APT73.13.C283A.NTS>
ATGAAACATCACCACCACCACCATGGCACATCTGAAAACTTGTAT
TTCCAGGGCATGGACGAGGTGTATGCGGCAGTGGAACAGACTTCTCGACTTCTGG
ACGTTCCCTGTTCTCCTGACCGATTTGAGCCCGTTTGGAAGGCCTTTGGTGATCAG
CTGCCTGATTCTCACCTGGTCTTCTCTATGGCTGCTGGTGAGGCTCATCGAGGTGA
GCTGGACTTTGACTTCTCTCTTCGACCTGAGGGTGCTGATCCTTACACTACCGCTC
TGGAGCACGGCTTCATTGAGCCTACTGATCATCCTGTCGGTTCTGTCCTTGCTGAG
GTTGGTAAGCGATTCGCCATCGCCTCTTACGGTGTCGAATACGGTGTTGTCGGTG
GCTTCAAGAAGTCTTACGCCTTCTTCCCCCTGGACGACTTTCCCCCTCTTGCTCAG
TTTGCTGAGGTCCCCTCTGTTCCTCCTTGTTTAGCTGGTCACGTTGAGACCCTTAC
CCGACTGGGATTTGACGACAAGGTCTCGGCCATTGGCGTCAACTACCGAAAGAA
CACCCTGAACGTCTACCTGGCTGCTTCTGCTGTTGACACTGGCGATAAGCTGGCT
CTGCTTCGAGCTTTCGGTTACCCTGAACCTGATGCCCGAGTTCGACAGTTCATCGA
GCGATCCTTCCGTCTTTACCCCACCTTCAACTGGGATTCTTCTGCCGCTGAGCGAA
TCTGCTTCTCCGTCAAGACACAGCAACCTGGTGAGCTTCCTGCACCTCATGATGA
GCCTACTGAGGCTTTCGCTCGACAGGTTCCTCACGTTTACGAAGGTGGTCGAGAG
TTCGTCTCTGCTGTTGCTCTTGCTCCTTCTGGTGCCTCCTACTACAAGCTTGCTGCC
TACTACCAGAAGGCTAGAAGGGCGCTCGGTTAG
SEQ ID NO: 108 <APT73.13.C283G.NTS>
ATGAAACATCACCACCACCACCATGGCACATCTGAAAACTTGTAT
TTCCAGGGCATGGACGAGGTGTATGCGGCAGTGGAACAGACTTCTCGACTTCTGG
ACGTTCCCTGTTCTCCTGACCGATTTGAGCCCGTTTGGAAGGCCTTTGGTGATCAG
CTGCCTGATTCTCACCTGGTCTTCTCTATGGCTGCTGGTGAGGCTCATCGAGGTGA
GCTGGACTTTGACTTCTCTCTTCGACCTGAGGGTGCTGATCCTTACACTACCGCTC
TGGAGCACGGCTTCATTGAGCCTACTGATCATCCTGTCGGTTCTGTCCTTGCTGAG
GTTGGTAAGCGATTCGCCATCGCCTCTTACGGTGTCGAATACGGTGTTGTCGGTG
GCTTCAAGAAGTCTTACGCCTTCTTCCCCCTGGACGACTTTCCCCCTCTTGCTCAG
TTTGCTGAGGTCCCCTCTGTTCCTCCTTGTTTAGCTGGTCACGTTGAGACCCTTAC
CCGACTGGGATTTGACGACAAGGTCTCGGCCATTGGCGTCAACTACCGAAAGAA
CACCCTGAACGTCTACCTGGCTGCTTCTGCTGTTGACACTGGCGATAAGCTGGCT
CTGCTTCGAGCTTTCGGTTACCCTGAACCTGATGCCCGAGTTCGACAGTTCATCGA
GCGATCCTTCCGTCTTTACCCCACCTTCAACTGGGATTCTTCTGCCGCTGAGCGAA
TCTGCTTCTCCGTCAAGACACAGCAACCTGGTGAGCTTCCTGCACCTCATGATGA
GCCTACTGAGGCTTTCGCTCGACAGGTTCCTCACGTTTACGAAGGTGGTCGAGAG
TTCGTCTCTGCTGTTGCTCTTGCTCCTTCTGGTGCCTCCTACTACAAGCTTGCTGCC
TACTACCAGAAGGCTAGAAGGGGTCTCGGTTAG
SEQ ID NO: 109 <APT73.14.NTS>
ATGAAACATCACCACCACCACCATGGCACATCTGAAAACTTGTAT
TTCCAGGGCATGGACGAGGTGTATGCGGCAGTGGAACAGACTTCTCGACTTCTGG
ACGTTCCCTGTTCTCCTGACCGATTTGAGCCCGTTTGGAAGGCCTTTGGTGATCAG
CTGCCTGATTCTCACCTGGTCTTCTCTATGGCTGCTGGTGAGGCTCATCGAGGTGA
GCTGGACTTTGACTTCTCTCTTCGACCTGAGGGTGCTGATCCTTACACTACCGCTC
TGGAGCACGGCTTCATTGAGCCTACTGATCATCCTGTCGGTTCTGTCCTTGCTGAG
GTTGGTAAGCGATTCGCCATCGCCTCTTACGGTGTCGAATACGGTGTTGTCGGTG
GCTTCAAGAAGTCTTACGCCTTCTTCCCCCTGGACGACTTTCCCCCTCTTGCTCAG
TTTGCTGAGGTCCCCTCTGTTCCTCCTTGTTTAGCTGGTCACGTTGAGACCCTTAC
CCGACTGGGATTTGACGACAAGGTCTCGGCCATTGGCGTCAACTACCGAAAGAA
CACCCTGAACGTCTACCTGGCTGCTTCTGCTGTTGACACTGGCGATAAGCTGGCT
CTGCTTCGAGCTTTCGGTTACCCTGAACCTGATGCCCGAGTTCGACAGTTCATCGA
GCGATCCTTCCGTCTTTACCCCACCTTCAACTGGGATTCTTCTGCCGCTGAGCGAA
TCTGCTTCTCCGTCTTTACACAGCAACCTGGTGAGCTTCCTGCACCTCATGATGAG
CCTACTGAGGCTTTCGCTCGACAGGTTCCTCACGTTTACGAAGGTGGTCGAGAGT
TCGTCTCTGCTGTTGCTCTTGCTCCTTCTGGTGCCTCCTACTACAAGCTTGCTGCCT
ACTACCAGAAGGCTAGAAGGTGCCTCGGTTAG
SEQ ID NO: 110 <APT73.15.NTS>
ATGAAACATCACCACCACCACCATGGCACATCTGAAAACTTGTAT
TTCCAGGGCATGGACGAGGTGTATGCGGCAGTGGAACAGACTTCTCGACTTCTGG
ACGTTCCCTGTTCTCCTGACCGATTTGAGCCCGTTTGGAAGGCCTTTGGTGATCAG
CTGCCTGATTCTCACCTGGTCTTCTCTATGGCTGCTGGTGAGGCTCATCGAGGTGA
GCTGGACTTTGACTTCTCTCTTCGACCTGAGGGTGCTGATCCTTACACTACCGCTC
TGGAGCACGGCTTCATTGAGCCTACTGATCATCCTGTCGGTTCTGTCCTTGCTGAG
GTTGGTAAGCGATTCGCCATCGCCTCTTACGGTGTCGAATACGGTGTTGTCGGTG
GCTTCAAGAAGTCTTACGCCTTCTTCCCCCTGGACGACTTTCCCCCTCTTGCTCAG
TTTGCTGAGGTCCCCTCTGTTCCTCCTTGTTTAGCTGGTCACGTTGAGACCCTTAC
CCGACTGGGATTTGACGACAAGGTCTCGGCCATTGGCGTCAACTACCGAAAGAA
CACCCTGAACGTCTACCTGGCTGCTTCTGCTGTTGACACTGGCGATAAGCTGGCT
CTGCTTCGAGCTTTCGGTTACCCTGAACCTGATGCCCGAGTTCGACAGTTCATCGA
GCGATCCTTCCGTCTTTACCCCACCTTCAACTGGGATTCTTCTGCCGCTGAGCGAA
TCTGCTTCGCGGTCAAGACACAGCAACCTGGTGAGCTTCCTGCACCTCATGATGA
GCCTACTGAGGCTTTCGCTCGACAGGTTCCTCACGTTTACGAAGGTGGTCGAGAG
TTCGTCTCTGCTGTTGCTCTTGCTCCTTCTGGTGCCTCCTACTACAAGCTTGCTGCC
TACTACCAGAAGGCTAGAAGGTGCCTCGGTTAG
SEQ ID NO: 111 <APT73.16.NTS>
ATGAAACATCACCACCACCACCATGGCACATCTGAAAACTTGTAT
TTCCAGGGCATGGACGAGGTGTATGCGGCAGTGGAACAGACTTCTCGACTTCTGG
ACGTTCCCTGTTCTCCTGACCGATTTGAGCCCGTTTGGAAGGCCTTTGGTGATCAG
CTGCCTGATTCTCACCTGGTCTTCTCTATGGCTGCTGGTGAGGCTCATCGAGGTGA
GCTGGACTTTGACTTCTCTCTTCGACCTGAGGGTGCTGATCCTTACACTACCGCTC
TGGAGCACGGCTTCATTGAGCCTACTGATCATCCTGTCGGTTCTGTCCTTGCTGAG
GTTGGTAAGCGATTCGCCATCGCCTCTTACGGTGTCGAATACGGTGTTGTCGGTG
GCTTCAAGAAGTCTTACGCCTTCTTCCCCCTGGACGACTTTCCCCCTCTTGCTCAG
TTTGCTGAGGTCCCCTCTGTTCCTCCTTGTTTAGCTGGTCACGTTGAGACCCTTAC
CCGACTGGGATTTGACGACAAGGTCTCGGCCATTGGCGTCAACTACCGAAAGAA
CACCCTGAACGTCTACCTGGCTGCTTCTGCTGTTGACACTGGCGATAAGCTGGCT
CTGCTTCGAGCTTTCGGTTACCCTGAACCTGATGCCCGAGTTCGACAGTTCATCGA
GCGATCCTTCCGTCTTTACCCCACCTTCAACTGGGATTCTTCTGCCGCTGAGCGAA
TCTGCTTCTCCGTCAAGACACAGCAACCTGGTGAGCTTCCTGCACCTCATGATGA
GCCTACTGAGGCTTTCGCTCGACAGGTTCCTCACGTTTACGAAGGTGGTCGAGAG
TTCGTCTCTGGTGTTGCTCTTGCTCCTTCTGGTGCCTCCTACTACAAGCTTGCTGCC
TACTACCAGAAGGCTAGAAGGTGCCTCGGTTAG
SEQ ID NO: 112 <APT73.17.NTS>
ATGAAACATCACCACCACCACCATGGCACATCTGAAAACTTGTAT
TTCCAGGGCATGGACGAGGTGTATGCGGCAGTGGAACAGACTTCTCGACTTCTGG
ACGTTCCCTGTTCTCCTGACCGATTTGAGCCCGTTTGGAAGGCCTTTGGTGATCAG
CTGCCTGATTCTCACCTGGTCTTCTCTATGGCTGCTGGTGAGGCTCATCGAGGTGA
GCTGGACTTTGACTTCTCTCTTCGACCTGAGGGTGCTGATCCTTACACTACCGCTC
TGGAGCACGGCTTCATTGAGCCTACTGATCATCCTGTCGGTTCTGTCCTTGCTGAG
GTTGGTAAGCGATTCGCCATCGCCTCTTACGGTGTCGAATACGGTGTTGTCGGTG
GCTTCAAGAAGTCTTACGCCTTCTTCCCCCTGGACGACTTTCCCCCTCTTGCTCAG
TTTGCTGAGGTCCCCTCTGTTCCTCCTTGTTTAGCTGGTCACGTTGAGACCCTTAC
CCGACTGGGATTTGACGACAAGGTCTCGGCCATTGGCGTCAACTACCGAAAGAA
CACCCTGAACGTCTACCTGGCTGCTTCTGCTGTTGACACTGGCGATAAGCTGGCT
CTGCTTCGAGCTTTCGGTTACCCTGAACCTGATGCCCGAGTTCGACAGTTCATCGA
GCGATCCTTCCGTCTTTACCCCACCTTCAACTGGGATTCTTCTGCCGCTGAGCGAA
TCTGCTTCTCCGTCAAGACACAGCAACCTGGTGAGCTTCCTGCACCTCATGATGA
GCCTACTGAGGCTTTCGCTCGACAGGTTCCTCACGTTTACGAAGGTGGTCGAGAG
TTCGTCTCTGCTGTTGCTCTTGCTCCTTCTGGTGCCTCCTACTACAAGCTTGCTGCC
CTTTACCAGAAGGCTAGAAGGTGCCTCGGTTAG
SEQ ID NO: 113 <APT73.18.NTS>
ATGAAACATCACCACCACCACCATGGCACATCTGAAAACTTGTAT
TTCCAGGGCATGGACGAGGTGTATGCGGCAGTGGAACAGACTTCTCGACTTCTGG
ACGTTCCCTGTTCTCCTGACCGATTTGAGCCCGTTTGGAAGGCCTTTGGTGATCAG
CTGCCTGATTCTCACCTGGTCTTCTCTATGGCTGCTGGTGAGGCTCATCGAGGTGA
GCTGGACTTTGACTTCTCTCTTCGACCTGAGGGTGCTGATCCTTACACTACCGCTC
TGGAGCACGGCTTCATTGAGCCTACTGATCATCCTGTCGGTTCTGTCCTTGCTGAG
GTTGGTAAGCGATTCGCCATCGCCTCTTACGGTGTCGAATACGGTGTTGTCGGTG
GCTTCAAGAAGTCTTACGCCTTCTTCCCCCTGGACGACTTTCCCCCTCTTGCTCAG
TTTGCTGAGGTCCCCTCTGTTCCTCCTTGTTTAGCTGGTCACGTTGAGACCCTTAC
CCGACTGGGATTTGACGACAAGGTCTCGGCCATTGGCGTCAACTACCGAAAGAA
CACCCTGAACGTCTACCTGGCTGCTTCTGCTGTTGACACTGGCGATAAGCTGGCT
CTGCTTCGAGCTTTCGGTTACCCTGAACCTGATGCCCGAGTTCGACAGTTCATCGA
GCGATCCTTCCGTCTTTACCCCACCTTCAACTGGGATTCTTCTGCCGCTGAGCGAA
TCTGCTTCTCCGTCAAGACACAGCAACCTGGTGAGCTTCCTGCACCTCATGATGA
GCCTACTGAGGCTTTCGCTCGACAGGTTCCTCACGTTTACGAAGGTGGTCGAGAG
TTCGTCTCTGCTGTTGCTCTTGCTCCTTCTGGTGCCTCCTACTACAAGCTTGCTGCC
TGTTACCAGAAGGCTAGAAGGTGCCTCGGTTAG
SEQ ID NO: 114 <APT73.19.NTS>
ATGAAACATCACCACCACCACCATGGCACATCTGAAAACTTGTAT
TTCCAGGGCATGGACGAGGTGTATGCGGCAGTGGAACAGACTTCTCGACTTCTGG
ACGTTCCCTGTTCTCCTGACCGATTTGAGCCCGTTTGGAAGGCCTTTGGTGATCAG
CTGCCTGATTCTCACCTGGTCTTCTCTATGGCTGCTGGTGAGGCTCATCGAGGTGA
GCTGGACTTTGACTTCTCTCTTCGACCTGAGGGTGCTGATCCTTACACTACCGCTC
TGGAGCACGGCTTCATTGAGCCTACTGATCATCCTGTCGGTTCTGTCCTTGCTGAG
GTTGGTAAGCGATTCGCCATCGCCTCTTACGGTGTCGAATACGGTGTTGTCGGTG
GCTTCAAGAAGTCTTACGCCTTCTTCCCCCTGGACGACTTTCCCCCTCTTGCTCAG
TTTGCTGAGGTCCCCTCTGTTCCTCCTTGTTTAGCTGGTCACGTTGAGACCCTTAC
CCGACTGGGATTTGACGACAAGGTCTCGGCCATTGGCGTCAACTACCGAAAGAA
CACCCTGAACGTCTACCTGGCTGCTTCTGCTGTTGACACTGGCGATAAGCTGGCT
CTGCTTCGAGCTTTCGGTTACCCTGAACCTGATGCCCGAGTTCGACAGTTCATCGA
GCGATCCTTCCGTCTTTACCCCACCTTCAACTGGGATTCTTCTGCCGCTGAGCGAA
TCTGCTTCTCCGTCAAGACACAGCAACCTGGTGAGCTTCCTGCACCTCATGATGA
GCCTACTGAGGCTTTCGCTCGACAGGTTCCTCACGTTTACGAAGGTGGTCGAGAG
TTCGTCTCTGCTGTTGCTCTTGCTCCTTCTGGTGCCTCCTACTACAAGCTTGCTGCC
AGTTACCAGAAGGCTAGAAGGTGCCTCGGTTAG
SEQ ID NO: 115 <APT73.20.NTS>
ATGAAACATCACCACCACCACCATGGCACATCTGAAAACTTGTAT
TTCCAGGGCATGGACGAGGTGTATGCGGCAGTGGAACAGACTTCTCGACTTCTGG
ACGTTCCCTGTTCTCCTGACCGATTTGAGCCCGTTTGGAAGGCCTTTGGTGATCAG
CTGCCTGATTCTCACCTGGTCTTCTCTATGGCTGCTGGTGAGGCTCATCGAGGTGA
GCTGGACTTTGACTTCTCTCTTCGACCTGAGGGTGCTGATCCTTACACTACCGCTC
TGGAGCACGGCTTCATTGAGCCTACTGATCATCCTGTCGGTTCTGTCCTTGCTGAG
GTTGGTAAGCGATTCGCCATCGCCTCTTACGGTGTCGAATACGGTGTTGTCGGTG
GCTTCAAGAAGTCTTACGCCTTCTTCCCCCTGGACGACTTTCCCCCTCTTGCTCAG
TTTGCTGAGGTCCCCTCTGTTCCTCCTTGTTTAGCTGGTCACGTTGAGACCCTTAC
CCGACTGGGATTTGACGACAAGGTCTCGGCCATTGGCGTCAACTACCGAAAGAA
CACCCTGAACGTCTACCTGGCTGCTTCTGCTGTTGACACTGGCGATAAGCTGGCT
CTGCTTCGAGCTTTCGGTTACCCTGAACCTGATGCCCGAGTTCGACAGTTCATCGA
GCGATCCTTCCGTCTTTACCCCACCTTCAACTGGGATTCTTCTGCCGCTGAGCGAA
TCTGCTTCTCCGTCAAGACACAGCAACCTGGTGAGCTTCCTGCACCTCATGATGA
GCCTACTGAGGCTTTCGCTCGACAGGTTCCTCACGTTTACGAAGGTGGTCGAGAG
TTCGTCTCTGCTGTTGCTCTTGCTCCTTCTGGTGCCTCCTACTACAAGCTTGCTGCC
TACTACCAGAAGGCTAGAAGGCCGCTCGGTTAG
SEQ ID NO: 116 <APT73.21.NTS>
ATGAAACATCACCACCACCACCATGGCACATCTGAAAACTTGTAT
TTCCAGGGCATGGACGAGGTGTATGCGGCAGTGGAACAGACTTCTCGACTTCTGG
ACGTTCCCTGTTCTCCTGACCGATTTGAGCCCGTTTGGAAGGCCTTTGGTGATCAG
CTGCCTGATTCTCACCTGGTCTTCTCTATGGCTGCTGGTGAGGCTCATCGAGGTGA
GCTGGACTTTGACTTCTCTCTTCGACCTGAGGGTGCTGATCCTTACACTACCGCTC
TGGAGCACGGCTTCATTGAGCCTACTGATCATCCTGTCGGTTCTGTCCTTGCTGAG
GTTGGTAAGCGATTCGCCATCGCCTCTTACGGTGTCGAATACGGTGTTGTCGGTG
GCTTCAAGAAGTCTTACGCCTTCTTCCCCCTGGACGACTTTCCCCCTCTTGCTCAG
TTTGCTGAGGTCCCCTCTGTTCCTCCTTGTTTAGCTGGTCACGTTGAGACCCTTAC
CCGACTGGGATTTGACGACAAGGTCTCCTGCATCGGCGTCAACTACCGAAAGAAC
ACCCTGAACGTCTACCTGGCTGCTTCTGCTGTTGATACCGGCGATAAGCTGGCTCT
GCTTCGAGCTTTCGGTTACCCTGAACCTGATGCCCGAGTTCGACAGTTCATCGAG
CGATCTTTCCGGCTTTACCCCACCTTCAACTGGGATTCTTCTGCTGCCGAGCGAAT
CTGCTTCTCCGTCTTTACTCAGCAGCCTGGTGAGCTTCCTGCACCTCATGATGAAC
CTACCGAGGCTTTCGCTCGACAGGTTCCTCATGTTTACGAGGGTGGTCGAGAGTT
CGTCTCTGCTGTTGCTCTTGCTCCTTCTGGTGCCTCCTACTACAAGTTAGCTGCCT
GCTACCAGAAGGCTCGACGATGTCTGGGTTAG
SEQ ID NO: 117 <APT73.22.NTS>
ATGAAACATCACCACCACCACCATGGCACATCTGAAAACTTGTAT
TTCCAGGGCATGGACGAGGTGTATGCGGCAGTGGAACAGACTTCTCGACTTCTGG
ACGTTCCCTGTTCTCCTGACCGATTTGAGCCCGTTTGGAAGGCCTTTGGTGATCAG
CTGCCTGATTCTCACCTGGTCTTCTCTATGGCTGCTGGTGAGGCTCATCGAGGTGA
GCTGGACTTTGACTTCTCTCTTCGACCTGAGGGTGCTGATCCTTACACTACCGCTC
TGGAGCACGGCTTCATTGAGCCTACTGATCATCCTGTCGGTTCTGTCCTTGCTGAG
GTTGGTAAGCGATTCGCCATCGCCTCTTACGGTGTCGAATACGGTGTTGTCGGTG
GCTTCAAGAAGTCTTACGCCTTCTTCCCCCTGGACGACTTTCCCCCTCTTGCTCAG
TTTGCTGAGGTCCCCTCTGTTCCTCCTTGTTTAGCTGGTCACGTTGAGACCCTTAC
CCGACTGGGATTTGACGACAAGGTCTCGGCCATTGGCGTCAACTACCGAAAGAA
CACCCTGAACGTCTACCTGGCTGCTTCTGCTGTTGACACTGGCGATAAGCTGGCT
CTGCTTCGAGCTTTCGGTTACCCTGAACCTGATGCCCGAGTTCGACAGTTCATCGA
GCGATCTTTCCGGCTTTACCCCACCTTCAACTGGGATTCTTCTGCTGCCGAGCGAA
TCTGCTTCTCCGTCTTTACTCAGCAGCCTGGTGAGCTTCCTGCACCTCATGATGAA
CCTACCGAGGCTTTCGCTCGACAGGTTCCTCATGTTTACGAGGGTGGTCGAGAGT
TCGTCTCTGGTGTTGCTCTTGCTCCTTCTGGTGCCTCCTACTACAAGTTAGCTGCC
CTGTACCAGAAGGCTCGACGATGTCTGGGTTAG
SEQ ID NO: 118 <APT73.23.NTS>
ATGAAACATCACCACCACCACCATGGCACATCTGAAAACTTGTAT
TTCCAGGGCATGGACGAGGTGTATGCGGCAGTGGAACAGACTTCTCGACTTCTGG
ACGTTCCCTGTTCTCCTGACCGATTTGAGCCCGTTTGGAAGGCCTTTGGTGATCAG
CTGCCTGATTCTCACCTGGTCTTCTCTATGGCTGCTGGTGAGGCTCATCGAGGTGA
GCTGGACTTTGACTTCTCTCTTCGACCTGAGGGTGCTGATCCTTACACTACCGCTC
TGGAGCACGGCTTCATTGAGCCTACTGATCATCCTGTCGGTTCTGTCCTTGCTGAG
GTTGGTAAGCGATTCGCCATCGCCTCTTACGGTGTCGAATACGGTGTTGTCGGTG
GCTTCAAGAAGTCTTACGCCTTCTTCCCCCTGGACGACTTTCCCCCTCTTGCTCAG
TTTGCTGAGGTCCCCTCTGTTCCTCCTTGTTTAGCTGGTCACGTTGAGACCCTTAC
CCGACTGGGATTTGACGACAAGGTCTCGGCCATTGGCGTCAACTACCGAAAGAA
CACCCTGAACGTCTACCTGGCTGCTTCTGCTGTTGACACTGGCGATAAGCTGGCT
CTGCTTCGAGCTTTCGGTTACCCTGAACCTGATGCCCGAGTTCGACAGTTCATCGA
GCGATCTTTCCGGCTTTACCCCACCTTCAACTGGGATTCTTCTGCTGCCGAGCGAA
TCTGCTTCTCTGTCCACACTCAGCAACCTGGTGAGCTTCCTGCACCTCATGATGAA
CCTACCGAGGCTTTCGCTAGACAGGTTCCTCACGTTTACGAGGGTGGTCGAGAGT
TCGTCTCTGGTGTTGCTCTTGCTCCTTCTGGTGCCTCTTACTACAAGCTTGCTGCCC
TGTACCAGAAGGCTCGACGATGTCTGGGTTAG
SEQ ID NO: 119 <APT73.24.NTS>
ATGAAACATCACCACCACCACCATGGCACATCTGAAAACTTGTAT
TTCCAGGGCATGGACGAGGTGTATGCGGCAGTGGAACAGACTTCTCGACTTCTGG
ACGTTCCCTGTTCTCCTGACCGATTTGAGCCCGTTTGGAAGGCCTTTGGTGATCAG
CTGCCTGATTCTCACCTGGTCTTCTCTATGGCTGCTGGTGAGGCTCATCGAGGTGA
GCTGGACTTTGACTTCTCTCTTCGACCTGAGGGTGCTGATCCTTACACTACCGCTC
TGGAGCACGGCTTCATTGAGCCTACTGATCATCCTGTCGGTTCTGTCCTTGCTGAG
GTTGGTAAGCGATTCGCCATCGCCTCTTACGGTGTCGAATACGGTGTTGTCGGTG
GCTTCAAGAAGTCTTACGCCTTCTTCCCCCTGGACGACTTTCCCCCTCTTGCTCAG
TTTGCTGAGGTCCCCTCTGTTCCTCCTTGTTTAGCTGGTCACGTTGAGACCCTTAC
CCGACTGGGATTTGACGACAAGGTCTCGGCCATTGGCGTCAACTACCGAAAGAA
CACCCTGAACGTCTACCTGGCTGCTTCTGCTGTTGACACTGGCGATAAGCTGGCT
CTGCTTCGAGCTTTCGGTTACCCTGAACCTGATGCCCGAGTTCGACAGTTCATCGA
GCGATCTTTCCGGCTTTACCCCACCTTCAACTGGGATTCTTCTGCTGCCGAGCGAA
TCTGCTTCTCTGTCCGAACTCAGCAACCTGGTGAGCTTCCTGCACCTCATGATGAA
CCTACCGAGGCTTTCGCTAGACAGGTTCCTCACGTTTACGAGGGTGGTCGAGAGT
TCGTCTCTGGTGTTGCTCTTGCTCCTTCTGGTGCCTCTTACTACAAGCTTGCTGCCC
TGTACCAGAAGGCTCGACGATGTCTGGGTTAG
SEQ ID NO: 120 <APT73.25.NTS>
ATGAAACATCACCACCACCACCATGGCACATCTGAAAACTTGTAT
TTCCAGGGCATGGACGAGGTGTATGCGGCAGTGGAACAGACTTCTCGACTTCTGG
ACGTTCCCTGTTCTCCTGACCGATTTGAGCCCGTTTGGAAGGCCTTTGGTGATCAG
CTGCCTGATTCTCACCTGGTCTTCTCTATGGCTGCTGGTGAGGCTCATCGAGGTGA
GCTGGACTTTGACTTCTCTCTTCGACCTGAGGGTGCTGATCCTTACACTACCGCTC
TGGAGCACGGCTTCATTGAGCCTACTGATCATCCTGTCGGTTCTGTCCTTGCTGAG
GTTGGTAAGCGATTCGCCATCGCCTCTTACGGTGTCGAATACGGTGTTGTCGGTG
GCTTCAAGAAGTCTTACGCCTTCTTCCCCCTGGACGACTTTCCCCCTCTTGCTCAG
TTTGCTGAGGTCCCCTCTGTTCCTCCTTGTTTAGCTGGTCACGTTGAGACCCTTAC
CCGACTGGGATTTGACGACAAGGTCTCGGCCATTGGCGTCAACTACCGAAAGAA
CACCCTGAACGTCTACCTGGCTGCTTCTGCTGTTGACACTGGCGATAAGCTGGCT
CTGCTTCGAGCTTTCGGTTACCCTGAACCTGATGCCCGAGTTCGACAGTTCATCGA
GCGATCTTTCCGGCTTTACCCCACCTTCAACTGGGATTCTTCTGCTGCCGAGCGAA
TCTGCTTCTCTGTCCACACTCAGCAACCTGGTGAGCTTCCTGCACCTCATGATGAA
CCTACCGAGGCTTTCGCTAGACAGGTTCCTCACGTTTACGAGGGTGGTCGAGAGT
TCGTCTCTGGTGTTGCTCTTGCTCCTTCTGGTGCCTCTTACTACAAGCTTGCTGCCG
TCTACCAGAAGGCTCGACGATGTCTGGGTTAG
SEQ ID NO: 121 <APT73.26.NTS>
ATGAAACATCACCACCACCACCATGGCACATCTGAAAACTTGTAT
TTCCAGGGCATGGACGAGGTGTATGCGGCAGTGGAACAGACTTCTCGACTTCTGG
ACGTTCCCTGTTCTCCTGACCGATTTGAGCCCGTTTGGAAGGCCTTTGGTGATCAG
CTGCCTGATTCTCACCTGGTCTTCTCTATGGCTGCTGGTGAGGCTCATCGAGGTGA
GCTGGACTTTGACTTCTCTCTTCGACCTGAGGGTGCTGATCCTTACACTACCGCTC
TGGAGCACGGCTTCATTGAGCCTACTGATCATCCTGTCGGTTCTGTCCTTGCTGAG
GTTGGTAAGCGATTCGCCATCGCCTCTTACGGTGTCGAATACGGTGTTGTCGGTG
GCTTCAAGAAGTCTTACGCCTTCTTCCCCCTGGACGACTTTCCCCCTCTTGCTCAG
TTTGCTGAGGTCCCCTCTGTTCCTCCTTGTTTAGCTGGTCACGTTGAGACCCTTAC
CCGACTGGGATTTGACGACAAGGTCTCGGCCATTGGCGTCAACTACCGAAAGAA
CACCCTGAACGTCTACCTGGCTGCTTCTGCTGTTGACACTGGCGATAAGCTGGCT
CTGCTTCGAGCTTTCGGTTACCCTGAACCTGATGCCCGAGTTCGACAGTTCATCGA
GCGATCTTTCCGGCTTTACCCCACCTTCAACTGGGATTCTTCTGCTGCCGAGCGAA
TCTGTTTCGCTGTCCGAACTCAGCAACCTGGTGAGCTTCCTGCACCTCATGATGAA
CCTACCGAGGCTTTTGCTCGACAGGTTCCTCACGTTTACGAGGGTGGTCGAGAGT
TCGTCTCTGCTGTTGCTCTTGCTCCTTCTGGTGCCTCTTACTACAAGCTTGCTGCCC
TGTACCAGAAGGCTCGACGATGTCTGGGTTAG
SEQ ID NO: 122 <APT73.27.NTS>
ATGAAACATCACCACCACCACCATGGCACATCTGAAAACTTGTAT
TTCCAGGGCATGGACGAGGTGTATGCGGCAGTGGAACAGACTTCTCGACTTCTGG
ACGTTCCCTGTTCTCCTGACCGATTTGAGCCCGTTTGGAAGGCCTTTGGTGATCAG
CTGCCTGATTCTCACCTGGTCTTCTCTATGGCTGCTGGTGAGGCTCATCGAGGTGA
GCTGGACTTTGACTTCTCTCTTCGACCTGAGGGTGCTGATCCTTACACTACCGCTC
TGGAGCACGGCTTCATTGAGCCTACTGATCATCCTGTCGGTTCTGTCCTTGCTGAG
GTTGGTAAGCGATTCGCCATCGCCTCTTACGGTGTCGAATACGGTGTTGTCGGTG
GCTTCAAGAAGTCTTACGCCTTCTTCCCCCTGGACGACTTTCCCCCTCTTGCTCAG
TTTGCTGAGGTCCCCTCTGTTCCTCCTTGTTTAGCTGGTCACGTTGAGACCCTTAC
CCGACTGGGATTTGACGACAAGGTCTCGGCCATTGGCGTCAACTACCGAAAGAA
CACCCTGAACGTCTACCTGGCTGCTTCTGCTGTTGACACTGGCGATAAGCTGGCT
CTGCTTCGAGCTTTCGGTTACCCTGAACCTGATGCCCGAGTTCGACAGTTCATCGA
GCGATCTTTCCGGCTTTACCCCACCTTCAACTGGGATTCTTCTGCTGCCGAGCGAA
TCTGTTTCGCTGTCCGAACTCAGCAACCTGGTGAGCTTCCTGCACCTCATGATGAA
CCTACCGAGGCTTTTGCTCGACAGGTTCCTCACGTTTACGAGGGTGGTCGAGAGT
TCGTCTCTGCTGTTGCTCTTGCTCCTTCTGGTGCCTCTTACTACAAGCTTGCTGCCT
GCTACCAGAAGGCTCGACGATGTCTGGGTTAG
SEQ ID NO: 123 <APT73.28.NTS>
ATGAAACATCACCACCACCACCATGGCACATCTGAAAACTTGTAT
TTCCAGGGCATGGACGAGGTGTATGCGGCAGTGGAACAGACTTCTCGACTTCTGG
ACGTTCCCTGTTCTCCTGACCGATTTGAGCCCGTTTGGAAGGCCTTTGGTGATCAG
CTGCCTGATTCTCACCTGGTCTTCTCTATGGCTGCTGGTGAGGCTCATCGAGGTGA
GCTGGACTTTGACTTCTCTCTTCGACCTGAGGGTGCTGATCCTTACACTACCGCTC
TGGAGCACGGCTTCATTGAGCCTACTGATCATCCTGTCGGTTCTGTCCTTGCTGAG
GTTGGTAAGCGATTCGCCATCGCCTCTTACGGTGTCGAATACGGTGTTGTCGGTG
GCTTCAAGAAGTCTTACGCCTTCTTCCCCCTGGACGACTTTCCCCCTCTTGCTCAG
TTTGCTGAGGTCCCCTCTGTTCCTCCTTGTTTAGCTGGTCACGTTGAGACCCTTAC
CCGACTGGGATTTGACGACAAGGTCTCGGCCATTGGCGTCAACTACCGAAAGAA
CACCCTGAACGTCTACCTGGCTGCTTCTGCTGTTGACACTGGCGATAAGCTGGCT
CTGCTTCGAGCTTTCGGTTACCCTGAACCTGATGCCCGAGTTCGACAGTTCATCGA
GCGATCTTTCCGGCTTTACCCCACCTTCAACTGGGATTCTTCTGCTGCCGAGCGAA
TCTGTTTCGCCGTCTTTACTCAGCAGCCTGGTGAGCTTCCTGCACCTCATGATGAA
CCTACCGAGGCTTTTGCTCGACAGGTTCCTCACGTTTACGAGGGTGGTCGAGAGT
TCGTCTCTGCTGTTGCTCTTGCTCCTTCTGGTGCCTCTTACTACAAGCTTGCTGCCT
CCTACCAGAAGGCTCGACGATGTCTGGGTTAG
SEQ ID NO: 124 <APT73.29.NTS>
ATGAAACATCACCACCACCACCATGGCACATCTGAAAACTTGTAT
TTCCAGGGCATGGACGAGGTGTATGCGGCAGTGGAACAGACTTCTCGACTTCTGG
ACGTTCCCTGTTCTCCTGACCGATTTGAGCCCGTTTGGAAGGCCTTTGGTGATCAG
CTGCCTGATTCTCACCTGGTCTTCTCTATGGCTGCTGGTGAGGCTCATCGAGGTGA
GCTGGACTTTGACTTCTCTCTTCGACCTGAGGGTGCTGATCCTTACACTACCGCTC
TGGAGCACGGCTTCATTGAGCCTACTGATCATCCTGTCGGTTCTGTCCTTGCTGAG
GTTGGTAAGCGATTCGCCATCGCCTCTTACGGTGTCGAATACGGTGTTGTCGGTG
GCTTCAAGAAGTCTTACGCCTTCTTCCCCCTGGACGACTTTCCCCCTCTTGCTCAG
TTTGCTGAGGTCCCCTCTGTTCCTCCTTGTTTAGCTGGTCACGTTGAGACCCTTAC
CCGACTGGGATTTGACGACAAGGTCTCGGCCATTGGCGTCAACTACCGAAAGAA
CACCCTGAACGTCTACCTGGCTGCTTCTGCTGTTGACACTGGCGATAAGCTGGCT
CTGCTTCGAGCTTTCGGTTACCCTGAACCTGATGCCCGAGTTCGACAGTTCATCGA
GCGATCTTTCCGGCTTTACCCCACCTTCAACTGGGATTCTTCTGCTGCCGAGCGAA
TCTGTTTCGCTGTCCACACTCAGCAACCTGGTGAGCTTCCTGCACCTCATGATGAA
CCTACCGAGGCTTTTGCTCGACAGGTTCCTCACGTTTACGAGGGTGGTCGAGAGT
TCGTCTCTGCTGTTGCTCTTGCTCCTTCTGGTGCCTCTTACTACAAGCTTGCTGCCG
TCTACCAGAAGGCTCGACGATGTCTGGGTTAG
SEQ ID NO: 125 <APT73.30.NTS>
ATGAAACATCACCACCACCACCATGGCACATCTGAAAACTTGTAT
TTCCAGGGCATGGACGAGGTGTATGCGGCAGTGGAACAGACTTCTCGACTTCTGG
ACGTTCCCTGTTCTCCTGACCGATTTGAGCCCGTTTGGAAGGCCTTTGGTGATCAG
CTGCCTGATTCTCACCTGGTCTTCTCTATGGCTGCTGGTGAGGCTCATCGAGGTGA
GCTGGACTTTGACTTCTCTCTTCGACCTGAGGGTGCTGATCCTTACACTACCGCTC
TGGAGCACGGCTTCATTGAGCCTACTGATCATCCTGTCGGTTCTGTCCTTGCTGAG
GTTGGTAAGCGATTCGCCATCGCCTCTTACGGTGTCGAATACGGTGTTGTCGGTG
GCTTCAAGAAGTCTTACGCCTTCTTCCCCCTGGACGACTTTCCCCCTCTTGCTCAG
TTTGCTGAGGTCCCCTCTGTTCCTCCTTGTTTAGCTGGTCACGTTGAGACCCTTAC
CCGACTGGGATTTGACGACAAGGTCTCGGCCATTGGCGTCAACTACCGAAAGAA
CACCCTGAACGTCTACCTGGCTGCTTCTGCTGTTGACACTGGCGATAAGCTGGCT
CTGCTTCGAGCTTTCGGTTACCCTGAACCTGATGCCCGAGTTCGACAGTTCATCGA
GCGATCTTTCCGGCTTTACCCCACCTTCAACTGGGATTCTTCTGCTGCCGAGCGAA
TCTGTTTCGCTGTCCACACTCAGCAACCTGGTGAGCTTCCTGCACCTCATGATGAA
CCTACCGAGGCTTTTGCTCGACAGGTTCCTCACGTTTACGAGGGTGGTCGAGAGT
TCGTCTCTGGTGTTGCTCTTGCTCCTTCTGGTGCCTCTTACTACAAGCTTGCTGCCC
TGTACCAGAAGGCTCGACGATGTCTGGGTTAG
SEQ ID NO: 126 <APT73.31.NTS>
ATGAAACATCACCACCACCACCATGGCACATCTGAAAACTTGTAT
TTCCAGGGCATGGACGAGGTGTATGCGGCAGTGGAACAGACTTCTCGACTTCTGG
ACGTTCCCTGTTCTCCTGACCGATTTGAGCCCGTTTGGAAGGCCTTTGGTGATCAG
CTGCCTGATTCTCACCTGGTCTTCTCTATGGCTGCTGGTGAGGCTCATCGAGGTGA
GCTGGACTTTGACTTCTCTCTTCGACCTGAGGGTGCTGATCCTTACACTACCGCTC
TGGAGCACGGCTTCATTGAGCCTACTGATCATCCTGTCGGTTCTGTCCTTGCTGAG
GTTGGTAAGCGATTCGCCATCGCCTCTTACGGTGTCGAATACGGTGTTGTCGGTG
GCTTCAAGAAGTCTTACGCCTTCTTCCCCCTGGACGACTTTCCCCCTCTTGCTCAG
TTTGCTGAGGTCCCCTCTGTTCCTCCTTGTTTAGCTGGTCACGTTGAGACCCTTAC
CCGACTGGGATTTGACGACAAGGTCTCGGCCATTGGCGTCAACTACCGAAAGAA
CACCCTGAACGTCTACCTGGCTGCTTCTGCTGTTGACACTGGCGATAAGCTGGCT
CTGCTTCGAGCTTTCGGTTACCCTGAACCTGATGCCCGAGTTCGACAGTTCATCGA
GCGATCTTTCCGGCTTTACCCCACCTTCAACTGGGATTCTTCTGCTGCCGAGCGAA
TCTGTTTCGCTGTCCGAACTCAGCAACCTGGTGAGCTTCCTGCACCTCATGATGAA
CCTACCGAGGCTTTTGCTCGACAGGTTCCTCACGTTTACGAGGGTGGTCGAGAGT
TCGTCTCTGGTGTTGCTCTTGCTCCTTCTGGTGCCTCTTACTACAAGCTTGCTGCCG
TCTACCAGAAGGCTCGACGATGTCTGGGTTAG
SEQ ID NO: 127 <APT73.32.NTS>
ATGAAACATCACCACCACCACCATGGCACATCTGAAAACTTGTAT
TTCCAGGGCATGGACGAGGTGTATGCGGCAGTGGAACAGACTTCTCGACTTCTGG
ACGTTCCCTGTTCTCCTGACCGATTTGAGCCCGTTTGGAAGGCCTTTGGTGATCAG
CTGCCTGATTCTCACCTGGTCTTCTCTATGGCTGCTGGTGAGGCTCATCGAGGTGA
GCTGGACTTTGACTTCTCTCTTCGACCTGAGGGTGCTGATCCTTACACTACCGCTC
TGGAGCACGGCTTCATTGAGCCTACTGATCATCCTGTCGGTTCTGTCCTTGCTGAG
GTTGGTAAGCGATTCGCCATCGCCTCTTACGGTGTCGAATACGGTGTTGTCGGTG
GCTTCAAGAAGTCTTACGCCTTCTTCCCCCTGGACGACTTTCCCCCTCTTGCTCAG
TTTGCTGAGGTCCCCTCTGTTCCTCCTTGTTTAGCTGGTCACGTTGAGACCCTTAC
CCGACTGGGATTTGACGACAAGGTCTCGGCCATTGGCGTCAACTACCGAAAGAA
CACCCTGAACGTCTACCTGGCTGCTTCTGCTGTTGACACTGGCGATAAGCTGGCT
CTGCTTCGAGCTTTCGGTTACCCTGAACCTGATGCCCGAGTTCGACAGTTCATCGA
GCGATCTTTCCGGCTTTACCCCACCTTCAACTGGGATTCTTCTGCTGCCGAGCGAA
TCTGTTTCGCCGTCAAGACACAGCAACCTGGTGAGCTTCCTGCACCTCATGATGA
ACCTACCGAGGCTTTTGCTCGACAGGTTCCTCACGTTTACGAGGGTGGTCGAGAG
TTCGTCTCTGCTGTTGCTCTTGCTCCTTCTGGTGCCTCTTACTACAAGCTTGCTGCC
TCCTACCAGAAGGCTCGACGACCTCTTGGTTAG
SEQ ID NO: 128 <APT73.33.NTS>
ATGAAACATCACCACCACCACCATGGCACATCTGAAAACTTGTAT
TTCCAGGGCATGGACGAGGTGTATGCGGCAGTGGAACAGACTTCTCGACTTCTGG
ACGTTCCCTGTTCTCCTGACCGATTTGAGCCCGTTTGGAAGGCCTTTGGTGATCAG
CTGCCTGATTCTCACCTGGTCTTCTCTATGGCTGCTGGTGAGGCTCATCGAGGTGA
GCTGGACTTTGACTTCTCTCTTCGACCTGAGGGTGCTGATCCTTACACTACCGCTC
TGGAGCACGGCTTCATTGAGCCTACTGATCATCCTGTCGGTTCTGTCCTTGCTGAG
GTTGGTAAGCGATTCGCCATCGCCTCTTACGGTGTCGAATACGGTGTTGTCGGTG
GCTTCAAGAAGTCTTACGCCTTCTTCCCCCTGGACGACTTTCCCCCTCTTGCTCAG
TTTGCTGAGGTCCCCTCTGTTCCTCCTTGTTTAGCTGGTCACGTTGAGACCCTTAC
CCGACTGGGATTTGACGACAAGGTCTCGGCCATTGGCGTCAACTACCGAAAGAA
CACCCTGAACGTCTACCTGGCTGCTTCTGCTGTTGACACTGGCGATAAGCTGGCT
CTGCTTCGAGCTTTCGGTTACCCTGAACCTGATGCCCGAGTTCGACAGTTCATCGA
GCGATCTTTCCGGCTTTACCCCACCTTCAACTGGGATTCTTCTGCTGCCGAGCGAA
TCTGTTTCGCCGTCAAGACACAGCAACCTGGTGAGCTTCCTGCACCTCATGATGA
ACCTACCGAGGCTTTTGCTCGACAGGTTCCTCACGTTTACGAGGGTGGTCGAGAG
TTCGTCTCTGCTGTTGCTCTTGCTCCTTCTGGTGCCTCTTACTACAAGCTTGCTGCC
GCTTACCAGAAGGCTCGACGACCTCTTGGTTAG
SEQ ID NO: 129 <APT73.34.NTS>
ATGAAACATCACCACCACCACCATGGCACATCTGAAAACTTGTAT
TTCCAGGGCATGGACGAGGTGTATGCGGCAGTGGAACAGACTTCTCGACTTCTGG
ACGTTCCCTGTTCTCCTGACCGATTTGAGCCCGTTTGGAAGGCCTTTGGTGATCAG
CTGCCTGATTCTCACCTGGTCTTCTCTATGGCTGCTGGTGAGGCTCATCGAGGTGA
GCTGGACTTTGACTTCTCTCTTCGACCTGAGGGTGCTGATCCTTACACTACCGCTC
TGGAGCACGGCTTCATTGAGCCTACTGATCATCCTGTCGGTTCTGTCCTTGCTGAG
GTTGGTAAGCGATTCGCCATCGCCTCTTACGGTGTCGAATACGGTGTTGTCGGTG
GCTTCAAGAAGTCTTACGCCTTCTTCCCCCTGGACGACTTTCCCCCTCTTGCTCAG
TTTGCTGAGGTCCCCTCTGTTCCTCCTTGTTTAGCTGGTCACGTTGAGACCCTTAC
CCGACTGGGATTTGACGACAAGGTCTCGGCCATTGGCGTCAACTACCGAAAGAA
CACCCTGAACGTCTACCTGGCTGCTTCTGCTGTTGACACTGGCGATAAGCTGGCT
CTGCTTCGAGCTTTCGGTTACCCTGAACCTGATGCCCGAGTTCGACAGTTCATCGA
GCGATCTTTCCGGCTTTACCCCACCTTCAACTGGGATTCTTCTGCTGCCGAGCGAA
TCTGTTTCGCCGTCAAGACACAGCAACCTGGTGAGCTTCCTGCACCTCATGATGA
ACCTACCGAGGCTTTTGCTCGACAGGTTCCTCACGTTTACGAGGGTGGTCGAGAG
TTCGTCTCTGCTGTTGCTCTTGCTCCTTCTGGTGCCTCTTACTACAAGCTTGCTGCC
TGCTACCAGAAGGCTCGACGAGAGCTTGGTTAG
SEQ ID NO: 130 <APT73.35.NTS>
ATGAAACATCACCACCACCACCATGGCACATCTGAAAACTTGTAT
TTCCAGGGCATGGACGAGGTGTATGCGGCAGTGGAACAGACTTCTCGACTTCTGG
ACGTTCCCTGTTCTCCTGACCGATTTGAGCCCGTTTGGAAGGCCTTTGGTGATCAG
CTGCCTGATTCTCACCTGGTCTTCTCTATGGCTGCTGGTGAGGCTCATCGAGGTGA
GCTGGACTTTGACTTCTCTCTTCGACCTGAGGGTGCTGATCCTTACACTACCGCTC
TGGAGCACGGCTTCATTGAGCCTACTGATCATCCTGTCGGTTCTGTCCTTGCTGAG
GTTGGTAAGCGATTCGCCATCGCCTCTTACGGTGTCGAATACGGTGTTGTCGGTG
GCTTCAAGAAGTCTTACGCCTTCTTCCCCCTGGACGACTTTCCCCCTCTTGCTCAG
TTTGCTGAGGTCCCCTCTGTTCCTCCTTGTTTAGCTGGTCACGTTGAGACCCTTAC
CCGACTGGGATTTGACGACAAGGTCTCGGCCATTGGCGTCAACTACCGAAAGAA
CACCCTGAACGTCTACCTGGCTGCTTCTGCTGTTGACACTGGCGATAAGCTGGCT
CTGCTTCGAGCTTTCGGTTACCCTGAACCTGATGCCCGAGTTCGACAGTTCATCGA
GCGATCTTTCCGGCTTTACCCCACCTTCAACTGGGATTCTTCTGCTGCCGAGCGAA
TCTGTTTCGCCGTCAAGACACAGCAACCTGGTGAGCTTCCTGCACCTCATGATGA
ACCTACCGAGGCTTTTGCTCGACAGGTTCCTCACGTTTACGAGGGTGGTCGAGAG
TTCGTCTCTGCTGTTGCTCTTGCTCCTTCTGGTGCCTCTTACTACAAGCTTGCTGCC
TCCTACCAGAAGGCTCGACGAGAGCTTGGTTAG
SEQ ID NO: 131 <APT73.36.NTS>
ATGAAACATCACCACCACCACCATGGCACATCTGAAAACTTGTAT
TTCCAGGGCATGGACGAGGTGTATGCGGCAGTGGAACAGACTTCTCGACTTCTGG
ACGTTCCCTGTTCTCCTGACCGATTTGAGCCCGTTTGGAAGGCCTTTGGTGATCAG
CTGCCTGATTCTCACCTGGTCTTCTCTATGGCTGCTGGTGAGGCTCATCGAGGTGA
GCTGGACTTTGACTTCTCTCTTCGACCTGAGGGTGCTGATCCTTACACTACCGCTC
TGGAGCACGGCTTCATTGAGCCTACTGATCATCCTGTCGGTTCTGTCCTTGCTGAG
GTTGGTAAGCGATTCGCCATCGCCTCTTACGGTGTCGAATACGGTGTTGTCGGTG
GCTTCAAGAAGTCTTACGCCTTCTTCCCCCTGGACGACTTTCCCCCTCTTGCTCAG
TTTGCTGAGGTCCCCTCTGTTCCTCCTTGTTTAGCTGGTCACGTTGAGACCCTTAC
CCGACTGGGATTTGACGACAAGGTCTCGGCCATTGGCGTCAACTACCGAAAGAA
CACCCTGAACGTCTACCTGGCTGCTTCTGCTGTTGACACTGGCGATAAGCTGGCT
CTGCTTCGAGCTTTCGGTTACCCTGAACCTGATGCCCGAGTTCGACAGTTCATCGA
GCGATCTTTCCGGCTTTACCCCACCTTCAACTGGGATTCTTCTGCTGCCGAGCGAA
TCTGTTTCGCCGTCAAGACACAGCAACCTGGTGAGCTTCCTGCACCTCATGATGA
ACCTACCGAGGCTTTTGCTCGACAGGTTCCTCACGTTTACGAGGGTGGTCGAGAG
TTCGTCTCTGGTGTTGCTCTTGCTCCTTCTGGTGCCTCTTACTACAAGCTTGCTGCC
TGCTACCAGAAGGCTCGACGACCTCTTGGTTAG
SEQ ID NO: 132 <APT73.37.NTS>
ATGAAACATCACCACCACCACCATGGCACATCTGAAAACTTGTAT
TTCCAGGGCATGGACGAGGTGTATGCGGCAGTGGAACAGACTTCTCGACTTCTGG
ACGTTCCCTGTTCTCCTGACCGATTTGAGCCCGTTTGGAAGGCCTTTGGTGATCAG
CTGCCTGATTCTCACCTGGTCTTCTCTATGGCTGCTGGTGAGGCTCATCGAGGTGA
GCTGGACTTTGACTTCTCTCTTCGACCTGAGGGTGCTGATCCTTACACTACCGCTC
TGGAGCACGGCTTCATTGAGCCTACTGATCATCCTGTCGGTTCTGTCCTTGCTGAG
GTTGGTAAGCGATTCGCCATCGCCTCTTACGGTGTCGAATACGGTGTTGTCGGTG
GCTTCAAGAAGTCTTACGCCTTCTTCCCCCTGGACGACTTTCCCCCTCTTGCTCAG
TTTGCTGAGGTCCCCTCTGTTCCTCCTTGTTTAGCTGGTCACGTTGAGACCCTTAC
CCGACTGGGATTTGACGACAAGGTCTCGGCCATTGGCGTCAACTACCGAAAGAA
CACCCTGAACGTCTACCTGGCTGCTTCTGCTGTTGACACTGGCGATAAGCTGGCT
CTGCTTCGAGCTTTCGGTTACCCTGAACCTGATGCCCGAGTTCGACAGTTCATCGA
GCGATCTTTCCGGCTTTACCCCACCTTCAACTGGGATTCTTCTGCTGCCGAGCGAA
TCTGTTTCGCCGTCAAGACACAGCAACCTGGTGAGCTTCCTGCACCTCATGATGA
ACCTACCGAGGCTTTTGCTCGACAGGTTCCTCACGTTTACGAGGGTGGTCGAGAG
TTCGTCTCTGGTGTTGCTCTTGCTCCTTCTGGTGCCTCTTACTACAAGCTTGCTGCC
GTCTACCAGAAGGCTCGACGACCTCTTGGTTAG
SEQ ID NO: 133 <APT73.44.NTS>
ATGAAACATCACCACCACCACCATGGCACATCTGAAAACTTGTAT
TTCCAGGGCATGGACGAGGTGTATGCGGCAGTGGAACAGACTTCTCGACTTCTGG
ACGTTCCCTGTTCTCCTGACCGATTTGAGCCCGTTTGGAAGGCCTTTGGTGATCAG
CTGCCTGATTCTCACCTGGTCTTCTCTATGGCTGCTGGTGAGGCTCATCGAGGTGA
GCTGGACTTTGACTTCTCTCTTCGACCTGAGGGTGCTGATCCTTACACTACCGCTC
TGGAGCACGGCTTCATTGAGCCTACTGATCATCCTGTCGGTTCTGTCCTTGCTGAG
GTTGGTAAGCGATTCGCCATCGCCTCTTACGGTGTCGAATACGGTGTTGTCGGTG
GCTTCAAGAAGTCTTACGCCTTCTTCCCCCTGGACGACTTTCCCCCTCTTGCTCAG
TTTGCTGAGGTCCCCTCTGTTCCTCCTTGTTTAGCTGGTCACGTTGAGACCCTTAC
CCGACTGGGATTTGACGACAAGGTCTCGGCCATTGGCGTCAACTACCGAAAGAA
CACCCTGAACGTCTACCTGGCTGCTTCTGCTGTTGACACTGGCGATAAGCTGGCT
CTGCTTCGAGCTTTCGGTTACCCTGAACCTGATGCCCGAGTTCGACAGTTCATCGA
GCGATCTTTCCGGCTTTACCCCACCTTCAACTGGGATTCTTCTGCTGCCGAGCGAA
TCTGCTTCTCCGTCGCGACTCAGCAGCCTGGTGAGCTTCCTGCACCTCATGATGAA
CCTACCGAGGCTTTCGCTCGACAGGTTCCTCATGTTTACGAGGGTGGTCGAGAGT
TCGTCTCTGCTGTTGCTCTTGCTCCTTCTGGTGCCTCCTACTACAAGTTAGCTGCCC
TGTACCAGAAGGCTCGACGATGTCTGGGTTAG
SEQ ID NO: 134 <APT73.45.NTS>
ATGAAACATCACCACCACCACCATGGCACATCTGAAAACTTGTAT
TTCCAGGGCATGGACGAGGTGTATGCGGCAGTGGAACAGACTTCTCGACTTCTGG
ACGTTCCCTGTTCTCCTGACCGATTTGAGCCCGTTTGGAAGGCCTTTGGTGATCAG
CTGCCTGATTCTCACCTGGTCTTCTCTATGGCTGCTGGTGAGGCTCATCGAGGTGA
GCTGGACTTTGACTTCTCTCTTCGACCTGAGGGTGCTGATCCTTACACTACCGCTC
TGGAGCACGGCTTCATTGAGCCTACTGATCATCCTGTCGGTTCTGTCCTTGCTGAG
GTTGGTAAGCGATTCGCCATCGCCTCTTACGGTGTCGAATACGGTGTTGTCGGTG
GCTTCAAGAAGTCTTACGCCTTCTTCCCCCTGGACGACTTTCCCCCTCTTGCTCAG
TTTGCTGAGGTCCCCTCTGTTCCTCCTTGTTTAGCTGGTCACGTTGAGACCCTTAC
CCGACTGGGATTTGACGACAAGGTCTCGGCCATTGGCGTCAACTACCGAAAGAA
CACCCTGAACGTCTACCTGGCTGCTTCTGCTGTTGACACTGGCGATAAGCTGGCT
CTGCTTCGAGCTTTCGGTTACCCTGAACCTGATGCCCGAGTTCGACAGTTCATCGA
GCGATCTTTCCGGCTTTACCCCACCTTCAACTGGGATTCTTCTGCTGCCGAGCGAA
TCTGCTTCTCCGTCAGTACTCAGCAGCCTGGTGAGCTTCCTGCACCTCATGATGAA
CCTACCGAGGCTTTCGCTCGACAGGTTCCTCATGTTTACGAGGGTGGTCGAGAGT
TCGTCTCTGCTGTTGCTCTTGCTCCTTCTGGTGCCTCCTACTACAAGTTAGCTGCCT
GTTACCAGAAGGCTCGACGATGTCTGGGTTAG
SEQ ID NO: 135 <APT73.46.NTS>
ATGAAACATCACCACCACCACCATGGCACATCTGAAAACTTGTAT
TTCCAGGGCATGGACGAGGTGTATGCGGCAGTGGAACAGACTTCTCGACTTCTGG
ACGTTCCCTGTTCTCCTGACCGATTTGAGCCCGTTTGGAAGGCCTTTGGTGATCAG
CTGCCTGATTCTCACCTGGTCTTCTCTATGGCTGCTGGTGAGGCTCATCGAGGTGA
GCTGGACTTTGACTTCTCTCTTCGACCTGAGGGTGCTGATCCTTACACTACCGCTC
TGGAGCACGGCTTCATTGAGCCTACTGATCATCCTGTCGGTTCTGTCCTTGCTGAG
GTTGGTAAGCGATTCGCCATCGCCTCTTACGGTGTCGAATACGGTGTTGTCGGTG
GCTTCAAGAAGTCTTACGCCTTCTTCCCCCTGGACGACTTTCCCCCTCTTGCTCAG
TTTGCTGAGGTCCCCTCTGTTCCTCCTTGTTTAGCTGGTCACGTTGAGACCCTTAC
CCGACTGGGATTTGACGACAAGGTCTCGGCCATTGGCGTCAACTACCGAAAGAA
CACCCTGAACGTCTACCTGGCTGCTTCTGCTGTTGACACTGGCGATAAGCTGGCT
CTGCTTCGAGCTTTCGGTTACCCTGAACCTGATGCCCGAGTTCGACAGTTCATCGA
GCGATCTTTCCGGCTTTACCCCACCTTCAACTGGGATTCTTCTGCTGCCGAGCGAA
TCTGCTTCTCCGTCTTTACTCAGCAGCCTGGTGAGCTTCCTGCACCTCATGATGAA
CCTACCGAGGCTTTCGCTCGACAGGTTCCTCATGTTTACGAGGGTGGTCGAGAGT
TCGTCTCTGCTGTTGCTCTTGCTCCTTCTGGTGCCTCCTACTACAAGTTAGCTGCC
ACGTACCAGAAGGCTCGACGATGTCTGGGTTAG
SEQ ID NO: 136 <APT73.47.NTS>
ATGAAACATCACCACCACCACCATGGCACATCTGAAAACTTGTAT
TTCCAGGGCATGGACGAGGTGTATGCGGCAGTGGAACAGACTTCTCGACTTCTGG
ACGTTCCCTGTTCTCCTGACCGATTTGAGCCCGTTTGGAAGGCCTTTGGTGATCAG
CTGCCTGACTCTCATCTGGTCTTCTCTATGGCTGCTGGTGAGGCTCATCGAGGTGA
GCTGGACTTTGACTTCTCCCTTCGACCTGAAGGTGCTGATCCTTACACTACCGCTC
TGGAGCACGGCTTCATTGAGCCTACTGATCACCCTGTCGGTTCTGTTCTTGCTGAG
GTTGGTAAGCGATTCGCCATCGCCTCTTACGGTGTCGAATACGGTGTCGTCGGTG
GTTTCAAGAAGTCTTACGCCTTCTTCCCCCTGGACGACTTTCCTCCTCTTGCTCAG
TTCGCTGAGGTCCCCTCTGTTCCTCCTTGTTTAGCTGGTCACGTTGAGACTCTGAC
TCGACTGGGTTTTGACGACAAGGTCTCCATCATCGGCGTCAACTACCGGAAGAAC
ACCCTGAACGTCTACCTGGCCGCTTCTGCTGTTGATACTGGTGATAAGCTGGCTCT
GCTGCGAGCTTTCGGTTACCCTGAACCTGACGCTCGAGTTCGACAGTTCATTGAG
CGATCTTTCCGGCTGTACCCCACCTTCAACTGGGACTCTTCTGCTGCTGAGCGAAT
CTGTTTCGCTGTTCACACTCAGCAGCCTGGTGAGCTTCCTGCTCCTCATGATGAGC
CTACTGAGGCTTTTGCTCGACAAGTTCCTCACGTCTACGAGGGTGGTCGAGAGTT
CGTCTCTGGTGTTGCTCTTGCTCCTTCTGGTGCTTCTTACTACAAGCTGGCTGCCCT
GTACCAGAAGGCTCGACGATGTCTGGGTTAG
SEQ ID NO: 137 <APT73.49.NTS>
ATGAAACATCACCACCACCACCATGGCACATCTGAAAACTTGTAT
TTCCAGGGCATGGACGAGGTGTATGCGGCAGTGGAACAGACTTCTCGACTTCTGG
ACGTTCCCTGTTCTCCTGACCGATTTGAGCCCGTTTGGAAGGCCTTTGGTGATCAG
CTGCCTGACTCTCATCTGGTCTTCTCTATGGCTGCTGGTGAGGCTCATCGAGGTGA
GCTGGACTTTGACTTCTCCCTTCGACCTGAAGGTGCTGATCCTTACACTACCGCTC
TGGAGCACGGCTTCATTGAGCCTACTGATCACCCTGTCGGTTCTGTTCTTGCTGAG
GTTGGTAAGCGATTCGCCATCGCCTCTTACGGTGTCGAATACGGTGTCGTCGGTG
GTTTCAAGAAGTCTTACGCCTTCTTCCCCCTGGACGACTTTCCTCCTCTTGCTCAG
TTCGCTGAGGTCCCCTCTGTTCCTCCTTGTTTAGCTGGTCACGTTGAGACTCTGAC
TCGACTGGGTTTTGACGACAAGGTCTCCGCCATCGGCGTCAACTACCGAAAGAAC
ACCCTGAACGTTTACCTGGCTGCTTCTGCTGTTGATACCGGCGACAAGCTGGCTCT
GCTTCGAGCTTTCGGTTACCCTGAACCTGATGCTAGAGTTCGACAGTTCATCGAG
CGGTCCTTCCGGCTTTACCCCACCTTCAACTGGGACTCTTCTGCTGCTGAACGAAT
CTGTTTCGCTGTCATGACCCAGCAGCCTGGTGAGCTTCCTGCTCCTCATGATGAGC
CTACTGAGGCTTTTGCTAGACAGGTTCCTCACGTCTACGAGGGTGGTCGAGAGTT
CGTCTCTGGTGTTGCTCTTGCTCCTTCTGGTGCTTCTTACTACAAGCTGGCTGCCCT
GTACCAGAAGGCTCGACGATGTCTGGGTTAG
SEQ ID NO: 138 <APT73.52.NTS>
ATGAAACATCACCACCACCACCATGGCACATCTGAAAACTTGTAT
TTCCAGGGCATGGACGAGGTGTATGCGGCAGTGGAACAGACTTCTCGACTTCTGG
ACGTTCCCTGTTCTCCTGACCGATTTGAGCCCGTTTGGAAGGCCTTTGGTGATCAG
CTGCCTGACTCTCATCTGGTCTTCTCTATGGCTGCTGGTGAGGCTCATCGAGGTGA
GCTGGACTTTGACTTCTCCCTTCGACCTGAAGGTGCTGATCCTTACACTACCGCTC
TGGAGCACGGCTTCATTGAGCCTACTGATCACCCTGTCGGTTCTGTTCTTGCTGAG
GTTGGTAAGCGATTCGCCATCGCCTCTTACGGTGTCGAATACGGTGTCGTCGGTG
GTTTCAAGAAGTCTTACGCCTTCTTCCCCCTGGACGACTTTCCTCCTCTTGCTCAG
TTCGCTGAGGTCCCCTCTGTTCCTCCTTGTTTAGCTGGTCACGTTGAGACTCTGAC
TCGACTGGGTTTTGACGACAAGGTCTCCGCCATCGGCGTCAACTACCGAAAGAAC
ACCCTGAACGTTTACCTGGCTGCTTCTGCTGTTGATACCGGCGACAAGCTGGCTCT
GCTTCGAGCTTTCGGTTACCCTGAACCTGATGCTAGAGTTCGACAGTTCATCGAG
CGGTCCTTCCGGCTTTACCCCACCTTCAACTGGGACTCTTCTGCTGCTGAACGAAT
CTGTTTCGCTGTCCACACTCAGCAGCCTGGTGAGCTTCCTGCTCCTCATGATGAGC
CTACTGAGGCTTTTGCTAGACAGGTTCCTCACGTCTACGAGGGTGGTCGAGAGTT
CGTCTCTGGTGTTGCTCTTGCTCCTTCTGGTGCTTCTTACTACAAGCTGGCTGCCG
AGTACCAGAAGGCTCGACGATGTCTGGGTTAG
SEQ ID NO: 139 <APT73.53.NTS>
ATGAAACATCACCACCACCACCATGGCACATCTGAAAACTTGTAT
TTCCAGGGCATGGACGAGGTGTATGCGGCAGTGGAACAGACTTCTCGACTTCTGG
ACGTTCCCTGTTCTCCTGACCGATTTGAGCCCGTTTGGAAGGCCTTTGGTGATCAG
CTGCCTGACTCTCATCTGGTCTTCTCTATGGCTGCTGGTGAGGCTCATCGAGGTGA
GCTGGACTTTGACTTCTCCCTTCGACCTGAAGGTGCTGATCCTTACACTACCGCTC
TGGAGCACGGCTTCATTGAGCCTACTGATCACCCTGTCGGTTCTGTTCTTGCTGAG
GTTGGTAAGCGATTCGCCATCGCCTCTTACGGTGTCGAATACGGTGTCGTCGGTG
GTTTCAAGAAGTCTTACGCCTTCTTCCCCCTGGACGACTTTCCTCCTCTTGCTCAG
TTCGCTGAGGTCCCCTCTGTTCCTCCTTGTTTAGCTGGTCACGTTGAGACTCTGAC
TCGACTGGGTTTTGACGACAAGGTCTCCGCCATCGGCGTCAACTACCGAAAGAAC
ACCCTGAACGTTTACCTGGCTGCTTCTGCTGTTGATACCGGCGACAAGCTGGCTCT
GCTTCGAGCTTTCGGTTACCCTGAACCTGATGCTAGAGTTCGACAGTTCATCGAG
CGGTCCTTCCGGCTTTACCCCACCTTCAACTGGGACTCTTCTGCTGCTGAACGAAT
CTGTTTCGCTGTCCACACTCAGCAGCCTGGTGAGCTTCCTGCTCCTCATGATGAGC
CTACTGAGGCTTTTGCTAGACAGGTTCCTCACGTCTACGAGGGTGGTCGAGAGTT
CGTCTCTGGTGTTGCTCTTGCTCCTTCTGGTGCTTCTTACTACAAGCTGGCTGCCC
ACTACCAGAAGGCTCGACGATGTCTGGGTTAG
SEQ ID NO: 140 <APT73.54.NTS>
ATGAAACATCACCACCACCACCATGGCACATCTGAAAACTTGTAT
TTCCAGGGCATGGACGAGGTGTATGCGGCAGTGGAACAGACTTCTCGACTTCTGG
ACGTTCCCTGTTCTCCTGACCGATTTGAGCCCGTTTGGAAGGCCTTTGGTGATCAG
CTGCCTGACTCTCATCTGGTCTTCTCTATGGCTGCTGGTGAGGCTCATCGAGGTGA
GCTGGACTTTGACTTCTCCCTTCGACCTGAAGGTGCTGATCCTTACACTACCGCTC
TGGAGCACGGCTTCATTGAGCCTACTGATCACCCTGTCGGTTCTGTTCTTGCTGAG
GTTGGTAAGCGATTCGCCATCGCCTCTTACGGTGTCGAATACGGTGTCGTCGGTG
GTTTCAAGAAGTCTTACGCCTTCTTCCCCCTGGACGACTTTCCTCCTCTTGCTCAG
TTCGCTGAGGTCCCCTCTGTTCCTCCTTGTTTAGCTGGTCACGTTGAGACTCTGAC
TCGACTGGGTTTTGACGACAAGGTCTCCGCCATCGGCGTCAACTACCGAAAGAAC
ACCCTGAACGTTTACCTGGCTGCTTCTGCTGTTGATACCGGCGACAAGCTGGCTCT
GCTTCGAGCTTTCGGTTACCCTGAACCTGATGCTAGAGTTCGACAGTTCATCGAG
CGGTCCTTCCGGCTTTACCCCACCTTCAACTGGGACTCTTCTGCTGCTGAACGAAT
CTGTTTCGCTGTCCACACTCAGCAGCCTGGTGAGCTTCCTGCTCCTCATGATGAGC
CTACTGAGGCTTTTGCTAGACAGGTTCCTCACGTCTACGAGGGTGGTCGAGAGTT
CGTCTCTGGTGTTGCTCTTGCTCCTTCTGGTGCTTCTTACTACAAGCTGGCTGCCA
TGTACCAGAAGGCCCGACGATGTCTGGGTTAG
SEQ ID NO: 141 <APT73.58.NTS>
ATGAAACATCACCACCACCACCATGGCACATCTGAAAACTTGTAT
TTCCAGGGCATGGACGAGGTGTATGCGGCAGTGGAACAGACTTCTCGACTTCTGG
ACGTTCCCTGTTCTCCTGACCGATTTGAGCCCGTTTGGAAGGCCTTTGGTGATCAG
CTGCCTGACTCTCATCTGGTCTTCTCTATGGCTGCTGGTGAGGCTCATCGAGGTGA
GCTGGACTTTGACTTCTCCCTTCGACCTGAAGGTGCTGATCCTTACACTACCGCTC
TGGAGCACGGCTTCATTGAGCCTACTGATCACCCTGTCGGTTCTGTTCTTGCTGAG
GTTGGTAAGCGATTCGCCATCGCCTCTTACGGTGTCGAATACGGTGTCGTCGGTG
GTTTCAAGAAGTCTTACGCCTTCTTCCCCCTGGACGACTTTCCTCCTCTTGCTCAG
TTCGCTGAGGTCCCCTCTGTTCCTCCTTGTTTAGCTGGTCACGTTGAGACTCTGAC
TCGACTGGGTTTTGACGACAAGGTCTCCGCCATCGGCGTCAACTACCGAAAGAAC
ACCCTGAACGTTTACCTGGCTGCTTCTGCTGTTGATACCGGCGACAAGCTGGCTCT
GCTTCGAGCTTTCGGTTACCCTGAACCTGATGCTAGAGTTCGACAGTTCATCGAG
CGGTCCTTCCGGCTTTACCCCACCTTCAACTGGGACTCTTCTGCTGCTGAACGAAT
CTGTTTCGCTGTCCACACTCAGCAGCCTGGTGAGCTTCCTGCTCCTCATGATGAGC
CTACTGAGGCTTTTGCTAGACAGGTTCCTCACGTCTACGAGGGTGGTCGAGAGTT
CGTCTCTGGTGTTGCTCTTGCTCCTTCTGGTGCTTCTTACTACAAGCTGGCTGCCCT
GTACCAGAAGGCTCGACGAAAGCTGGGTTAG
SEQ ID NO: 142 <APT73.59.NTS>
ATGAAACATCACCACCACCACCATGGCACATCTGAAAACTTGTAT
TTCCAGGGCATGGACGAGGTGTATGCGGCAGTGGAACAGACTTCTCGACTTCTGG
ACGTTCCCTGTTCTCCTGACCGATTTGAGCCCGTTTGGAAGGCCTTTGGTGATCAG
CTGCCTGACTCTCATCTGGTCTTCTCTATGGCTGCTGGTGAGGCTCATCGAGGTGA
GCTGGACTTTGACTTCTCCCTTCGACCTGAAGGTGCTGATCCTTACACTACCGCTC
TGGAGCACGGCTTCATTGAGCCTACTGATCACCCTGTCGGTTCTGTTCTTGCTGAG
GTTGGTAAGCGATTCGCCATCGCCTCTTACGGTGTCGAATACGGTGTCGTCGGTG
GTTTCAAGAAGTCTTACGCCTTCTTCCCCCTGGACGACTTTCCTCCTCTTGCTCAG
TTCGCTGAGGTCCCCTCTGTTCCTCCTTGTTTAGCTGGTCACGTTGAGACTCTGAC
TCGACTGGGTTTTGACGACAAGGTCTCCGCCATCGGCGTCAACTACCGAAAGAAC
ACCCTGAACGTTTACCTGGCTGCTTCTGCTGTTGATACCGGCGACAAGCTGGCTCT
GCTTCGAGCTTTCGGTTACCCTGAACCTGATGCTAGAGTTCGACAGTTCATCGAG
CGGTCCTTCCGGCTTTACCCCACCTTCAACTGGGACTCTTCTGCTGCTGAACGAAT
CTGTTTCGCTGTCCACACTCAGCAGCCTGGTGAGCTTCCTGCTCCTCATGATGAGC
CTACTGAGGCTTTTGCTAGACAGGTTCCTCACGTCTACGAGGGTGGTCGAGAGTT
CGTCTCTGGTGTTGCTCTTGCTCCTTCTGGTGCTTCTTACTACAAGCTGGCTGCCCT
GTACCAGAAGGCTCGACGAATGCTGGGTTAG
SEQ ID NO: 143 <APT73.64.NTS>
ATGAAACATCACCACCACCACCATGGCACATCTGAAAACTTGTAT
TTCCAGGGCATGGACGAGGTGTATGCGGCAGTGGAACAGACTTCTCGACTTCTGG
ACGTTCCCTGTTCTCCTGACCGATTTGAGCCCGTTTGGAAGGCCTTTGGTGATCAG
CTGCCTGACTCTCATCTGGTCTTCTCTATGGCTGCTGGTGAGGCTCATCGAGGTGA
GCTGGACTTTGACTTCTCCCTTCGACCTGAAGGTGCTGATCCTTACACTACCGCTC
TGGAGCACGGCTTCATTGAGCCTACTGATCACCCTGTCGGTTCTGTTCTTGCTGAG
GTTGGTAAGCGATTCGCCATCGCCTCTTACGGTGTCGAATACGGTGTCGTCGGTG
GTTTCAAGAAGTCTTACGCCTTCTTCCCCCTGGACGACTTTCCTCCTCTTGCTCAG
TTCGCTGAGGTCCCCTCTGTTCCTCCTTGTTTAGCTGGTCACGTTGAGACTCTGAC
TCGACTGGGTTTTGACGACAAGGTCTCCTGCATCGGCGTCAACTACCGGAAGAAC
ACCCTGAACGTTTACCTGGCTGCTTCTGCTGTTGATACCGGCGATAAGCTGGCTCT
GCTGCGAGCTTTCGGTTACCCTGAACCTGATGCTAGAGTTCGACAGTTCATCGAG
CGATCCTTCCGGCTGTACCCCACCTTCAACTGGGACTCTTCTGCTGCTGAACGAAT
CTGTTTCGCTGTTCACACTCAGCAGCCTGGCGAGCTTCCTGCTCCTCATGATGAGC
CTACTGAGGCTTTTGCTAGACAAGTTCCTCACGTCTACGAGGGTGGTCGGGAGTT
CGTCTCTGGTGTTGCTCTTGCTCCTTCTGGTGCTTCTTACTACAAGCTTGCTGCCCT
GTACCAGAAGGCCCGACGATGTCTGGGTTAG
SEQ ID NO: 144 <APT73.72.NTS>
ATGAAACATCACCACCACCACCATGGCACATCTGAAAACTTGTAT
TTCCAGGGCATGGACGAGGTGTATGCGGCAGTGGAACAGACTTCTCGACTTCTGG
ACGTTCCCTGTTCTCCTGACCGATTTGAGCCCGTTTGGAAGGCCTTTGGTGATCAG
CTGCCTGACTCTCATCTGGTCTTCTCTATGGCTGCTGGTGAGGCTCATCGAGGTGA
GCTGGACTTTGACTTCTCCCTTCGACCTGAAGGTGCTGATCCTTACACTACCGCTC
TGGAGCACGGCTTCATTGAGCCTACTGATCACCCTGTCGGTTCTGTTCTTGCTGAG
GTTGGTAAGCGATTCGCCATCGCCTCTTACGGTGTCGAATACGGTGTCGTCGGTG
GTTTCAAGAAGTCTTACGCCTTCTTCCCCCTGGACGACTTTCCTCCTCTTGCTCAG
TTCGCTGAGGTCCCCTCTGTTCCTCCTTGTTTAGCTGGTCACGTTGAGACTCTGAC
TCGACTGGGTTTTGACGACAAGGTCTCCGCCATCGGCGTCAACTACCGAAAGAAC
ACCCTGAACGTTTACCTGGCTGCTTCTGCTGTTGATACCGGCGACAAGCTGGCTCT
GCTTCGAGCTTTCGGTTACCCTGAACCTGATGCTAGAGTTCGACAGTTCATCGAG
CGGTCCTTCCGGCTTTACCCCACCTTCAACTGGGACTCTTCTGCTGCTGAACGAAT
CTGTTTCGCTGTCCACACTCAGCAGCCTGGTGAGCTTCCTGCTCCTCATGATGAGC
CTACTGAGGCTTTTGCTAGACAGGTTCCTCACGTCTACGAGGGTGGTCGAGAGTT
CGTCTCTGGTGTTGCTCTTGCTCCTTCTGGTGCTTCTTACTACAAGCTGGCTGCCG
AGTACCAGAAGCCGCGACGATGTCTGGGTTAG
SEQ ID NO: 145 <APT73.73.NTS>
ATGAAACATCACCACCACCACCATGGCACATCTGAAAACTTGTAT
TTCCAGGGCATGGACGAGGTGTATGCGGCAGTGGAACAGACTTCTCGACTTCTGG
ACGTTCCCTGTTCTCCTGACCGATTTGAGCCCGTTTGGAAGGCCTTTGGTGATCAG
CTGCCTGACTCTCATCTGGTCTTCTCTATGGCTGCTGGTGAGGCTCATCGAGGTGA
GCTGGACTTTGACTTCTCCCTTCGACCTGAAGGTGCTGATCCTTACACTACCGCTC
TGGAGCACGGCTTCATTGAGCCTACTGATCACCCTGTCGGTTCTGTTCTTGCTGAG
GTTGGTAAGCGATTCGCCATCGCCTCTTACGGTGTCGAATACGGTGTCGTCGGTG
GTTTCAAGAAGTCTTACGCCTTCTTCCCCCTGGACGACTTTCCTCCTCTTGCTCAG
TTCGCTGAGGTCCCCTCTGTTCCTCCTTGTTTAGCTGGTCACGTTGAGACTCTGAC
TCGACTGGGTTTTGACGACAAGGTCTCCGCCATCGGCGTCAACTACCGAAAGAAC
ACCCTGAACGTTTACCTGGCTGCTTCTGCTGTTGATACCGGCGACAAGCTGGCTCT
GCTTCGAGCTTTCGGTTACCCTGAACCTGATGCTAGAGTTCGACAGTTCATCGAG
CGGTCCTTCCGGCTTTACCCCACCTTCAACTGGGACTCTTCTGCTGCTGAACGAAT
CTGTTTCGCTGTCCACACTCAGCAGCCTGGTGAGCTTCCTGCTCCTCATGATGAGC
CTACTGAGGCTTTTGCTAGACAGGTTCCTCACGTCTACGAGGGTGGTCGAGAGTT
CGTCTCTGGTGTTGCTCTTGCTCCTTCTGGTGCTTCTTACTACAAGCTGGCTGCCG
AGTACCAGAAGGAGCGACGATGTCTGGGTTAG
SEQ ID NO: 146 <APT73.74.NTS>
ATGAAACATCACCACCACCACCATGGCACATCTGAAAACTTGTAT
TTCCAGGGCATGGACGAGGTGTATGCGGCAGTGGAACAGACTTCTCGACTTCTGG
ACGTTCCCTGTTCTCCTGACCGATTTGAGCCCGTTTGGAAGGCCTTTGGTGATCAG
CTGCCTGACTCTCATCTGGTCTTCTCTATGGCTGCTGGTGAGGCTCATCGAGGTGA
GCTGGACTTTGACTTCTCCCTTCGACCTGAAGGTGCTGATCCTTACACTACCGCTC
TGGAGCACGGCTTCATTGAGCCTACTGATCACCCTGTCGGTTCTGTTCTTGCTGAG
GTTGGTAAGCGATTCGCCATCGCCTCTTACGGTGTCGAATACGGTGTCGTCGGTG
GTTTCAAGAAGTCTTACGCCTTCTTCCCCCTGGACGACTTTCCTCCTCTTGCTCAG
TTCGCTGAGGTCCCCTCTGTTCCTCCTTGTTTAGCTGGTCACGTTGAGACTCTGAC
TCGACTGGGTTTTGACGACAAGGTCTCCATCATCGGCGTCAACTACCGGAAGAAC
ACCCTGAACGTCTACCTGGCCGCTTCTGCTGTTGATACTGGTGATAAGCTGGCTCT
GCTGCGAGCTTTCGGTTACCCTGAACCTGACGCTCGAGTTCGACAGTTCATTGAG
CGATCTTTCCGGCTGTACCCCACCTTCAACTGGGACTCTTCTGCTGCTGAGCGAAT
CTGTTTCGCTGTTCACACTCAGCAGCCTGGTGAGCTTCCTGCTCCTCATGATGAGC
CTACTGAGGCTTTTGCTCGACAAGTTCCTCACGTCTACGAGGGTGGTCGAGAGTT
CGTCTCTGGTGTTGCTCTTGCTCCTTCTGGTGCTTCTTACTACAAGCTGGCTGCCCT
GTACCAGAAGGCTCGACGATGTCTGCATGGTTAG
SEQ ID NO: 147 <APT73.75.NTS>
ATGAAACATCACCACCACCACCATGGCACATCTGAAAACTTGTAT
TTCCAGGGCATGGACGAGGTGTATGCGGCAGTGGAACAGACTTCTCGACTTCTGG
ACGTTCCCTGTTCTCCTGACCGATTTGAGCCCGTTTGGAAGGCCTTTGGTGATCAG
CTGCCTGACTCTCATCTGGTCTTCTCTATGGCTGCTGGTGAGGCTCATCGAGGTGA
GCTGGACTTTGACTTCTCCCTTCGACCTGAAGGTGCTGATCCTTACACTACCGCTC
TGGAGCACGGCTTCATTGAGCCTACTGATCACCCTGTCGGTTCTGTTCTTGCTGAG
GTTGGTAAGCGATTCGCCATCGCCTCTTACGGTGTCGAATACGGTGTCGTCGGTG
GTTTCAAGAAGTCTTACGCCTTCTTCCCCCTGGACGACTTTCCTCCTCTTGCTCAG
TTCGCTGAGGTCCCCTCTGTTCCTCCTTGTTTAGCTGGTCACGTTGAGACTCTGAC
TCGACTGGGTTTTGACGACAAGGTCTCCATCATCGGCGTCAACTACCGGAAGAAC
ACCCTGAACGTCTACCTGGCCGCTTCTGCTGTTGATACTGGTGATAAGCTGGCTCT
GCTGCGAGCTTTCGGTTACCCTGAACCTGACGCTCGAGTTCGACAGTTCATTGAG
CGATCTTTCCGGCTGTACCCCACCTTCAACTGGGACTCTTCTGCTGCTGAGCGAAT
CTGTTTCGCTGTTCACACTCAGCAGCCTGGTGAGCTTCCTGCTCCTCATGATGAGC
CTACTGAGGCTTTTGCTCGACAAGTTCCTCACGTCTACGAGGGTGGTCGAGAGTT
CGTCTCTGGTGTTGCTCTTGCTCCTTCTGGTGCTTCTTACTACAAGCTGGCTGCCCT
GTACCAGAAGGCTCGACGATGTCTGGGTGGTTAG
SEQ ID NO: 148 <APT73.77.NTS>
ATGAAACATCACCACCACCACCATGGCACATCTGAAAACTTGTAT
TTCCAGGGCATGGACGAGGTGTATGCGGCAGTGGAACAGACTTCTCGACTTCTGG
ACGTTCCCTGTTCTCCTGACCGATTTGAGCCCGTTTGGAAGGCCTTTGGTGATCAG
CTGCCTGACTCTCATCTGGTCTTCTCTATGGCTGCTGGTGAGGCTCATCGAGGTGA
GCTGGACTTTGACTTCTCCCTTCGACCTGAAGGTGCTGATCCTTACACTACCGCTC
TGGAGCACGGCTTCATTGAGCCTACTGATCACCCTGTCGGTTCTGTTCTTGCTGAG
GTTGGTAAGCGATTCGCCATCGCCTCTTACGGTGTCGAATACGGTGTCGTCGGTG
GTTTCAAGAAGTCTTACGCCTTCTTCCCCCTGGACGACTTTCCTCCTCTTGCTCAG
TTCGCTGAGGTCCCCTCTGTTCCTCCTTGTTTAGCTGGTCACGTTGAGACTCTGAC
TCGACTGGGTTTTGACGACAAGGTCTCCATCATCGGCGTCAACTACCGGAAGAAC
ACCCTGAACGTCTACCTGGCCGCTTCTGCTGTTGATACTGGTGATAAGCTGGCTCT
GCTGCGAGCTTTCGGTTACCCTGAACCTGACGCTCGAGTTCGACAGTTCATTGAG
CGATCTTTCCGGCTGTACCCCACCTTCAACTGGGACTCTTCTGCTGCTGAGCGAAT
CTGTTTCGCTGTTCACACTCAGCAGCCTGGTGAGCTTCCTGCTCCTCATGATGAGC
CTACTGAGGCTTTTGCTCGACAAGTTCCTCACGTCTACGAGGGTGGTCGAGAGTT
CGTCTCTGGTGTTGCTCTTGCTCCTTCTGGTGCTTCTTACTACAAGCTGGCTGCCCT
GTACCAGAAGGCTCGACGATGTCTGGATGGTTAG
SEQ ID NO: 149 <APT88.NTS>
ATGAAACATCACCACCACCACCATGGCACATCTGAAAACTTGTAT
TTCCAGGGCATGCAGAGACGATGGAGTGTGGTGGGTGTTCCTGCTGAACCTGGTG
CTGGTGCTGTTAGAGGTAGATGGCCTGTTAAGTGTCGATCTGACGGTGGTTCTTG
GCTTCAGCGAGCTCCCTCTGGTAGACAAGCTGGTTGTGCTCGAGTTGTTGGTGCTT
GTCGAGCTGATCGACTGAACTTCCTGGAGGAACTGATGGCTGGTCCTGCTGGTCT
TGATGAGGTCTATGCTGCTGTTGAGCGAACCTCTCGACTGCTGGATGTTCCCTGTT
CTCCTGACCGATTTGAGCCCGTTTGGAAGGCTTTTGGTGACCAGCTGCCCGATTCT
CACCTGGTCTTCTCTATGGCTGCTGGTGAGGCTCATAGAGGTGAACTGGACTTCG
ACTTCTCCCTTCGACCTGAGGGTGCTGATCCTTACACTACCGCTCTGGAACACGGT
TTCATTGAGCCTACTGACCACCCTGTCGGTTCTGTCCTTGCTGAGGTCAACAAGCG
ATGCGAGATCGCCTCTTATGGTGTTGAGTACGGTGTCGTCGGTGGCTTCAAGAAG
TCCTACGCCTTCTTCCCTCTGGACGACTTTCCTCCTTTGGCTGAGTTTGCCCGAAT
CCCCTCTGTTCCTCCTTGTCTTGCTGGTCACGTTGACACTCTTACCCGACTTGGTCT
GGACGACAAGGTCTCTGCCATTGGCGTCAACTACCGGAAGAACACCCTGAACGTT
TACCTTGCTGCCTCTGCTGTTGCTACTGACGACAAGCTGGCTCTGCTGCGAGCTTT
CGGTTACCCTGAACCTGATGCTAGAGTTCGACAGTTCATCGAGCGATCCTTCTCC
CTGTACCCCACCTTCAACTGGGATTCCTCTGCTGCTGAGCGAATCTGCTTCTCTGT
CAAGACACAGCAGCCTGGTGAGCTTCCTGCACCTCATGATGAGCCTACTGAGGCT
TTCGCTCGAGAAGTTCCTCACGTTTACGAGGGTGGTCGAGAGTTCGTCTCTGCTGT
TGCTCTTGCTCCTTCTGGTGCTGCTTACTACAAGCTTGCTGCCTACTACCAGAAGG
CCAGAGGTGCCTCTAATGCCGCTTTTGCCGCTAAGCGAGAAGATGCTGCTGCTGG
TTAG
SEQ ID NO: 150 <APT89.NTS>
ATGAAACATCACCACCACCACCATGGCACATCTGAAAACTTGTAT
TTCCAGGGCATGGACGAGGTGTATGCGGCAGTGGAACGAACTTCTCGACTTCTGG
ACGTTCCCTGTTCTCCTGACCGATTTGAGCCCGTTTGGAAGGCCTTTGGTGATCAG
CTGCCTGATTCTCACCTGGTCTTCTCTATGGCTGCTGGTGAGGCTCATCGAGGTGA
GCTGGACTTTGACTTCTCTCTTCGACCTGAGGGTGCTGATCCTTACACTACCGCTC
TGGAGCACGGCTTCATTGAGCCTACTGATCATCCTGTCGGTTCTGTCCTTGCTGAG
GTCAACAAGCGATGCGAGATCGCCTCTTACGGTGTCGAATACGGTGTCGTTGGTG
GCTTCAAGAAGTCCTACGCCTTCTTCCCTCTGGACGACTTTCCCCCTCTTGCTGAG
TTTGCCCGAATCCCCTCTGTTCCTCCTTGTTTAGCTGGTCACGTTGACACTCTGAC
CCGACTTGGTCTGGACGACAAGGTCTCTGCCATTGGCGTCAACTACCGAAAGAAC
ACCCTGAACGTCTACCTTGCTGCTTCTGCCGTTGCTACCGACGATAAGCTGGCTCT
GCTTCGAGCTTTCGGTTACCCTGAACCTGATGCTAGAGTGCGACAGTTCATCGAG
CGATCCTTCTCCCTGTACCCCACCTTCAACTGGGATTCTTCTGCTGCTGAGCGAAT
CTGCTTCTCCGTCAAGACACAGCAGCCTGGTGAGCTTCCTGCACCTCATGATGAA
CCTACTGAGGCTTTCGCTCGAGAGGTTCCTCACGTTTACGAGGGTGGTCGAGAGT
TCGTCTCTGCTGTTGCTTTAGCTCCTTCTGGTGCCGCTTACTACAAGCTTGCTGCCT
ACTACCAGAAGGCCAGAGGTGCCTCTAATGCTGCTTTTGCTGCCAAGCGAGAGGA
TGCTGCTGCTGGTTAG
SEQ ID NO: 151 <APT89.F116L.NTS>
ATGAAACATCACCACCACCACCATGGCACATCTGAAAACTTGTAT
TTCCAGGGCATGGACGAGGTGTATGCGGCAGTGGAACGAACTTCTCGACTTCTGG
ACGTTCCCTGTTCTCCTGACCGATTTGAGCCCGTTTGGAAGGCCTTTGGTGATCAG
CTGCCTGATTCTCACCTGGTCTTCTCTATGGCTGCTGGTGAGGCTCATCGAGGTGA
GCTGGACTTTGACTTCTCTCTTCGACCTGAGGGTGCTGATCCTTACACTACCGCTC
TGGAGCACGGCTTCATTGAGCCTACTGATCATCCTGTCGGTTCTGTCCTTGCTGAG
GTCAACAAGCGATGCGAGATCGCCTCTTACGGTGTCGAATACGGTGTCGTTGGTG
GCTTCAAGAAGTCCTACGCCCTGTTCCCTCTGGACGACTTTCCCCCTCTTGCTGAG
TTTGCCCGAATCCCCTCTGTTCCTCCTTGTTTAGCTGGTCACGTTGACACTCTGAC
CCGACTTGGTCTGGACGACAAGGTCTCTGCCATTGGCGTCAACTACCGAAAGAAC
ACCCTGAACGTCTACCTTGCTGCTTCTGCCGTTGCTACCGACGATAAGCTGGCTCT
GCTTCGAGCTTTCGGTTACCCTGAACCTGATGCTAGAGTGCGACAGTTCATCGAG
CGATCCTTCTCCCTGTACCCCACCTTCAACTGGGATTCTTCTGCTGCTGAGCGAAT
CTGCTTCTCCGTCAAGACACAGCAGCCTGGTGAGCTTCCTGCACCTCATGATGAA
CCTACTGAGGCTTTCGCTCGAGAGGTTCCTCACGTTTACGAGGGTGGTCGAGAGT
TCGTCTCTGCTGTTGCTTTAGCTCCTTCTGGTGCCGCTTACTACAAGCTTGCTGCCT
ACTACCAGAAGGCCAGAGGTGCCTCTAATGCTGCTTTTGCTGCCAAGCGAGAGGA
TGCTGCTGCTGGTTAG
SEQ ID NO: 152 <APT89.F116A.NTS>
ATGAAACATCACCACCACCACCATGGCACATCTGAAAACTTGTAT
TTCCAGGGCATGGACGAGGTGTATGCGGCAGTGGAACGAACTTCTCGACTTCTGG
ACGTTCCCTGTTCTCCTGACCGATTTGAGCCCGTTTGGAAGGCCTTTGGTGATCAG
CTGCCTGATTCTCACCTGGTCTTCTCTATGGCTGCTGGTGAGGCTCATCGAGGTGA
GCTGGACTTTGACTTCTCTCTTCGACCTGAGGGTGCTGATCCTTACACTACCGCTC
TGGAGCACGGCTTCATTGAGCCTACTGATCATCCTGTCGGTTCTGTCCTTGCTGAG
GTCAACAAGCGATGCGAGATCGCCTCTTACGGTGTCGAATACGGTGTCGTTGGTG
GCTTCAAGAAGTCCTACGCCGCGTTCCCTCTGGACGACTTTCCCCCTCTTGCTGAG
TTTGCCCGAATCCCCTCTGTTCCTCCTTGTTTAGCTGGTCACGTTGACACTCTGAC
CCGACTTGGTCTGGACGACAAGGTCTCTGCCATTGGCGTCAACTACCGAAAGAAC
ACCCTGAACGTCTACCTTGCTGCTTCTGCCGTTGCTACCGACGATAAGCTGGCTCT
GCTTCGAGCTTTCGGTTACCCTGAACCTGATGCTAGAGTGCGACAGTTCATCGAG
CGATCCTTCTCCCTGTACCCCACCTTCAACTGGGATTCTTCTGCTGCTGAGCGAAT
CTGCTTCTCCGTCAAGACACAGCAGCCTGGTGAGCTTCCTGCACCTCATGATGAA
CCTACTGAGGCTTTCGCTCGAGAGGTTCCTCACGTTTACGAGGGTGGTCGAGAGT
TCGTCTCTGCTGTTGCTTTAGCTCCTTCTGGTGCCGCTTACTACAAGCTTGCTGCCT
ACTACCAGAAGGCCAGAGGTGCCTCTAATGCTGCTTTTGCTGCCAAGCGAGAGGA
TGCTGCTGCTGGTTAG
SEQ ID NO: 153 <APT89.S155A.NTS>
ATGAAACATCACCACCACCACCATGGCACATCTGAAAACTTGTAT
TTCCAGGGCATGGACGAGGTGTATGCGGCAGTGGAACGAACTTCTCGACTTCTGG
ACGTTCCCTGTTCTCCTGACCGATTTGAGCCCGTTTGGAAGGCCTTTGGTGATCAG
CTGCCTGATTCTCACCTGGTCTTCTCTATGGCTGCTGGTGAGGCTCATCGAGGTGA
GCTGGACTTTGACTTCTCTCTTCGACCTGAGGGTGCTGATCCTTACACTACCGCTC
TGGAGCACGGCTTCATTGAGCCTACTGATCATCCTGTCGGTTCTGTCCTTGCTGAG
GTCAACAAGCGATGCGAGATCGCCTCTTACGGTGTCGAATACGGTGTCGTTGGTG
GCTTCAAGAAGTCCTACGCCTTCTTCCCTCTGGACGACTTTCCCCCTCTTGCTGAG
TTTGCCCGAATCCCCTCTGTTCCTCCTTGTTTAGCTGGTCACGTTGACACTCTGAC
CCGACTTGGTCTGGACGACAAGGTCGCGGCCATTGGCGTCAACTACCGAAAGAA
CACCCTGAACGTCTACCTTGCTGCTTCTGCCGTTGCTACCGACGATAAGCTGGCTC
TGCTTCGAGCTTTCGGTTACCCTGAACCTGATGCTAGAGTGCGACAGTTCATCGA
GCGATCCTTCTCCCTGTACCCCACCTTCAACTGGGATTCTTCTGCTGCTGAGCGAA
TCTGCTTCTCCGTCAAGACACAGCAGCCTGGTGAGCTTCCTGCACCTCATGATGA
ACCTACTGAGGCTTTCGCTCGAGAGGTTCCTCACGTTTACGAGGGTGGTCGAGAG
TTCGTCTCTGCTGTTGCTTTAGCTCCTTCTGGTGCCGCTTACTACAAGCTTGCTGCC
TACTACCAGAAGGCCAGAGGTGCCTCTAATGCTGCTTTTGCTGCCAAGCGAGAGG
ATGCTGCTGCTGGTTAG
SEQ ID NO: 154 <APT89.A260S.NTS>
ATGAAACATCACCACCACCACCATGGCACATCTGAAAACTTGTAT
TTCCAGGGCATGGACGAGGTGTATGCGGCAGTGGAACGAACTTCTCGACTTCTGG
ACGTTCCCTGTTCTCCTGACCGATTTGAGCCCGTTTGGAAGGCCTTTGGTGATCAG
CTGCCTGATTCTCACCTGGTCTTCTCTATGGCTGCTGGTGAGGCTCATCGAGGTGA
GCTGGACTTTGACTTCTCTCTTCGACCTGAGGGTGCTGATCCTTACACTACCGCTC
TGGAGCACGGCTTCATTGAGCCTACTGATCATCCTGTCGGTTCTGTCCTTGCTGAG
GTCAACAAGCGATGCGAGATCGCCTCTTACGGTGTCGAATACGGTGTCGTTGGTG
GCTTCAAGAAGTCCTACGCCTTCTTCCCTCTGGACGACTTTCCCCCTCTTGCTGAG
TTTGCCCGAATCCCCTCTGTTCCTCCTTGTTTAGCTGGTCACGTTGACACTCTGAC
CCGACTTGGTCTGGACGACAAGGTCTCTGCCATTGGCGTCAACTACCGAAAGAAC
ACCCTGAACGTCTACCTTGCTGCTTCTGCCGTTGCTACCGACGATAAGCTGGCTCT
GCTTCGAGCTTTCGGTTACCCTGAACCTGATGCTAGAGTGCGACAGTTCATCGAG
CGATCCTTCTCCCTGTACCCCACCTTCAACTGGGATTCTTCTGCTGCTGAGCGAAT
CTGCTTCTCCGTCAAGACACAGCAGCCTGGTGAGCTTCCTGCACCTCATGATGAA
CCTACTGAGGCTTTCGCTCGAGAGGTTCCTCACGTTTACGAGGGTGGTCGAGAGT
TCGTCTCTAGTGTTGCTTTAGCTCCTTCTGGTGCCGCTTACTACAAGCTTGCTGCC
TACTACCAGAAGGCCAGAGGTGCCTCTAATGCTGCTTTTGCTGCCAAGCGAGAGG
ATGCTGCTGCTGGTTAG
SEQ ID NO: 155 <APT89.1.NTS>
ATGAAACATCACCACCACCACCATGGCACATCTGAAAACTTGTAT
TTCCAGGGCATGGACGAGGTGTATGCGGCAGTGGAACGAACTTCTCGACTTCTGG
ACGTTCCCTGTTCTCCTGACCGATTTGAGCCCGTTTGGAAGGCCTTTGGTGATCAG
CTGCCTGATTCTCACCTGGTCTTCTCTATGGCTGCTGGTGAGGCTCATCGAGGTGA
GCTGGACTTTGACTTCTCTCTTCGACCTGAGGGTGCTGATCCTTACACTACCGCTC
TGGAGCACGGCTTCATTGAGCCTACTGATCATCCTGTCGGTTCTGTCCTTGCTGAG
GTCAACAAGCGATGCGAGATCGCCTCTTACGGTGTCGAATACGGTGTCGTTGGTG
GCTTCAAGAAGTCCTACGCCTTCTTCCCTCTGGACGACTTTCCCCCTCTTGCTGAG
TTTGCCCGAATCCCCTCTGTTCCTCCTTGTTTAGCTGGTCACGTTGACACTCTGAC
CCGACTTGGTCTGGACGACAAGGTCTCTGCCATTGGCGTCAACTACCGAAAGAAC
ACCCTGAACGTCTACCTTGCTGCTTCTGCCGTTGCTACCGACGATAAGCTGGCTCT
GCTTCGAGCTTTCGGTTACCCTGAACCTGATGCTAGAGTGCGACAGTTCATCGAG
CGATCCTTCCGTCTGTACCCCACCTTCAACTGGGATTCTTCTGCTGCTGAGCGAAT
CTGCTTCTCCGTCAAGACACAGCAGCCTGGTGAGCTTCCTGCACCTCATGATGAA
CCTACTGAGGCTTTCGCTCGAGAGGTTCCTCACGTTTACGAGGGTGGTCGAGAGT
TCGTCTCTGCTGTTGCTTTAGCTCCTTCTGGTGCCGCTTACTACAAGCTTGCTGCCT
ACTACCAGAAGGCCAGAGGTGCCTCTAATGCTGCTTTTGCTGCCAAGCGAGAGGA
TGCTGCTGCTGGTTAG
SEQ ID NO: 156 <APT89.2.NTS>
ATGAAACATCACCACCACCACCATGGCACATCTGAAAACTTGTAT
TTCCAGGGCATGGACGAGGTGTATGCGGCAGTGGAACGAACTTCTCGACTTCTGG
ACGTTCCCTGTTCTCCTGACCGATTTGAGCCCGTTTGGAAGGCCTTTGGTGATCAG
CTGCCTGATTCTCACCTGGTCTTCTCTATGGCTGCTGGTGAGGCTCATCGAGGTGA
GCTGGACTTTGACTTCTCTCTTCGACCTGAGGGTGCTGATCCTTACACTACCGCTC
TGGAGCACGGCTTCATTGAGCCTACTGATCATCCTGTCGGTTCTGTCCTTGCTGAG
GTCAACAAGCGATGCGAGATCGCCTCTTACGGTGTCGAATACGGTGTCGTTGGTG
GCTTCAAGAAGTCCTACGCCTTCTTCCCTCTGGACGACTTTCCCCCTCTTGCTGAG
TTTGCCCGAATCCCCTCTGTTCCTCCTTGTTTAGCTGGTCACGTTGACACTCTGAC
CCGACTTGGTCTGGACGACAAGGTCTCTGCCATTGGCGTCAACTACCGAAAGAAC
ACCCTGAACGTCTACCTTGCTGCTTCTGCCGTTGCTACCGACGATAAGCTGGCTCT
GCTTCGAGCTTTCGGTTACCCTGAACCTGATGCTAGAGTGCGACAGTTCATCGAG
CGATCCTTCCGTCTGTACCCCACCTTCAACTGGGATTCTTCTGCTGCTGAGCGAAT
CTGCTTCTCCGTCAAGACACAGCAGCCTGGTGAGCTTCCTGCACCTCATGATGAA
CCTACTGAGGCTTTCGCTCGAGAGGTTCCTCACGTTTACGAGGGTGGTCGAGAGT
TCGTCTCTGCTGTTGCTTTAGCTCCTTCTGGTGCCGCTTACTACAAGCTTGCTGCCT
ACTACCAGAAGGCCAGAAGGTGCCTCGGTTAG
SEQ ID NO: 157 <APT89.6.NTS>
ATGAAACATCACCACCACCACCATGGCACATCTGAAAACTTGTAT
TTCCAGGGCATGGACGAGGTGTATGCGGCAGTGGAACGAACTTCTCGACTTCTGG
ACGTTCCCTGTTCTCCTGACCGATTTGAGCCCGTTTGGAAGGCCTTTGGTGATCAG
CTGCCTGATTCTCACCTGGTCTTCTCTATGGCTGCTGGTGAGGCTCATCGAGGTGA
GCTGGACTTTGACTTCTCTCTTCGACCTGAGGGTGCTGATCCTTACACTACCGCTC
TGGAGCACGGCTTCATTGAGCCTACTGATCATCCTGTCGGTTCTGTCCTTGCTGAG
GTCAACAAGCGATGCGAGATCGCCTCTTACGGTGTCGAATACGGTGTCGTTGGTG
GCTTCAAGAAGTCCTACGCCTTCTTCCCTCTGGACGACTTTCCCCCTCTTGCTGAG
TTTGCCCGAATCCCCTCTGTTCCTCCTTGTTTAGCTGGTCACGTTGACACTCTGAC
CCGACTTGGTCTGGACGACAAGGTCTCTGCCATTGGCGTCAACTACCGAAAGAAC
ACCCTGAACGTCTACCTTGCTGCTTCTGCCGTTGCTACCGACGATAAGCTGGCTCT
GCTTCGAGCTTTCGGTTACCCTGAACCTGATGCTAGAGTGCGACAGTTCATCGAG
CGATCCTTCAAGCTGTACCCCACCTTCAACTGGGATTCTTCTGCTGCTGAGCGAAT
CTGCTTCTCCGTCAAGACACAGCAGCCTGGTGAGCTTCCTGCACCTCATGATGAA
CCTACTGAGGCTTTCGCTCGAGAGGTTCCTCACGTTTACGAGGGTGGTCGAGAGT
TCGTCTCTGCTGTTGCTTTAGCTCCTTCTGGTGCCGCTTACTACAAGCTTGCTGCCT
ACTACCAGAAGGCCAGAGGTGCCTCTAATGCTGCTTTTGCTGCCAAGCGAGAGGA
TGCTGCTGCTGGTTAG

1. A recombinant polypeptide comprising an amino acid sequence with at least 70% identity to SEQ ID NO: 1, 2, 3, or 4, wherein the amino acid sequence comprises at least one amino acid modification as compared to SEQ ID NO: 1, 3, or 4, wherein the recombinant polypeptide has a rate of formation of cannabigerolic acid (CBGA) from olivetolic acid (OA) and geranyl diphosphate (GPP) that is greater than the rate of formation of CBGA from OA and GPP by a polypeptide having the amino acid sequence of SEQ ID NO: 4 under the same conditions.

2. (canceled)

3. (canceled)

4. The recombinant polypeptide of claim 1, comprising an amino acid sequence identical to SEQ ID NO: 1 with one to twenty amino acid substitutions and, optionally, one to twenty amino acids deleted from the C-terminus, wherein the amino acid substitutions are located in SEQ ID NO: 1 at positions selected from 49, 51, 52, 53, 65, 67, 68, 69, 111, 113, 122, 124, 125, 126, 164, 165, 166, 167, 170, 214, 215, 217, 219, 229, 231, 233, 235, 268, 269, 270, 282, 283, 284, 285, 286, 292, 293, 294, 295, 296, 297, and 298.

5. The recombinant polypeptide of claim 1, comprising an amino acid sequence identical to SEQ ID NO: 2 with one to twenty amino acid substitutions and, optionally, one to twenty amino acids deleted from the C-terminus, wherein the amino acid substitutions are located in SEQ ID NO: 2 at positions selected from 38, 39, 40, 41, 42, 43, 44, 55, 57, 58, 59, 101, 103, 111, 112, 114, 115, 116, 117, 155, 156, 157, 160, 167, 169, 204, 205, 207, 209, 219, 221, 223, 224, 225, 258, 259, 260, 272, 273, 274, 275, 276, 282, 283, 284, 285, 286, 287, and 288.

6. The recombinant polypeptide of claim 1, comprising an amino acid sequence identical to SEQ ID NO: 3 with one to twenty amino acid substitutions and, optionally, one to twenty amino acids deleted from the C-terminus, wherein the amino acid substitutions are located in SEQ ID NO: 3 at positions selected from 108, 109, 110, 111, 112, 113, 114, 125, 127, 128, 129, 171, 173, 181, 182, 184, 185, 186, 187, 225, 226, 227, 230, 237, 239, 274, 275, 277, 279, 289, 291, 293, 294, 295, 328, 329, 330, 342, 343, 344, 345, 346, 352, 353, 354, 355, 356, 357 and 358.

7. The recombinant polypeptide of claim 1, comprising an amino acid sequence identical to SEQ ID NO: 4 with one to twenty amino acid substitutions and, optionally, one to twenty amino acids deleted from the C-terminus, wherein the amino acid substitutions are located in SEQ ID NO: 4 at positions selected from 38, 39, 40, 41, 42, 43, 44, 55, 57, 58, 59, 101, 103, 111, 112, 114, 115, 116, 117, 155, 156, 157, 160, 167, 169, 204, 205, 207, 209, 219, 221, 223, 224, 225, 258, 259, 260, 272, 273, 274, 275, 276, 282, 283 284, 285, 286, 287, and 288.

8. (canceled)

9. The recombinant polypeptide of claim 1, wherein the recombinant polypeptide comprises an amino acid sequence at least 90% identical to a sequence selected from SEQ ID NOs: 23-79 and 82-88.

10. (canceled)

11. (canceled)

12. (canceled)

13. The recombinant polypeptide of claim 1, wherein the recombinant polypeptide converts GPP to at least one product in addition to CBGA, wherein at least 50% of the one or more products converted by the recombinant polypeptide is CBGA.

14. (canceled)

15. (canceled)

16. (canceled)

17. The recombinant polypeptide of claim 1, wherein the recombinant polypeptide has a rate of formation of i) F-CBGA from OA and FPP, ii) cannabigerovarinic acid (CBGVA) from divarinic acid (DIV) and GPP; or iii) F-CBGVA from DIV and FPP that is greater than the rate of formation of F-CBGA from OA and FPP, cannabigerovarinic acid (CBGVA) from divarinic acid (DIV) and GPP; or F-CBGVA from DIV and FPP by a polypeptide consisting of SEQ ID NO: 4 under the same conditions.

18. (canceled)

19. (canceled)

20. (canceled)

21. (canceled)

22. (canceled)

23. (canceled)

24. (canceled)

25. (canceled)

26. A cell comprising an exogenous nucleotide sequence coding for a recombinant polypeptide comprising an amino acid sequence having at least 70% identity to SEQ ID NO: 1, 2, 3, or 4, wherein the recombinant polypeptide has a rate of formation of cannabigerolic acid (CBGA) from olivetolic acid (OA) and geranyl diphosphate (GPP) that is greater than the rate of formation of CBGA from OA and GPP by a polypeptide having the amino acid sequence of SEQ ID NO: 4 under the same conditions.

27. (canceled)

28. (canceled)

29. (canceled)

30. The cell of claim 26, comprising an exogenous nucleotide sequence coding for a recombinant polypeptide comprising an amino acid sequence identical to SEQ ID NO: 1 with one to twenty amino acid substitutions and, optionally, one to twenty amino acids deleted from the C-terminus, wherein the amino acid substitutions are located in SEQ ID NO: 1 at positions selected from 49, 51, 52, 53, 65, 67, 68, 69, 111, 113, 122, 124, 125, 126, 164, 165, 166, 167, 170, 214, 215, 217, 219, 229, 231, 233, 235, 268, 269, 270, 282, 283, 284, 285, 286, 292, 293, 294, 295, 296, 297, and 298.

31. The cell of claim 26, comprising an exogenous nucleotide sequence coding for a recombinant polypeptide comprising an amino acid sequence identical to SEQ ID NO: 2 with one to twenty amino acid substitutions and, optionally, one to twenty amino acids deleted from the C-terminus, wherein the amino acid substitutions are located in SEQ ID NO: 2 at positions selected from 38, 39, 40, 41, 42, 43, 44, 55, 57, 58, 59, 101, 103, 111, 112, 114, 115, 116, 117, 155, 156, 157, 160, 167, 169, 204, 205, 207, 209, 219, 221, 223, 224, 225, 258, 259, 260, 272, 273, 274, 275, 276, 282, 283, 284, 285, 286, 287, and 288.

32. The cell of claim 26, comprising an exogenous nucleotide sequence coding for a recombinant polypeptide comprising an amino acid sequence identical to SEQ ID NO: 3 with one to twenty amino acid substitutions and, optionally, one to twenty amino acids deleted from the C-terminus, wherein the amino acid substitutions are located in SEQ ID NO: 3 at positions selected from 108, 109, 110, 111, 112, 113, 114, 125, 127, 128, 129, 171, 173, 181, 182, 184, 185, 186, 187, 225, 226, 227, 230, 237, 239, 274, 275, 277, 279, 289, 291, 293, 294, 295, 328, 329, 330, 342, 343, 344, 345, 346, 352, 353, 354, 355, 356, 357 and 358.

33. The cell of claim 26, comprising an exogenous nucleotide sequence coding for a recombinant polypeptide comprising an amino acid sequence identical to SEQ ID NO: 4 with one to twenty amino acid substitutions and, optionally, one to twenty amino acids deleted from the C-terminus, wherein the amino acid substitutions are located in SEQ ID NO: 4 at positions selected from 38, 39, 40, 41, 42, 43, 44, 55, 57, 58, 59, 101, 103, 111, 112, 114, 115, 116, 117, 155, 156, 157, 160, 167, 169, 204, 205, 207, 209, 219, 221, 223, 224, 225, 258, 259, 260, 272, 273, 274, 275, 276, 282, 283 284, 285, 286, 287, and 288.

34. (canceled)

35. The cell of claim 26, wherein the recombinant polypeptide comprises an amino acid sequence at least 90% identical to a sequence selected from SEQ ID NOs: 23-79 and 82-88.

36. (canceled)

37. (canceled)

38. (canceled)

39. The cell of claim 26, wherein the recombinant polypeptide converts GPP to at least one product in addition to CBGA, wherein at least 50% of the one or more products converted by the recombinant polypeptide is CBGA.

40. (canceled)

41. (canceled)

42. (canceled)

43. The cell of claim 26, wherein the recombinant polypeptide has a rate of formation of i) F-CBGA from OA and FPP, ii) cannabigerovarinic acid (CBGVA) from divarinic acid (DIV) and GPP; or iii) F-CBGVA from DIV and FPP that is greater than the rate of formation of F-CBGA from OA and FPP, CBGVA from divarinic acid (DIV) and GPP, or F-CBGVA from DIV and FPP by a polypeptide consisting of SEQ ID NO: 4 under the same conditions.

44. (canceled)

45. (canceled)

46. (canceled)

47. (canceled)

48. The cell of claim 26, wherein the cell comprises:
an olivetolic acid pathway comprising a polyketide cyclase optionally encoded by an exogenous nucleotide;
a geranyl pyrophosphate (GPP) pathway comprising a non-native or mutant component, wherein the GPP pathway comprises geranyl pyrophosphate, optionally encoded by an exogenous nucleotide;
a farnesyl pyrophosphate (FPP) pathway comprising a non-native or mutant component, wherein the FPP pathway comprises farnesyl pyrophosphate synthase, optionally encoded by an exogenous nucleotide; and/or
a divarinic acid (DIV) pathway comprising divarinic acid synthase optionally encoded by an exogenous nucleotide.

49. (canceled)

50. (canceled)

51. (canceled)

52. (canceled)

53. (canceled)

54. (canceled)

55. (canceled)

56. (canceled)

57. (canceled)

58. (canceled)

59. (canceled)

60. The cell of claim 26, wherein the cell is capable of producing a cannabinoid selected from tetrahydrocannabinol, cannabidiol, cannabigerol, or an acid, derivative, or analogue thereof.

61. The cell of claim 60, wherein production of the cannabinoid is under control of an inducible promoter.

62. (canceled)

63. The cell of claim 62, wherein the cell is a yeast, optionally wherein the yeast is an oleaginous yeast.

64. The cell of claim 62, wherein the cell is a bacteria, optionally wherein the bacteria is Escherichia coli.

65. (canceled)

66. A method of producing a cannabinoid or an acid, derivative, or analogue thereof, the method comprising providing a cell comprising an exogenous nucleotide sequence coding for a recombinant polypeptide comprising an amino acid sequence having at least 70% identity to SEQ ID NO: 1, 2, 3, or 4, wherein the recombinant polypeptide has a rate of formation of cannabigerolic acid (CBGA) from olivetolic acid (OA) and geranyl diphosphate (GPP) that is greater than the rate of formation of CBGA from OA and GPP by a polypeptide having the amino acid sequence of SEQ ID NO: 4 under the same conditions, and culturing the cell to produce the cannabinoid or acid, derivative, or analogue thereof.

67. (canceled)

68. The method of claim 66, wherein the amino acid sequence comprises at least one amino acid modification as compared to SEQ ID NO: 1, 3, or 4.

69. (canceled)

70. The method of claim 66, wherein the amino acid sequence is identical to SEQ ID NO: 1 with one to twenty amino acid substitutions and, optionally, one to twenty amino acids deleted from the C-terminus, wherein the amino acid substitutions are located in SEQ ID NO: 1 at positions selected from 49, 51, 52, 53, 65, 67, 68, 69, 111, 113, 122, 124, 125, 126, 164, 165, 166, 167, 170, 214, 215, 217, 219, 229, 231, 233, 235, 268, 269, 270, 282, 283, 284, 285, 286, 292, 293, 294, 295, 296, 297, and 298.

71. The method of claim 66, wherein the amino acid sequence is identical to SEQ ID NO: 2 with one to twenty amino acid substitutions and, optionally, one to twenty amino acids deleted from the C-terminus, wherein the amino acid substitutions are located in SEQ ID NO: 2 at positions selected from 38, 39, 40, 41, 42, 43, 44, 55, 57, 58, 59, 101, 103, 111, 112, 114, 115, 116, 117, 155, 156, 157, 160, 167, 169, 204, 205, 207, 209, 219, 221, 223, 224, 225, 258, 259, 260, 272, 273, 274, 275, 276, 282, 283 284, 285, 286, 287, and 288.

72. The method of claim 66, wherein the amino acid sequence is identical to SEQ ID NO: 3 with one to twenty amino acid substitutions and, optionally, one to twenty amino acids deleted from the C-terminus, wherein the amino acid substitutions are located in SEQ ID NO: 3 at positions selected from 108, 109, 110, 111, 112, 113, 114, 125, 127, 128, 129, 171, 173, 181, 182, 184, 185, 186, 187, 225, 226, 227, 230, 237, 239, 274, 275, 277, 279, 289, 291, 293, 294, 295, 328, 329, 330, 342, 343, 344, 345, 346, 352, 353, 354, 355, 356, 357, and 358.

73. The method of claim 66, wherein the amino acid sequence is identical to SEQ ID NO: 4 with one to twenty amino acid substitutions and, optionally, one to twenty amino acids deleted from the C-terminus, wherein the amino acid substitutions are located in SEQ ID NO: 4 at positions selected from 38, 39, 40, 41, 42, 43, 44, 55, 57, 58, 59, 101, 103, 111, 112, 114, 115, 116, 117, 155, 156, 157, 160, 167, 169, 204, 205, 207, 209, 219, 221, 223, 224, 225, 258, 259, 260, 272, 273, 274, 275, 276, 282, 283, 284, 285, 286, 287, and 288.

74. (canceled)

75. (canceled)

76. (canceled)

77. The method of claim 66, wherein the sequence is selected from SEQ ID NOs: 29-36, 43, 56, 67, 69, 70, and 74.

78. (canceled)

79. The method of claim 66, wherein the recombinant polypeptide converts GPP to at least one product in addition to CBGA, wherein at least 50% of the one or more products converted by the recombinant polypeptide is CBGA.

80. (canceled)

81. (canceled)

82. (canceled)

83. The method of claim 66, wherein the recombinant polypeptide has a rate of formation of i) F-CBGA from OA and FPP, ii) cannabigerovarinic acid (CBGVA) from divarinic acid (DIV) and GPP; or iii) F-CBGVA from DIV and FPP that is greater than the rate of formation of F-CBGA from OA and FPP, CBGVA from divarinic acid (DIV) and GPP, or F-CBGVA from DIV and FPP by a polypeptide consisting of SEQ ID NO: 4 under the same conditions.

84. (canceled)

85. (canceled)

86. (canceled)

87. The method of claim 66, wherein the cell comprises:
an olivetolic acid pathway comprising a polyketide cyclase optionally encoded by an exogenous nucleotide;
a geranyl pyrophosphate (GPP) pathway comprising a non-native or mutant component, wherein the GPP pathway comprises geranyl pyrophosphate, optionally encoded by an exogenous nucleotide;
a farnesyl pyrophosphate (FPP) pathway comprising a non-native or mutant component, wherein the FPP pathway comprises farnesyl pyrophosphate synthase, optionally encoded by an exogenous nucleotide; and/or
a divarinic acid (DIV) pathway comprising divarinic acid synthase optionally encoded by an exogenous nucleotide.

88. (canceled)

89. (canceled)

90. (canceled)

91. (canceled)

92. (canceled)

93. (canceled)

94. (canceled)

95. (canceled)

96. (canceled)

97. (canceled)

98. (canceled)

99. The method of claim 66, wherein the cannabinoid or analogue thereof is selected from cannabigerolic acid, tetrahydrocannabinol, cannabidiol, cannabigerol, or an acid, derivative or analogue thereof.

100. The method of claim 99, wherein production of the cannabinoid or acid, derivative or analogue thereof is under control of an inducible promoter.

101. (canceled)

102. The method of claim 66, wherein the cell is a yeast, optionally wherein the yeast is an oleaginous yeast.

103. The method of claim 66, wherein the cell is a bacteria, optionally wherein the bacteria is Escherichia coli.

104. The method of claim 66, further comprising a step of purifying or isolating the cannabinoid or derivative or analogue thereof from the culture.
